(19) United States
(12) Patent Application Publication
(10) Pub. No.: US 2026/0055102 A1
(43) Pub. Date: Feb. 26, 2026

(54) PYRIDO-PYRAZOLES AS INHIBITORS OF DDR'S FOR THE TREATMENT OF FIBROTIC DISORDERS AND CANCER

(71) Applicant: Redx Pharma Limited, Macclesfield (GB)

(72) Inventors: Nicolas E.S. Guisot, Macclesfield (GB); Inder Bhamra, Macclesfield (GB); Clifford D. Jones, Macclesfield (GB); James Ryan, Macclesfield (GB); Michal S. Andra, Macclesfield (GB); Ana Varela Rodriguez, Macclesfield (GB); Camille Gignoux, Macclesfield (GB)

(21) Appl. No.: 19/104,750

(22) PCT Filed: Aug. 22, 2023
(86) PCT No.: PCT/GB2023/052182

§ 371 Date: Feb. 19, 2025

(30) Foreign Application Priority Data
Aug. 22, 2022 (GB) 2212185.9
Feb. 7, 2023 (GB) 2301690.0

(51) Int. Cl. C07D 471/04; A61K 31/437; A61K 31/444; A61K 31/4545; A61K 31/496; A61K 31/497; A61K 31/501; A61K 31/506; A61K 31/5377; C07D 519/00

(52) U.S. Cl. CPC C07D 471/04; A61K 31/437; A61K 31/444; A61K 31/4545; A61K 31/496; A61K 31/497; A61K 31/501; A61K 31/506; A61K 31/5377; C07D 519/00

(57) ABSTRACT

This invention relates to novel compounds and pharmaceutical compositions comprising the novel compounds. More specifically, the invention relates to compounds useful as inhibitors of discoidin domain receptor 1 (DDR1) and discoidin domain receptor 2 (DDR2). The compounds are particularly useful in the treatment of cancer and fibrotic diseases.

This invention relates to novel compounds and pharmaceutical compositions comprising the novel compounds. More specifically, the invention relates to compounds useful as inhibitors of discoidin domain receptor 1 (DDR1) and discoidin domain receptor 2 (DDR2). The compounds are particularly useful in the treatment of cancer and fibrotic diseases

### BACKGROUND

Discoidin Domain Receptors (DDRs) DDR1 and DDR2 are type 1 transmembrane Receptor Tyrosine Kinases (RTKs) with collagen receptor functionality (Vogel et al, Mol. Cell, 1997). DDRs contain characteristic collagen binding discoidin domains in the N-terminal extracellular domain. These domains are proceeded by an extracellular juxtamembrane domain, a single transmembrane domain, a cytosolic juxtamembrane domain and a catalytic kinase domain prior to a short C-terminal tail. Five isoforms of DDR1 (DRR1a-e) have been identified which arise from alternative splicing of the cytoplasmic region. No alternative isoforms of DDR2 have been identified. DDR1 and DDR2 have broadly (but not completely) mutually exclusive expression profiles in epithelial cell and stroma respectively. DDRs are activated by binding to collagens with broad specificity but with distinct preference for certain collagen types. Upon activation DDRs are known to regulate cell adhesion, proliferation and remodelling of the extracellular matrix. It is recognised that DDRs are upregulated in response to cellular activity and many forms of tissue injury and as such DDRs are implicated in diseases including cancer, atherosclerosis as well as diseases characterised by fibrosis and inflammation. Inhibitors of DDR kinase activity may be of benefit as therapeutic agents in these disease areas.

DDR1 and DDR2 overexpression and/or activation has been linked to multiple forms of cancer as summarised in a recent review (Elkamhawy et al, Int. J. Mol. Sci., 2021). Studies have shown that elevated DDR expression levels and/or mutations can be found in a number of cancer cell lines as well as primary tumour tissues including lung, pancreas, prostate, breast, brain, ovary, liver and others. DDR1 was found to be a prognostic marker for non-small-cell lung carcinoma (NSCLC) patients. A recent study demonstrated that siRNA-mediated downregulation of DDR1 suppressed melanoma cell malignancy, migration, invasion, and survival. DDR1 protein was also found to be expressed in 63% of serous ovarian cancer tissue, but not in normal ovarian surface epithelium. Involvement of DDR1 in glioblastoma cell invasion and epithelial-mesenchymal transition (EMT) has also been demonstrated. DDR1 expression was found in 50.5% of gastric cancer tissues. DDR1 was found to control triple-negative breast cancer growth by modulating tumour-infiltrating CD4+ and CD8+ T cells. There is also strong evidence indicating that DDR2 could be a potential biomarker and a molecular target for a variety of cancers. For instance, DDR2 overexpression was reported to contribute to NSCLC, thyroid carcinoma, Hodgkin's lymphoma, nasopharyngeal carcinomas, prostate cancer, as well as to head and neck squamous cell carcinoma. According to studies DDR2 contributes to breast cancer metastasis by stabilizing the SNAIL1 protein. DDR2 has also been shown to be a favourable independent predictor of recurrence and outcome in primary breast cancers. In addition to the essential roles of the wild type of DDR in cancer pathology and prognosis, various mutations of DDR1 and/or DDR2 have also been reported in numerous types of cancer cells, for instance, G1486T(DDR1) and A496S(DDR1) in lung cancer, N502S(DDR1), A533S(DDR1), and A803V(DDR1) in acute myeloid leukemia (AML), and S768R(DDR2) in squamous cell carcinoma. DDRs also play a role in cancer growth by controlling how tumour cells interact with their surrounding collagen matrix. This role of DDRs becomes more prominent when considering their role as extracellular matrix receptors. The extracellular matrix (ECM) confers structural properties to tissues around the tumour, as well as regulating cell proliferation, survival, migration, and invasion. The physiological interactions between tumour cells and their immediate microenvironment, represented by the extracellular matrix, are disrupted in metastatic cancers. As a key component of the tumour extracellular matrix, type I collagen shows high density and distorted architecture in malignant cancer, linking it to tumour formation and metastasis. Therefore, the discovery of DDRs as collagen receptors represents a new target in the regulation of tumour progression.

DDRs also appear to play a central role in the modulation of inflammation and fibrosis. Modulation of fibrosis and inflammation has been demonstrated in several organs including lung and kidney. In lung DDR-1 deficient mice show reduced bleomycin induced pulmonary injury (Vogel et al, Am. J. Respir. Crit. Care Med., 2006) and both DDR1 and DDR2 have been demonstrated to have increased expression in patients with fibrotic lung disease (Bian et al, ERJ Open Res., 2016). In kidney, DDR1 expression is elevated in patients with lupus nephritis and Goodpasture's syndrome as well as mouse models of glomerulonephritis (Kerroch et al, FASEB journal, 2012) and in the tubules of mice that have undergone unilateral ureteral obstruction (UUO) (Guerrot et al, Am. J. Pathol., 2011). Several studies have demonstrated that DDR1-null mice are protected from angiotensin II-mediated proteinuria, glomerular fibrosis and inflammation as well as showing reduced collagen deposition, tubular macrophage infiltration and pro-inflammatory cytokine levels following the UUO procedure. Finally, COL3A3 KO mice (the mouse model for human Alport syndrome, crossed on to DDR1-null mice have mice have reduced renal fibrosis and inflammation as a consequence of reduced TGF-β mediated signalling and reduced levels of the pro-inflammatory cytokine IL-6 (Dorison, Cell Adhesion and Migration, 2018).

Small molecule inhibitors of DDR1 and DDR2 kinase activity have been disclosed in the prior art and inhibitory activity of DDR1 and/or DDR2 has been demonstrated to give rise to efficacious effects in mouse models of cancer and fibrotic disease (Richter et al, ACS Chem. Biol., 2019; Wang et al, J. Med. Chem., 2018; Zhu et al, J. Med. Chem., 2019). Such reports support the hypothesis that inhibitors of DDR kinase activity may be of benefit as therapeutic agents for the treatment of human cancer and fibrotic disease.

Furthermore, it is an aim of certain embodiments of this invention to provide new compounds useful in treating diseases such as cancer and fibrotic diseases. The compounds may be inhibitors of DDR1 and/or DDR2. It is an aim of certain embodiments of this invention to provide compounds which have comparable activity to existing DDR1 and/or DDR2 inhibitors. It is an aim of certain embodiments of this invention to provide compounds which have increased activity relative to existing DDR1 and/or DDR2 inhibitors.

Certain embodiments of the present invention satisfy some or all of the above aims.

### BRIEF SUMMARY OF THE DISCLOSURE

The present invention relates to a compound of formula (I) or a pharmaceutically acceptable salt thereof:

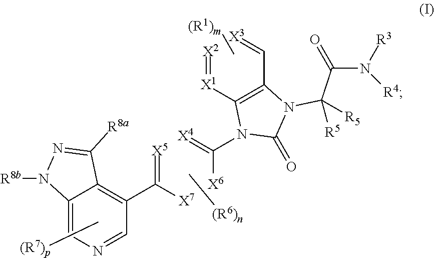

wherein
X¹, X² and X³ are each independently selected from carbon and nitrogen, wherein at least two of X¹, X² and X³ are carbon;
X⁴, X⁵, X⁶ and X⁷ are each independently selected from carbon and nitrogen, wherein at least two of X⁴, X⁵, X⁶ and X⁷ are carbon;
R¹ is independently selected at each occurrence from halo, nitro, cyano, NR⁹R¹⁰, OR¹¹, SR⁹, SO₂NR⁹R⁹, SO₂R⁹, CO₂R⁹, C(O)R⁹, CONR⁹R⁹, C₁-C₄-alkyl, C₁-C₄-alkyl substituted with NR⁹R¹⁰, C₁-C₄-alkyl substituted with OR¹¹, C₂-C₄-alkenyl, C₂-C₄-alkynyl, C₁-C₄-haloalkyl and cyclopropyl;
R³ is independently selected from H and C₁-C₄-alkyl;
R⁴ is independently selected from C₁-C₆-alkyl, C₁-C₆-haloalkyl, C₀-C₄-alkylene-R^{4a}; wherein R^{4a} is independently selected from: C₃-C₈-cycloalkyl, phenyl, 5-, 6-, 9- or 10-membered heteroaryl and 4- to 10-membered heterocycloalkyl; wherein said heterocycloalkyl or heteroaryl group may be monocyclic or bicyclic; wherein said cycloalkyl or heterocycloalkyl group is optionally substituted with a single R¹² group and/or from 1 to 4 R¹³ groups and wherein said phenyl or heteroaryl group is optionally substituted with a single R¹² group and/or from 1 to 3 R¹⁴ groups;
or R³ and R⁴, together with the nitrogen atom to which they are attached together form a 4- to 10-membered heterocycloalkyl group or a 5-, or 9-membered heteroaryl group; wherein said heterocycloalkyl or heteroaryl group may be monocyclic or bicyclic; wherein said heterocycloalkyl group is optionally substituted with a single R¹² group and/or from 1 to 4 R¹³ groups and wherein said heteroaryl group is optionally substituted with a single R¹² group and/or from 1 to 3 R¹⁴ groups;
R⁵ is independently at each occurrence selected from H, halo and C₁-C₄-alkyl, or the two R⁵ groups and the carbon atom to which they are attached may together form a C₃-C₆ cycloalkyl ring;
R⁶ is independently at each occurrence selected from halo, nitro, cyano, NR⁹R¹⁰, OR¹¹, SR⁹, SO₂NR⁹R⁹, SO₂R⁹, CO₂R⁹, C(O)R⁹, CONR⁹R⁹, C₁-C₄-alkyl, C₁-C₄-alkyl substituted with NR⁹R¹⁰, C₁-C₄-alkyl substituted with OR¹¹, C₂-C₄-alkenyl, C₂-C₄-alkynyl, C₁-C₄-haloalkyl and cyclopropyl;
R⁷ is independently at each occurrence selected from halo, nitro, cyano, NR⁹R¹⁰, OR¹¹, SR⁹, SO₂NR⁹R⁹, SO₂R⁹, CO₂R⁹, C(O)R⁹, CONR⁹R⁹, C₁-C₄-alkyl, C₁-C₄-alkyl substituted with NR⁹R¹⁰, C₁-C₄-alkyl substituted with OR¹¹, C₂-C₄-alkenyl, C₂-C₄-alkynyl, C₁-C₄-haloalkyl and cyclopropyl;
R^{8a} is independently selected from H, halo, nitro, cyano, NR⁹R¹⁰, OR¹¹, SR⁹, SO₂NR⁹R⁹, SO₂R⁹, CO₂R⁹, C(O)R⁹, CONR⁹R⁹, C₁-C₄-alkyl, C₁-C₄-alkyl substituted with NR⁹R¹⁰, C₁-C₄-alkyl substituted with OR¹¹, C₂-C₄-alkenyl, C₂-C₄-alkynyl, C₁-C₄-haloalkyl and C₀-C₄-alkylene-R^{8c};
R^{8b} is independently selected from H, C₁-C₄-alkyl, C₁-C₄-alkyl substituted with CONR⁹R⁹, C₂-C₄-alkyl substituted with NR⁹R¹⁰, C₂-C₄-alkyl substituted with OR¹¹, C₃-C₄-alkenyl, C₃-C₄-alkynyl, C₁-C₄-haloalkyl and C₀-C₄-alkylene-R^{8c};
R^{8c} is independently selected from C₃-C₆-cycloalkyl and 3- to 7-membered heterocycloalkyl; wherein said heterocycloalkyl group is attached to the C₀-C₄-alkylene via a carbon atom in the heterocycloalkyl ring; wherein said cycloalkyl or heterocycloalkyl group is optionally substituted with from 1 to 4 R¹³ groups;
R⁹ is independently at each occurrence selected from H and C₁-C₄-alkyl; or two R⁹ groups, together with the nitrogen atom to which they are attached together form a C₅-C₈-heterocycloalkyl group optionally substituted with from 0 to 4 R¹³ groups;
R¹⁰ is independently at each occurrence selected from H, C₁-C₄-alkyl, C(O)—C₁-C₄-alkyl and S(O)₂—C₁-C₄-alkyl; or R⁹ and R¹⁰, together with the nitrogen atom to which they are attached together form a C₅-C₈-heterocycloalkyl group optionally substituted with from 0 to 4 R¹³ groups;
R¹¹ is independently at each occurrence selected from H, C₁-C₄-alkyl, C(O)—C₁-C₄-alkyl and C₁-C₄-haloalkyl;
R¹² is independently selected from C₃-C₆-cycloalkyl, phenyl, 5- or 6-membered heteroaryl and 3- to 6-membered-heterocycloalkyl; wherein said cycloalkyl or heterocycloalkyl group is optionally substituted with from 1 to 4 R¹³ groups and wherein said phenyl or heteroaryl group is optionally substituted with from 1 to 3 R¹⁴ groups;
R¹³ is independently at each occurrence selected from ═O, halo, nitro, cyano, NR⁸R⁹, OR¹⁴, SR⁸, SO₂NR⁸R⁸, CO₂R⁸, C(O)R⁸, CONR⁸R⁸, C₁-C₄-alkyl, C₁-C₄-alkyl substituted with OR¹¹, C₁-C₄-alkyl substituted with NR⁹R¹⁰, C₂-C₄-alkenyl, C₂-C₄-alkynyl, C₁-C₄-haloalkyl, C₆-Clo-aryl, and C₃-C₆-cycloalkyl;
R¹⁴ is independently at each occurrence selected from halo, nitro, cyano, NR⁸R⁹, OR¹⁰, SR⁸, SO₂R⁸, SO₂NR⁸R⁸, CO₂R⁸, C(O)R⁸, CONR⁸R⁸, C₁-C₄-alkyl, C₂-C₄-alkenyl, C₂-C₄-alkynyl, C₁-C₄-haloalkyl, C₁-C₄-alkyl substituted with OR¹¹, C₁-C₄-alkyl substituted with NR⁸R⁹ and cyclopropyl; m is an integer selected from 0, 1, 2, 3 and 4;
n is an integer selected from 0, 1, 2, 3 and 4;
p is an integer selected from 0, 1 and 2;
wherein any of the aforementioned alkyl, alkylene or cyclopropyl groups is optionally substituted, where chemically possible, by 1 to 5 substituents which are each independently at each occurrence selected from the group consisting of: halo, oxo, fluoro, nitro, cyano, NR^{a}R^{b}, OR^{a}, SR^{a}, CO₂R^{a}, C(O)R^{a}, CONR^{a}R^{a}, C₁-C₄-alkyl, C₁-C₄-haloalkyl and cyclopropyl; wherein R^{a} is independently at each occurrence selected from H, C₁-C₄-alkyl and C₁-C₄-haloalkyl; and R^{b} is independently at each occurrence selected from H, C₁-C₄-alkyl, C(O)—C₁-C₄-alkyl and S(O)₂-C₁-C₄-alkyl.

In an embodiment, the compound of formula (I) is a compound of formula (11):

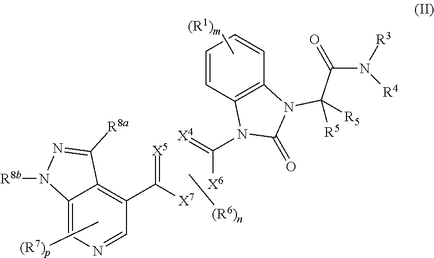

wherein X⁴, X⁵, X⁶, X⁷, R¹, R³, R⁴, R⁵, R⁶, R⁷, R^{8a}, R^{8b}, m, n and p are as described above for compounds of formula (I).

In an embodiment, the compound of formula (I) is a compound of formula (III):

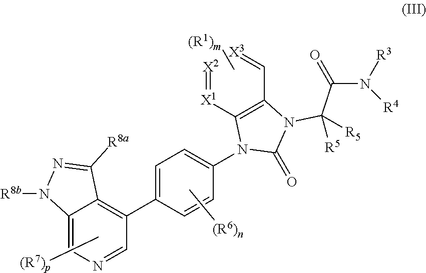

wherein X¹, X², X³, R¹, R³, R⁴, R⁵, R⁶, R⁷, R^{8a}, R^{8b}, m, n and p are as described above for compounds of formula (I).

In an embodiment, the compound of formula (I) is a compound of formula (IV):

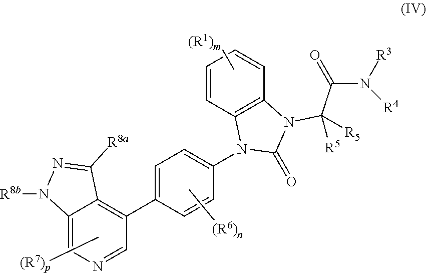

wherein R¹, R³, R⁴, R⁵, R⁶, R⁷, R^{8a}, R^{8b}, m, n and p are as described above for compounds of formula (I).

In an embodiment, the compound of formula (I) is a compound of formula (V):

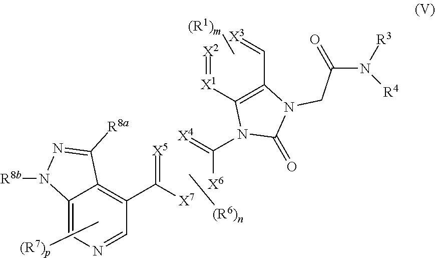

wherein X¹, X², X³, X⁴, X⁵, X⁶, X⁷, R¹, R³, R⁴, R⁶, R⁷, R^{8a}, R^{8b}, m, n and p are as described above for compounds of formula (I).

In an embodiment, the compound of formula (I) is a compound of formula (VI):

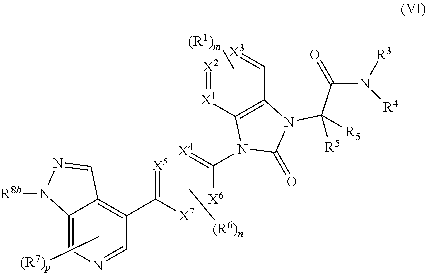

wherein X¹, X², X³, X⁴, X⁵, X⁶, X⁷, R¹, R³, R⁴, R⁵, R⁶, R⁷, R^{8b}, m, n and p are as described above for compounds of formula (I).

In an embodiment, the compound of formula (I) is a compound of formula (VII):

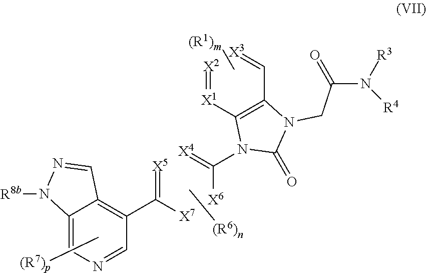

wherein X¹, X², X³, X⁴, X⁵, X⁶, X⁷, R¹, R³, R⁴, R⁶, R⁷, R^{8b}, m, n and p are as described above for compounds of formula (I).

In an embodiment, the compound of formula (I) is a compound of formula (VIII):

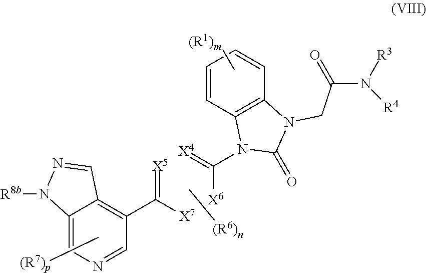

wherein X⁴, X⁵, X⁶, X⁷, R¹, R³, R⁴, R⁶, R⁷, R^{8b}, m, n and p are as described above for compounds of formula (I).

In an embodiment, the compound of formula (I) is a compound of formula (IX):

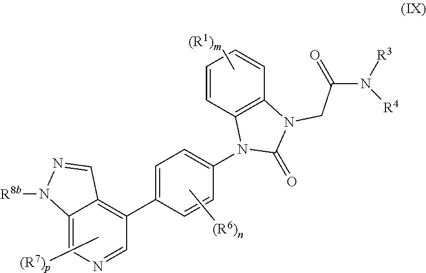

wherein R¹, R³, R⁴, R⁶, R⁷, R^{8b}, m, n and p are as described above for compounds of formula (I).

In an embodiment, the compound of formula (I) is a compound of formula (X):

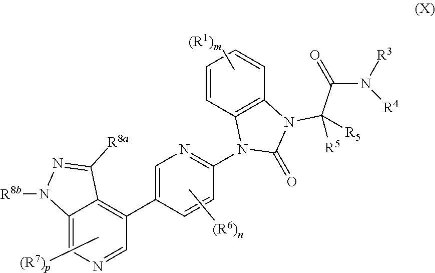

wherein R¹, R³, R⁴, R⁵, R⁶, R⁷, R^{8a}, R^{8b}, m and p are as described above for compounds of formula (I), and n is an integer selected from 0, 1, 2 and 3.

In an embodiment, the compound of formula (I) is a compound of formula (XI):

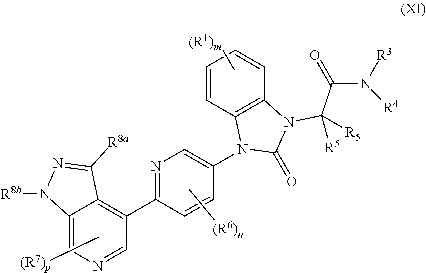

wherein R¹, R³, R⁴, R⁵, R⁶, R⁷, R^{8a}, R^{8b}, m and p are as described above for compounds of formula (I), and n is an integer selected from 0, 1, 2 and 3.

In an embodiment, the compound of formula (I) is a compound of formula (XII):

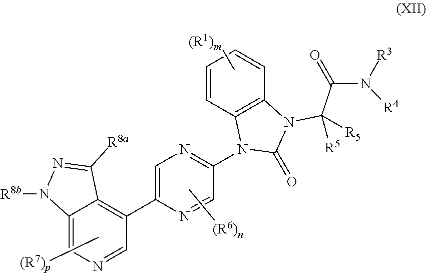

wherein R¹, R³, R⁴, R⁵, R⁶, R⁷, R^{8a}, R^{8b}, m and p are as described above for compounds of formula (I), and n is an integer selected from 0, 1 and 2.

The following embodiments apply to compounds of any of formulae (I)-(XII). These embodiments are independent and interchangeable. Any one embodiment may be combined with any other embodiment, where chemically allowed. In other words, any of the features described in the following embodiments may (where chemically allowable) be combined with the features described in one or more other embodiments. In particular, where a compound is exemplified or illustrated in this specification, any two or more of the embodiments listed below, expressed at any level of generality, which encompass that compound may be combined to provide a further embodiment which forms part of the present disclosure.

It may be that X¹ is carbon. It may be that X² is carbon. It may be that X³ is carbon It may be that X¹ and X² are both carbon. It may be that X² and X³ are both carbon. It may be that X¹ and X³ are both carbon. It may be that X¹, X² and X³ are each carbon.

It may be that a single one of X¹, X² and X³ is nitrogen. It may be that X¹ is nitrogen. It may be that X² is nitrogen. It may be that X³ is nitrogen.

It may be that X⁴ is carbon. It may be that X⁵ is carbon. It may be that X⁶ is carbon. It may be that X⁷ is carbon. It may be that X⁴ and X⁵ are both carbon. It may be that X⁴ and X⁶ are both carbon. It may be that X⁴ and X⁷ are both carbon. It may be that X⁵ and X⁶ are both carbon. It may be that X⁵ and X⁷ are both carbon. It may be that X⁶ and X⁷ are both carbon. It may be that X⁴, X⁵ and X⁶ are each carbon. It may be that X⁴, X⁵ and X⁷ are each carbon. It may be that X⁴, X⁶ and X⁷ are each carbon. It may be that X⁵, X⁶ and X⁷ are each carbon. It may be that X⁴, X⁵, X⁶ and X⁷ are each carbon.

It may be that at least one of X⁴, X⁵, X⁶, and X⁷ is nitrogen. It may be that a single one of X⁴, X⁵, X⁶, and X⁷ is nitrogen. It may be that X⁵ is nitrogen. It may be that X⁴ is nitrogen. It may be that two of X⁴, X⁵, X⁶, and X⁷ are nitrogen. It may be that X⁴ and X⁵ are each nitrogen. It may be that X⁴ and X⁷ are each nitrogen. It may be that X⁵ and X⁷ are each nitrogen. It may be that X⁴ and X⁶ are each nitrogen.

m may be selected from 0 and 1. m may be 0. m may be 1. m may be 2. m may be 3. m may be 4.

R¹ may be independently at each occurrence selected from halo, nitro, cyano, OR¹¹, C₁-C₄-alkyl, C₁-C₄-alkyl substituted with NR⁹R¹⁰, C₁-C₄-alkyl substituted with OR¹¹, C₁-C₄-haloalkyl and cyclopropyl. R¹ may be independently at each occurrence selected from halo, OR¹¹, NR⁹R¹⁰, cyano, C₁-C₄-alkyl and C₁-C₄-haloalkyl. R¹ may be independently at each occurrence selected from halo, OR¹¹, C₁-C₄-alkyl, C₁-C₄-haloalkyl and cyclopropyl. R¹ may be independently at each occurrence selected from halo and C₁-C₄-alkyl. R¹ may be independently at each occurrence halo, e.g. fluoro. R¹ may be independently at each occurrence C₁-C₄-alkyl, e.g. methyl.

It may be that X¹ is not CH. It may be that X¹ is selected from nitrogen and a carbon substituted with R¹. It may be that m is at least 1, X¹ is carbon and an R¹ group is attached to the X¹ carbon. It may be that m is 1, X¹ is carbon and the R¹ group is attached to the X¹ carbon. Compounds having an R¹ group attached to the X¹ carbon can show increased activity relative to compounds without an R¹ group attached to the X¹ carbon. In these embodiments, the R¹ group attached to X¹ may be C₁-C₄-alkyl, e.g. methyl.

R⁵ may be independently at each occurrence selected from H, fluoro and C₁-C₄-alkyl. R⁵ may be independently at each occurrence selected from H, fluoro and C₁-C₄-alkyl, or the two R⁵ groups and the carbon atom to which they are attached may together form a C₃-C₆ cycloalkyl ring. R⁵ may be independently at each occurrence selected from H and C₁-C₄-alkyl, or the two R⁵ groups and the carbon atom to which they are attached may together form a C₃-C₆ cycloalkyl ring. R⁵ may be independently at each occurrence selected from H and C₁-C₄-alkyl, e.g. methyl.

It may be that R⁵ is at each occurrence H. It may be that R⁵ is at one occurrence H and at the other occurrence C₁-C₄-alkyl, e.g. methyl. It may be that R⁵ is at each occurrence C₁-C₄-alkyl, e.g. methyl.

n may be selected from 0 and 1. n may be 0. n may be 1. n may be 2. n may be 3. n may be 4.

R⁶ may be independently selected from halo, nitro, cyano, OR¹¹, C₁-C₄-alkyl, C₁-C₄-alkyl substituted with NR⁹R¹⁰, C₁-C₄-alkyl substituted with OR¹¹, C₁-C₄-haloalkyl and cyclopropyl. R⁶ may be independently selected from halo, OR¹¹, C₁-C₄-alkyl, C₁-C₄-haloalkyl and cyclopropyl. R⁶ may be independently selected from halo and C₁-C₄-alkyl. If present, R⁶ may be halo, e.g. fluoro. R⁶ may be C₁-C₄-alkyl, e.g. methyl.

p may be selected from 0 and 1. p may be 0. p may be 1. p may be 2.

R⁷ may be independently selected from halo, nitro, cyano, OR¹¹, C₁-C₄-alkyl, C₁-C₄-alkyl substituted with NR⁹R¹⁰, C₁-C₄-alkyl substituted with OR¹¹, C₁-C₄-haloalkyl and cyclopropyl. R⁷ may be independently selected from halo, OR¹¹, C₁-C₄-alkyl, C₁-C₄-haloalkyl and cyclopropyl. R⁷ may be independently selected from halo and C₁-C₄-alkyl. If present, R⁷ may be halo, e.g. fluoro. R⁷ may be C₁-C₄-alkyl, e.g. methyl.

R^{8a} may be H. R^{8a} may be selected from halo, C₁-C₄-alkyl, C₁-C₄-alkyl substituted with NR⁹R¹⁰, C₁-C₄-alkyl substituted with OR¹¹, C₁-C₄-haloalkyl and C₀-C₄-alkylene-R^{8c}. R^{8a} may be selected from H, halo, C₁-C₄-alkyl, C₁-C₄-haloalkyl and cyclopropyl. R^{8a} may be selected from H, C₁-C₄-alkyl, C₁-C₄-haloalkyl and cyclopropyl. R^{8a} may be selected from C₁-C₄-alkyl (e.g. methyl) and C₁-C₄-haloalkyl (e.g. CF₃). R^{8a} may be C₁-C₄-alkyl (e.g. methyl).

R^{8b} may be H. R^{8b} may be selected from C₁-C₄-alkyl, C₁-C₄-alkyl substituted with CONR⁹R⁹, C₂-C₄-alkyl substituted with NR⁹R¹⁰, C₂-C₄-alkyl substituted with OR¹¹, C₁-C₄-haloalkyl and C₀-C₄-alkylene-R^{8c}. R^{8b} may be selected from H, C₁-C₄-alkyl and cyclopropyl. R^{8b} may be C₁-C₄-alkyl (e.g. methyl).

R^{8b} may be independently selected from H, C₁-C₄-alkyl, C₂-C₄-alkyl substituted with NR⁹R¹⁰, C₂-C₄-alkyl substituted with OR¹¹, C₃-C₄-alkenyl, C₃-C₄-alkynyl, C₁-C₄-haloalkyl and C₀-C₄-alkylene-R^{8c}.

It may be that R^{8a} is H and R^{8b} is C₁-C₄-alkyl (e.g. methyl). It may be that R^{8a} and R^{8b} are each H. It may be that R^{8a} and R^{8b} are each C₁-C₄-alkyl (e.g. methyl).

It may be that R⁹ is independently at each occurrence selected from H and C₁-C₄-alkyl.

It may be that R¹⁰ is independently at each occurrence selected from H, C₁-C₄-alkyl, C(O)—C₁-C₄-alkyl and S(O)₂—C₁-C₄-alkyl. It may be that R¹⁰ is independently at each occurrence selected from H and C₁-C₄-alkyl.

It may be that R¹¹ is independently at each occurrence selected from H, C₁-C₄-alkyl and C₁-C₄-haloalkyl. It may be that R¹¹ is independently at each occurrence selected from H and C₁-C₄-alkyl.

It may be that R¹¹ is independently at each occurrence C₁-C₄-alkyl, e.g. methyl.

R¹² may be independently selected from 5- or 6-membered heteroaryl, wherein said heteroaryl group is optionally substituted with from 1 to 3 R¹⁴ groups. R¹² may be independently selected from 5-membered heteroaryl, e.g. imidazole, wherein said heteroaryl group is optionally substituted with from 1 to 3 R¹⁴ groups.

R¹³ may be independently at each occurrence selected from oxo, fluoro, OR¹¹, CO₂R⁹, CO₂NR⁹R⁹, C₁-C₄-alkyl, C₁-C₄-alkyl substituted with NR⁹R¹⁰, C₁-C₄-alkyl substituted with OR¹¹, C₁-C₄-haloalkyl and cyclopropyl. R¹³ may be independently at each occurrence selected from oxo, OR¹¹, C₁-C₄-alkyl and cyclopropyl. R¹³ may be independently at each occurrence selected from oxo and C₁-C₄-alkyl. R¹³ may be independently at each occurrence C₁-C₄-alkyl, e.g. methyl.

R¹⁴ may be independently at each occurrence selected from halo, nitro, cyano, OR¹¹, C₁-C₄-alkyl, C₁-C₄-alkyl substituted with NR⁸R⁹, C₁-C₄-alkyl substituted with OR¹¹, C₁-C₄-haloalkyl and cyclopropyl. R¹⁴ may be independently at each occurrence selected from halo, OR¹¹, C₁-C₄-alkyl, C₁-C₄-haloalkyl and cyclopropyl. R¹⁴ may be independently at each occurrence selected from halo and C₁-C₄-alkyl. R¹⁴ may be independently at each occurrence halo, e.g. fluoro. R¹⁴ may be independently at each occurrence C₁-C₄-alkyl, e.g. methyl.

It may be that:
R³ is H; and
R⁴ is independently selected from C₁-C₆-alkyl, C₁-C₆-haloalkyl, C₀-C₄-alkylene-R^{4a}; wherein R^{4a} is independently selected from: C₃-C₈-cycloalkyl, phenyl, 5-, 6-, 9- or 10-membered heteroaryl and 4- to 10-membered heterocycloalkyl; wherein said heterocycloalkyl or heteroaryl group may be monocyclic or bicyclic; wherein said cycloalkyl or heterocycloalkyl group is optionally substituted with a single R¹² group and/or from 1 to 4 R¹³ groups and wherein said phenyl or heteroaryl group is optionally substituted with a single R¹² group and/or from 1 to 3 R¹⁴ groups;
or R³ and R⁴, together with the nitrogen atom to which they are attached together form a 4- to 10-membered heterocycloalkyl group or a 5-, or 9-membered heteroaryl group; wherein said heterocycloalkyl group is optionally substituted with a single R¹² group and/or from 1 to 4 R¹³ groups and wherein said heteroaryl group is optionally substituted with a single R¹² group and/or from 1 to 3 R¹⁴ groups.

It may be that:
R³ is H; and
R⁴ is independently selected from C₁-C₆-alkyl, C₁-C₆-haloalkyl, C₀-C₄-alkylene-R^{4a}; wherein R^{4a} is independently selected from: C₃-C₈-cycloalkyl, phenyl, 5-, 6-, 9- or 10-membered heteroaryl and 4- to 10-membered heterocycloalkyl; wherein said heterocycloalkyl or heteroaryl group may be monocyclic or bicyclic; wherein said cycloalkyl or heterocycloalkyl group is optionally substituted with from 1 to 4 R¹³ groups and wherein said phenyl or heteroaryl group is optionally substituted with from 1 to 3 R¹⁴ groups;
or R³ and R⁴, together with the nitrogen atom to which they are attached together form a 4- to 10-membered heterocycloalkyl group or a 5-, or 9-membered heteroaryl group; wherein said heterocycloalkyl group is optionally substituted with from 1 to 4 R¹³ groups and wherein said heteroaryl group is optionally substituted with from 1 to 3 R¹⁴ groups.

It may be that R³ is H.

It may be that R⁴ is selected from C₁-C₆-alkyl, C₁-C₆-haloalkyl and C₀-C₄-alkylene-R^{4a}.

It may be that R⁴ is selected from C₁-C₆-alkyl and C₁-C₆-haloalkyl. It may be that R⁴ is selected from C₂-C₃-alkyl and C₂-C₃-haloalkyl. It may be that R⁴ is C₁-C₄-haloalkyl. It may be that R⁴ is C₂-C₃-haloalkyl. It may be that R⁴ is 2,2,2-trifluoroethyl.

Illustrative R⁴ groups include:

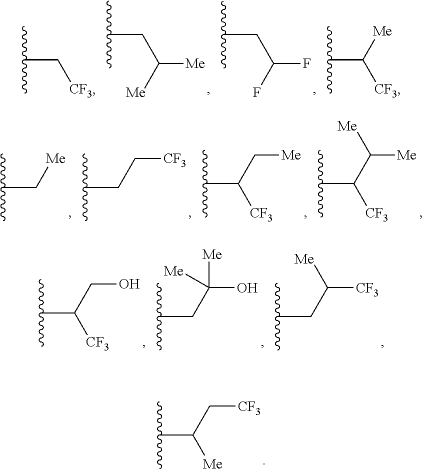

It may be that R⁴ is C₀-C₄-alkylene-R^{4a}. It may be that R⁴ is CH₂—R^{4a}. It may be that R⁴ is R^{4a}.

It may be that R^{4a} is independently selected from C₃-C₈-cycloalkyl, phenyl, 5-, 6-, 9- or 10-membered heteroaryl, 4- to 10-membered heterocycloalkyl, wherein said heterocycloalkyl or heteroaryl group may be monocyclic or bicyclic; wherein said cycloalkyl or heterocycloalkyl group is optionally substituted with from 1 to 4 R¹³ groups and wherein said phenyl or heteroaryl group is optionally substituted with from 1 to 3 R¹⁴ groups.

It may be that R^{4a} is selected from C₃-C₈-cycloalkyl and 4- to 10-membered heterocycloalkyl, wherein said cycloalkyl or heterocycloalkyl group is optionally substituted with a single R¹² group and/or from 1 to 4 R¹³ groups. It may be that R^{4a} is selected from C₃-C₈-cycloalkyl and 4- to 10-membered heterocycloalkyl, wherein said cycloalkyl or heterocycloalkyl group is optionally substituted with from 1 to 4 R¹³ groups.

It may be that R⁴ is selected from CH₂—C₃-C₈-cycloalkyl and CH₂-4- to 10-membered heterocycloalkyl, wherein said cycloalkyl or heterocycloalkyl group is optionally substituted with a single R¹² group and/or from 1 to 4 R¹³ groups. It may be that R⁴ is selected from CH₂—C₃-C₈-cycloalkyl and CH₂-4- to 10-membered heterocycloalkyl, wherein said cycloalkyl or heterocycloalkyl group is optionally substituted with from 1 to 4 R¹³ groups.

It may be that R⁴ is selected from C₃-C₈-cycloalkyl and 4- to 10-membered heterocycloalkyl, wherein said cycloalkyl or heterocycloalkyl group is optionally substituted with a single R¹² group and/or from 1 to 4 R¹³ groups. It may be that R⁴ is selected from C₃-C₈-cycloalkyl and 4- to 10-membered heterocycloalkyl, wherein said cycloalkyl or heterocycloalkyl group is optionally substituted with from 1 to 4 R¹³ groups.

Illustrative R⁴ groups include:

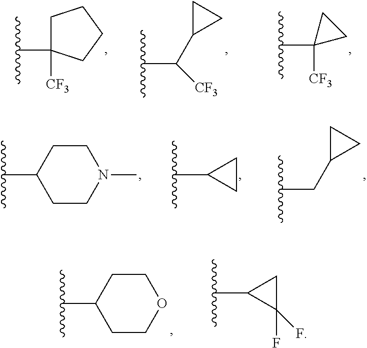

It may be that R^{4a} is independently selected from: phenyl and 5- or 6-membered heteroaryl; wherein said phenyl or heteroaryl group is optionally substituted with a single R¹² group and/or from 1 to 3 R¹⁴ groups. It may be that R^{4a} is independently selected from: phenyl and 5- or 6-membered heteroaryl; wherein said phenyl or heteroaryl group is optionally substituted with from 1 to 3 R¹⁴ groups. It may be that R^{4a} is independently phenyl; wherein said phenyl group is optionally substituted with a single R¹² group and/or from 1 to 3 R¹⁴ groups. It may be that R^{4a} is independently phenyl; wherein said phenyl group is optionally substituted with from 1 to 3 R¹⁴ groups. It may be that R^{4a} is independently 5- or 6-membered heteroaryl; wherein said heteroaryl group is optionally substituted with a single R¹² group and/or from 1 to 3 R¹⁴ groups. It may be that R^{4a} is independently 5- or 6-membered heteroaryl; wherein said heteroaryl group is optionally substituted with from 1 to 3 R¹⁴ groups.

It may be that R⁴ is independently selected from CH₂-phenyl or CH₂-5- or 6-membered heteroaryl wherein said phenyl or heteroaryl group is optionally substituted with a single R¹² group and/or from 1 to 3 R¹⁴ groups. It may be that R⁴ is independently selected from CH₂-phenyl or CH₂-5- or 6-membered heteroaryl wherein said phenyl or heteroaryl group is optionally substituted with from 1 to 3 R¹⁴ groups. It may be that R⁴ is independently CH₂-phenyl wherein said phenyl is optionally substituted with a single R¹² group and/or from 1 to 3 R¹⁴ groups. It may be that R⁴ is independently CH₂-phenyl wherein said phenyl group is optionally substituted with from 1 to 3 R¹⁴ groups. It may be that R⁴ is independently CH₂-5- or 6-membered heteroaryl wherein said heteroaryl group is optionally substituted with a single R¹² group and/or from 1 to 3 R¹⁴ groups. It may be that R⁴ is independently CH₂-5- or 6-membered heteroaryl wherein said heteroaryl group is optionally substituted with from 1 to 3 R¹⁴ groups.

It may be that R⁴ is independently selected from phenyl or 5- or 6-membered heteroaryl wherein said phenyl or heteroaryl group is optionally substituted with a single R¹² group and/or from 1 to 3 R¹⁴ groups. It may be that R⁴ is independently selected from phenyl or 5- or 6-membered heteroaryl wherein said phenyl or heteroaryl group is optionally substituted with from 1 to 3 R¹⁴ groups. It may be that R⁴ is independently phenyl wherein said phenyl group is optionally substituted with a single R¹² group and/or from 1 to 3 R¹⁴ groups. It may be that R⁴ is independently phenyl wherein said phenyl group is optionally substituted with from 1 to 3 R¹⁴ groups. It may be that R⁴ is independently 5- or 6-membered heteroaryl wherein said heteroaryl group is optionally substituted with a single R¹² group and/or from 1 to 3 R¹⁴ groups. It may be that R⁴ is independently 5- or 6-membered heteroaryl wherein said heteroaryl group is optionally substituted with from 1 to 3 R¹⁴ groups.

It may be that R⁴ is independently selected from phenyl or 6-membered heteroaryl wherein said phenyl or 6-membered heteroaryl group is substituted at the meta position with 1 R¹⁴ group. It may be that the R¹⁴ group is selected from C₁-C₄-alkyl substituted with OR¹¹, C₁-C₄-alkyl and C₁-C₄-haloalkyl.

It may be that the R¹⁴ group at the meta position is C₁-C₄-alkyl substituted with OR¹¹ e.g. —(CH₃)₂—OH. It may be that the R¹⁴ group at the meta position is C₁-C₄-haloalkyl e.g. CF₃.

It may be that R⁴ is a 6-membered heteroaryl group. It may be that R⁴ is phenyl. It may be that R⁴ is phenyl substituted at the meta position with 1 R¹⁴ group. It may be that R¹⁴ is R^{14a}. Illustrative R⁴ groups include:

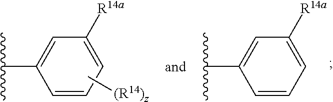

wherein R^{14a} is selected from halo, nitro, cyano, NR⁸R⁹, OR¹⁰, SR⁸, SO₂R⁸, SO₂NR⁸R⁸, CO₂R⁸, C(O)R⁸, CONR⁸R⁸, C₁-C₄-alkyl, C₂-C₄-alkenyl, C₂-C₄-alkynyl, C₁-C₄-haloalkyl, C₁-C₄-alkyl substituted with OR¹¹, C₁-C₄-alkyl substituted with NR⁸R⁹ and cyclopropyl; and wherein z is an integer selected from 1 and 2.

Illustrative R⁴ groups include:

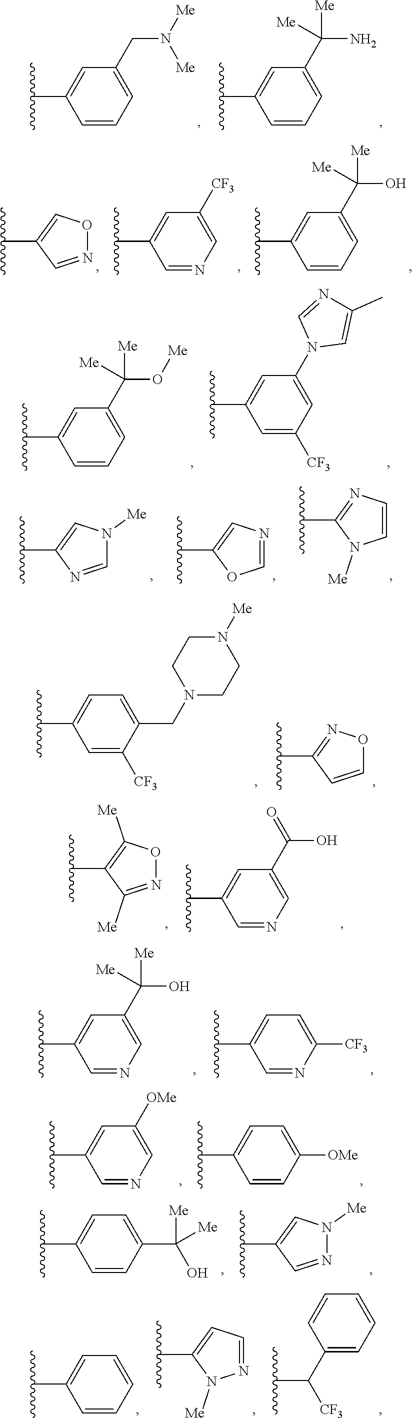

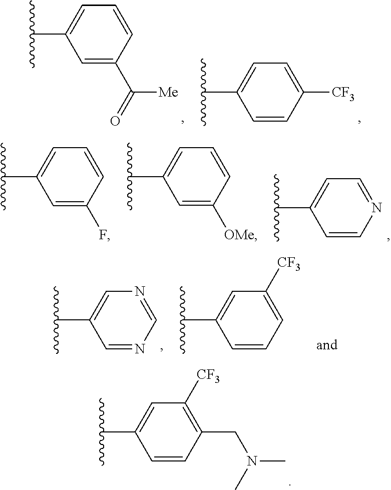

It may be that R³ and R⁴, together with the nitrogen atom to which they are attached together form a 4- to 10-membered heterocycloalkyl group or a 5-, or 9-membered heteroaryl group; wherein said heterocycloalkyl group is optionally substituted with a single R¹² group and/or from 1 to 4 R¹³ groups and wherein said heteroaryl group is optionally substituted with a single R¹² group and/or from 1 to 3 R¹⁴ groups. It may be that R³ and R⁴, together with the nitrogen atom to which they are attached together form a 4- to 10-membered heterocycloalkyl group or a 5-, or 9-membered heteroaryl group; wherein said heterocycloalkyl group is optionally substituted with from 1 to 4 R¹³ groups and wherein said heteroaryl group is optionally substituted with from 1 to 3 R¹⁴ groups.

It may be that R³ and R⁴, together with the nitrogen atom to which they are attached together form a 5-, or 9-membered heteroaryl group; wherein heteroaryl group is optionally substituted with a single R¹² group and/or from 1 to 3 R¹⁴ groups. It may be that R³ and R⁴, together with the nitrogen atom to which they are attached together form a 5-, or 9-membered heteroaryl group; wherein said heteroaryl group is optionally substituted with from 1 to 3 R¹⁴ groups. It may be that R³ and R⁴, together with the nitrogen atom to which they are attached together form a 5-membered heteroaryl group; wherein heteroaryl group is optionally substituted with a single R¹² group and/or from 1 to 3 R¹⁴ groups. It may be that R³ and R⁴, together with the nitrogen atom to which they are attached together form a 5-membered heteroaryl group; wherein said heteroaryl group is optionally substituted with from 1 to 3 R¹⁴ groups.

Illustrative NR³R⁴ groups include:

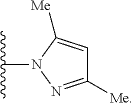 [SMILES: *n1nc(C)cc1C]

It may be that R³ and R⁴, together with the nitrogen atom to which they are attached together form a 4- to 10-membered heterocycloalkyl group; wherein said heterocycloalkyl group is optionally substituted with a single R¹² group and/or from 1 to 4 R¹³ groups. It may be that R³ and R⁴, together with the nitrogen atom to which they are attached together form a 4- to 10-membered heterocycloalkyl group; wherein said heterocycloalkyl group is optionally substituted with from 1 to 4 R¹³ groups. It may be that said heterocycloalkyl group is 7- to 10-membered bicyclic heterocycloalkyl group. It may be that said heterocyclic group is 7- to 10-membered bridged bicyclic heterocycloalkyl group. It may be that said heterocyclic group is a monocyclic 4- to 7-membered heterocycloalkyl group. It may be that said heterocyclic group is a monocyclic 5- to 6-membered heterocycloalkyl group. It may be that said heterocyclic group is pyrrolidine. It may be that said heterocyclic group is piperidine. It may be that said heterocyclic group is morpholine. It may be that said heterocyclic group is piperazine. For the absence of doubt the heterocycloalkyl groups mentioned in this paragraph are optionally substituted with a single R¹² group and/or from 1 to 4 R¹³ groups. It may be that the heterocycloalkyl groups mentioned in this paragraph are optionally substituted with from 1 to 4 R¹³ groups. It may be that said heterocyclic group is pyrrolidine and is substituted at the 2 position with 1 R¹³ group. It may be that said heterocyclic group is pyrrolidine and is substituted at the 3 position with 1 R¹³ group. It may be that said heterocyclic group is piperidine and is substituted at the 2 position with 1 R¹³ group. It may be that said heterocyclic group is piperidine and is substituted at the 3 position with 1 R¹³ group. It may be that said heterocyclic group is piperidine and is substituted at the 4 position with 1 R¹³ group. It may be that said heterocyclic group is morpholine and is substituted at the 2 position with 1 R¹³ group. It may be that said heterocyclic group is morpholine and is substituted at the 3 position with 1 R¹³ group. It may be that the R¹³ group is selected from C₁-C₄-alkyl, C₁-C₄-alkyl substituted with OR¹¹ e.g. —(CH₃)₂—OH, and C₁-C₄-haloalkyl, e.g. —CF₃.

Illustrative NR³R⁴ groups include:

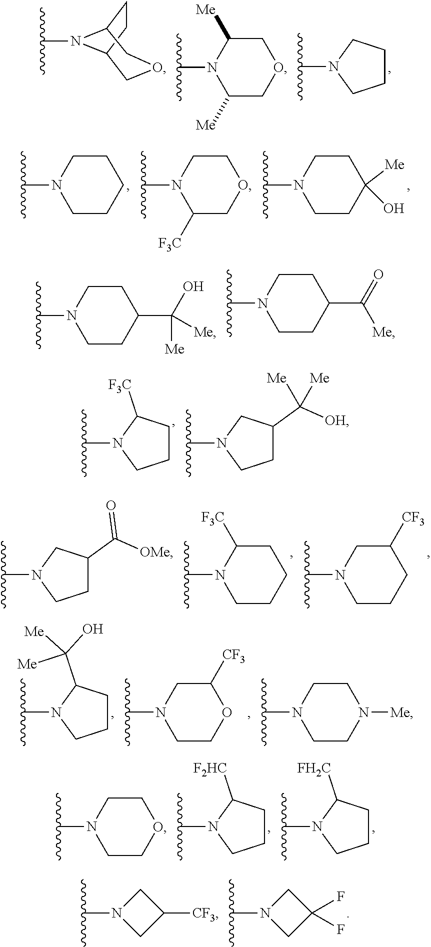

It may be that R³ and R⁴ are selected such that NR³R⁴ comprises a CHF₂ group or a CF₃ group.

It may be that NR³R⁴ has the formula

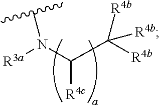

wherein
a is an integer selected from 1 and 2;
R^{4b} is at each occurrence selected from H and F; wherein at least one R^{4b} group is F;
R^{3a} is independently selected from H and C₁-C₄-alkyl;
R^{4c} is independently at each occurrence selected from H, C₁-C₄-alkyl and C₄-C₆-cycloalkyl; or
R^{3a} and a single R^{4c}, together with the carbon and nitrogen to which they are attached, form a 4- to 6-membered heterocycloalkyl group.

a may be 1. a may be 2.

It may be that NR³R⁴ has the formula

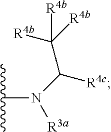

wherein
R^{4b} is at each occurence selected from H and F; wherein at least one R^{4b} group is F;
R^{3a} is independently selected from H and C₁-C₄-alkyl;
R^{4c} is independently selected from H, C₁-C₄-alkyl and C₄-C₆-cycloalkyl; or
R^{3a} and R^{4c}, together with the carbon and nitrogen to which they are attached, form a 4- to 6-membered heterocycloalkyl group.

It may be that at least two R^{4b} groups are F. It may be that two R^{4b} groups are F and one R^{4b} group is H. It may be that each R^{4b} group is F.

It may be that R^{3a} is H.

It may be that R^{3a} is independently selected from H and C₁-C₄-alkyl; and R^{4c} is independently selected from H, C₁-C₄-alkyl and C₄-C₆-cycloalkyl. It may be that R^{3a} is H; and R^{4c} is independently selected from H, C₁-C₄-alkyl and C₄-C₆-cycloalkyl. Said alkyl or cycloalkyl group may be unsubstituted.

It may be that R^{4c} is at each occurrence selected from C₁-C₄-alkyl and H. It may be that R^{4c} is at each occurence H.

It may be that R^{3a} and R^{4c}, together with the carbon and nitrogen to which they are attached, form a 4- to 6-membered heterocycloalkyl group. It may be that R^{3a} and R^{4c}, together with the carbon and nitrogen to which they are attached, form a 5-membered heterocycloalkyl group. It may be that R^{3a} and R^{4c}, together with the carbon and nitrogen to which they are attached, form a 6-membered heterocycloalkyl group. Said heterocycloalkyl group might be unsubstituted.

The compound of formula (I) may be selected from:

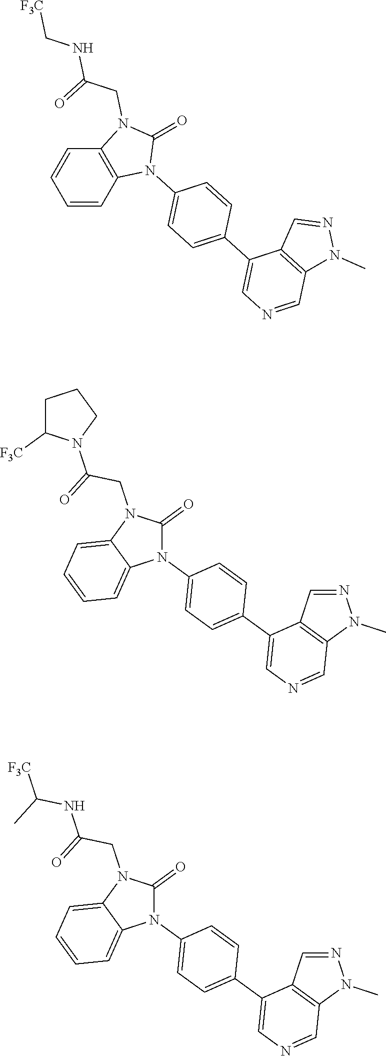

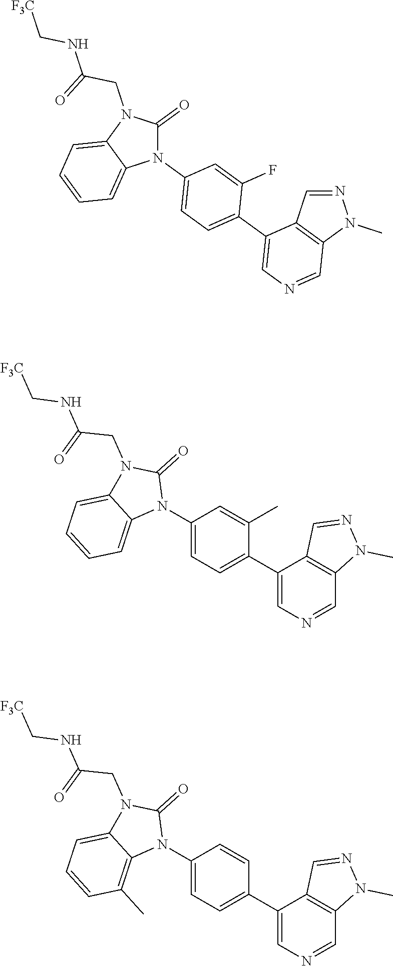 [SMILES: Cn1ncc2c(-c3ccc(-n4c(=O)n(CC(=O)NCC(F)(F)F)c5ccccc54)cc3F)cncc21 ; Cc1cc(-n2c(=O)n(CC(=O)NCC(F)(F)F)c3ccccc32)ccc1-c1cncc2c1cnn2C ; Cc1cccc2c1n(-c1ccc(-c3cncc4c3cnn4C)cc1)c(=O)n2CC(=O)NCC(F)(F)F]

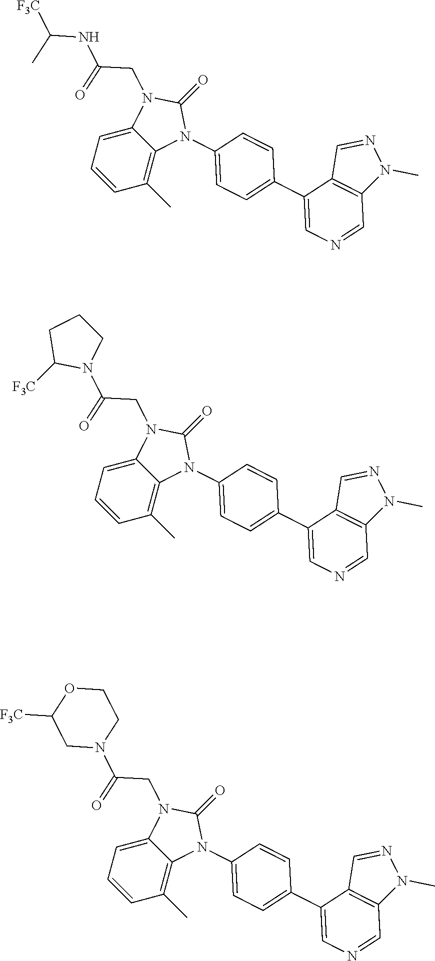 [SMILES: Cc1cccc2c1n(-c1ccc(-c3cncc4c3cnn4C)cc1)c(=O)n2CC(=O)NC(C)C(F)(F)F ; Cc1cccc2c1n(-c1ccc(-c3cncc4c3cnn4C)cc1)c(=O)n2CC(=O)N1CCCC1C(F)(F)F ; Cc1cccc2c1n(-c1ccc(-c3cncc4c3cnn4C)cc1)c(=O)n2CC(=O)N1CCOC(C(F)(F)F)C1]

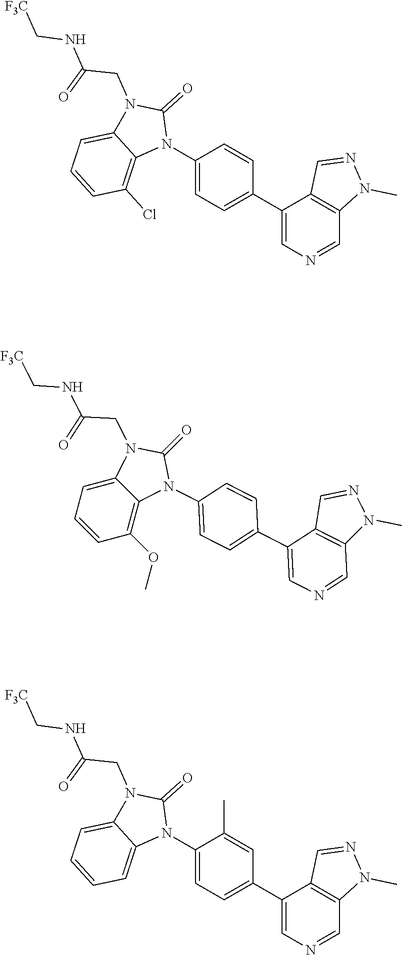 [SMILES: Cn1ncc2c(-c3ccc(-n4c(=O)n(CC(=O)NCC(F)(F)F)c5cccc(Cl)c54)cc3)cncc21 ; COc1cccc2c1n(-c1ccc(-c3cncc4c3cnn4C)cc1)c(=O)n2CC(=O)NCC(F)(F)F ; Cc1cc(-c2cncc3c2cnn3C)ccc1-n1c(=O)n(CC(=O)NCC(F)(F)F)c2ccccc21]

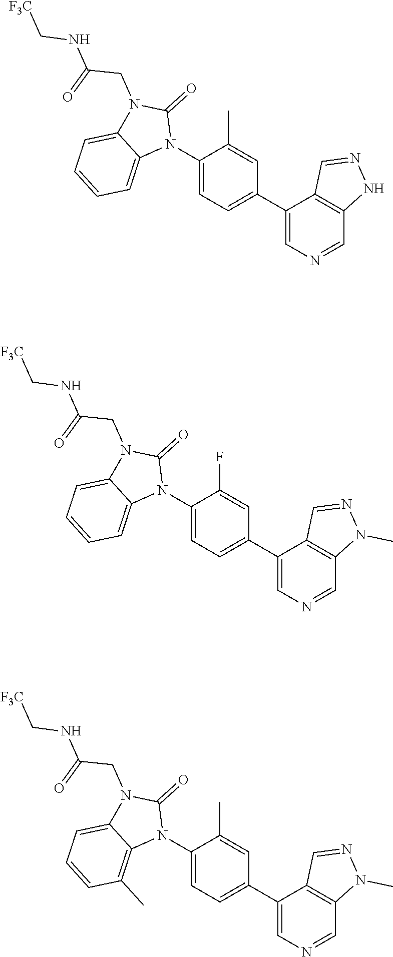 [SMILES: Cc1cc(-c2cncc3[nH]ncc23)ccc1-n1c(=O)n(CC(=O)NCC(F)(F)F)c2ccccc21 ; Cn1ncc2c(-c3ccc(-n4c(=O)n(CC(=O)NCC(F)(F)F)c5ccccc54)c(F)c3)cncc21 ; Cc1cc(-c2cncc3c2cnn3C)ccc1-n1c(=O)n(CC(=O)NCC(F)(F)F)c2cccc(C)c21]

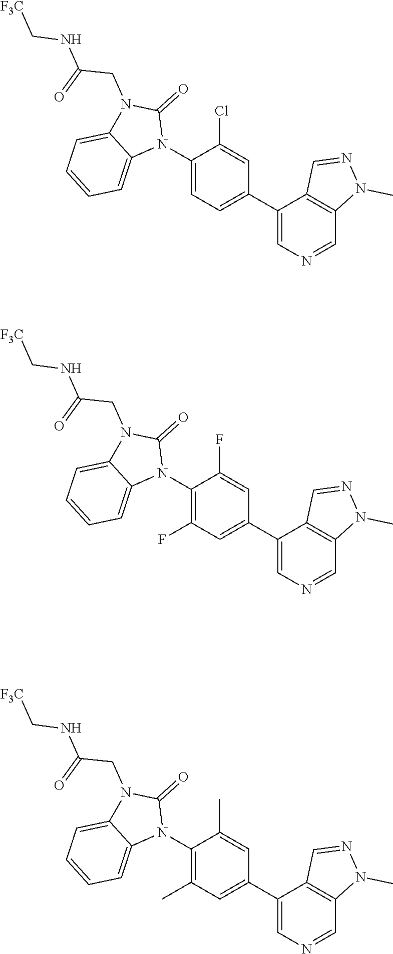 [SMILES: Cn1ncc2c(-c3ccc(-n4c(=O)n(CC(=O)NCC(F)(F)F)c5ccccc54)c(Cl)c3)cncc21 ; Cn1ncc2c(-c3cc(F)c(-n4c(=O)n(CC(=O)NCC(F)(F)F)c5ccccc54)c(F)c3)cncc21 ; Cc1cc(-c2cncc3c2cnn3C)cc(C)c1-n1c(=O)n(CC(=O)NCC(F)(F)F)c2ccccc21]

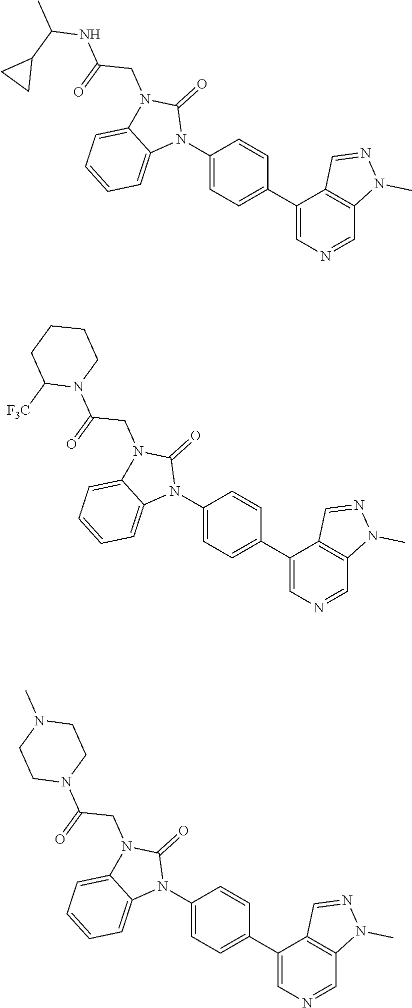 [SMILES: CC(NC(=O)Cn1c(=O)n(-c2ccc(-c3cncc4c3cnn4C)cc2)c2ccccc21)C1CC1 ; Cn1ncc2c(-c3ccc(-n4c(=O)n(CC(=O)N5CCCCC5C(F)(F)F)c5ccccc54)cc3)cncc21 ; CN1CCN(C(=O)Cn2c(=O)n(-c3ccc(-c4cncc5c4cnn5C)cc3)c3ccccc32)CC1]

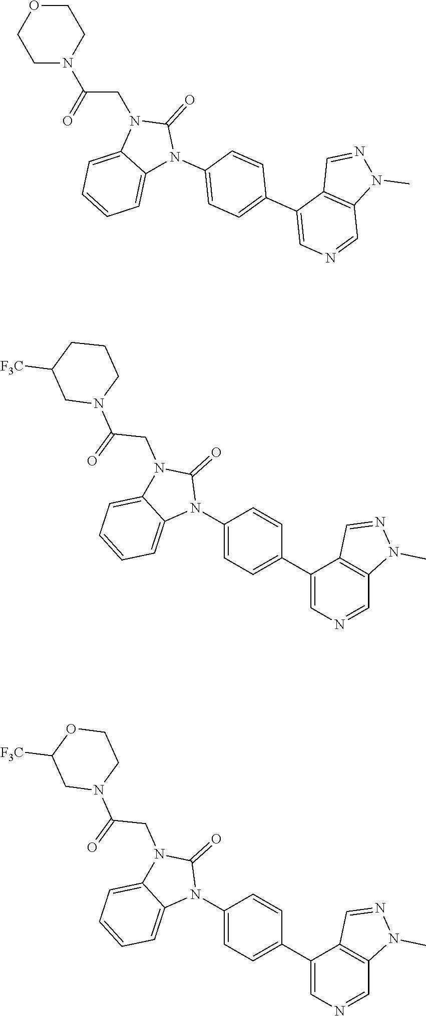 [SMILES: Cn1ncc2c(-c3ccc(-n4c(=O)n(CC(=O)N5CCOCC5)c5ccccc54)cc3)cncc21 ; Cn1ncc2c(-c3ccc(-n4c(=O)n(CC(=O)N5CCCC(C(F)(F)F)C5)c5ccccc54)cc3)cncc21 ; Cn1ncc2c(-c3ccc(-n4c(=O)n(CC(=O)N5CCOC(C(F)(F)F)C5)c5ccccc54)cc3)cncc21]

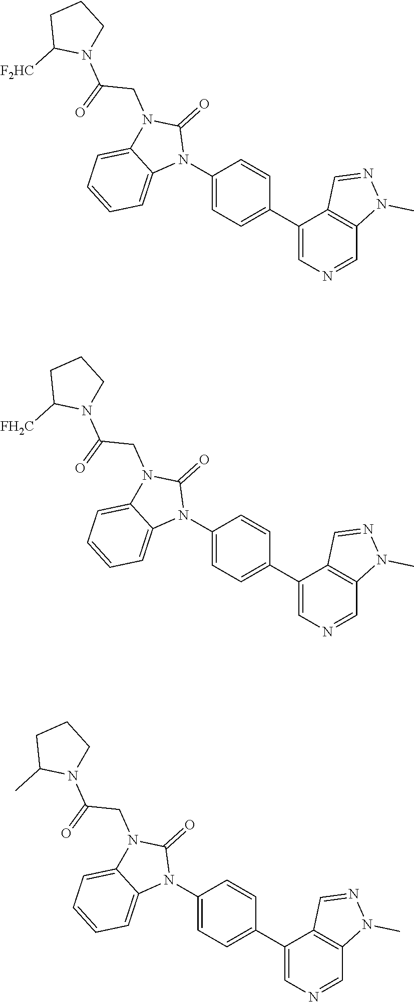

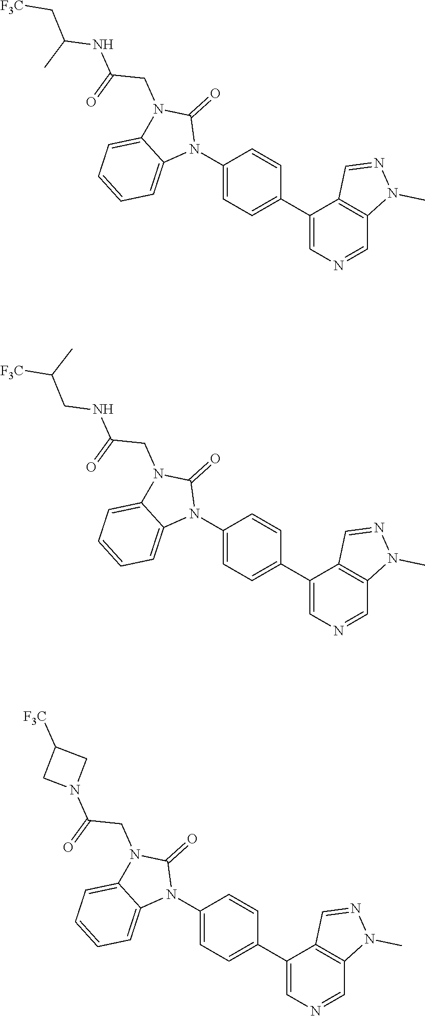 [SMILES: CC(CC(F)(F)F)NC(=O)Cn1c(=O)n(-c2ccc(-c3cncc4c3cnn4C)cc2)c2ccccc21 ; CC(CNC(=O)Cn1c(=O)n(-c2ccc(-c3cncc4c3cnn4C)cc2)c2ccccc21)C(F)(F)F ; Cn1ncc2c(-c3ccc(-n4c(=O)n(CC(=O)N5CC(C(F)(F)F)C5)c5ccccc54)cc3)cncc21]

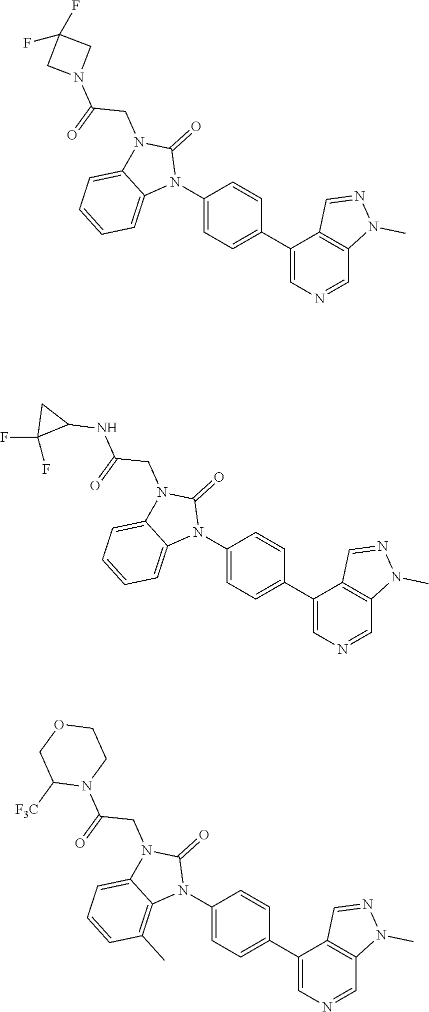 [SMILES: Cn1ncc2c(-c3ccc(-n4c(=O)n(CC(=O)N5CC(F)(F)C5)c5ccccc54)cc3)cncc21 ; Cn1ncc2c(-c3ccc(-n4c(=O)n(CC(=O)NC5CC5(F)F)c5ccccc54)cc3)cncc21 ; Cc1cccc2c1n(-c1ccc(-c3cncc4c3cnn4C)cc1)c(=O)n2CC(=O)N1CCOCC1C(F)(F)F]

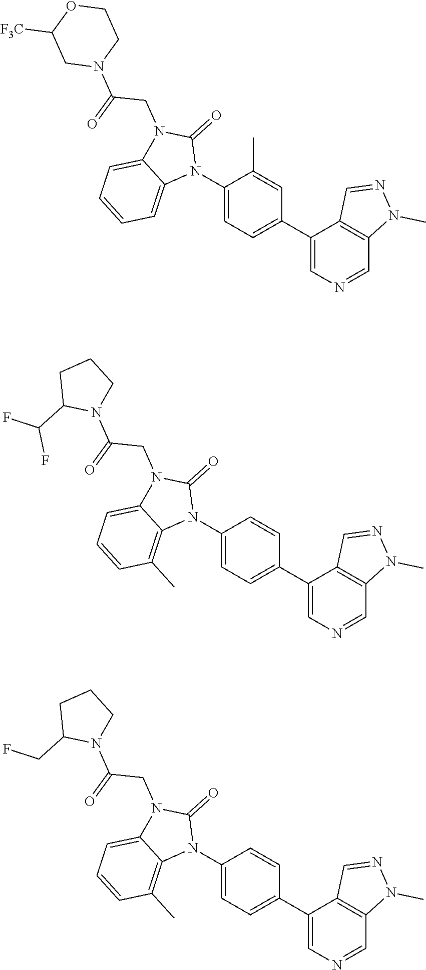 [SMILES: Cc1cc(-c2cncc3c2cnn3C)ccc1-n1c(=O)n(CC(=O)N2CCOC(C(F)(F)F)C2)c2ccccc21 ; Cc1cccc2c1n(-c1ccc(-c3cncc4c3cnn4C)cc1)c(=O)n2CC(=O)N1CCCC1C(F)F ; Cc1cccc2c1n(-c1ccc(-c3cncc4c3cnn4C)cc1)c(=O)n2CC(=O)N1CCCC1CF]

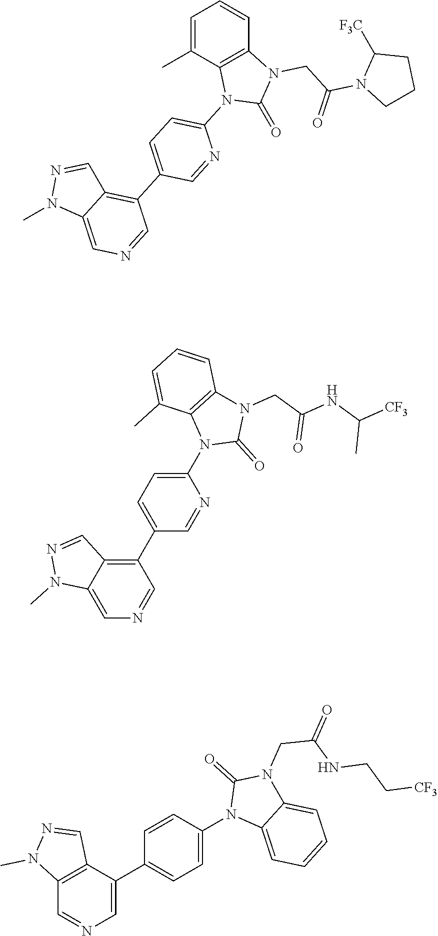 [SMILES: Cc1cccc2c1n(-c1ccc(-c3cncc4c3cnn4C)cn1)c(=O)n2CC(=O)N1CCCC1C(F)(F)F ; Cc1cccc2c1n(-c1ccc(-c3cncc4c3cnn4C)cn1)c(=O)n2CC(=O)NC(C)C(F)(F)F ; Cn1ncc2c(-c3ccc(-n4c(=O)n(CC(=O)NCCC(F)(F)F)c5ccccc54)cc3)cncc21]

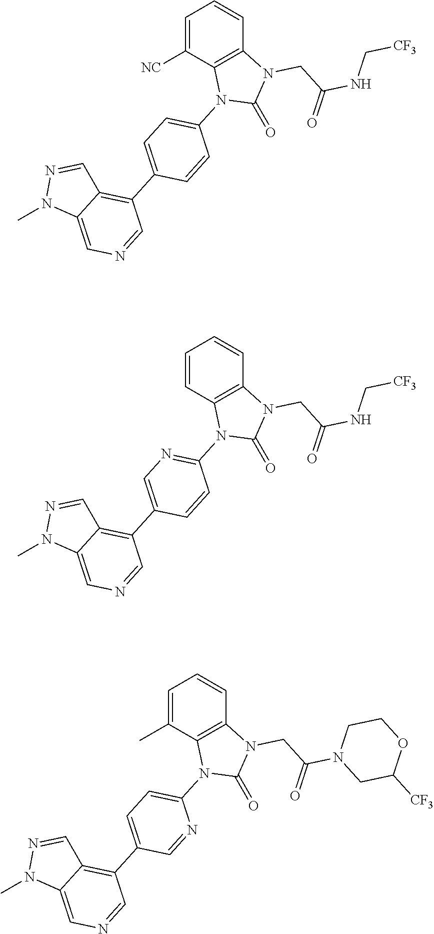 [SMILES: Cn1ncc2c(-c3ccc(-n4c(=O)n(CC(=O)NCC(F)(F)F)c5cccc(C#N)c54)cc3)cncc21 ; Cn1ncc2c(-c3ccc(-n4c(=O)n(CC(=O)NCC(F)(F)F)c5ccccc54)nc3)cncc21 ; Cc1cccc2c1n(-c1ccc(-c3cncc4c3cnn4C)cn1)c(=O)n2CC(=O)N1CCOC(C(F)(F)F)C1]

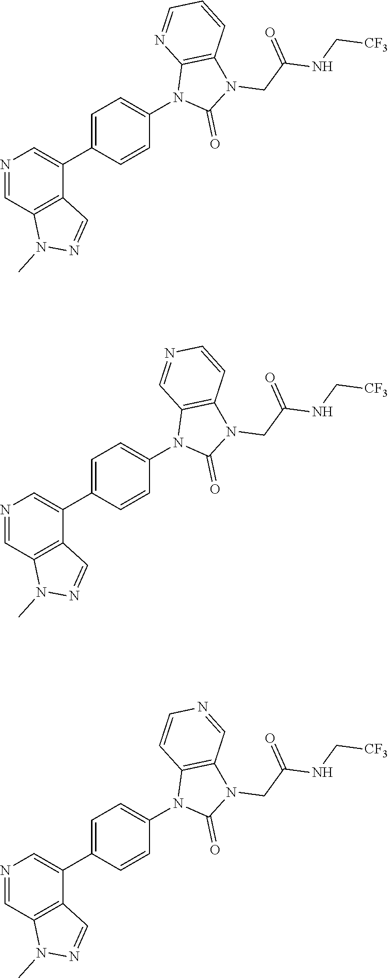 [SMILES: Cn1ncc2c(-c3ccc(-n4c(=O)n(CC(=O)NCC(F)(F)F)c5cccnc54)cc3)cncc21 ; Cn1ncc2c(-c3ccc(-n4c(=O)n(CC(=O)NCC(F)(F)F)c5ccncc54)cc3)cncc21 ; Cn1ncc2c(-c3ccc(-n4c(=O)n(CC(=O)NCC(F)(F)F)c5cnccc54)cc3)cncc21]

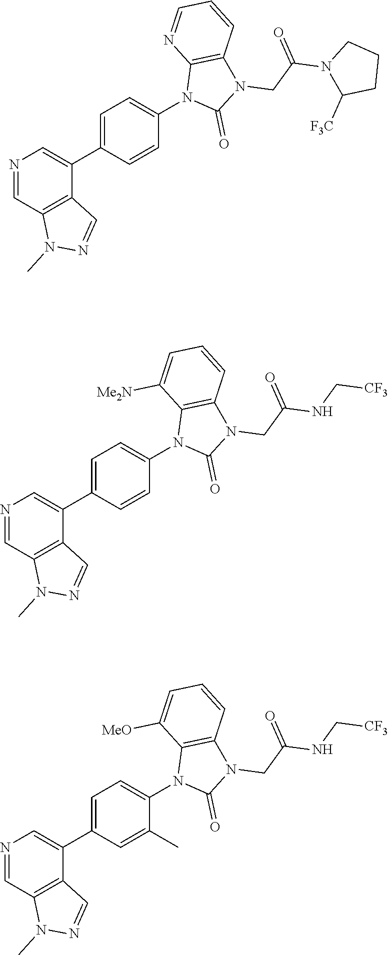 [SMILES: Cn1ncc2c(-c3ccc(-n4c(=O)n(CC(=O)N5CCCC5C(F)(F)F)c5cccnc54)cc3)cncc21 ; CN(C)c1cccc2c1n(-c1ccc(-c3cncc4c3cnn4C)cc1)c(=O)n2CC(=O)NCC(F)(F)F ; COc1cccc2c1n(-c1ccc(-c3cncc4c3cnn4C)cc1C)c(=O)n2CC(=O)NCC(F)(F)F]

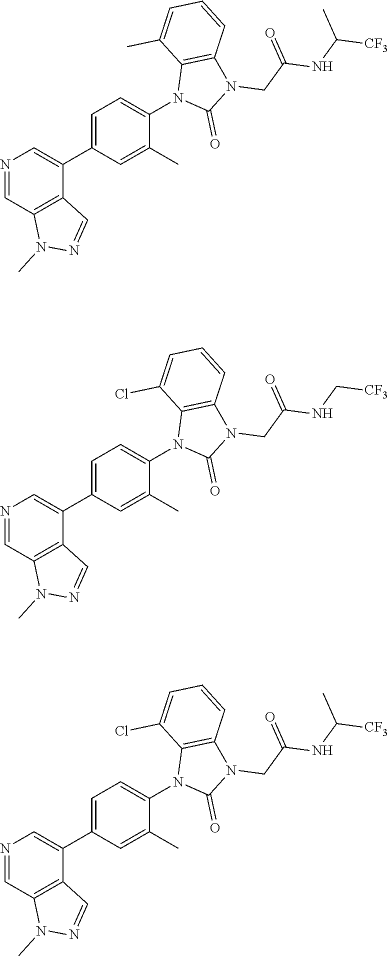 [SMILES: Cc1cc(-c2cncc3c2cnn3C)ccc1-n1c(=O)n(CC(=O)NC(C)C(F)(F)F)c2cccc(C)c21 ; Cc1cc(-c2cncc3c2cnn3C)ccc1-n1c(=O)n(CC(=O)NCC(F)(F)F)c2cccc(Cl)c21 ; Cc1cc(-c2cncc3c2cnn3C)ccc1-n1c(=O)n(CC(=O)NC(C)C(F)(F)F)c2cccc(Cl)c21]

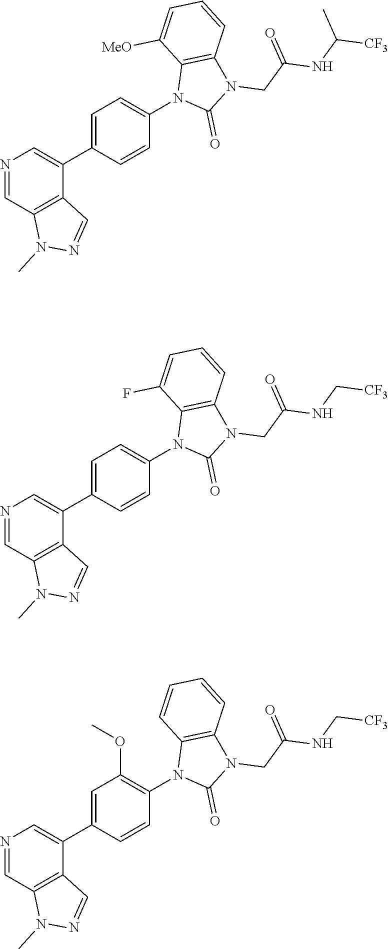 [SMILES: COc1cccc2c1n(-c1ccc(-c3cncc4c3cnn4C)cc1)c(=O)n2CC(=O)NC(C)C(F)(F)F ; Cn1ncc2c(-c3ccc(-n4c(=O)n(CC(=O)NCC(F)(F)F)c5cccc(F)c54)cc3)cncc21 ; COc1cc(-c2cncc3c2cnn3C)ccc1-n1c(=O)n(CC(=O)NCC(F)(F)F)c2ccccc21]

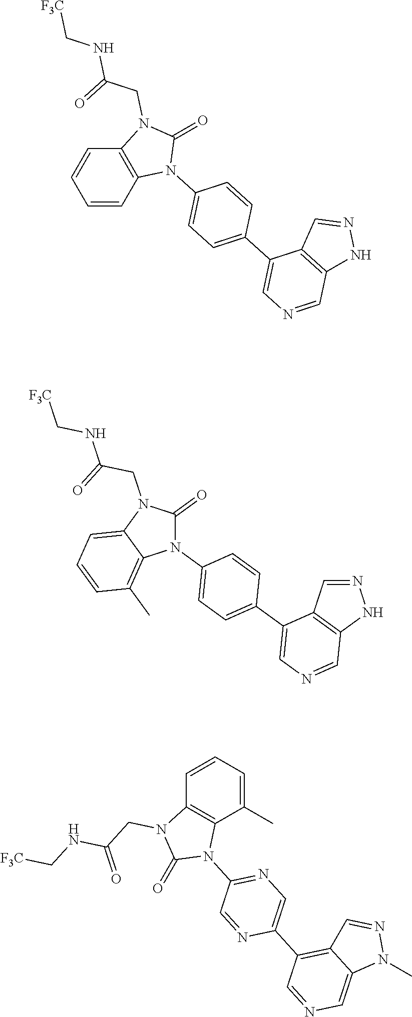 [SMILES: O=C(Cn1c(=O)n(-c2ccc(-c3cncc4[nH]ncc34)cc2)c2ccccc21)NCC(F)(F)F ; Cc1cccc2c1n(-c1ccc(-c3cncc4[nH]ncc34)cc1)c(=O)n2CC(=O)NCC(F)(F)F ; Cc1cccc2c1n(-c1cnc(-c3cncc4c3cnn4C)cn1)c(=O)n2CC(=O)NCC(F)(F)F]

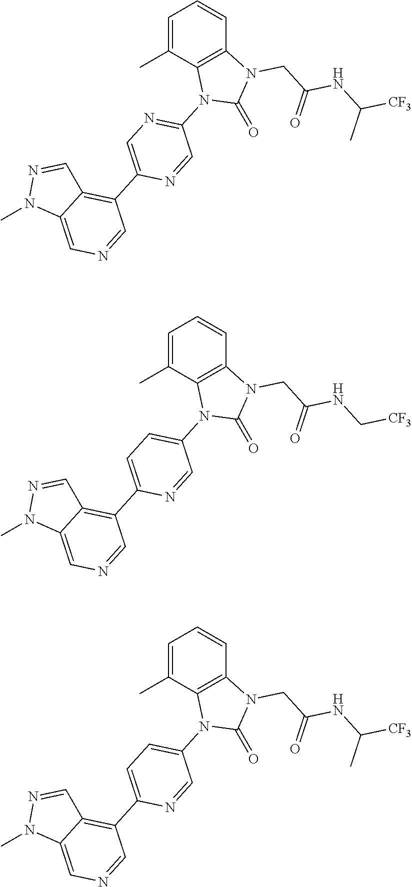 [SMILES: Cc1cccc2c1n(-c1cnc(-c3cncc4c3cnn4C)cn1)c(=O)n2CC(=O)NC(C)C(F)(F)F ; Cc1cccc2c1n(-c1ccc(-c3cncc4c3cnn4C)nc1)c(=O)n2CC(=O)NCC(F)(F)F ; Cc1cccc2c1n(-c1ccc(-c3cncc4c3cnn4C)nc1)c(=O)n2CC(=O)NC(C)C(F)(F)F]

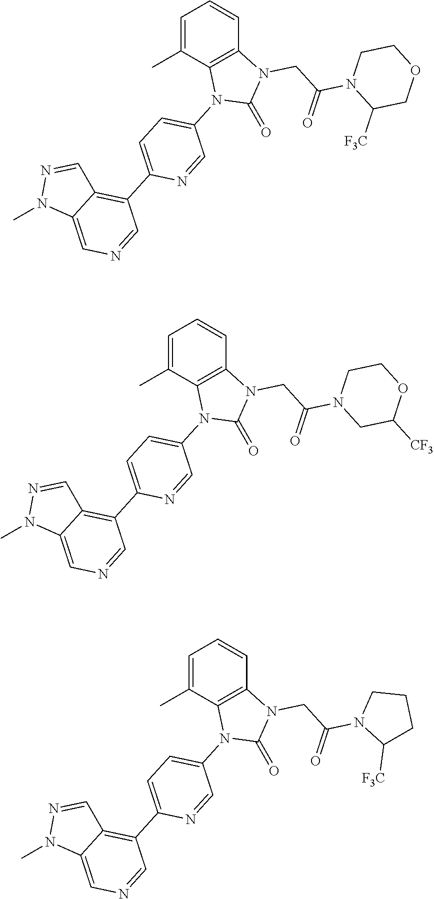 [SMILES: Cc1cccc2c1n(-c1ccc(-c3cncc4c3cnn4C)nc1)c(=O)n2CC(=O)N1CCOCC1C(F)(F)F ; Cc1cccc2c1n(-c1ccc(-c3cncc4c3cnn4C)nc1)c(=O)n2CC(=O)N1CCOC(C(F)(F)F)C1 ; Cc1cccc2c1n(-c1ccc(-c3cncc4c3cnn4C)nc1)c(=O)n2CC(=O)N1CCCC1C(F)(F)F]

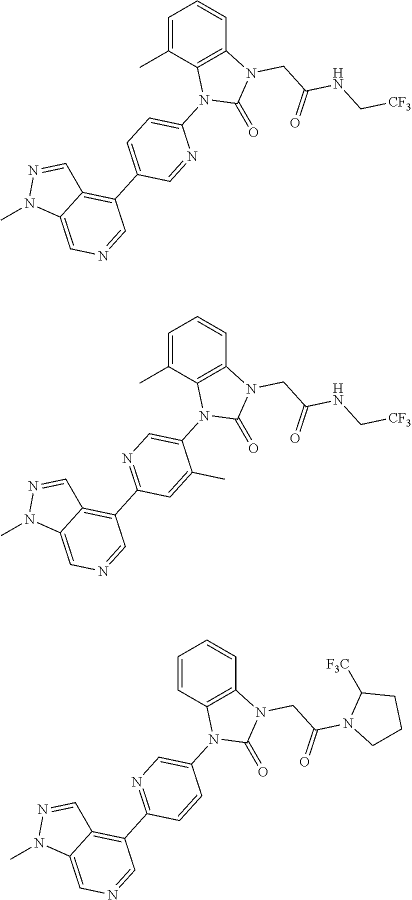 [SMILES: Cc1cccc2c1n(-c1ccc(-c3cncc4c3cnn4C)cn1)c(=O)n2CC(=O)NCC(F)(F)F ; Cc1cc(-c2cncc3c2cnn3C)ncc1-n1c(=O)n(CC(=O)NCC(F)(F)F)c2cccc(C)c21 ; Cn1ncc2c(-c3ccc(-n4c(=O)n(CC(=O)N5CCCC5C(F)(F)F)c5ccccc54)cn3)cncc21]

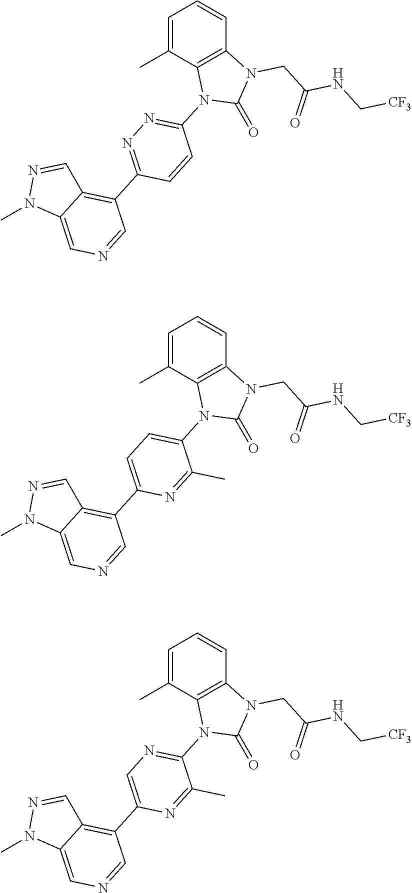 [SMILES: Cc1cccc2c1n(-c1ccc(-c3cncc4c3cnn4C)nn1)c(=O)n2CC(=O)NCC(F)(F)F ; Cc1nc(-c2cncc3c2cnn3C)ccc1-n1c(=O)n(CC(=O)NCC(F)(F)F)c2cccc(C)c21 ; Cc1nc(-c2cncc3c2cnn3C)cnc1-n1c(=O)n(CC(=O)NCC(F)(F)F)c2cccc(C)c21]

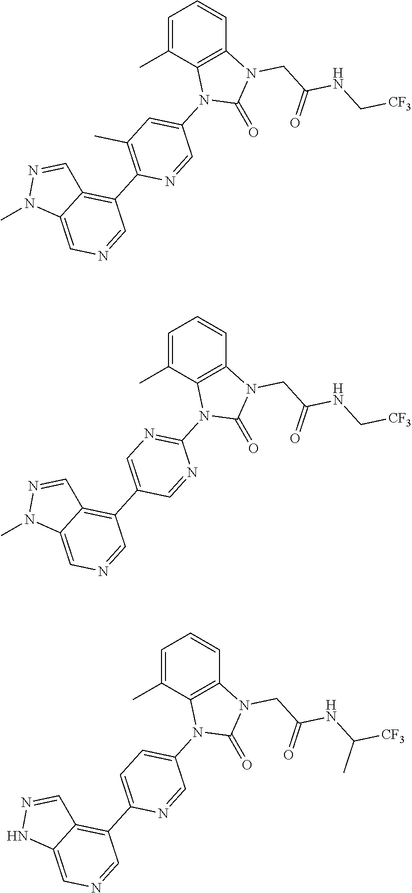 [SMILES: Cc1cc(-n2c(=O)n(CC(=O)NCC(F)(F)F)c3cccc(C)c32)cnc1-c1cncc2c1cnn2C ; Cc1cccc2c1n(-c1ncc(-c3cncc4c3cnn4C)cn1)c(=O)n2CC(=O)NCC(F)(F)F ; Cc1cccc2c1n(-c1ccc(-c3cncc4[nH]ncc34)nc1)c(=O)n2CC(=O)NC(C)C(F)(F)F]

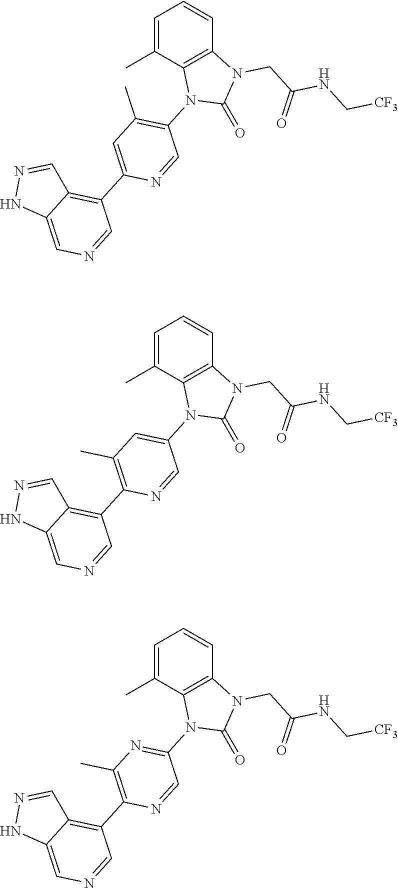 [SMILES: Cc1cc(-c2cncc3[nH]ncc23)ncc1-n1c(=O)n(CC(=O)NCC(F)(F)F)c2cccc(C)c21 ; Cc1cc(-n2c(=O)n(CC(=O)NCC(F)(F)F)c3cccc(C)c32)cnc1-c1cncc2[nH]ncc12 ; Cc1nc(-n2c(=O)n(CC(=O)NCC(F)(F)F)c3cccc(C)c32)cnc1-c1cncc2[nH]ncc12]

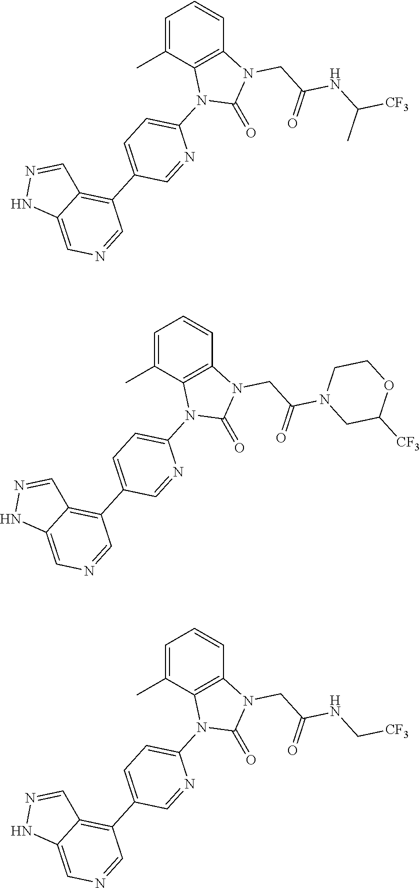 [SMILES: Cc1cccc2c1n(-c1ccc(-c3cncc4[nH]ncc34)cn1)c(=O)n2CC(=O)NC(C)C(F)(F)F ; Cc1cccc2c1n(-c1ccc(-c3cncc4[nH]ncc34)cn1)c(=O)n2CC(=O)N1CCOC(C(F)(F)F)C1 ; Cc1cccc2c1n(-c1ccc(-c3cncc4[nH]ncc34)cn1)c(=O)n2CC(=O)NCC(F)(F)F]

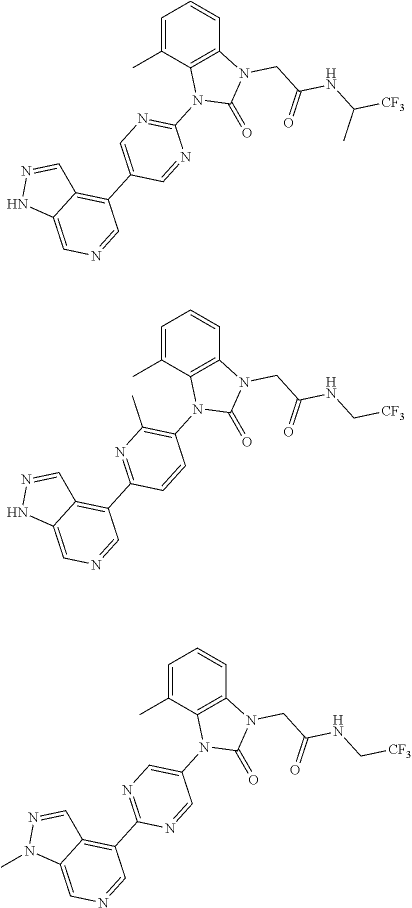 [SMILES: Cc1cccc2c1n(-c1ncc(-c3cncc4[nH]ncc34)cn1)c(=O)n2CC(=O)NC(C)C(F)(F)F ; Cc1nc(-c2cncc3[nH]ncc23)ccc1-n1c(=O)n(CC(=O)NCC(F)(F)F)c2cccc(C)c21 ; Cc1cccc2c1n(-c1cnc(-c3cncc4c3cnn4C)nc1)c(=O)n2CC(=O)NCC(F)(F)F]

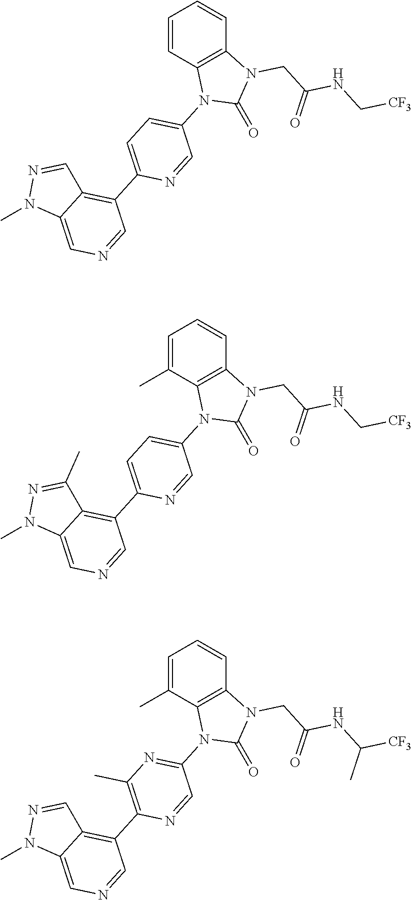 [SMILES: Cn1ncc2c(-c3ccc(-n4c(=O)n(CC(=O)NCC(F)(F)F)c5ccccc54)cn3)cncc21 ; Cc1nn(C)c2cncc(-c3ccc(-n4c(=O)n(CC(=O)NCC(F)(F)F)c5cccc(C)c54)cn3)c12 ; Cc1nc(-n2c(=O)n(CC(=O)NC(C)C(F)(F)F)c3cccc(C)c32)cnc1-c1cncc2c1cnn2C]

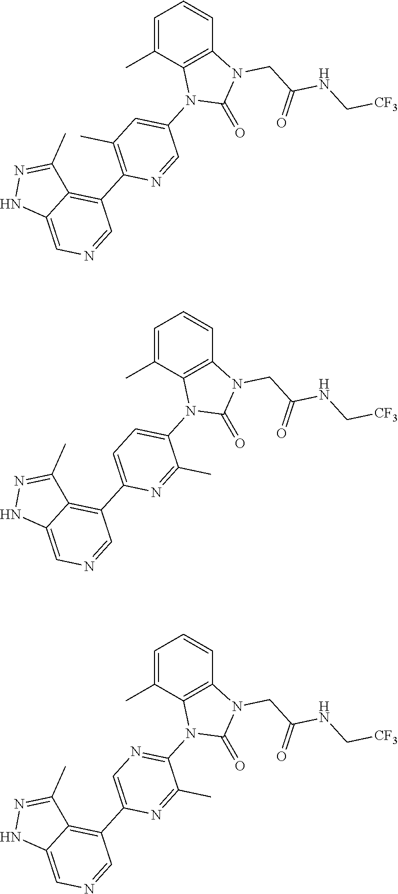 [SMILES: Cc1cc(-n2c(=O)n(CC(=O)NCC(F)(F)F)c3cccc(C)c32)cnc1-c1cncc2[nH]nc(C)c12 ; Cc1nc(-c2cncc3[nH]nc(C)c23)ccc1-n1c(=O)n(CC(=O)NCC(F)(F)F)c2cccc(C)c21 ; Cc1nc(-c2cncc3[nH]nc(C)c23)cnc1-n1c(=O)n(CC(=O)NCC(F)(F)F)c2cccc(C)c21]

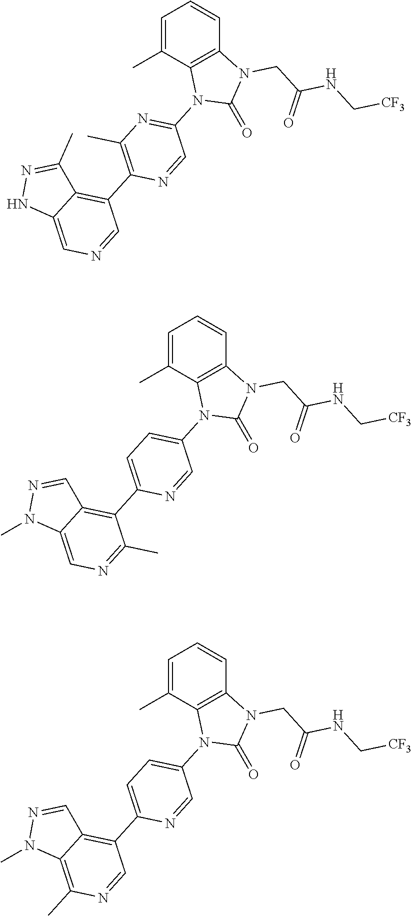 [SMILES: Cc1nc(-n2c(=O)n(CC(=O)NCC(F)(F)F)c3cccc(C)c32)cnc1-c1cncc2[nH]nc(C)c12 ; Cc1ncc2c(cnn2C)c1-c1ccc(-n2c(=O)n(CC(=O)NCC(F)(F)F)c3cccc(C)c32)cn1 ; Cc1ncc(-c2ccc(-n3c(=O)n(CC(=O)NCC(F)(F)F)c4cccc(C)c43)cn2)c2cnn(C)c12]

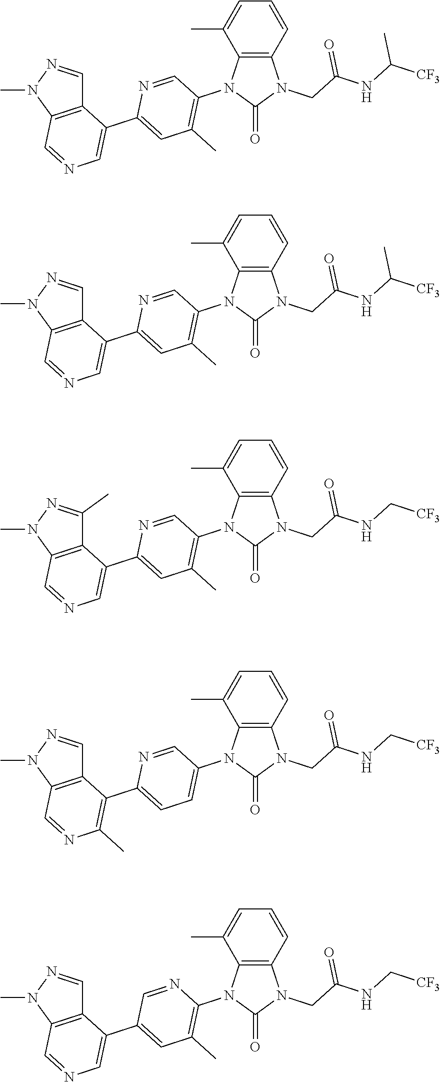 [SMILES: Cc1cc(-c2cncc3c2cnn3C)ncc1-n1c(=O)n(CC(=O)NC(C)C(F)(F)F)c2cccc(C)c21 ; Cc1cc(-c2cncc3c2cnn3C)ncc1-n1c(=O)n(CC(=O)NC(C)C(F)(F)F)c2cccc(C)c21 ; Cc1cc(-c2cncc3c2c(C)nn3C)ncc1-n1c(=O)n(CC(=O)NCC(F)(F)F)c2cccc(C)c21 ; Cc1ncc2c(cnn2C)c1-c1ccc(-n2c(=O)n(CC(=O)NCC(F)(F)F)c3cccc(C)c32)cn1 ; Cc1cc(-c2cncc3c2cnn3C)cnc1-n1c(=O)n(CC(=O)NCC(F)(F)F)c2cccc(C)c21]

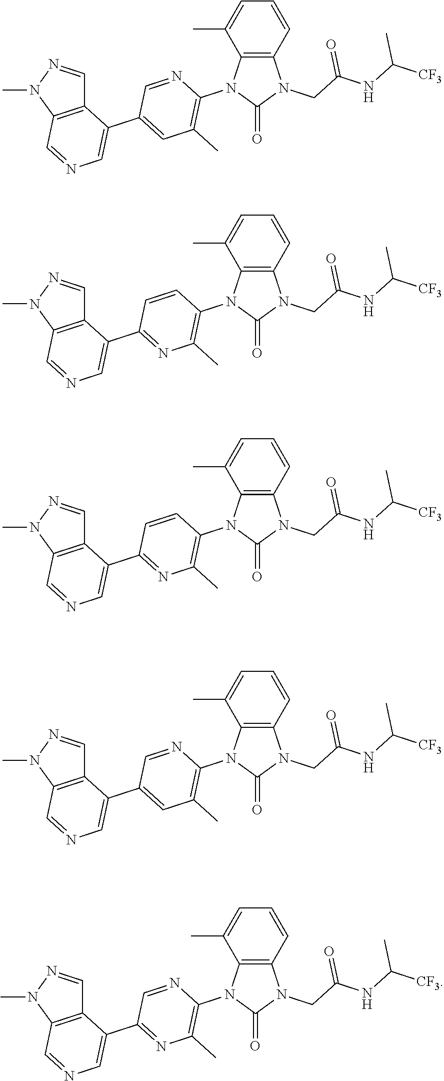 [SMILES: Cc1cc(-c2cncc3c2cnn3C)cnc1-n1c(=O)n(CC(=O)NC(C)C(F)(F)F)c2cccc(C)c21 ; Cc1nc(-c2cncc3c2cnn3C)ccc1-n1c(=O)n(CC(=O)NC(C)C(F)(F)F)c2cccc(C)c21 ; Cc1nc(-c2cncc3c2cnn3C)ccc1-n1c(=O)n(CC(=O)NC(C)C(F)(F)F)c2cccc(C)c21 ; Cc1cc(-c2cncc3c2cnn3C)cnc1-n1c(=O)n(CC(=O)NC(C)C(F)(F)F)c2cccc(C)c21 ; Cc1nc(-c2cncc3c2cnn3C)cnc1-n1c(=O)n(CC(=O)NC(C)C(F)(F)F)c2cccc(C)c21]

The compound of formula (I) may be selected from

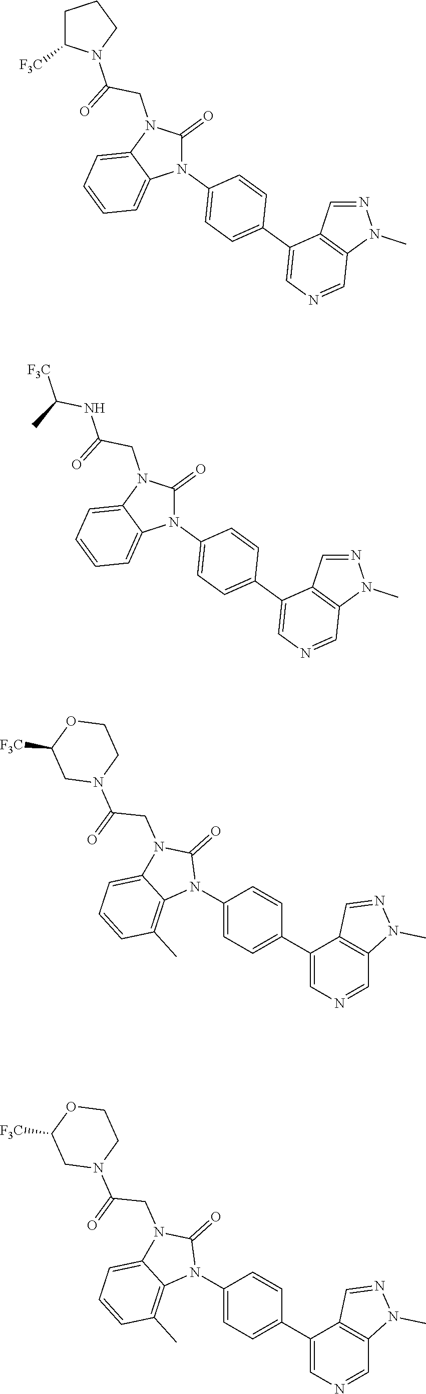

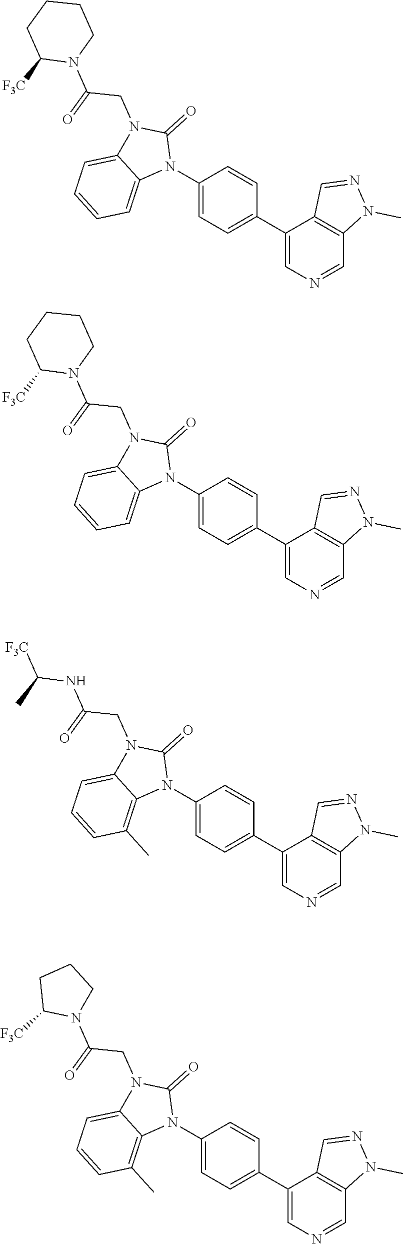

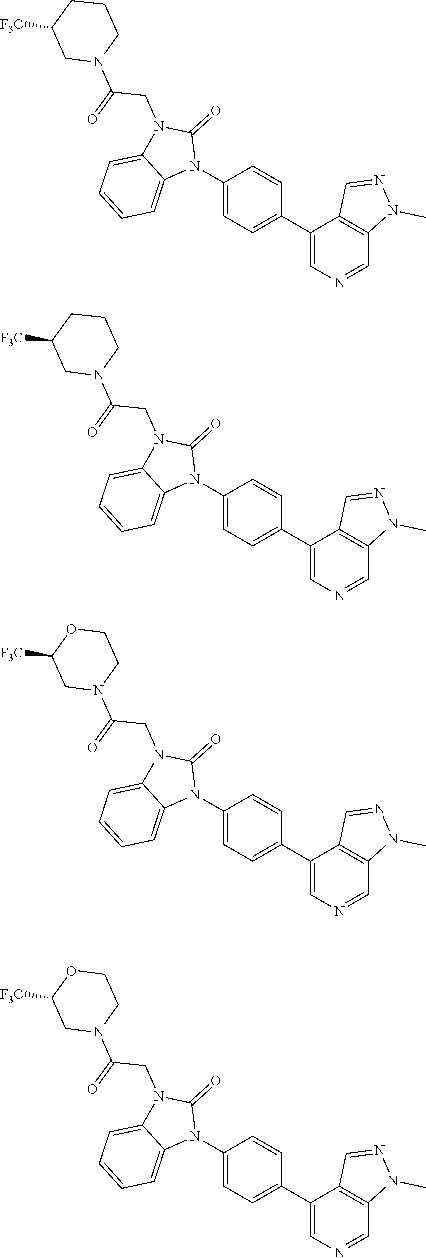 [SMILES: Cn1ncc2c(-c3ccc(-n4c(=O)n(CC(=O)N5CCC[C@@H](C(F)(F)F)C5)c5ccccc54)cc3)cncc21 ; Cn1ncc2c(-c3ccc(-n4c(=O)n(CC(=O)N5CCC[C@H](C(F)(F)F)C5)c5ccccc54)cc3)cncc21 ; Cn1ncc2c(-c3ccc(-n4c(=O)n(CC(=O)N5CCO[C@H](C(F)(F)F)C5)c5ccccc54)cc3)cncc21 ; Cn1ncc2c(-c3ccc(-n4c(=O)n(CC(=O)N5CCO[C@@H](C(F)(F)F)C5)c5ccccc54)cc3)cncc21]

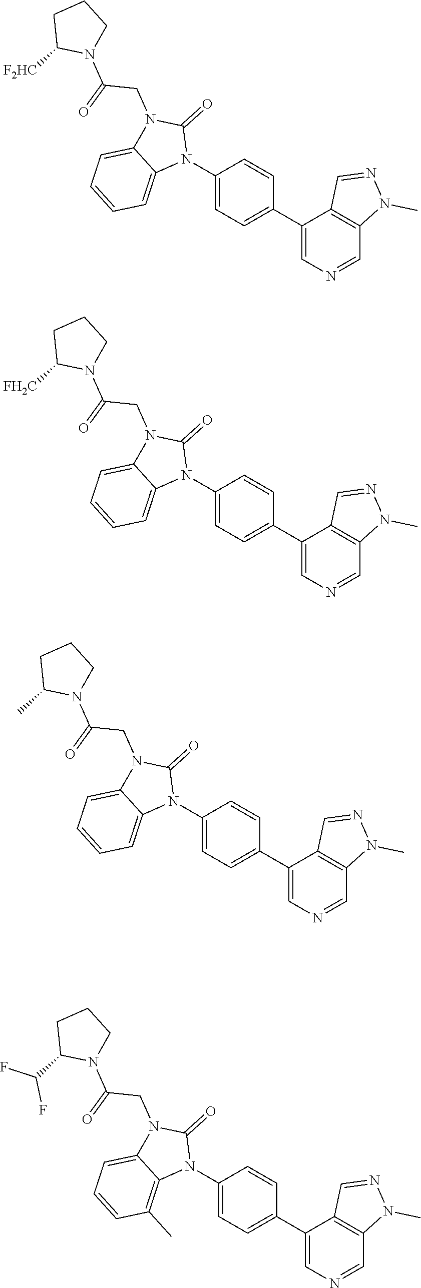

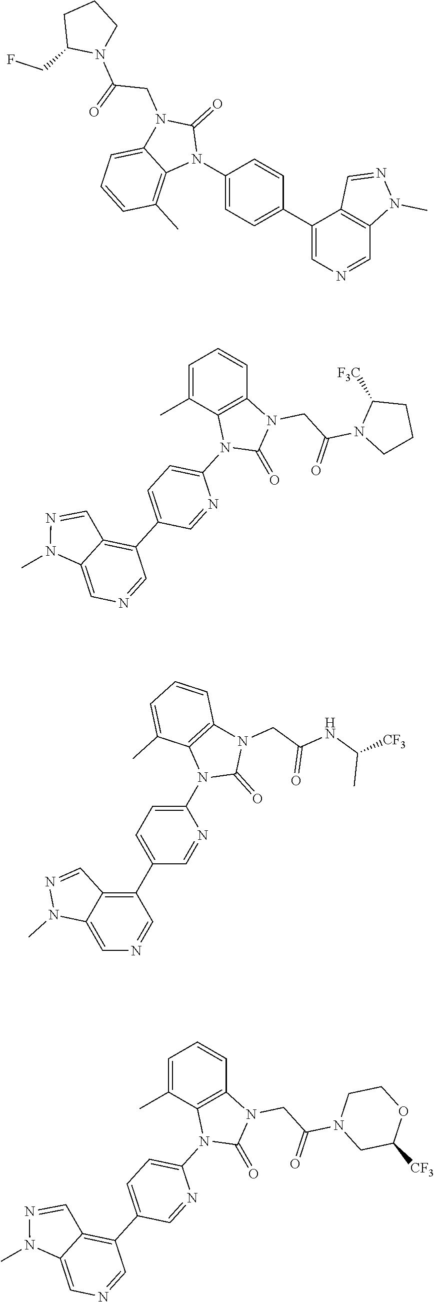

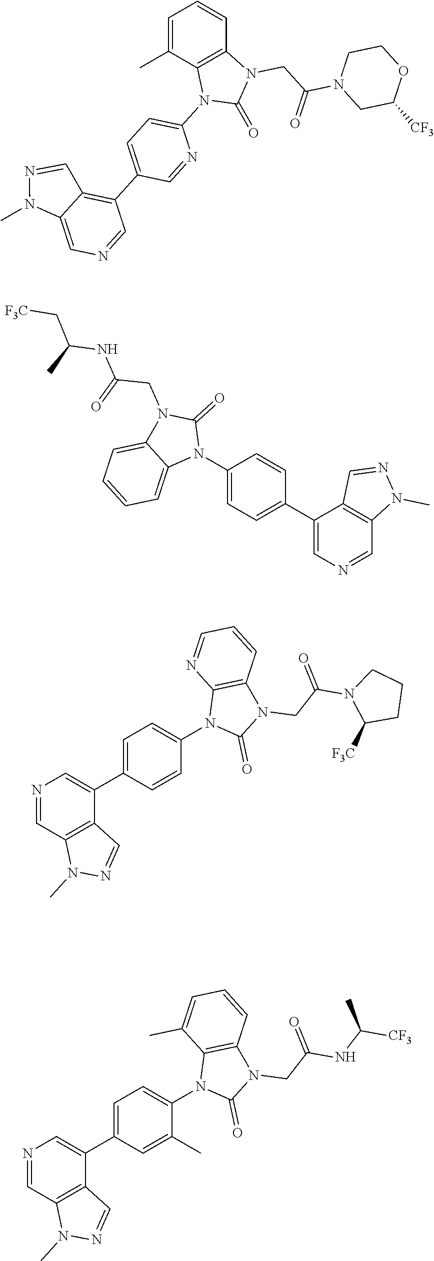 [SMILES: Cc1cccc2c1n(-c1ccc(-c3cncc4c3cnn4C)cn1)c(=O)n2CC(=O)N1CCO[C@H](C(F)(F)F)C1 ; C[C@@H](CC(F)(F)F)NC(=O)Cn1c(=O)n(-c2ccc(-c3cncc4c3cnn4C)cc2)c2ccccc21 ; Cn1ncc2c(-c3ccc(-n4c(=O)n(CC(=O)N5CCC[C@H]5C(F)(F)F)c5cccnc54)cc3)cncc21 ; Cc1cc(-c2cncc3c2cnn3C)ccc1-n1c(=O)n(CC(=O)N[C@@H](C)C(F)(F)F)c2cccc(C)c21]

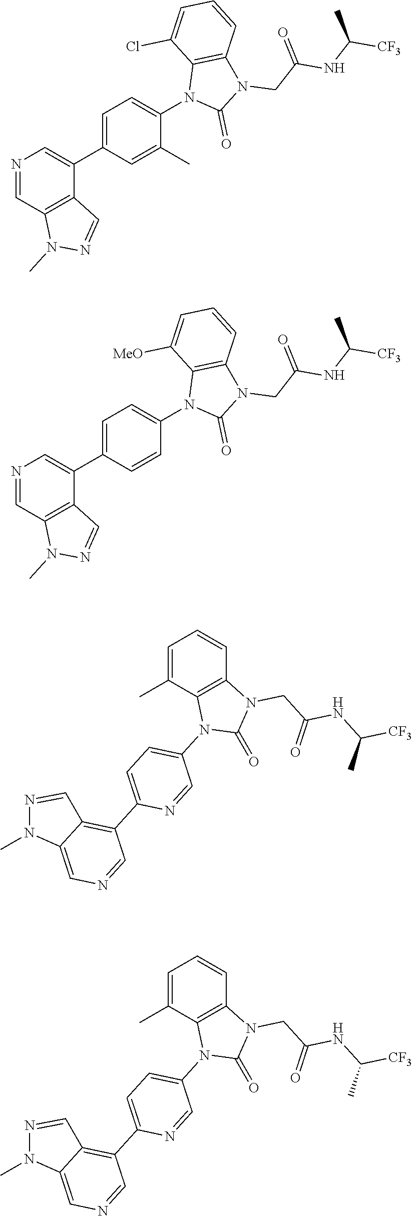 [SMILES: Cc1cc(-c2cncc3c2cnn3C)ccc1-n1c(=O)n(CC(=O)N[C@@H](C)C(F)(F)F)c2cccc(Cl)c21 ; COc1cccc2c1n(-c1ccc(-c3cncc4c3cnn4C)cc1)c(=O)n2CC(=O)N[C@@H](C)C(F)(F)F ; Cc1cccc2c1n(-c1ccc(-c3cncc4c3cnn4C)nc1)c(=O)n2CC(=O)N[C@H](C)C(F)(F)F ; Cc1cccc2c1n(-c1ccc(-c3cncc4c3cnn4C)nc1)c(=O)n2CC(=O)N[C@@H](C)C(F)(F)F]

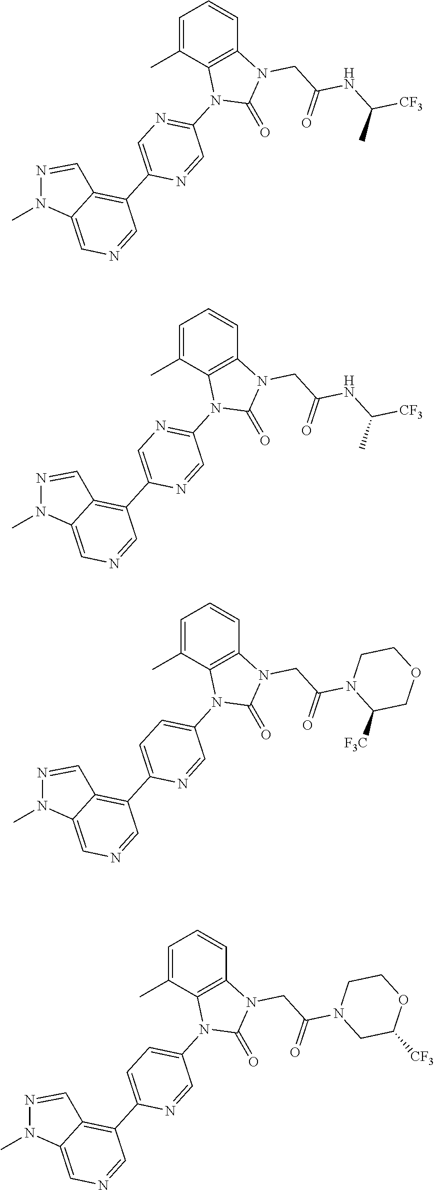 [SMILES: Cc1cccc2c1n(-c1cnc(-c3cncc4c3cnn4C)cn1)c(=O)n2CC(=O)N[C@H](C)C(F)(F)F ; Cc1cccc2c1n(-c1cnc(-c3cncc4c3cnn4C)cn1)c(=O)n2CC(=O)N[C@@H](C)C(F)(F)F ; Cc1cccc2c1n(-c1ccc(-c3cncc4c3cnn4C)nc1)c(=O)n2CC(=O)N1CCOC[C@H]1C(F)(F)F ; Cc1cccc2c1n(-c1ccc(-c3cncc4c3cnn4C)nc1)c(=O)n2CC(=O)N1CCO[C@H](C(F)(F)F)C1]

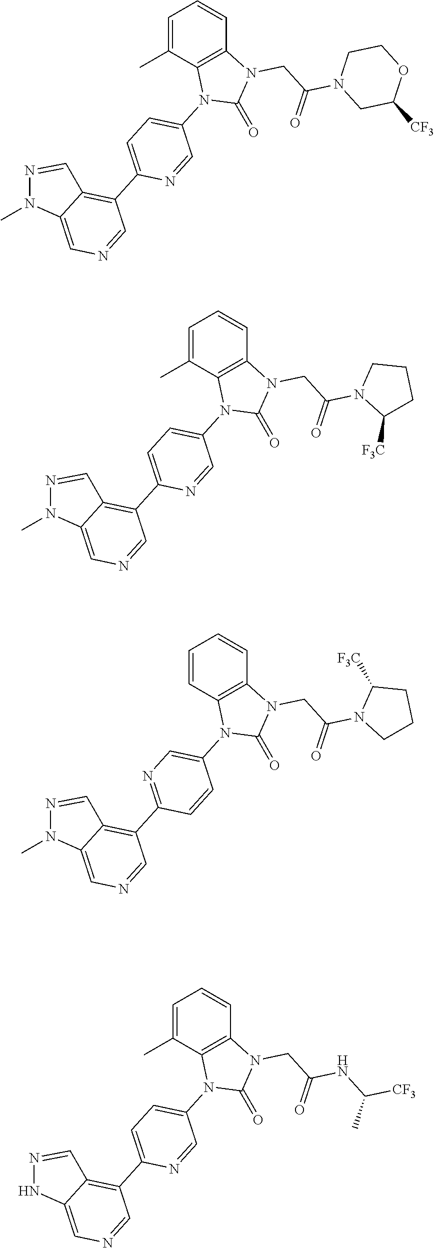 [SMILES: Cc1cccc2c1n(-c1ccc(-c3cncc4c3cnn4C)nc1)c(=O)n2CC(=O)N1CCO[C@@H](C(F)(F)F)C1 ; Cc1cccc2c1n(-c1ccc(-c3cncc4c3cnn4C)nc1)c(=O)n2CC(=O)N1CCC[C@H]1C(F)(F)F ; Cn1ncc2c(-c3ccc(-n4c(=O)n(CC(=O)N5CCC[C@H]5C(F)(F)F)c5ccccc54)cn3)cncc21 ; Cc1cccc2c1n(-c1ccc(-c3cncc4[nH]ncc34)nc1)c(=O)n2CC(=O)N[C@@H](C)C(F)(F)F]

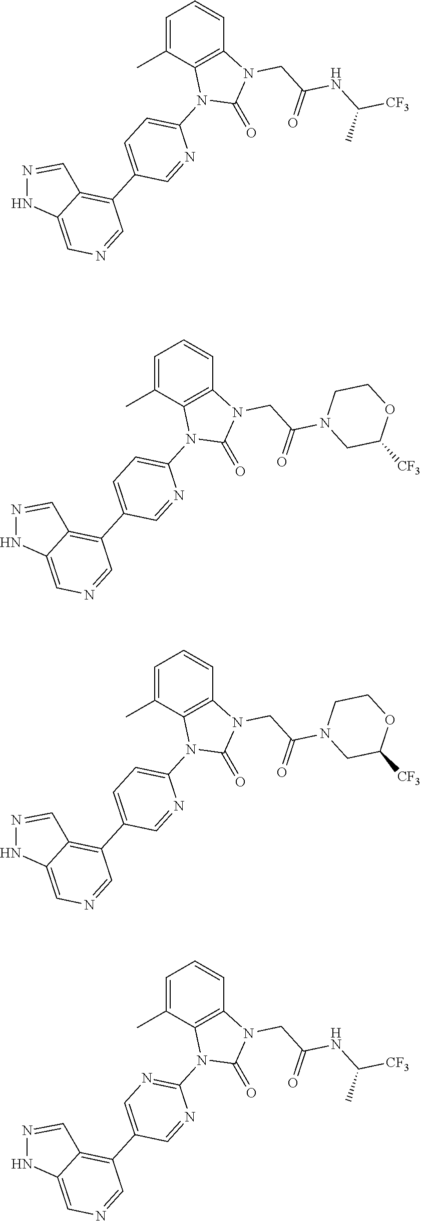 [SMILES: Cc1cccc2c1n(-c1ccc(-c3cncc4[nH]ncc34)cn1)c(=O)n2CC(=O)N[C@@H](C)C(F)(F)F ; Cc1cccc2c1n(-c1ccc(-c3cncc4[nH]ncc34)cn1)c(=O)n2CC(=O)N1CCO[C@H](C(F)(F)F)C1 ; Cc1cccc2c1n(-c1ccc(-c3cncc4[nH]ncc34)cn1)c(=O)n2CC(=O)N1CCO[C@@H](C(F)(F)F)C1 ; Cc1cccc2c1n(-c1ncc(-c3cncc4[nH]ncc34)cn1)c(=O)n2CC(=O)N[C@@H](C)C(F)(F)F]

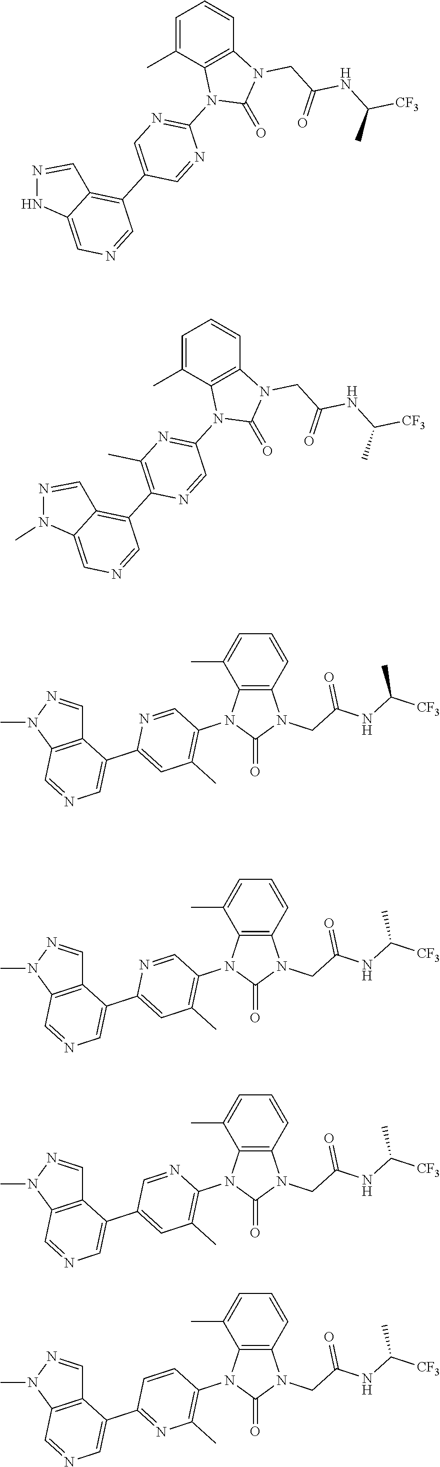 [SMILES: Cc1cccc2c1n(-c1ncc(-c3cncc4[nH]ncc34)cn1)c(=O)n2CC(=O)N[C@H](C)C(F)(F)F ; Cc1nc(-n2c(=O)n(CC(=O)N[C@@H](C)C(F)(F)F)c3cccc(C)c32)cnc1-c1cncc2c1cnn2C ; Cc1cc(-c2cncc3c2cnn3C)ncc1-n1c(=O)n(CC(=O)N[C@@H](C)C(F)(F)F)c2cccc(C)c21 ; Cc1cc(-c2cncc3c2cnn3C)ncc1-n1c(=O)n(CC(=O)N[C@H](C)C(F)(F)F)c2cccc(C)c21 ; Cc1cc(-c2cncc3c2cnn3C)cnc1-n1c(=O)n(CC(=O)N[C@H](C)C(F)(F)F)c2cccc(C)c21 ; Cc1nc(-c2cncc3c2cnn3C)ccc1-n1c(=O)n(CC(=O)N[C@H](C)C(F)(F)F)c2cccc(C)c21]

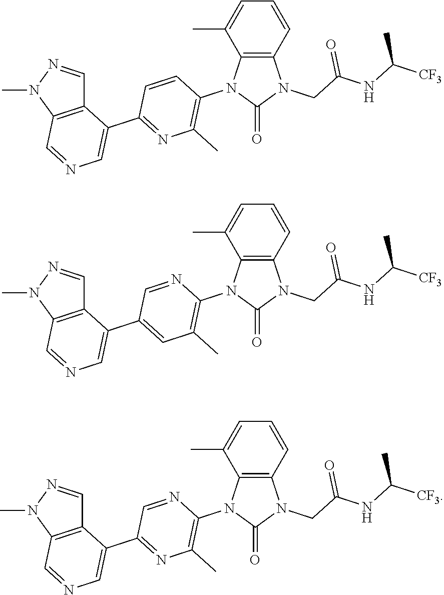 [SMILES: Cc1nc(-c2cncc3c2cnn3C)ccc1-n1c(=O)n(CC(=O)N[C@@H](C)C(F)(F)F)c2cccc(C)c21 ; Cc1cc(-c2cncc3c2cnn3C)cnc1-n1c(=O)n(CC(=O)N[C@@H](C)C(F)(F)F)c2cccc(C)c21 ; Cc1nc(-c2cncc3c2cnn3C)cnc1-n1c(=O)n(CC(=O)N[C@@H](C)C(F)(F)F)c2cccc(C)c21]

It may be that the compound of formula (I) is not

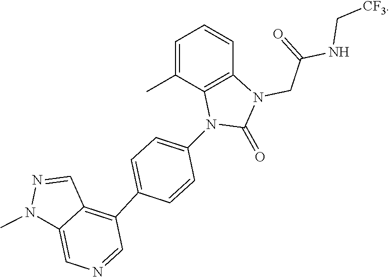 [SMILES: Cc1cccc2c1n(-c1ccc(-c3cncc4c3cnn4C)cc1)c(=O)n2CC(=O)NCC(F)(F)F]

In an aspect of the invention there is provided the compounds of the present invention for use as a medicament.

In accordance with another aspect, there is provided a compound of the present invention for use in the treatment of a condition which is modulated by DDR1 and/or DDR2. A compound of any formula disclosed herein may be for use in the treatment of a condition treatable by the inhibition of DDR1 and/or DDR2.

In another aspect of the invention, there is provided a compound of the present invention for use in the treatment of a disease or disorder selected from: renal conditions, liver conditions, inflammatory conditions, cardiovascular conditions, acute and chronic organ transplant rejection, fibrotic diseases and cancer.

In an aspect of the invention there is provided a method of treating a disease or disorder which is modulated by DDR1 and/or DDR2 wherein the method comprises administering a therapeutic amount of a compound of the invention, to a patient in need thereof.

The method of treatment may be a method of treating a condition treatable by the inhibition of DDR1 and/or DDR2.

The invention also provides a method of treating a disease or disorder selected from: renal conditions, liver conditions, inflammatory conditions, cardiovascular conditions, acute and chronic organ transplant rejection, fibrotic diseases and cancer wherein the method comprises administering a therapeutic amount of a compound of any formula disclosed herein, to a patient in need thereof.

Renal conditions include, acute kidney injury and chronic renal disease with and without proteinuria including end-stage renal disease (ESRD). This includes decreased creatinine clearance and decreased glomerular filtration rate, micro albuminuria, albuminuria and proteinuria, glomerulosclerosis with expansion of reticulated mesangial matrix with or without significant hypercellularity (particularly diabetic nephropathy and amyloidosis), focal thrombosis of glomerular capillaries (particularly thrombotic microangiopathies), global fibrinoid necrosis, ischemic lesions, malignant nephrosclerosis (such as ischemic retraction, reduced renal blood flow and renal arteriopathy), swelling and proliferation of intracapillary (endothelial and mesangial) and/or extracapillary cells (crescents) like in glomerular nephritis entities, focal segmental glomerular sclerosis, IgA nephropathy, vasculitis/systemic diseases as well as acute and chronic kidney transplant rejection. Early and advanced Alport syndrome are also included amongst renal conditions.

Inflammatory conditions include, arthritis, osteoarthritis, multiple sclerosis, systemic lupus erythematodes, inflammatory bowel disease, abnormal evacuation disorder and the like as well as inflammatory airways diseases such as idiopathic pulmonary fibrosis (IPF), chronic obstructive pulmonary disease (COPD) or chronic asthma. Further conditions of the respiratory system include other diffuse parenchymal lung diseases of different etiologies including iatrogenic drug-induced fibrosis, occupational and/or environmental induced fibrosis, systemic diseases and vasculitis, granulomatous diseases (sarcoidosis, hypersensitivity pneumonia), collagen vascular disease, radiation induced fibrosis.

Vascular conditions include atherosclerosis, thrombotic vascular disease as well as thrombotic microangiopathies, proliferative arteriopathy (such as swollen myointimal cells surrounded by mucinous extracellular matrix and nodular thickening), atherosclerosis, decreased vascular compliance (such as stiffness, reduced ventricular compliance and reduced vascular compliance), endothelial dysfunction and the like.

Cardiovascular conditions include acute coronary syndrome, coronary heart disease, myocardial infarction, arterial and pulmonary hypertension, cardiac arrhythmia such as atrial fibrillation, stroke and other vascular damage.

Fibrotic diseases include, but are not limited to myocardial and vascular fibrosis, renal fibrosis, liver fibrosis, pulmonary fibrosis, skin fibrosis, scleroderma and encapsulating peritonitis, systemic sclerosis, Alport syndrome, Chronic kidney disease, NASH, Interstitial lung diseases and Systemic Sclerosis.

In certain embodiments compounds of the invention are for use in the treatment of or are used in a method of treatment of cancer. Examples include but are not limited to: liver cancer, bladder cancer, hepatoma, squamous carcinoma of the lung, non-small cell lung cancer, adenocarcinoma of the lung, small-cell lung cancer, various types of head and neck cancer, breast cancer, colon cancer, colorectal cancer, cancer of the peritoneum, hepatocellular cancer, gastrointestinal cancer, esophageal cancer, endometrial or uterine carcinoma, salivary gland carcinoma, squamous cell cancer, pituitary cancer, astrocytoma, soft tissue sarcoma, pancreatic cancer, glioblastoma, cervical cancer, ovarian cancer, kidney cancer, liver cancer, prostate cancer, vulval cancer, thyroid cancer, hepatic carcinoma, brain cancer, endometrial cancer, testis cancer, cholangiocarcinoma, gallbladder carcinoma, gastric cancer and melanoma. In certain embodiments, the cancer is selected from bladder cancer, pancreatic cancer, breast cancer, lung cancer, ovarian cancer and glioblastoma.

In another aspect of the invention there is provided a pharmaceutical composition, wherein the composition comprises a compound of the invention and pharmaceutically acceptable excipients.

In an embodiment the pharmaceutical composition may be a combination product comprising an additional pharmaceutically active agent.

In an aspect of the present invention there is provided the use of a compound of the invention in the manufacture of a medicament for use in the treatment of any condition disclosed herein.

### DETAILED DESCRIPTION

Given below are definitions of terms used in this application. Any term not defined herein takes the normal meaning as the skilled person would understand the term.

The term “halo” refers to one of the halogens, group 17 of the periodic table. In particular, the term refers to fluorine, chlorine, bromine and iodine. Preferably, the term refers to chlorine or fluorine.

The term “alkyl” refers to a linear or branched hydrocarbon chain. For example, the term “C₁₋₆ alkyl” refers to a linear or branched hydrocarbon chain containing 1, 2, 3, 4, 5 or 6 carbon atoms, for example methyl, ethyl, n-propyl, iso-propyl, n-butyl, sec-butyl, tert-butyl, n-pentyl and n-hexyl. “Alkylene” groups may likewise be linear or branched and is divalent, i.e. it attached at two positions to other portions of the molecule. Furthermore, an alkylene group may, for example, correspond to one of those alkyl groups listed in this paragraph. The alkyl and alkylene groups may be unsubstituted or substituted by one or more substituents.

The term “haloalkyl” refers to a hydrocarbon chain substituted with at least one halogen atom independently chosen at each occurrence, for example fluorine, chlorine, bromine and iodine. For example, the term “C₁₋₆ haloalkyl” refers to a linear or branched hydrocarbon chain containing 1, 2, 3, 4, 5 or 6 carbon atoms substituted with at least one halogen. The halogen atom may be present at any position on the hydrocarbon chain. For example, C₁₋₆ haloalkyl may refer to chloromethyl, fluoromethyl, trifluoromethyl, chloroethyl e.g. 1-chloromethyl and 2-chloroethyl, trichloroethyl e.g. 1,2,2-trichloroethyl, 2,2,2-trichloroethyl, fluoroethyl e.g. 1-fluoromethyl and 2-fluoroethyl, trifluoroethyl e.g. 1,2,2-trifluoroethyl and 2,2,2-trifluoroethyl, chloropropyl, trichloropropyl, fluoropropyl, trifluoropropyl. The term “fluoroalkyl” refers to a hydrocarbon chain substituted with at least one fluorine atom.

The term “alkenyl” refers to a branched or linear hydrocarbon chain containing at least one double bond. For example, the term “C₂₋₆ alkenyl” refers to a branched or linear hydrocarbon chain containing at least one double bond and having 2, 3, 4, 5 or 6 carbon atoms. The double bond(s) may be present as the E or Z isomer. The double bond may be at any possible position of the hydrocarbon chain. For example, the “C₂₋₆ alkenyl” may be ethenyl, propenyl, butenyl, butadienyl, pentenyl, pentadienyl, hexenyl and hexadienyl.

The term “alkynyl” refers to a branched or linear hydrocarbon chain containing at least one triple bond. For example, the term “C₂₋₆ alkynyl” refers to a branched or linear hydrocarbon chain containing at least one triple bond and having 2, 3, 4, 5 or 6 carbon atoms. The triple bond may be at any possible position of the hydrocarbon chain. For example, the “C₂₋₆ alkynyl” may be ethynyl, propynyl, butynyl, pentynyl and hexynyl.

The term “heteroalkyl” refers to a branched or linear hydrocarbon chain containing at least one heteroatom selected from N, O and S positioned between any carbon in the chain or at an end of the chain. For example, the term “C₁₋₆ heteroalkyl” refers to a branched or linear hydrocarbon chain containing 1, 2, 3, 4, 5, or 6 carbon atoms and at least one heteroatom selected from N, O and S positioned between any carbon in the chain or at an end of the chain. For example, the hydrocarbon chain may contain one or two heteroatoms. The C₁₋₆ heteroalkyl may be bonded to the rest of the molecule through a carbon or a heteroatom. For example, the “C₁₋₆ heteroalkyl” may be C₁₋₆ N-alkyl, C₁₋₆ N,N-alkyl, or C₁₋₆ O-alkyl.

The term “heterocycle” refers to a saturated, unsaturated or aromatic ring system containing at least one heteroatom selected from N, O or S. A “heterocyclic” system may contain 1, 2, 3 or 4 heteroatoms, for example 1 or 2. A “heterocyclic” system may be monocyclic or a fused polycyclic ring system, for example, bicyclic or tricyclic. A “heterocyclic” moiety may contain from 3 to 14 carbon atoms, for example, 3 to 8 carbon atoms in a monocyclic system and 7 to 14 carbon atoms in a polycyclic system. “Heterocyclic” encompasses heterocycloalkyl moieties, heterocycloalkenyl moieties and heteroaryl moieties. For example, the heterocyclic group may be: oxirane, aziridine, azetidine, oxetane, tetrahydrofuran, pyrrolidine, imidazolidine, succinimide, pyrazolidine, oxazolidine, isoxazolidine, thiazolidine, isothiazolidine, piperidine, morpholine, thiomorpholine, piperazine, and tetrahydropyran. Heteroaryl includes groups such as pyridones and N-alkyl-pyridones.

The term “C₃₋₈ cycloalkyl” refers to a saturated hydrocarbon ring system containing 3, 4, 5, 6, 7 or 8 carbon atoms. For example, the “C₃₋₈ cycloalkyl” may be cyclopropyl, cyclobutyl, cyclopentyl, cyclohexyl, cycloheptyl and cyclooctyl.

The term “C₃₋₈ cycloalkenyl” refers to an unsaturated hydrocarbon ring system containing 3, 4, 5, 6, 7 or 8 carbon atoms that is not aromatic. The ring may contain more than one double bond provided that the ring system is not aromatic. For example, the “C₃₋₈ cycloalkyl” may be cyclopropenyl, cyclobutenyl, cyclopentenyl, cyclopentadienyl, cyclohexenyl, cyclohexadienly, cycloheptenyl, cycloheptadiene, cyclooctenyl and cycloatadienyl.

The term “heterocycloalkyl” refers to a saturated hydrocarbon ring system containing carbon atoms and at least one heteroatom within the ring selected from N, O and S. For example, there may be 1, 2 or 3 heteroatoms, optionally 1 or 2. The “heterocycloalkyl” may be bonded to the rest of the molecule through any carbon atom or heteroatom. The “heterocycloalkyl” may have one or more, e.g. one or two, bonds to the rest of the molecule: these bonds may be through any of the atoms in the ring. For example, the “heterocycloalkyl” may be a “C₃₋₈ heterocycloalkyl”. The term “C₃₋₈ heterocycloalkyl” refers to a saturated hydrocarbon ring system containing 3, 4, 5, 6, 7 or 8 atoms at least one of the atoms being a heteroatom within the ring selected from N, O and S. The “heterocycloalkyl” may be oxirane, aziridine, azetidine, oxetane, tetrahydrofuran, pyrrolidine, imidazolidine, succinimide, pyrazolidine, oxazolidine, isoxazolidine, thiazolidine, isothiazolidine, piperidine, morpholine, thiomorpholine, piperazine, and tetrahydropyran.

The term “aromatic” when applied to a substituent as a whole means a single ring or polycyclic ring system with 4n+2 electrons in a conjugated π system within the ring or ring system where all atoms contributing to the conjugated π system are in the same plane.

The term “aryl” refers to an aromatic hydrocarbon ring system. The ring system has 4n+2 electrons in a conjugated π system within a ring where all atoms contributing to the conjugated π system are in the same plane. For example, the “aryl” may be phenyl and naphthyl. The aryl system itself may be substituted with other groups.

The term “heteroaryl” refers to an aromatic hydrocarbon ring system with at least one heteroatom within a single ring or within a fused ring system, selected from O, N and S. The ring or ring system has 4n+2 electrons in a conjugated π system where all atoms contributing to the conjugated π system are in the same plane. For example, the “heteroaryl” may be imidazole, oxazole, isoxazole, thiazole, isothiazole, thiene, furan, thianthrene, pyrrole, benzimidazole, pyrazole, pyrazine, pyridine, pyrimidine and indole.

A bond terminating in a “ [symbol] ” represents that the bond is connected to another atom that is not shown in the structure. A bond terminating inside a cyclic structure and not terminating at an atom of the ring structure represents that the bond may be connected to any of the atoms in the ring structure where allowed by valency.

A bond drawn as a solid line and a dotted line represents a bond which can be either a single bond or a double bond, where chemically possible. For example, the bond drawn below could be a single bond or a double bond.

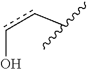

Where a moiety is substituted, it may be substituted at any point on the moiety where chemically possible and consistent with atomic valency requirements. The moiety may be substituted by one or more substituents, e.g. 1, 2, 3 or 4 substituents; optionally there are 1 or 2 substituents on a group. Where there are two or more substituents, the substituents may be the same or different.

Substituents are only present at positions where they are chemically possible, the person skilled in the art being able to decide (either experimentally or theoretically) without inappropriate effort which substitutions are chemically possible and which are not.

Ortho, meta and para substitution are well understood terms in the art. For the absence of doubt, “ortho” substitution is a substitution pattern where adjacent carbons possess a substituent, whether a simple group, for example the fluoro group in the example below, or other portions of the molecule, as indicated by the bond ending in “ [symbol] ”

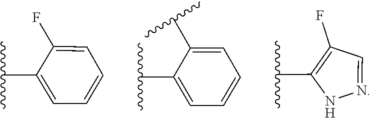

“Meta” substitution is a substitution pattern where two substituents are on carbons one carbon removed from each other, i.e with a single carbon atom between the substituted carbons. In other words there is a substituent on the second atom away from the atom with another substituent. For example the groups below are meta substituted.

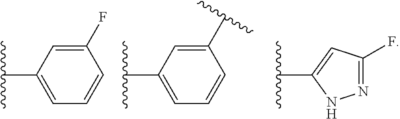

“Para” substitution is a substitution pattern where two substituents are on carbons two carbons removed from each other, i.e with two carbon atoms between the substituted carbons. In other words there is a substituent on the third atom away from the atom with another substituent. For example the groups below are para substituted.

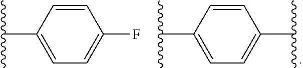

Throughout the description the disclosure of a compound also encompasses pharmaceutically acceptable salts, solvates and stereoisomers thereof.

Where a compound has a stereocentre, both (R) and (S) stereoisomers are contemplated by the invention, equally mixtures of stereoisomers or a racemic mixture are completed by the present application. Where a compound of the invention has two or more stereocentres any combination of (R) and (S) stereoisomers is contemplated. The combination of (R) and (S) stereoisomers may result in a diastereomeric mixture or a single diastereoisomer. The compounds of the invention may be present as a single stereoisomer or may be mixtures of stereoisomers, for example racemic mixtures and other enantiomeric mixtures, and diasteroemeric mixtures. Where the mixture is a mixture of enantiomers the enantiomeric excess may be any of those disclosed above. Where the compound is a single stereoisomer the compounds may still contain other diasteroisomers or enantiomers as impurities. Hence a single stereoisomer does not necessarily have an enantiomeric excess (e.e.) or diastereomeric excess (d.e.) of 100% but could have an e.e. or d.e. of about at least 85%, at least 60% or less. For example, the e.e. or d.e. may be 90% or more, 90% or more, 80% or more, 70% or more, 60% or more, 50% or more, 40% or more, 30% or more, 20% or more, or 10% or more.

The invention contemplates pharmaceutically acceptable salts of the compounds of the invention. These may include the acid addition and base salts of the compounds. These may be acid addition and base salts of the compounds. In addition the invention contemplates solvates of the compounds. These may be hydrates or other solvated forms of the compound.

Suitable acid addition salts are formed from acids which form non-toxic salts. Examples include the acetate, aspartate, benzoate, besylate, bicarbonate/carbonate, bisulfate/sulfate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 1,5-naphthalenedisulfonate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulfate and hemicalcium salts. For a review on suitable salts, see “Handbook of Pharmaceutical Salts: Properties, Selection, and Use” by Stahl and Wermuth (Wiley-VCH, Weinheim, Germany, 2002).

Pharmaceutically acceptable salts of compounds of formula (I) may be prepared by one or more of three methods:
(i) by reacting the compound of the invention with the desired acid or base;
(ii) by removing an acid- or base-labile protecting group from a suitable precursor of the compound of the invention or by ring-opening a suitable cyclic precursor, for example, a lactone or lactam, using the desired acid or base; or
(iii) by converting one salt of the compound of the invention to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of the invention may exist in both unsolvated and solvated forms. The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

Included within the scope of the invention are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionised, partially ionised, or non-ionised. For a review of such complexes, see J Pharm Sci, 64 (8), 1269-1288 by Haleblian (August 1975).

Hereinafter all references to compounds of any formula include references to salts, solvates and complexes thereof and to solvates and complexes of salts thereof.

The compounds of the invention include compounds of a number of formula as herein defined, including all polymorphs and crystal habits thereof, prodrugs and isomers thereof (including optical, geometric and tautomeric isomers) as hereinafter defined and isotopically-labelled compounds of the invention.

The present invention also includes all pharmaceutically acceptable isotopically-labelled compounds of the invention wherein one or more atoms are replaced by atoms having the same atomic number, but an atomic mass or mass number different from the atomic mass or mass number most commonly found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Before purification, the compounds of the present invention may exist as a mixture of enantiomers depending on the synthetic procedure used. The enantiomers can be separated by conventional techniques known in the art. Thus the invention covers individual enantiomers as well as mixtures thereof.

For some of the steps of the process of preparation of the compounds of the invention, it may be necessary to protect potential reactive functions that are not wished to react, and to cleave said protecting groups in consequence. In such a case, any compatible protecting radical can be used. In particular methods of protection and deprotection such as those described by T. W. GREENE (Protective Groups in Organic Synthesis, A. Wiley-Interscience Publication, 1981) or by P. J. Kocienski (Protecting groups, Georg Thieme Verlag, 1994), can be used. All of the above reactions and the preparations of novel starting materials used in the preceding methods are conventional and appropriate reagents and reaction conditions for their performance or preparation as well as procedures for isolating the desired products will be well-known to those skilled in the art with reference to literature precedents and the examples and preparations hereto.

Also, the compounds of the present invention as well as intermediates for the preparation thereof can be purified according to various well-known methods, such as for example crystallization or chromatography.

One or more compounds of the invention may be combined with one or more pharmaceutical agents, for example anti-inflammatory agents, anti-fibrotic agents, chemotherapeutics, anti-cancer agents, immunosuppressants, anti-tumour vaccines, cytokine therapy, or tyrosine kinase inhibitors, for the treatment of conditions modulated by the inhibition of DDR, for example fibrotic diseases, auto-immune, inflammatory-fibrotic conditions, inflammatory conditions, central nervous system disorders, or cancer.

The method of treatment or the compound for use in the treatment of renal conditions, liver conditions, inflammatory conditions, cardiovascular conditions, acute and chronic organ transplant rejection, fibrotic diseases and cancer as defined hereinbefore may be applied as a sole therapy or be a combination therapy with an additional active agent.

The method of treatment or the compound for use in the treatment of renal conditions, liver conditions, inflammatory conditions, cardiovascular conditions, acute and chronic organ transplant rejection, fibrotic diseases and cancer. The additional active agents may be one or more active agents used to treat the condition being treated by the compound of the invention and additional active agent.

The additional active agents may include one or more of the following active agents:—
(i) steroids such as corticosteroids, including glucocorticoids and mineralocorticoids, for example aclometasone, aclometasone dipropionate, aldosterone, amcinonide, beclomethasone, beclomethasone dipropionate, betamethasone, betamethasone dipropionate, betamethasone sodium phosphate, betamethasone valerate, budesonide, clobetasone, clobetasone butyrate, clobetasol propionate, cloprednol, cortisone, cortisone acetate, cortivazol, deoxycortone, desonide, desoximetasone, dexamethasone, dexamethasone sodium phosphate, dexamethasone isonicotinate, difluorocortolone, fluclorolone, flumethasone, flunisolide, fluocinolone, fluocinolone acetonide, fluocinonide, fluocortin butyl, fluorocortisone, fluorocortolone, fluocortolone caproate, fluocortolone pivalate, fluorometholone, fluprednidene, fluprednidene acetate, flurandrenolone, fluticasone, fluticasone propionate, halcinonide, hydrocortisone, hydrocortisone acetate, hydrocortisone butyrate, hydrocortisone aceponate, hydrocortisone buteprate, hydrocortisone valerate, icomethasone, icomethasone enbutate, meprednisone, methylprednisolone, mometasone paramethasone, mometasone furoate monohydrate, prednicarbate, prednisolone, prednisone, tixocortol, tixocortol pivalate, triamcinolone, triamcinolone acetonide, triamcinolone alcohol and their respective pharmaceutically acceptable derivatives. A combination of steroids may be used, for example a combination of two or more steroids mentioned in this paragraph;
(ii) TNF inhibitors for example etanercept; monoclonal antibodies (e.g. infliximab (Remicade), adalimumab (Humira), certolizumab pegol (Cimzia), golimumab (Simponi)); fusion proteins (e.g. etanercept (Enbrel)); and 5-HT_{2A} agonists (e.g. 2,5-dimethoxy-4-iodoamphetamine, TCB-2, lysergic acid diethylamide (LSD), lysergic acid dimethylazetidide);
(iii) anti-inflammatory drugs, for example non-steroidal anti-inflammatory drugs;
(iv) dihydrofolate reductase inhibitors/antifolates, for example methotrexate, trimethoprim, brodimoprim, tetroxoprim, iclaprim, pemetrexed, ralitrexed and pralatrexate; and
(v) immunosuppressants for example cyclosporins, tacrolimus, sirolimus pimecrolimus, angiotensin II inhibitors (e.g. Valsartan, Telmisartan, Losartan, Irbesatan, Azilsartan, Olmesartan, Candesartan, Eprosartan) and ACE inhibitors e.g. sulfhydryl-containing agents (e.g. Captopril, Zofenopril), dicarboxylate-containing agents (e.g. Enalapril, Ramipril, Quinapril, Perindopril, Lisinopril, Benazepril, Imidapril, Zofenopril, Trandolapril), phosphate-containing agents (e.g. Fosinopril), casokinins, lactokinins and lactotripeptides.
(vi) Anti-fibrotic agents for example: Pirfenidone, Nintedanib, Anti-IL-13 monoclonal antibodies (e.g. Tralokinumab, QAX576, Lebrikizumab), simtuzumab, FG-3019, lysophosphatidic acid receptor antagonists (e.g. BMS-986020, AM966), LOXL2 inhibitors, BET bromodomain inhibitors (e.g. JQ1), HDAC inhibitors (e.g. Vorinostat), thrombin inhibitors (e.g. Dabigatran), FactorXa inhibitors (e.g. Apixban, Rivaroxaban) 15PGDH inhibitors, anti-avP6 monoclonal antibodies (e.g. BG00011), Anti-CTGF monoclonal antibodies (e.g. FG-3019), PAR1 inhibitors, Nox4 inhibitors and PAI-1 inhibitors.

The method of treatment or the compound for use in the treatment of cancer may involve, in addition to the compound of the invention, conventional surgery or radiotherapy or chemotherapy. Such chemotherapy may include one or more of the following categories of anti-tumor agents:
(i) antiproliferative/antineoplastic drugs and combinations thereof, such as alkylating agents (for example cis-platin, oxaliplatin, carboplatin, cyclophosphamide, nitrogen mustard, uracil mustard, bendamustin, melphalan, chlorambucil, chlormethine, busulphan, temozolamide, nitrosoureas, ifosamide, melphalan, pipobroman, triethylene-melamine, triethylenethiophoporamine, carmustine, lomustine, stroptozocin and dacarbazine); antimetabolites (for example gemcitabine and antifolates such as fluoropyrimidines like 5-fluorouracil and tegafur, raltitrexed, methotrexate, pemetrexed, cytosine arabinoside, floxuridine, cytarabine, 6-mercaptopurine, 6-thioguanine, fludarabine phosphate, pentostatine, and gemcitabine and hydroxyurea); antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); proteasome inhibitors, for example carfilzomib and bortezomib; interferon therapy; and topoisomerase inhibitors (for example epipodophyllotoxins like etoposide and teniposide, amsacrine, topotecan, mitoxantrone and camptothecin); bleomcin, dactinomycin, daunorubicin, doxorubicin, epirubicin, idarubicin, ara-C, paclitaxel (Taxol™), nabpaclitaxel, docetaxel, mithramycin, deoxyco-formycin, mitomycin-C, L-asparaginase, interferons (especially IFN-a), etoposide, and teniposide;
(ii) cytostatic agents such as antiestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride; and navelbene, CPT-II, anastrazole, letrazole, capecitabine, reloxafme, cyclophosphamide, ifosamide, and droloxafine;
(iii) anti-invasion agents, for example dasatinib and bosutinib (SKI-606), and metalloproteinase inhibitors, inhibitors of urokinase plasminogen activator receptor function or antibodies to Heparanase;
(iv) inhibitors of growth factor function: for example such inhibitors include growth factor antibodies and growth factor receptor antibodies, for example the anti-erbB2 antibody trastuzumab [Herceptin™], the anti-EGFR antibody panitumumab, the anti-erbB1 antibody cetuximab, tyrosine kinase inhibitors, for example inhibitors of the epidermal growth factor family (for example EGFR family tyrosine kinase inhibitors such as gefitinib, erlotinib, 6-acrylamido-N-(3-chloro-4-fluorophenyl)-7-(3-morpholinopropoxy)-quinazolin-4-amine (Cl 1033), erbB2 tyrosine kinase inhibitors such as lapatinib) and antibodies to costimulatory molecules such as CTLA-4, 4-IBB and PD-I, or antibodies to cytokines (IL-10, TGF-beta); inhibitors of the hepatocyte growth factor family; inhibitors of the insulin growth factor family; modulators of protein regulators of cell apoptosis (for example Bcl-2 inhibitors); inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib, tipifarnib and lonafarnib), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor, kinase inhibitors; aurora kinase inhibitors and cyclin dependent kinase inhibitors such as CDK2 and/or CDK4 inhibitors; and CCR2, CCR4 or CCR6 modulator;
(v) antiangiogenic agents such as those which inhibit the effects of vascular endothelial growth factor, for example the anti-vascular endothelial cell growth factor antibody bevacizumab (Avastin™); thalidomide; lenalidomide; and for example, a VEGF receptor tyrosine kinase inhibitor such as vandetanib, vatalanib, sunitinib, axitinib and pazopanib;
(vi) gene therapy approaches, including for example approaches to replace aberrant genes such as aberrant p53 or aberrant BRCA1 or BRCA2;
(vii) immunotherapy approaches, including for example antibody therapy such as alemtuzumab, rituximab, ibritumomab tiuxetan (Zevalin®) and ofatumumab; interferons such as interferon α; interleukins such as IL-2 (aldesleukin); interleukin inhibitors for example IRAK4 inhibitors; cancer vaccines including prophylactic and treatment vaccines such as HPV vaccines, for example Gardasil, Cervarix, Oncophage and Sipuleucel-T (Provenge); gp100; dendritic cell-based vaccines (such as Ad.p53 DC); and toll-like receptor modulators for example TLR-7 or TLR-9 agonists; and
(viii) cytotoxic agents for example fludaribine (fludara), cladribine, pentostatin (Nipent™);
(ix) steroids such as corticosteroids, including glucocorticoids and mineralocorticoids, for example aclometasone, aclometasone dipropionate, aldosterone, amcinonide, beclomethasone, beclomethasone dipropionate, betamethasone, betamethasone dipropionate, betamethasone sodium phosphate, betamethasone valerate, budesonide, clobetasone, clobetasone butyrate, clobetasol propionate, cloprednol, cortisone, cortisone acetate, cortivazol, deoxycortone, desonide, desoximetasone, dexamethasone, dexamethasone sodium phosphate, dexamethasone isonicotinate, difluorocortolone, fluclorolone, flumethasone, flunisolide, fluocinolone, fluocinolone acetonide, fluocinonide, fluocortin butyl, fluorocortisone, fluorocortolone, fluocortolone caproate, fluocortolone pivalate, fluorometholone, fluprednidene, fluprednidene acetate, flurandrenolone, fluticasone, fluticasone propionate, halcinonide, hydrocortisone, hydrocortisone acetate, hydrocortisone butyrate, hydrocortisone aceponate, hydrocortisone buteprate, hydrocortisone valerate, icomethasone, icomethasone enbutate, meprednisone, methylprednisolone, mometasone paramethasone, mometasone furoate monohydrate, prednicarbate, prednisolone, prednisone, tixocortol, tixocortol pivalate, triamcinolone, triamcinolone acetonide, triamcinolone alcohol and their respective pharmaceutically acceptable derivatives. A combination of steroids may be used, for example a combination of two or more steroids mentioned in this paragraph;
(x) targeted therapies, for example PI3Kd inhibitors, for example idelalisib and perifosine; PD-1, PD-L1, PD-L2 and CTL4-A modulators, antibodies and vaccines; other IDO inhibitors (such as indoximod); anti-PD-1 monoclonal antibodies (such as MK-3475 and nivolumab); anti-PD-L1 monoclonal antibodies (such as MEDI-4736 and RG-7446); anti-PD-L2 monoclonal antibodies; and anti-CTLA-4 antibodies (such as ipilimumab);
(xii) chimeric antigen receptors, anticancer vaccines and arginase inhibitors.

Such combination treatment may be achieved by way of the simultaneous, sequential or separate dosing of the individual components of the treatment. Such combination products employ the compounds of this invention within a therapeutically effective dosage range described hereinbefore and the other pharmaceutically-active agent within its approved dosage range.

Compounds of the invention may exist in a single crystal form or in a mixture of crystal forms or they may be amorphous. Thus, compounds of the invention intended for pharmaceutical use may be administered as crystalline or amorphous products. They may be obtained, for example, as solid plugs, powders, or films by methods such as precipitation, crystallization, freeze drying, or spray drying, or evaporative drying. Microwave or radio frequency drying may be used for this purpose.

For the above-mentioned compounds of the invention the dosage administered will, of course, vary with the compound employed, the mode of administration, the treatment desired and the disorder indicated. For example, if the compound of the invention is administered orally, then the daily dosage of the compound of the invention may be in the range from 0.01 micrograms per kilogram body weight (μg/kg) to 100 milligrams per kilogram body weight (mg/kg).

A compound of the invention, or pharmaceutically acceptable salt thereof, may be used on their own but will generally be administered in the form of a pharmaceutical composition in which the compounds of the invention, or pharmaceutically acceptable salt thereof, is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration of the compounds of the invention, the pharmaceutical composition which is used to administer the compounds of the invention will preferably comprise from 0.05 to 99% w (percent by weight) compounds of the invention, more preferably from 0.05 to 80% w compounds of the invention, still more preferably from 0.10 to 70% w compounds of the invention, and even more preferably from 0.10 to 50% w compounds of the invention, all percentages by weight being based on total composition.

The pharmaceutical compositions may be administered topically (e.g. to the skin) in the form, e.g., of creams, gels, lotions, solutions, suspensions, or systemically, e.g. by oral administration in the form of tablets, capsules, syrups, powders or granules; or by parenteral administration in the form of a sterile solution, suspension or emulsion for injection (including intravenous, subcutaneous, intramuscular, intravascular or infusion); by rectal administration in the form of suppositories; or by inhalation in the form of an aerosol.

For oral administration the compounds of the invention may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, the compounds of the invention may be admixed with, for example, a vegetable oil or polyethylene glycol. Hard gelatine capsules may contain granules of the compound using either the above-mentioned excipients for tablets. Also liquid or semisolid formulations of the compound of the invention may be filled into hard gelatine capsules. Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, sweetening agents (such as saccharine), preservative agents and/or carboxymethylcellulose as a thickening agent or other excipients known to those skilled in art.

For intravenous (parenteral) administration the compounds of the invention may be administered as a sterile aqueous or oily solution.

The size of the dose for therapeutic purposes of compounds of the invention will naturally vary according to the nature and severity of the conditions, the age and sex of the animal or patient and the route of administration, according to well-known principles of medicine.

Dosage levels, dose frequency, and treatment durations of compounds of the invention are expected to differ depending on the formulation and clinical indication, age, and co-morbid medical conditions of the patient.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

The compounds of the invention may be prepared according to or analogously to General Schemes 1 to 4. Alternatively, compounds of the invention may be prepared according to or analogously to the Examples 1 to 105.

### EXAMPLES AND SYNTHESIS

### Experimental Procedures

Solvents, reagents and starting materials were purchased from commercial vendors and used as received unless otherwise described. All reactions were performed at room temperature unless otherwise stated. Compound identity and purity confirmations were performed by LCMS UV using a Waters Acquity SQ Detector 2 (ACQ-SQD2 #LCA081). The diode array detector wavelength was set to aquire spectra at a wavelength of 254 nM and the MS was in positive and negative electrospray mode (m/z: 150-800). A 2 μL aliquot was injected onto a guard column (0.2 μm×2 mm filters) and UPLC column (C18, 50×2.1 mm, <2 μm) in sequence maintained at 40° C. The samples were eluted at a flow rate of 0.6 mL/min with a mobile phase system composed of A (0.1% (v/v) Formic Acid in Water) and B (0.1% (v/v) Formic Acid in Acetonitrile) according to the gradients outlined in Table 1 below (Methods 1 and 2). Retention times RT are reported in minutes. The following methods were also used on occasions when described throughout the experimental section, gradients are detailed in table 1. Method 3 utilised a Shimadzu 2020 series spectrometer equipped with a binary pump and diode array detector (acquisition wavelength 214 and 254 nm) and the MS was in positive and negative electrospray mode (m/z: 100-900). 2 μL Aliquot were injected onto an Agilent Poroshell 120 EC-C18 column (2.7 μm, 4.6×50 mm) maintained at 35° C. and eluted at 1.0 ml/min using mobile phase consisting of: A: 0.05% Formic acid in water (v/v), B: 0.05% Formic acid in ACN(v/v). Method 4 utilised a Agilent Technologies 1290 series spectrometer equipped with a binary pump and diode array detector (acquisition wavelength 214 and 254 nm) and the MS was in positive electrospray mode (m/z: 70-1000). 2 μL Aliquots were injected onto an Agilent Eclipse Plus RRHD C18, (1.8 μm, 3.0×50 mm) column maintained at 40° C. and eluted at 0.8 ml/min using mobile phase consisting of: A: 0.05% Formic acid in water (v/v), B: 0.05% Formic acid in ACN(v/v).

TABLE 1

| | | |
|---|---|---|
| Time (min) | % A | % B |

Method 1 (Long acidic)

| | | |
|---|---|---|
| 0 | 95 | 5 |
| 1.1 | 95 | 5 |
| 6.1 | 5 | 95 |
| 7 | 5 | 95 |
| 7.5 | 95 | 5 |
| 8 | 95 | 5 |

Method 2 (Short acidic)

| | | |
|---|---|---|
| 0 | 95 | 5 |
| 0.3 | 95 | 5 |
| 2 | 5 | 95 |
| 2.6 | 95 | 5 |
| 3 | 95 | 5 |

Method 3

| | | |
|---|---|---|
| 0.01 | 85 | 15 |
| 1.0 | 85 | 15 |
| 4.0 | 0 | 100 |
| 4.5 | 0 | 100 |
| 4.51 | 85 | 15 |
| 5.0 | 85 | 15 |

Method 4

| | | |
|---|---|---|
| 0.00 | 80 | 20 |
| 2.65 | 20 | 80 |
| 3.00 | 20 | 80 |

NMR was also used to characterise final compounds. NMR spectra were obtained on a Bruker AVIII 400 Nanobay with 5 mm BBFO probe. Optionally, compound Rf values on silica thin layer chromatography (TLC) plates were measured.

Compound purification was performed by flash column chromatography on silica or by preparative LCMS. LCMS purification was performed using a Waters 3100 Mass detector in positive and negative electrospray mode (m/z: 150-800) with a Waters 2489 UV/Vis detector. Samples were eluted at a flow rate of 20 mL/min on a XBridge™ prep C18 5 μM OBD 19×100 mm column with a mobile phase system composed of A (0.1% (v/v) Formic Acid in Water) and B (0.1% (v/v) Formic Acid in Acetonitrile) according to the gradient outlined in Table 2 below.

TABLE 2

| Time (min) | % A | % B |
|---|---|---|
| 0 | 90 | 10 |
| 1.5 | 90 | 10 |
| 11.7 | 5 | 95 |
| 13.7 | 5 | 95 |
| 14 | 90 | 90 |
| 15 | 90 | 90 |

### General Routes and Schemes

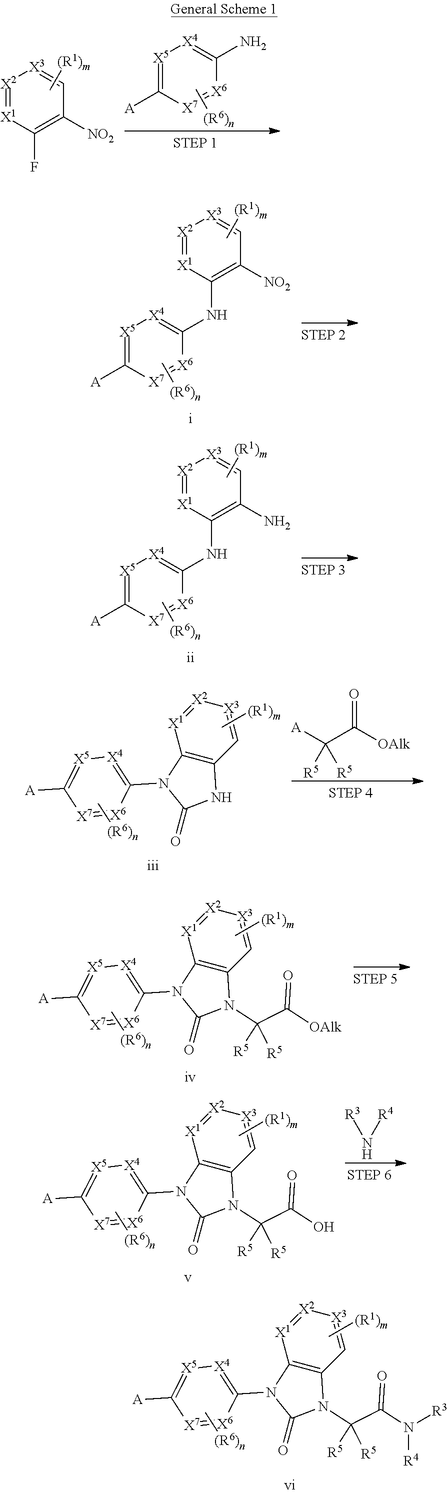

Compounds of formula (I) can be prepared form intermediates represented by vi in general scheme 1. In Step 1 an ortho-fluoronitroaryl compound undergoes a nucleophilic displacement reaction with an aryl or heteroarylamine (where A represents a halogen such as Cl, Br or I capable of participating in a metal mediated cross-coupling reaction such as a Suzuki or Stille reaction), in the presence of a base such as sodium hydride, to afford compounds of structure i. In step 2 the nitro group of i can be reduced by transition metal catalysis in the presence of hydrogen gas or using an alternative source of hydrogen such as ammonium chloride to produce compounds of structure ii. In step 3 reaction with a carbonyl equivalent (for example CDI) affords cyclized products represented by iii. In step 4 the free NH of iii is reacted with an appropriate α-halo ester, where A is a halogen such as Br and OAlk represents an alkoxy group such as ethoxide which is subsequently hydrolysed to the corresponding carboxylic acid in step 5 under basic conditions with KOH or LiOH, affording compounds of structure v. Reaction with an appropriate amine in step 6 using a carboxylic acid activating reagent such as HATU and a base such as DIPEA affords intermediates represented by vi.

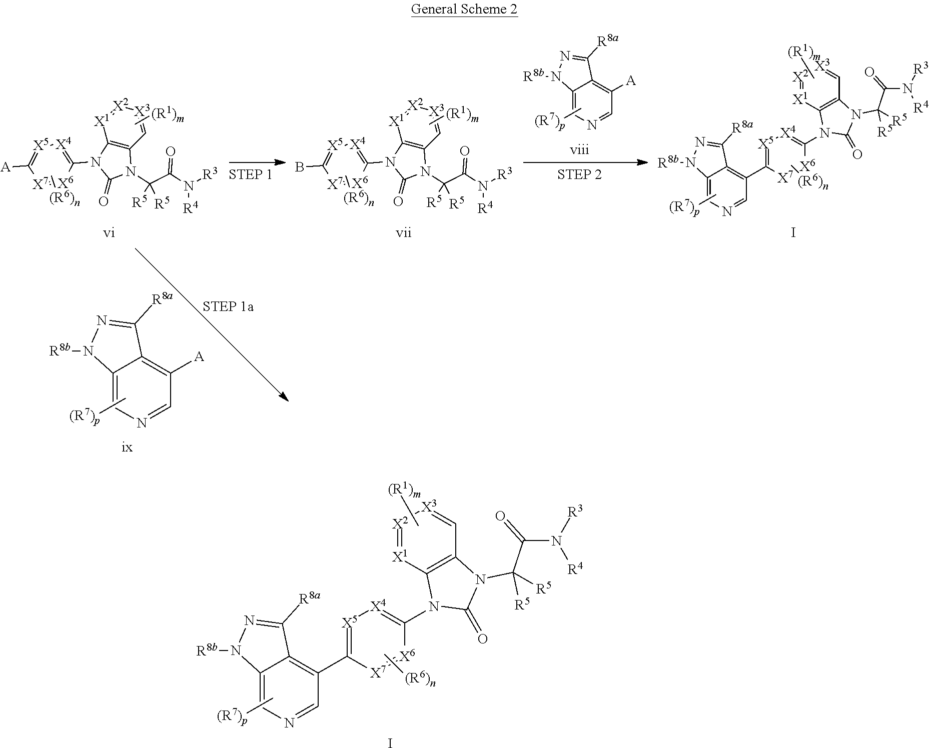

Compounds of formula I can be synthesised from intermediates represented by general structure vi via the two routes described in general scheme 2. According to step 1a, vi (in which A represents a halogen such as Br or another leaving group capable of participating in a metal catalysed cross-coupling reaction such as a triflate) undergoes transition metal catalysed cross-coupling with an azaindazole of structure ix in which B is a boronic acid, boronate ester or stannyl group (trialkyl tin) capable of participating in a Suzuki or Stille type reaction with a compound of structure vi. As an example A may be a boronic acid pinacol ester and B may be a bromide and these groups in step 1a undergo a Suzuki reaction catalysed by Pd(dppf)Cl₂-DCM in the presence of KOAc to afford a compound of formula I. Alternatively, according to step 1 the intermediate of structure vi can be converted into a boronate or stannane of formula vii via reaction with bis(pinacolato)diboron or tributyltin chloride respectively (where B is a boronic acid, boronate ester or stannyl group (trialkyl tin) and undergo subsequent Suzuki or Stille reaction in Step 2 with an azaindazole of structure viii where A is a halogen such as Br, Cl or I, affording compounds of formula (I).

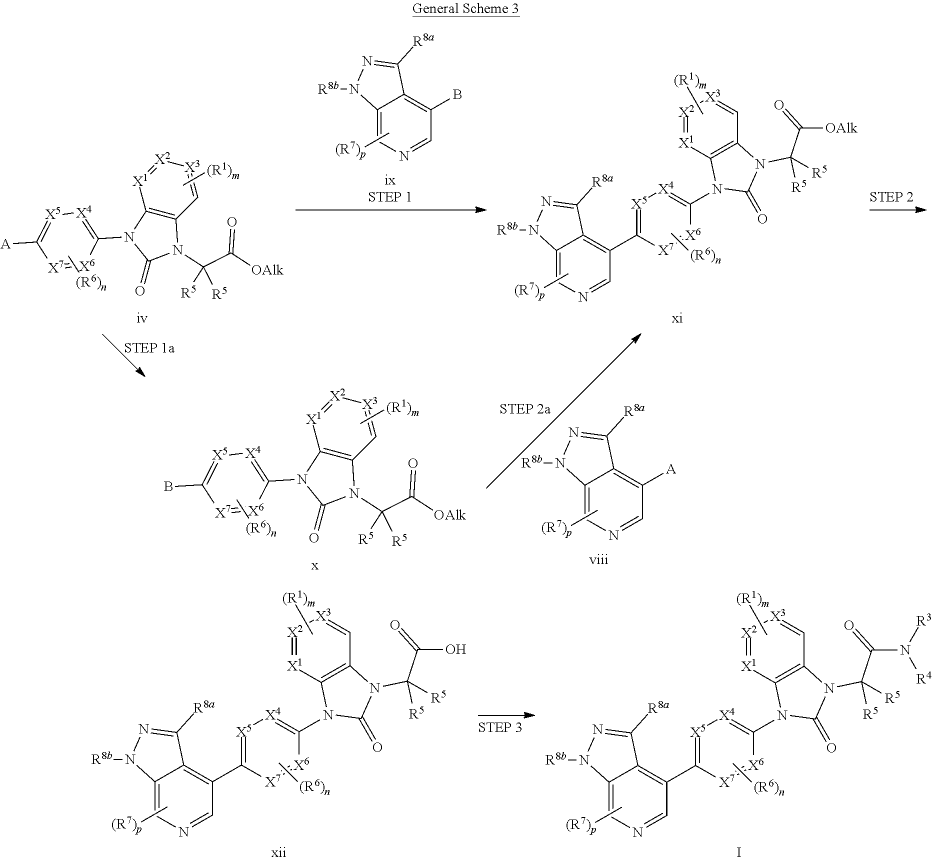

Compounds of formula (I) may also be accessed by re-ordering the steps from general schemes 1 and 2 as depicted in general scheme 3. The definitions of groups A, B and OAlk in structures shown in general scheme 3 are the same as those described for general schemes 1 and 2. In step 1 intermediates of structure iv undergo metal catalysed cross-coupling reactions such as Suzuki or Stille reactions with boronate or stannane derivatives of azaindazoles represented by ix. In step 2 the cross-coupled products (xi) undergo base mediated ester hydrolysis as previously described and then amide coupling in step 3 to afford compounds of formula (I). Alternatively the cross-coupling partners can be reversed by converting halide or triflate iv, to the corresponding boronate or stannane in step 1a and reacting this with a haloazaindazole of structure viii in step 2b to afford intermediates of structure xi.

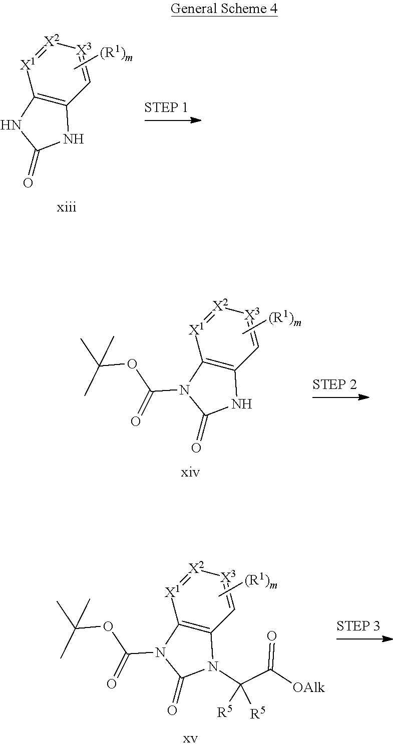

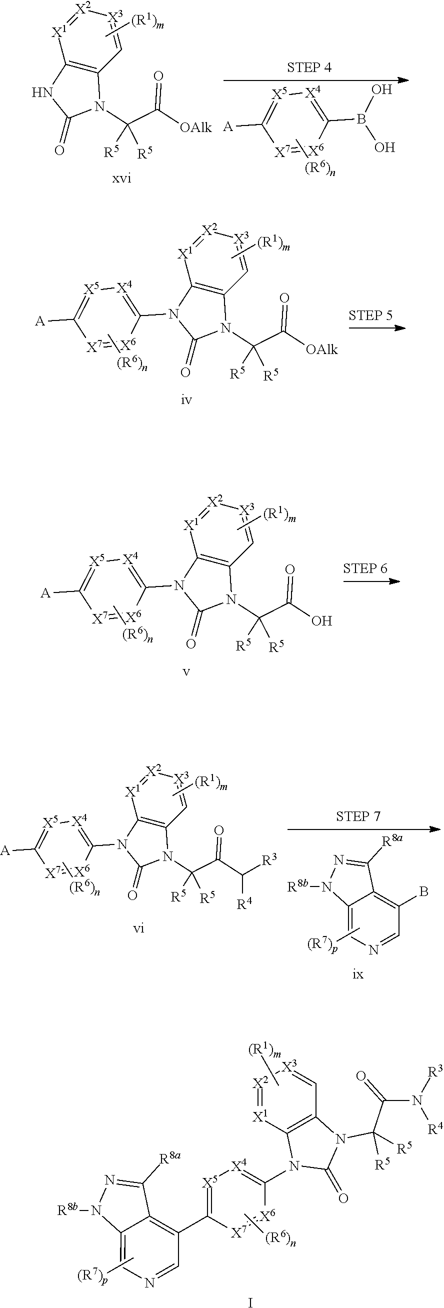

General scheme 4 outlines an alternative route to prepare intermediates of structure iv which are then transformed into intermediate vi (general Scheme 1) which can then be converted into compounds of formula (I) via the methods described in general scheme 2. In step 1 an appropriate benzimidazolone or azabenzimidazolone derivative (xiii) is mono-Boc protected using NaH and ditertbutyldicarbonate. In step 2 the remaining free NH of xiv is alkylated with an appropriate α-haloester. In step 3 the boc protecting group of xv is removed using TFA to allow for Chan-Lam type coupling of xvi and an appropriate aryl boronic acid using copper (II) acetate as a catalyst and air as a source of oxygen in step 4 to afford intermediates of structure iv.

In general schemes 2, 3 and 4 where the azaindazole substituent R^{8b} of compounds of formula I is H, steps 1a and 2 (general scheme 2) and steps 1 and 1a (general scheme 3) can be carried out using a 2-tetrayhdropyran group as a protecting group at the R^{8b} position. In these cases a final deprotection step may be required to remove the tetrahydropyran group and form the compounds with R^{8b}=H. The deprotection step can be carried out by reaction with hydrogen chloride in an organic solvent such as 1,4-dioxane. Furthermore, in cases where experimental procedures are not described below in the synthesis of intermediates, azaindazole derivatives as represented in general schemes 1-4 were prepared by methods previously described in publicly accessible scientific literature.

### Synthesis of Intermediates

### Synthesis of Intermediate I-4, ethyl 2-[3-(4-bromophenyl)-2-oxobenzimidazol-1-yl]acetate

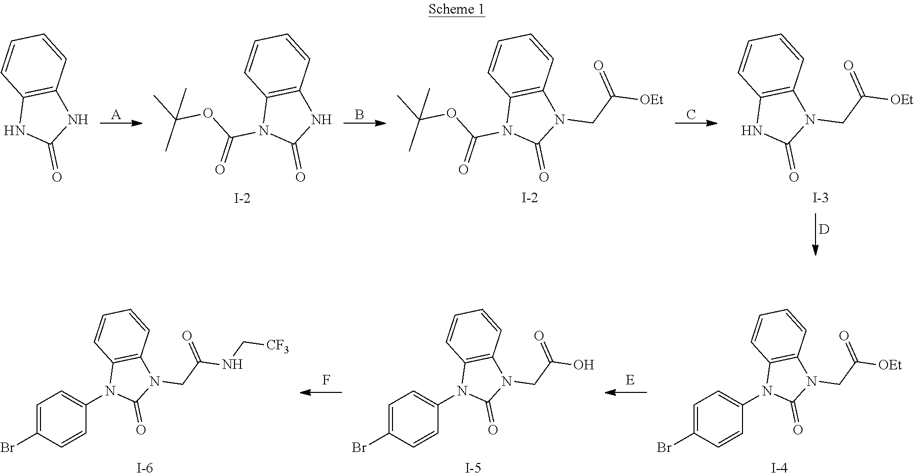

STEP A. Synthesis of Intermediate I-1, tert-butyl 2-oxo-3H-benzimidazole-1-carboxylate: To a solution of 2-hydroxybenzimidazole (3 g, 22 mmol) in DMF (90 mL) was added sodium hydride, 60% dispersed in mineral oil (900 mg, 22.5 mmol) and the reaction mixture was stirred at room temperature for 2 h. A solution of (BOC)₂O (4.9 g, 22 mmol) in DMF (10 mL) was added dropwise. The reaction mixture was stirred at room temperature for 16 h then it was evaporated to dryness. The residue was diluted with ethyl acetate and washed with saturated aqueous. NH₄Cl (2×30 mL). The layers were separated and the aqueous phase was extracted with ethyl acetate (2×30 mL). The organic layers were washed with brine (2×30 mL) then dried with Na₂SO₄ and evaporated to dryness to afford tert-butyl 2-oxo-3H-benzimidazole-1-carboxylate (Intermediate I-1, 5.7 g, 24 mmol, 100% yield) as an off-white solid. UPLC-MS (ES⁺, Method 2): 1.73 min, m/z 233.2 [M−H]⁻. ¹H NMR (400 MHz, DMSO-d₆): δ 11.24 (sbr, 1H), 7.64 (ddd, 0.5, 1.2, 8.0 Hz, 1H), 7.12 (dt, J=1.2, 7.6 Hz, 1H), 7.05 (dt, J=1.4, 7.8 Hz, 1H), 6.99 (ddd, J=0.5, 1.4, 7.7 Hz, 1H), 1.59 (s, 9H) ppm.

STEP B. Synthesis of Intermediate I-2, tert-butyl 3-(2-ethoxy-2-oxo-ethyl)-2-oxobenzimidazole-1-carboxylate: A mixture of Cs₂CO₃ (9.5 g, 29 mmol), tert-butyl 2-oxo-3H-benzimidazole-1-carboxylate (Intermediate I-1, 5.2 g, 22 mmol) and ethyl bromoacetate (2.7 mL, 25 mmol) in MeCN (120 mL) was heated to 80° C. and stirred for 2 h. The mixture was cooled to room temperature and the solvent was removed in vacuo. The residue was suspended in brine (50 mL) and H₂O (60 mL) and extracted with EtOAc (3×50 mL). The combined organic layers were dried with Na₂SO₄ and the solvent removed in vacuo. The crude product was dry loaded onto silica and purified by normal phase flash chromatography (SiO₂, 0 to 20% EtOAc in pet. ether) to give tert-butyl 3-(2-ethoxy-2-oxoethyl)-2-oxobenzimidazole-1-carboxylate (Intermediate I-2, 6.1 g, 19 mmol, 85% yield) as a yellow oil. UPLC-MS (ES⁺, Method 2): 1.90 min, m/z 321.1 [M+H]⁺. ¹H NMR (400 MHz, CDCl₃): δ 7.85 (dd, J=1.7, 7.5 Hz, 1H), 7.18 (dt, J=1.5, 7.6 Hz, 1H), 7.14 (dt, J=1.6, 7.7 Hz), 6.85 (dd, J=1.3, 7.7 Hz, 1H), 4.59 (s, 2H), 4.23 (q, J=7.15, 2H), 1.67 (s, 9H), 1.26 (t, J=7.14 Hz, 3H) ppm.

STEP C. Synthesis of Intermediate I-3, ethyl 2-(2-oxo-3H-benzimidazol-1-yl)acetate: To a solution of tert-butyl 3-(2-ethoxy-2-oxoethyl)-2-oxobenzimidazole-1-carboxylate (Intermediate I-2, 6.1 g, 19 mmol) in DCM (90 mL) was added trifluoroacetic acid (14.6 mL, 190 mmol) dropwise and the mixture was stirred at room temperature for 2 h. The reaction was quenched with saturated aqueous Na₂CO₃ (50 mL). The layers were separated and the aqueous layer was extracted with DCM (3×50 mL). The combined organic layers were dried with Na₂SO₄ and the solvent was removed in vacuo to give ethyl 2-(2-oxo-3H-benzimidazol-1-yl)acetate (Intermediate I-3, 3.7 g, 17 mmol, 89% yield) as a white solid, which was used in the next step without further purification. UPLC-MS (ES⁺, Method 2): 1.51 min, m/z 221.2 [M+H]⁺¹H NMR (400 MHz, DMSO-d₆): δ 10.95 (s, 1H), 7.06-7.11 (m, 1H), 6.96-7.03 (m, 3H), 4.66 (s, 2H), 4.15 (q, J=7.1 Hz, 2H), 1.21 (t, J=7.1 Hz, 3H) ppm.

STEP D. Synthesis of Intermediate I-4, ethyl 2-[3-(4-bromophenyl)-2-oxobenzimidazol-1-yl]acetate: In a round-bottom flask with a mounted condenser open to air 4-bromobenzeneboronic acid (4.1 g, 20 mmol), ethyl 2-(2-oxo-3H-benzimidazol-1-yl)acetate (Intermediate I-3, 2.2 g, 10 mmol) and copper (II) acetate (3.7 g, 20 mmol) were suspended in MeCN (90 mL). Et₃N (4.2 mL, 30 mmol) was added and the suspension was stirred at room temperature for 16 h. The solvent was removed in vacuo. The residue was dissolved in DCM (50 mL) and washed with H₂O (50 mL) and sat. aq. NH₄Cl (50 mL). The aqueous layer was extracted with DCM (3×50 mL). The combined organic layers were dried with Na₂SO₄ and concentrated in vacuo. The crude product was dry loaded onto silica and purified by phase flash chromatography (25 g SiO₂, 5 to 90% EtOAc in pet. ether) to give ethyl 2-[3-(4-bromophenyl)-2-oxobenzimidazol-1-yl]acetate (Intermediate I-4, 3.4 g, 9.2 mmol, 90% yield) as a white solid. UPLC-MS (ES⁺, Method 2): 1.88 min, m/z 375.0/376.9 [M+H]+. ¹H NMR (400 MHz, DMSO-d₆): δ 7.79 (td, J=2.5, 9.6, 2H), 7.54 (td, J=2.5, 9.6, 2H), 7.30 (d, J=7.6 Hz, 1H), 7.07-7.20 (m, 3H), 4.82 (s, 2H), 4.19 (q, J=7.1 Hz, 2H), 1.24 (t, J=7.1 Hz, 3H) ppm.

STEP E. Synthesis of Intermediate I-5, 2-[3-(4-bromophenyl)-2-oxobenzimidazol-1-yl]acetic acid: A suspension of ethyl 2-[3-(4-bromophenyl)-2-oxobenzimidazol-1-yl]acetate (Intermediate I-4, 3.3 g, 8.9 mmol) and KOH (1.5 g, 27 mmol) in ethanol (140 mL) was heated to 80° C. and stirred for 2 h. The reaction mixture was evaporated to dryness. The residue was dissolved in H₂O and slowly acidified by dropwise addition of 1 M HCl to pH 2-3, which generated a white precipitate which was collected by filtration to give 2-[3-(4-bromophenyl)-2-oxobenzimidazol-1-yl]acetic acid (Intermediate I-5, 3.0 g, 8.6 mmol, 97% yield) as white solid. UPLC-MS (ES⁺, Method 2): 1.63 min, m/z 346.9/348.9 [M+H]+¹H NMR (400 MHz, DMSO-d₆): δ 13.19 (s, 1H), 7.78 (td, J=2.5, 9.7 Hz, 2H), 7.54 (td, J=2.6, 9.7, 2H), 7.29 (dbr, J=7.6 Hz, 1H), 7.06-7.18 (m, 3H), 4.69 (s, 2H) ppm.

STEP F. Synthesis of Intermediate I-6, 2-[3-(4-bromophenyl)-2-oxobenzimidazol-1-yl]-N-(2,2,2-trifluoro-ethyl)acetamide: To a stirred solution of 2-[3-(4-bromophenyl)-2-oxo-benzimidazol-1-yl]acetic acid (Intermediate I-5, 1.7 g, 4.9 mmol) and trifluoroethylamine (0.5 mL, 6 mmol) in DMF (30 mL) were added HATU (2.2 g, 6 mmol) and DIPEA (1.7 mL, 9.8 mmol) and the mixture was stirred at room temperature for 2 h. The mixture was poured into brine (50 mL) and DCM (50 mL). The layers were separated and the aqueous layer was extracted with DCM, the combined organic layers were washed with sat. aq. NH₄Cl (3×30 mL) and brine, dried with Na₂SO₄, and concentrated in vacuo. The crude product was purified by flash chromatography (40 g SiO₂, 5 to 90% EtOAc in pet. ether) to give 2-[3-(4-bromophenyl)-2-oxobenzimidazol-1-yl]-N-(2,2,2-trifluoroethyl)acetamide (Intermediate I-6, 1.8 g, 4.1 mmol, 85% yield) as an off-white solid. UPLC-MS (ES⁺, Method 2): 1.86 min, m/z 428.0/429.9 [M+H]⁺. ¹H NMR (400 MHz, DMSO-d₆): δ 8.97 (t, J=6.3 Hz, 1H), 7.79 (td, J=2.5, 9.6 Hz, 2H), 7.55 (td, J=2.5, 9.6 Hz, 2H), 7.06-7.17 (m, 4H), 4.66 (s, 2H), 3.97 (dq, J=6.3, 9.8 Hz, 2H) ppm.

Intermediates synthesised following the same procedure as Intermediate I-6 (scheme 1) replacing 4-bromobenzeneboronic acid in Step 0 and/or trifluoroethylamine in Step F for the described building block are described in Table 3.

TABLE 3

| | Intermediate structure and | | ¹H-NMR (400 MHz, |
|---|---|---|---|
| Building blocks | name | LC-MS (ES⁺) | DMSO-d₆) |
| Step F: (2S)-2-trifluoro- methylpyrrolidine | 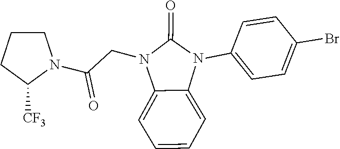 [SMILES: O=C(Cn1c(=O)n(-c2ccc(Br)cc2)c2ccccc21)N1CCC[C@H]1C(F)(F)F] | Method 4: 1.814 min, m/z, 468.0 [M + H]⁺ | δ 7.78 (d, J = 8.7 Hz, 2H), 7.54 (d, J = 8.7 Hz, 2H), 7.18 (t, J = 7.8 Hz, 1H), 7.16-7.03 (m, 3H), 4.88 (q, J = 12.8 Hz, 1H), 4.74 (mc, 1H), 3.78 (mc, 1H), 2.69 (s, 2H), 2.28-1.95 (m, 3H), 1.21 (t, J = 6.0 Hz, 1H) ppm. |
| | 1-(4-bromophenyl)-3-[2-oxo-2- | | |
| | [(2S)-2-(trifluoromethyl)pyrrolidin- | | |
| | 1-yl]ethyl]47enzimidazole-2-one | | |
| | Intermediate I-7 | | |
| Step F: (2S)-2-amino- 1,1,1- trifluoropropane hydrochloride | 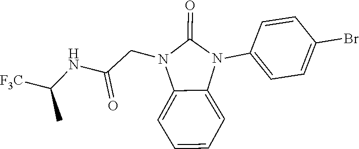 [SMILES: C[C@H](NC(=O)Cn1c(=O)n(-c2ccc(Br)cc2)c2ccccc21)C(F)(F)F] | Method 4: 1.98 min, m/z 442.0 [M + H]⁺ | δ 8.89 (d, J = 8.8 Hz, 1H), 7.77 (d, J = 8.8 Hz, 2H), 7.54 (d, J = 8.7 Hz, 2H), 7.18-7.05 (m, 4H), 4.63 (s, 2H), 4.60 (mc, 1H), 1.28 (d, J = 7.2 Hz, 3H) ppm. |
| | 2-[3-(4-bromophenyl)-2-oxo- | | |
| | benzimidazol-1-yl]-N-[(1S)-2,2,2- | | |
| | trifluoro-1-methylethyl]acetamide | | |
| | Intermediate 1-8 | | |

### Synthesis of Intermediate I-11, 2-[3-(4-bromo-3-fluorophenyl)-2-oxobenzimidazol-1-yl]-N-(2,2,2-trifluoroethyl)acetamide

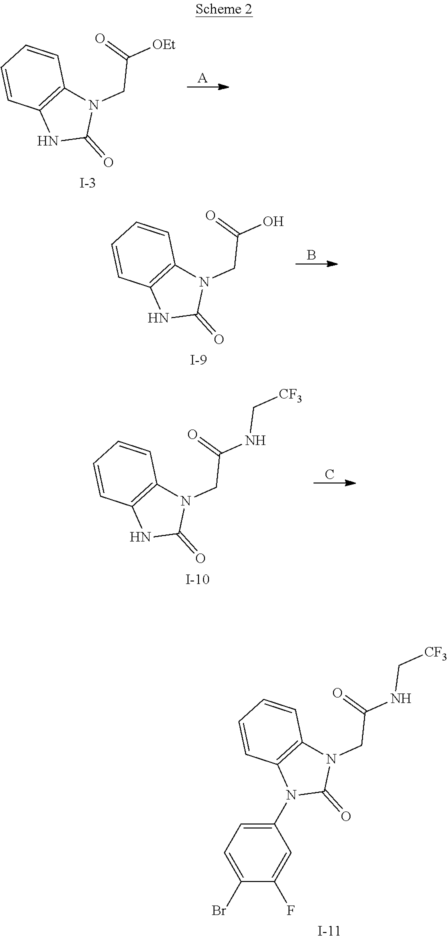

STEP A. Synthesis of Intermediate I-9, 2-(2-oxo-3H-benzimidazol-1-yl)acetic acid: A solution of tert-butyl 3-(2-eth oxy-2-oxoethyl)-2-oxo-2,3-dihydro-1H-benzo[d]imidazole-1-carboxylate (Intermediate I-3, 2.00 g, 6.25 mmol) and LiOH·H₂O (1.31 g, 31.2 mmol) in THE (10 mL) and H₂O (10 mL) was stirred at 25° C. for 3 h. The mixture was treated with HCl (1 M) (35 mL), was diluted with water and extracted with EtOAc (×3). The combined organic layers were washed with brine, dried with Na₂SO₄ and concentrated in vacuo to give 2-(2-oxo-3H-benzimidazol-1-yl)acetic acid (Intermediate I-9, 1.08 g, 5.62 mmol, 90% yield) as a white solid. LC-MS (ES⁺, Method 3): 4.10 min, m/z 193.15 [M+H]⁺¹H NMR (400 MHz, DMSO-d₆): δ 10.93 (s, 1H), 7.12 (mc, 1H), 7.06-7.01 (m, 2H), 4.59 (s, 2H) ppm.

STEP B. Synthesis of Intermediate I-10, 2-(2-oxo-3H-benzimidazol-1-yl)-N-(2,2,2-trifluoroethyl)acetamide: A mixture of 2-(2-oxo-2,3-dihydro-1H-benzimidazol-1-yl)acetic acid (I-9, 1.00 g, 5.20 mmol), 2,2,2-trifluoroethan-1-amine (567 mg, 5.7 mmol), EDCI (970 mg, 6.24 mmol), HOBT (843 mg, 6.24 mmol) and DIPEA (2.0 g, 15.6 mmol) in DMF (15 mL) was stirred at 25° C. overnight. The mixture was diluted with EtOAc and washed with water (×3). The combined organic layers were washed with brine, dried with Na₂SO₄ and concentrated in vacuo to give 2-(2-oxo-3H-benzimidazol-1-yl)-N-(2,2,2-trifluoroethyl)acetamide (Intermediate I-10, 1.00 g, 3.66 mmol, 70% yield). LC-MS (ES⁺, Method 4): 0.66 min, m/z, 274.0 [M+H]⁺. ¹H NMR (400 MHz, DMSO-d₆): δ 10.90 (s, 1H), 8.90 (t, J=6.2 Hz, 1H), 7.05-6.94 (m, 4H), 4.53 (s, 2H), 3.96 (dq, J=6.3, 9.7 Hz, 2H) ppm.

STEP C. Synthesis of Intermediate I-11, 2-[3-(4-bromo-3-fluorophenyl)-2-oxobenzimidazol-1-yl]-N-(2,2,2-trifluoroethyl)acetamide: A solution of ethyl 2-(2-oxo-3H-benzimidazol-1-yl)-N-(2,2,2-trifluoro-ethyl)acetamide (Intermediate I-10, 420 mg, 1.54 mmol), (4-bromo-3-fluoro-phenyl)boronic acid (480 mg, 2.19 mmol), NEt₃ (445 mg, 4.40 mmol), Cu(OAc)₂ (533 mg, 2.93 mmol) in MeCN (12 mL) was stirred 25° C. under N₂ overnight. The reaction was concentrated to dryness and the residue was taken up in EtOAc. The organic layer was washed with saturated aqueous NH₄Cl (×2) and brine (×2), dried with Na₂SO₄, concentrated in vacuo and purified by column chromatography (SiO₂, EtOAc in pet. ether, 5%-10%) to give 2-[3-(4-bromo-3-fluoro-phenyl)-2-oxobenzimidazol-1-yl]-N-(2,2,2-trifluoroethyl)acetamide (Intermediate I-11, 280 mg, 0.63 mmol, 29%). LC-MS (ES⁺, Method 4): 2.033 min, m/z, 447.95 [M+H]⁺. ¹H NMR (400 MHz, DMSO-d₆): δ 8.97 (t, J=6.2 Hz, 1H), 7.95 (t, J=8.3 Hz, 1H), 7.69 (dd, J=2.4, 10.0 Hz, 1H), 7.45 (dd, J=1.7, 8.6 Hz, 1H), 7.23 (d, J=7.6 Hz, 1H), 7.21-7.09 (m, 3H), 4.68 (s, 2H), 3.99 (dq, J=6.5, 9.8 Hz, 2H) ppm.

Intermediates synthesised following the same procedure as Intermediate I-11 (scheme 2) replacing (4-bromo-3-fluoro-phenyl)boronic acid for the described building block in Step C are described in Table 4.

TABLE 4

| | Intermediate structure and | | ¹H-NMR (400 MHz, |
|---|---|---|---|
| Building blocks | name | LC-MS (ES⁺) | DMSO-d₆) |
| (4-bromo-3- methylphenyl)boronic acid | 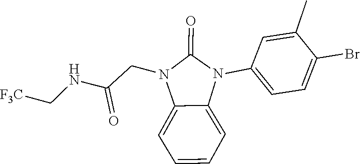 [SMILES: Cc1cc(-n2c(=O)n(CC(=O)NCC(F)(F)F)c3ccccc32)ccc1Br] | Method 4: 1.73 min, m/z 442/444 [M + H]⁺ | δ 8.95 (t, J = 6.2 Hz, 1H), 7.79 (d, J = 8.3 Hz, 1H), 7.64 (s, 1H), 7.38 (d, J = 8.6 Hz, 1H), 7.15-7.08 (m, 4H), 4.65 (s, 2H), 4.01-3.92 (m, 2H), 2.43 (s, 3H) ppm. |
| | 2-[3-(4-bromo-3-methyl-phenyl)- | | |
| | 2-oxobenzimidazol-1-yl]-N- | | |
| | (2,2,2-trifluoroethyl)acetamide | | |
| | Intermediate I-12 | | |
| (4-bromo-2- chlorophenyl)boronic acid | 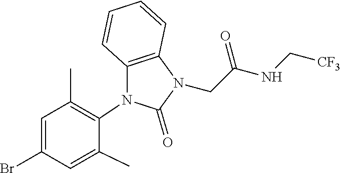 | Method 4: 1.93 min, m/z 461/463 [M + H]⁺ | |
| | 2-[3-(4-bromo-2-fluoro-phenyl)- | | |
| | 2-oxobenzimidazol-1-yl]-N- | | |
| | (2,2,2-trifluoroethyl)acetamide | | |
| | Intermediate 1-58 | | |
| (4-bromo-2,6- dimethylphenyl)boronic acid | 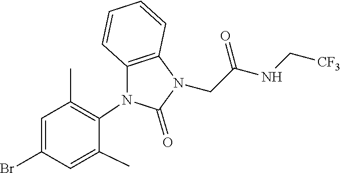 [SMILES: Cc1cc(Br)cc(C)c1-n1c(=O)n(CC(=O)NCC(F)(F)F)c2ccccc21] | Method 3: 4.22 min, m/z 456/458 [M + H]⁺ | |
| | 2-[3-(4-bromo-2,6-dimethyl- | | |
| | phenyl)-2-oxo-benzimidazol-1- | | |
| | yl]-N-(2,2,2- | | |
| | trifluoroethyl)acetamide | | |
| | Intermediate I-61 | | |
| | 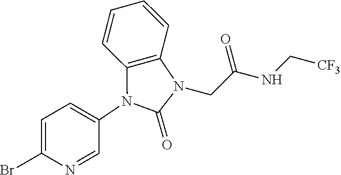 [SMILES: O=C(Cn1c(=O)n(-c2ccc(Br)nc2)c2ccccc21)NCC(F)(F)F] | Method 4: 1.59 min, m/z, 431/433 [M + H]⁺ | δ 8.97 (m, 1H), 8.67 (s, 1H), 8.03 (d, 1H) 7.89 (d, 1H), 7.22-7.09 (m, 4H), 4.67 (s, 2H), 3.97 (m, 2H) ppm. |
| | 2-[3-(6-bromo-3-pyridyl)-2-oxo- | | |
| | benzimidazol-1-yl]-N-(2,2,2- | | |
| | trifluoroethyl)acetamide | | |
| | Intermediate I-62 | | |

### Synthesis of Intermediate I-18, 2-[3-(4-bromophenyl)-4-methyl-2-oxobenzimidazol-1-yl]-N-(2,2,2-trifluoroethyl)acetamide

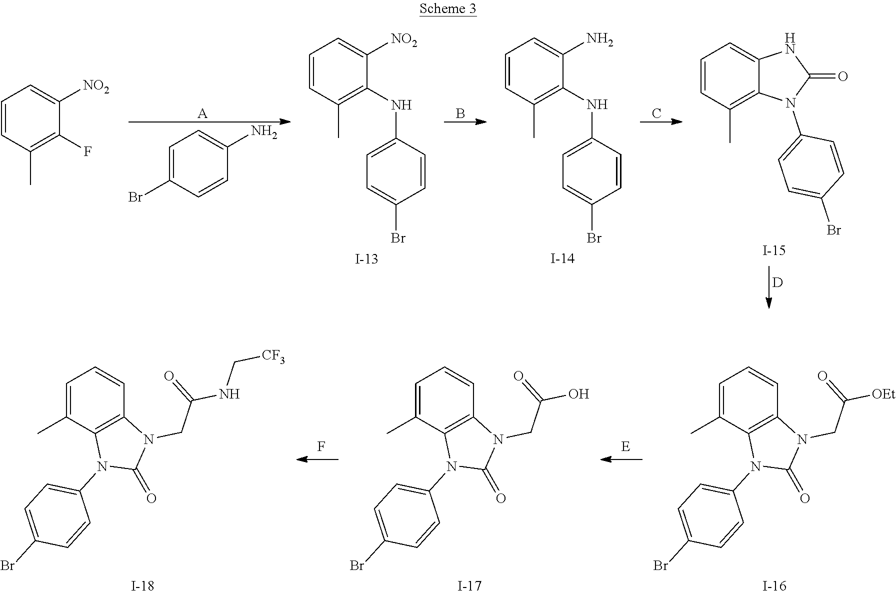

STEP A. Synthesis of Intermediate I-13, N-(4-bromophenyl)-2-methyl-6-nitroaniline: NaH (6.2 g, 261 mmol) was added to a solution of 4-bromoaniline (30 g, 174 mmol) in dry DMF (150 mL) at 0° C. under N₂. The mixture was stirred at that temperature for 30 min, then 2-fluoro-1-methyl-3-nitrobenzene (32.5 g, 209 mmol) was added and the mixture was stirred at room temperature overnight. The mixture was diluted with EtOAc (1.0 L) and washed with water (3×1.0 L) and brine (500 mL), dried with Na₂SO₄ and concentrated in vacuo. The crude product was purified by flash chromatography (120 g SiO₂, 1% EtOAc in pet. ether) to give N-(4-bromophenyl)-2-methyl-6-nitro-aniline (Intermediate I-13, 39.4 g, 86 mmol, 49% yield) as a white solid. ¹H NMR (400 MHz, DMSO-d₆): δ 8.03 (s, 1H), 7.70 (d, J=8.3 Hz, 1H), 7.63 (t, J=7.6 Hz, 1H), 7.54 (d, J=7.7 Hz, 1H), 7.20 (d, J=8.8 Hz, 2H), 6.43 (d, J=8.7 Hz, 2H), 2.09 (s, 3H) ppm.

STEP B. Synthesis of Intermediate I-14, N2-(4-bromophenyl)-3-methylphenyl-1,2-diamine: A mixture of N-(4-bromophenyl)-2-methyl-6-nitroaniline (Intermediate I-13, 34 g, 0.11 mol), Fe (31 g, 0.56 mol) and NH₄Cl (30 g, 0.56 mol) in EtOH (600 mL) and water (200 mL) was stirred at 80° C. for 2 h. The mixture was filtered through filter paper, washing with MeOH, and concentrated in vacuo to give N2-(4-bromophenyl)-3-methylphenyl-1,2-diamine (Intermediate I-14, 18.2 g, 66 mmol, 59% yield) as a brown oil. UPLC-MS (ES⁺, Method 2): 1.86 min, m/z 277.0 [M+H]⁺

STEP C. Synthesis of Intermediate I-15, 3-(4-bromophenyl)-4-methyl-1H-benzimidazol-2-one: A mixture of N2-(4-bromophenyl)-3-methylphenyl-1,2-diamine (Intermediate I-14, 3.7 g, 13.4 mmol) and CDI (6.5 g, 40 mmol) in DMF (40 mL) was stirred at 100° C. under N₂ atmosphere for 16 h. The mixture was diluted with EtOAc (200 mL) and was washed with water (3×100 mL) and brine (100 mL), dried with Na₂SO₄ and concentrated in vacuo to give 3-(4-bromophenyl)-4-methyl-1H-benzimidazol-2-one (Intermediate I-15, 5 g, 13 mmol, 98% yield) as a white solid which was used in the next step without further purification. UPLC-MS (ES⁺, Method 2): 2.17 min, m/z 303.0 [M+H]⁺. ¹H NMR (400 MHz, DMSO-d₆): δ 7.18 (s, 1H), 6.91 (d, J=8.4 Hz, 2H), 6.55 (d, J=8.6 Hz, 2H), 6.23-6.16 (m, 2H), 6.02 (d, J=6.8 Hz, 1H), 1.07 (s, 3H) ppm.

STEP D. Synthesis of Intermediate I-16, ethyl 2-[3-(4-bromophenyl)-4-methyl-2-oxobenzimidazol-1-yl]acetate: A mixture of 3-(4-bromophenyl)-4-methyl-1H-benzimidazol-2-one (Intermediate I-15, 4 g, 13 mmol), ethyl 2-bromoacetate (3.3 g, 20 mmol) and Cs₂CO₃ (8.6 g, 26 mmol) in MeCN (60 mL) was stirred at room temperature for 2 h. The mixture was concentrated in vacuo, water (200 mL) and EtOAc (200 mL) were added and the layers were separated. The aqueous layer was extracted with EtOAc (2×200 mL), the combined organic layers were washed with brine (100 mL), dried with Na₂SO₄ and concentrated in vacuo to give ethyl 2-[3-(4-bromophenyl)-4-methyl-2-oxobenzimidazol-1-yl]acetate (Intermediate I-16, 4.8 g, 10 mmol, 75% yield) as an off-white solid which was used in the next step without further purification. UPLC-MS (ES⁺, Method 2): 2.65 min, m/z 389.1 [M+H]⁺. ¹H NMR (400 MHz, DMSO-d₆): δ 7.76 (d, J=8.6 Hz, 2H), 7.43 (d, J=8.7 Hz, 2H), 7.13 (d, J=7.7 Hz, 1H), 7.05 (t, J=7.8 Hz, 1H), 6.89 (d, J=7.8 Hz, 1H), 4.78 (s, 2H), 4.19 (q, J=7.5 Hz, 2H), 1.85 (s, 3H), 1.24 (t, J=7.5 Hz, 3H) ppm.

STEP E. Synthesis of Intermediate I-17, 2-[3-(4-bromophenyl)-4-methyl-2-oxobenzimidazol-1-yl]acetic acid: A mixture of ethyl 2-[3-(4-bromophenyl)-4-methyl-2-oxobenzimidazol-1-yl]acetate (Intermediate I-16, 300 mg, 0.77 mmol) and LiOH·H₂O (97 mg, 2.32 mmol) in MeOH (2 mL), THE (2 mL) and H₂O (2 mL) was stirred at room temperature for 2 h. The reaction was treated with 6 M HCl (0.5 mL). The mixture was concentrated in vacuo, diluted with water (20 mL) and extracted with EtOAc (3×50 mL).

The combined organic layers were washed with brine (20 mL), dried with Na₂SO₄ and concentrated in vacuo to give 2-[3-(4-bromophenyl)-4-methyl-2-oxobenzimidazol-1-yl]acetic acid (Intermediate I-17, 270 mg, 0.75 mmol, 97% yield) as a white solid which was used in the next step without further purification.

UPLC-MS (ES⁺, Method 2): 2.09 min, m/z 361.0 [M+H]⁺¹H NMR (400 MHz, DMSO-d₆): δ 7.75 (d, J=8.6 Hz, 2H), 7.43 (d, J=8.6 Hz, 2H), 7.12 (d, J=7.8 Hz, 1H), 7.04 (t, J=7.8 Hz, 1H), 6.86 (d, J=7.6 Hz, 1H), 4.67 (s, 2H), 1.84 (s, 3H) ppm.

STEP F. Synthesis of Intermediate I-18, 2-[3-(4-bromophenyl)-4-methyl-2-oxobenzimidazol-1-yl]-N-(2,2,2-trifluoroethyl)acetamide: A mixture of 2-(3-(4-bromophenyl)-4-methyl-2-oxo-2,3-dihydro-1H-benzo[d]imidazol-1-yl)acetic acid (Intermediate I-17, 270 mg, 0.75 mmol), 2,2,2-trifluoroethan-1-amine (111.4 mg, 1.125 mmol), HATU (428 mg, 1.125 mmol) and DIPEA (291 mg, 2.25 mmol) in DMF (3 ml) was stirred at room temperature overnight and diluted with water. The precipitate was collected by filtration and dried in vacuo to give 2-[3-(4-bromophenyl)-4-methyl-2-oxobenzimidazol-1-yl]-N-(2,2,2-trifluoroethyl)acetamide (Intermediate I-18, 260 mg, 0.59 mmol, 79%) as off-white solid. LC-MS (ES⁺, Method 4): 2.42 min, m/z, 442.1 [M+H]. ¹H NMR (400 MHz, DMSO-d₆): δ 8.94 (t, J=6.2 Hz, 1H), 7.76 (d, J=8.6 Hz, 2H), 7.43 (d, J=8.6 Hz, 2H), 7.03 (t, J=7.6 Hz, 1H), 6.99 (d, J=7.2 Hz, 1H), 6.86 (d, J=7.2 Hz, 1H), 4.63 (s, 2H), 3.95 (dq, J=6.4, 16.2 Hz, 2H), 1.84 (s, 3H) ppm.

Intermediates synthesised following the same procedure as Intermediate I-18 (scheme 3) replacing 2-fluoro-1-methyl-3-nitrobenzene and 4-bromoaniline with the appropriate building blocks for Step A or replacing 2,2,2-trifluoroethan-1-amine with the appropriate building block for Step F are described in Table 5.

TABLE 5

| Building | Intermediate structure and | LC-MS | |
|---|---|---|---|
| blocks | name | (ES⁺) | ¹H NMR (400 MHz, DMSO-d₆) |
| Step F: (S)-2-Amino- 1,1,1- trifluoropropane hydrochloride | 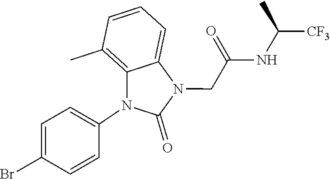 [SMILES: Cc1cccc2c1n(-c1ccc(Br)cc1)c(=O)n2CC(=O)N[C@@H](C)C(F)(F)F] | Method 3: 4.283 min, m/z 458.10 [M + H]⁺ | δ 8.86 (d, J = 8.8 Hz, 1H), 7.73 (d, J = 8.3 Hz, 2H), 7.40 (d, J = 8.7 Hz, 2H), 7.01 (t, J = 7.7 Hz, 1H), 6.95 (d, J = 7.8 Hz, 1H), 6.83 (d, J = 7.5 Hz, 1H), 4.62 (mc, 1H), 4.59 (s, 2H), 1.82 (s, 3H), 1.28 (d, J = 7.0 Hz, 3H) ppm. |
| | 2-[3-(4-bromophenyl)-4-methyl- | | |
| | 2-oxobenzimidazol-1-yl]-N-[(1S)- | | |
| | 2,2,2-trifluoro-1-methyl- | | |
| | ethyl]acetamide | | |
| | Intermediate I-19 | | |
| Step F: 2,2,2- trifluoroethan-1- amine | 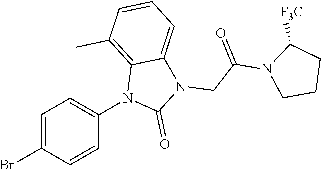 [SMILES: Cc1cccc2c1n(-c1ccc(Br)cc1)c(=O)n2CC(=O)N1CCC[C@H]1C(F)(F)F] | Method 3: 4.417 min, m/z 484.10 [M + H]⁺ | δ 7.74 (d, J = 8.6 Hz, 2H), 7.40 (d, J = 8.6 Hz, 2H), 7.00 (d, J = 4.9 Hz, 2H), 6.87-6.80 (m, 1H), 4.83 (s, 2H), 4.74 (mc, 1H), 3.86- 3.67 (m, 2H), 2.21-1.96 (m, 4H), 1.83 (s, 3H) ppm. |
| | 3-(4-bromophenyl)-4-methyl-1- | | |
| | [2-oxo-2-[(2S)-2- | | |
| | (trifluoromethyl)pyrrolidin-1- | | |
| | yl]ethyl]benzimidazol-2-one | | |
| | Intermediate I-20 | | |
| Step F: 2- (trifluoromethyl) morpholine | 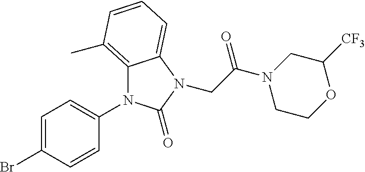 [SMILES: Cc1cccc2c1n(-c1ccc(Br)cc1)c(=O)n2CC(=O)N1CCOC(C(F)(F)F)C1] | Method 3: 4.249 min, m/z 498.00 [M + H]+ | δ 7.74 (d, J = 8.4 Hz, 2H), 7.40 (d, J = 8.4 Hz, 2H), 7.04-7.00 (m, 2H), 6.84-6.81 (m, 1H), 5.07- 4.77 (m, 2H), 4.40-3.87 (m, 4H), 3.71-3.67 (m, 1H), 3.45-3.40 (m, 1H), 2.94-2.89 (m. 1H) 1.83 (s, 3H) ppm. |
| | 3-(4-bromophenyl)-4-methyl-1- | | |
| | [2-oxo-2-[2- | | |
| | (trifluoromethyl)morpholin-4- | | |
| | yl]ethyl]benzimidazol-2-one | | |
| | Intermediate I-21 | | |
| | 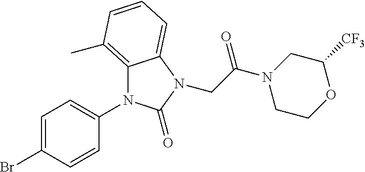 [SMILES: Cc1cccc2c1n(-c1ccc(Br)cc1)c(=O)n2CC(=O)N1CCO[C@@H](C(F)(F)F)C1] | Method 4: 1.852 min, m/z 497.90 [M + H]⁺ | δ 7.74 (d, J = 8.4 Hz, 2H), 7.40 (d, J = 8.4 Hz, 2H), 7.04-7.00 (m, 2H), 6.84-6.81 (m, 1H), 5.07- 4.77 (m, 2H), 4.40-3.87 (m, 4H), 3.71-3.67 (m, 1H), 3.45-3.40 (m, 1H), 2.94-2.89 (m. 1H) 1.83 (s, 3H) ppm. |
| | 3-(4-bromophenyl)-4-methyl-1- | | |
| | [2-oxo-2-[(2R)-2-(trifluoro- | | |
| | methyl)morpholin-4-yl]ethyl]- | | |
| | benzimidazol-2-one | | |
| | Intermediate I-22 | | |
| Step A: 1-chloro-2- fluoro-3- nitrobenzene and 4-Bromoaniline | 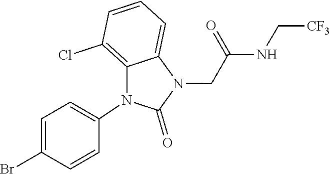 [SMILES: O=C(Cn1c(=O)n(-c2ccc(Br)cc2)c2c(Cl)cccc21)NCC(F)(F)F] | Method 3: 4.150 min, m/z 463.95 [M + H]⁺ | δ 8.93 (t, J = 6.3 Hz, 1H), 7.71 (d, J = 8.4 Hz, 2H), 7.40 (d, J = 8.4 Hz, 2H), 7.17-7.09 (m, 2H), 7.07 (dd, J = 1.7, 7.6 Hz, 1H), 4.66 (s, 2H), 3.96 (dq, J = 6.1, 9.7 Hz, 2H) ppm. |
| | 2-[3-(4-bromophenyl)-4-chloro- | | |
| | 2-oxo-benzimidazol-1-yl]-N- | | |
| | (2,2,2-trifluoroethyl)acetamide | | |
| | Intermediate I-23 | | |
| Step A: 2-fluoro-1- methoxy-3- nitrobenzene and 4-Bromoaniline | 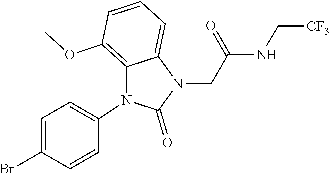 [SMILES: COc1cccc2c1n(-c1ccc(Br)cc1)c(=O)n2CC(=O)NCC(F)(F)F] | Method 4: 1.642 min, m/z 459.95 [M + H]⁺ | |
| | 2-[3-(4-bromophenyl)-4- | | |
| | methoxy-2-oxobenzimidazol-1- | | |
| | yl]-N-(2,2,2-trifluoroethyl)- | | |
| | acetamide | | |
| | Intermediate I-24 | | |
| Step A: 1-fluoro-2- nitrobenzene and 4-bromo-2- methylaniline | 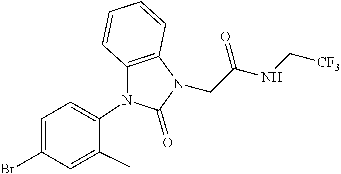 [SMILES: Cc1cc(Br)ccc1-n1c(=O)n(CC(=O)NCC(F)(F)F)c2ccccc21] | Method 3: 4.150 min, m/z 442.0 [M + H]⁺ | (400 MHz, CDCl3) 7.59 (dd, J = 0.5, 2.2 Hz, 1H), 7.50 (ddd, J = 0.5, 2.2, 8.3 Hz, 1H), 7.21-7.14 (m, 3H), 7.10 (dt, J = 1.5, 11.4 Hz, 1H), 6.89 (tbr, J = 6.8 Hz, 1H), 6.74 (d, J = 7.7 Hz, 1H), 4.64 (s, 2H), 3.92 (mc, 2H), 2.15 (s, 3H) ppm. |
| | 2-[3-(4-bromo-2-methyl-phenyl)- | | |
| | 2-oxobenzimidazol-1-yl]-N- | | |
| | (2,2,2-trifluoroethyl)acetamide | | |
| | Intermediate I-25 | | |
| Step A: 1-fluoro-2- nitrobenzene and 4-bromo-2- fluoroaniline | 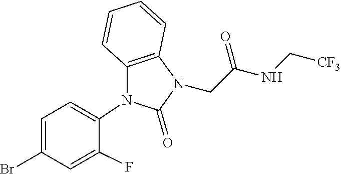 [SMILES: O=C(Cn1c(=O)n(-c2ccc(Br)cc2F)c2ccccc21)NCC(F)(F)F] | Method 4: 1.75 min, m/z, 445.95 [M + H]⁺ | δ 8.98 (t, J = 6.3 Hz, 1H), 7.89 (dd, J = 1.9, 9.9 Hz, 1H), 7.64 (dd, J = 1.5, 8.4 Hz, 1H), 7.58 (t, J = 8.1 Hz, 1H), 7.17-7.10 (m, 2H), 7.06 (dt, J = 2.3, 7.1 Hz, 1H), 6.87 (d, J = 7.7 Hz, 1H), 4.65 (s, 2H), 3.94 (dq, J = 6.3, 9.5 Hz, 2H) ppm. |
| | 2-[3-(4-bromo-2-fluoro-phenyl)- | | |
| | 2-oxobenzimidazol-1-yl]-N- | | |
| | (2,2,2-trifluoroethyl)acetamide | | |
| | Intermediate I-26 | | |
| Step A: 2-fluoro-1- methyl-3- nitrobenzene and 4-bromo-2- methylaniline | 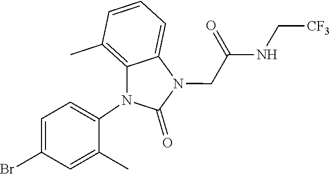 [SMILES: Cc1cc(Br)ccc1-n1c(=O)n(CC(=O)NCC(F)(F)F)c2cccc(C)c21] | Method 4: 2.527 min, m/z 456.0 [M + H]⁺ | δ 8.96 (t, J = 6.5 Hz, 1H), 7.70 (d, J = 1.7 Hz, 1H), 7.58 (dd, J = 1.9, 8.2 Hz, 1H), 7.36 (d, J = 8.3 Hz, 1H), 7.06-6.96 (m, 2H), 6.83 (dd, J = 2.3, 5.8 Hz, 1H), 4.65 (s, 2H), 3.99 (mc, 2H), 2.06 (s, 3H), 1.75 (s, 3H) ppm. |
| | 2-[3-(4-bromo-2-methylphenyl)- | | |
| | 4-methyl-2-oxobenzimidazol-1- | | |
| | yl]-N-(2,2,2- | | |
| | trifluoroethyl)acetamide | | |
| | Intermediate I-27 | | |
| Step A: 2-fluoro nitrobenzene and 4-bromo-2,6- difluoraniline | 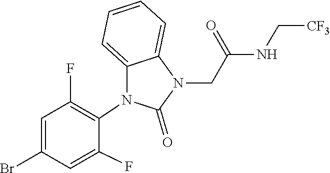 [SMILES: O=C(Cn1c(=O)n(-c2c(F)cc(Br)cc2F)c2ccccc21)NCC(F)(F)F] | Method 3: 3.92 min, m/z 464/466 [M + H]⁺ | δ 9.05 (m, 1H), 7.85 (d, 2H), 7.19-7.06 (m, 3H), 6.92 (d, 1H), 4.69 (s, 2H), 4.02 (m, 2H). |
| | 2-[3-(4-bromo-2,6-difluoro- | | |
| | phenyl)-2-oxo-benzimidazol-1- | | |
| | yl]-N-(2,2,2- | | |
| | trifluoroethyl)acetamide | | |
| | Intermediate 1-60 | | |

| Conditions | Structure and ID | LCMS (ES⁺) |
|---|---|---|
| Step A: 2-fluoro-1-methyl-3-nitro-benzene and 5-Amino-2-bromopyridine | 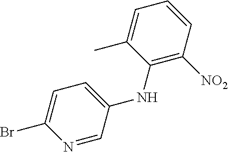 [SMILES: Cc1cccc([N+](=O)[O-])c1Nc1ccc(Br)nc1] | Method 3: 3.82 min, m/z, 310.15 [M + 2]+. |
| | Intermediate 1-67 | |
| Step A: 2-fluoro-1-methyl-3-nitro-benzene and 6-bromo-2-methyl-pyridin-3- amine | 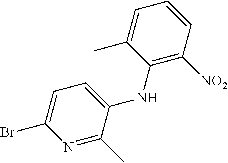 [SMILES: Cc1cccc([N+](=O)[O-])c1Nc1ccc(Br)nc1C] | Method 4: 1.84 min, m/z 322.0/324.0, [M]⁺ |
| | Intermediate 1-68 | |
| Step A: 2-fluoro-1-methyl-3-nitro-benzene and 6-bromo-4-methyl-pyridin-3- amine | 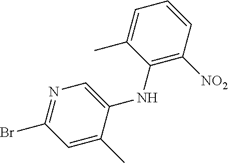 [SMILES: Cc1cc(Br)ncc1Nc1c(C)cccc1[N+](=O)[O-]] | Method 3: 4.05 min, m/z, 324.2 [M]⁺ |
| | Intermediate 1-69 | |
| Step A: 2-fluoro-1-methyl-3-nitro-benzene and 2-Amino-5-bromo-3- methylpyridine | 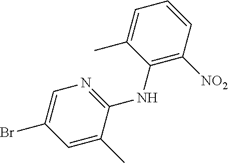 [SMILES: Cc1cc(Br)cnc1Nc1c(C)cccc1[N+](=O)[O-]] | Method 3: 4.60 min, m/z, 321.1 [M]⁻ |
| | Intermediate 1-70 | |
| Step A: 2-fluoro-1-methyl-3-nitro-benzene and 5-bromo-3-methyl-pyrazin-2- amine | 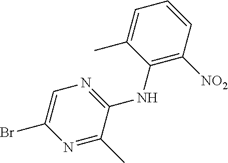 [SMILES: Cc1cccc([N+](=O)[O-])c1Nc1ncc(Br)nc1C] | Method 3 : 3.88 min, m/z, 325.2 [M]+ |
| | Intermediate 1-71 | |
| Step B | 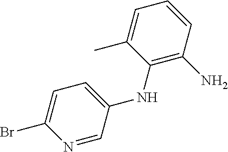 [SMILES: Cc1cccc(N)c1Nc1ccc(Br)nc1] | Method 3: 3.42 min, m/z, 278.2 [M]⁺ |
| | Intermediate I-72 | |
| Step B | 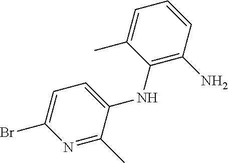 [SMILES: Cc1cccc(N)c1Nc1ccc(Br)nc1C] | Method 4: 1.97 min, m/z 292.1/294.0, [M]+/ [M + 2]+ |
| | Intermediate I-73 | |
| Step B | 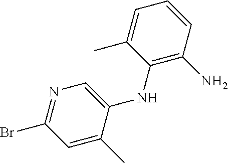 [SMILES: Cc1cc(Br)ncc1Nc1c(C)cccc1N] | Method 3: 3.52min, m/z, 292.2 [M]+ |
| | Intermediate I-74 | |
| Step B | 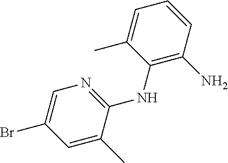 [SMILES: Cc1cc(Br)cnc1Nc1c(C)cccc1N] | Method 3: 4.22 min, m/z, 293.2 [M]+ |
| | Intermediate I-75 | |
| Step B | 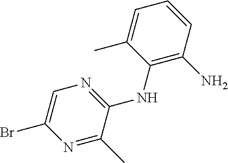 [SMILES: Cc1cccc(N)c1Nc1ncc(Br)nc1C] | Method 3: 3.05 min, m/z, 295.2 [M + 2]+ |
| | Intermediate I-76 | |
| Step C | 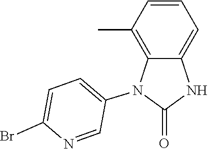 [SMILES: Cc1cccc2[nH]c(=O)n(-c3ccc(Br)nc3)c12] | Method 2: 1.51 min, m/z 304.0/ 305.9 [M]⁺/ [M + 2]⁺ |
| | Intermediate I-77 | |
| Step C | 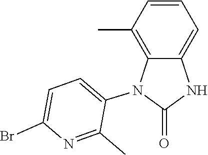 [SMILES: Cc1nc(Br)ccc1-n1c(=O)[nH]c2cccc(C)c21] | Method 4: 1.60 min, m/z, 318.0 [M]+ |
| | Intermediate 1-78 | |
| Step C | 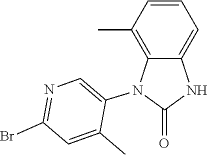 [SMILES: Cc1cc(Br)ncc1-n1c(=O)[nH]c2cccc(C)c21] | Method 3: 3.32 min, m/z, 318.1, [M]+ |
| | Intermediate I-79 | |
| Step C | 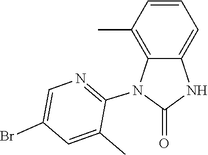 [SMILES: Cc1cc(Br)cnc1-n1c(=O)[nH]c2cccc(C)c21] | Method 3: 2.47 min, m/z, 317.1 [M + 2]+ |
| | Intermediate 1-80 | |
| Step C | 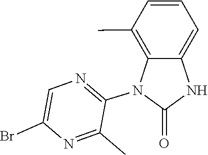 [SMILES: Cc1nc(Br)cnc1-n1c(=O)[nH]c2cccc(C)c21] | Method 3: 3.38 min, m/z, 321.2 [M]+ |
| | N Intermediate 1-81 | |

Intermediates analogous to Intermediate I-16 (scheme 3) were synthesised by following the same synthetic route and procedures, replacing 2-fluoro-1-methyl-3-nitrobenzene and 4-bromoaniline with the appropriate building blocks in Step A. Such intermediates are shown in Table 6.

TABLE 6

| | | | ¹H NMR (400 MHz, |
|---|---|---|---|
| Building blocks | Intermediate structure and name | LC-MS (ES⁺) | DMSO-d₆) |
| 2-fluoro-1- methyl-3- nitrobenzene and 5-bromo-2- aminopyrazine | 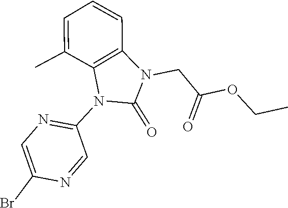 [SMILES: CCOC(=O)Cn1c(=O)n(-c2cnc(Br)cn2)c2c(C)cccc21] | Method 3: 4.15 min, m/z 391/393 [M + H]⁺ | δ 9.01 (s, 1H), 8.88 (s, 1H), 7.20-7.12 (m, 2H), 6.82 (d, 1H), 4.69 (s, 2H), 4.18 (q, 2H), 1.90 (s, 3H), 1.25 (t, 3H) ppm. |
| | ethyl 2-[4- | | |
| | methyl-3-[5-(1-methylpyrazolo[3,4- | | |
| | c]pyridin-4-yl)pyrazin-2-yl]-2-oxo- | | |
| | benzimidazol-1-yl]acetate 1-82 | | |
| | Intermediate 1-37 | | |
| 2-fluoro-1- methyl-3- nitrobenzene and 2-bromo-5- aminopyridine | 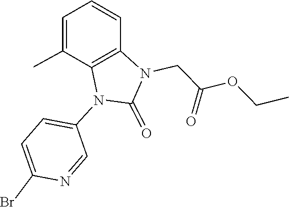 [SMILES: CCOC(=O)Cn1c(=O)n(-c2ccc(Br)nc2)c2c(C)cccc21] | Method 3: 4.00 min, m/z 390/392 [M + H]⁺ | δ 8.56 (m, 1H), 7.93, (m, 1H), 7.85 (d, 1H), 7.14-7.04 (m, 2H), 6.89 (m, 1H), 4.78 (s, 2H), 4.18 (q, 2H), 1.87 (s, 3H), 1.23 (t, 3H) ppm. |
| | ethyl 2-[3-(6- | | |
| | bromo-3-pyridyl)-4-methyl-2-oxo- | | |
| | benzimidazol-1-yl]acetate | | |
| | Intermediate 1-38 | | |
| 2-fluoro-1- methyl-3- nitrobenzene and 3-bromo-5- aminopyridine | 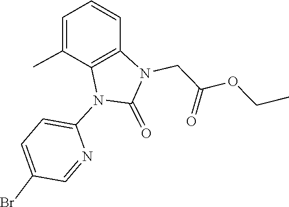 [SMILES: CCOC(=O)Cn1c(=O)n(-c2ccc(Br)cn2)c2c(C)cccc21] | Method 4: 2.07 min, m/z 390/392 [M + H]⁺ | δ 8.78 (m, 1H), 8.31, (m, 1H), 7.63 (d, 1H), 7.13-7.04 (m, 2H), 6.89 (m, 1H), 4.77 (s, 2H), 4.18 (q, 2H), 1.87 (s, 3H), 1.23 (t, 3H) ppm. |
| | ethyl 2-[3-(5- | | |
| | bromo-2-pyridyl)-4-methyl-2-oxo- | | |
| | benzimidazol-1-yl]acetate | | |
| | Intermediate 1-39 | | |
| 1-fluoro-2- nitrobenzene and 2-amino-5- bromopyridine | 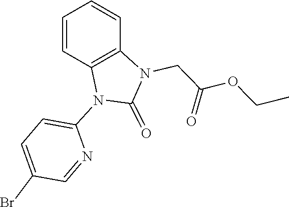 [SMILES: CCOC(=O)Cn1c(=O)n(-c2ccc(Br)cn2)c2ccccc21] | Method 3: 4.38 min, m/z 376/378 [M + H]⁺ | δ 8.74 (s, 1H), 8.26 (d. 1H), 8.09-8.00 (m, 2H), 7.32-7.13 (m, 3H), 4.83 (s, 2H), 4.21 (q, 2H), 1.23 (t, 3H) ppm. |
| | ethyl 2-[3-(5-bromo-2-pyridyl)-2-oxo- | | |
| | benzimidazol-1-yl]acetate | | |
| | Intermediate I-41 | | |
| 2-fluoro-1- methyl-3- nitrobenzene and 6-bromo-4- methyl-pyridin- 3-amine | 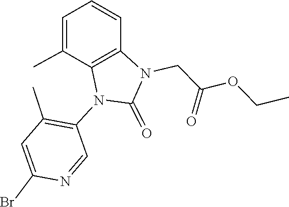 [SMILES: CCOC(=O)Cn1c(=O)n(-c2cnc(Br)cc2C)c2c(C)cccc21] | Method 4: 1.99 min, m/z 404/406 [M + H]⁺ | δ 8.32 (d. 1H), 7.52 (s, 1H), 7.26 (s, 1H), 7.07 (m, 1H), 6.84 (m, 1H) 4.66 (dd, 2H), 4.23 (q, 2H), 2.20 (s, 3H), 1.84 (s, 3H) 1.28 (t, 3H) ppm. |
| | ethyl 2-[3-(6-bromo-4-methyl-3- | | |
| | pyridyl)-4-methyl-2-oxo-benzimidazol- | | |
| | 1-yl]acetate | | |
| | Intermediate I-43 | | |
| 2-fluoro-1- methyl-3- nitrobenzene and 2-bromo-5- aminopyridine | 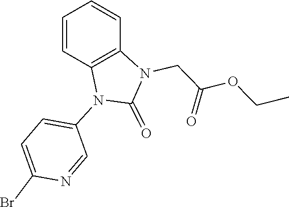 [SMILES: CCOC(=O)Cn1c(=O)n(-c2ccc(Br)nc2)c2ccccc21] | Method 3: 3.91 min, m/z 376/378 [M + H]⁺ | δ 8.68 (s, 1H), 8.05 (d. 1H), 87.91 (d, 1H), 7.32-7.13 (m, 4H), 4.84 (s, 2H), 4.21 (q, 2H), 1.23 (t, 3H) ppm. |
| | ethyl 2-[3-(6-bromo-3-pyridyl)-4- | | |
| | methyl-2-oxo-benzimidazol-1- | | |
| | yl]acetate | | |
| | Intermediate I-44 | | |
| 2-fluoro-1- methyl-3- nitrobenzene and 2-amino-5- bromopyrimidine | 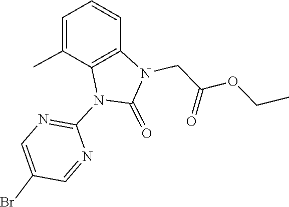 [SMILES: CCOC(=O)Cn1c(=O)n(-c2ncc(Br)cn2)c2c(C)cccc21] | Method 4: 1.41 min, m/z 391/393 [M + H]⁺ | δ 8.94 (s, 2H), 7.11 (m, 1H), 6.92 (m, 1H), 6.80 (m, 1H), 4.65 (s, 2H), 4.24 (q, 2H), 1.98 (s, 3H), 1.27 (t, 3H) ppm. |
| | ethyl 2-[3-(5-bromopyrimidin-2-yl)-4- | | |
| | methyl-2-oxo-benzimidazol-1- | | |
| | ylacetate | | |
| | Intermediate I-45 | | |
| 2-fluoro-1- methyl-3- nitrobenzene and 6-amino-3- bromopyridazine | 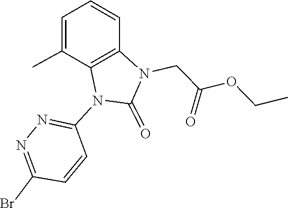 [SMILES: CCOC(=O)Cn1c(=O)n(-c2ccc(Br)nn2)c2c(C)cccc21] | Method 3: 3.88 min, m/z 391/393 [M + H]⁺ | δ 8.32 (s, 1H), 8.04 (s, 1H) 7.19-7.10 (m, 2H), 6.96 (m, 1H), 4.82 (s, 2H), 4.20 (q, 2H), 1.89 (s, 3H), 1.24 (t, 3H) ppm. |
| | ethyl 2-[3-(6- | | |
| | bromopyridazin-3-yl)-4-methyl-2-oxo- | | |
| | benzimidazol-1-yl]acetate | | |
| | Intermediate I-46 | | |
| 2-fluoro-3-nitro- benzonitrile and 4-bromoaniline | 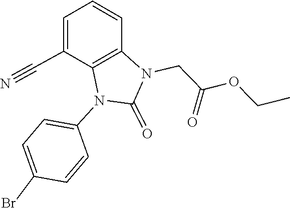 [SMILES: CCOC(=O)Cn1c(=O)n(-c2ccc(Br)cc2)c2c(C#N)cccc21] | Method 3: 4.12 min, m/z 401/403 [M + H]⁺ | δ 7.79 (d, 2H), 7.65 (s, 1H) 7.54 (d, 2H), 7.47 (d, 1H), 7.30 (m, 1H), 4.86 (s, 2H), 4.21 (q, 2H), 1.25 (t, 3H) ppm. |
| | ethyl 2-[3-(4- | | |
| | bromophenyl)-4-cyano-2-oxo- | | |
| | benzimidazol-1-yl]acetate | | |
| | Intermediate 1-47 | | |
| 2-fluoro-1- methyl-3- nitrobenzene and 6-bromo-2- methyl-pyridin- 3-amine | 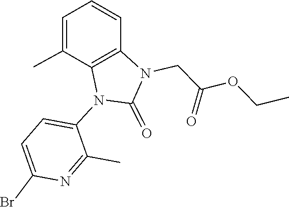 [SMILES: CCOC(=O)Cn1c(=O)n(-c2ccc(Br)nc2C)c2c(C)cccc21] | Method 4: 2.19 min, m/z 404/406 [M + H]⁺ | δ 7.88 (d, 1H), 7.70 (d, 1H) 7.14 (d, 1H), 7.03 (t, 1H), 6.87 (d, 1H), 4.82 (m, 2H), 4.19 (q, 2H), 2.24 (s, 3H), 1.78 (s, 3H), 1.23 (t, 3H) ppm. |
| | ethyl 2-[3-(6-bromo-2-methyl-3- | | |
| | pyridyl)-4-methyl-2-oxo-benzimidazol- | | |
| | 1-yl]acetate | | |
| | Intermediate 1-63 | | |
| 2-fluoro-1- methyl-3- nitrobenzene and 6-bromo-2- methylpyrazin-3- amine | 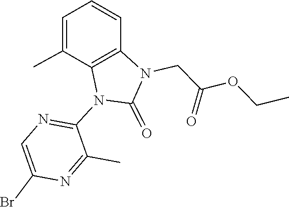 [SMILES: CCOC(=O)Cn1c(=O)n(-c2ncc(Br)nc2C)c2c(C)cccc21] | Method 3: 3.85 min, m/z 405/407 [M + H]⁺ | δ 8.88 (s, 1H), 7.19- 7.07 (m, 2H) 6.90 (m, 1H), 4.87 (m, 2H), 4.20 (q, 2H), 2.46 (s, 3H), 1.76 (s, 3H), 1.21 (t, 3H) ppm. |
| | ethyl 2-[3-(5-bromo-3-methyl-pyrazin-2-yl)- | | |
| | 4-methyl-2-oxo-benzimidazol-1-yl]acetate | | |
| | Intermediate 1-64 | | |
| 2-fluoro-1- methyl-3- nitrobenzene and 6-bromo-5- methylpyridin-3- amine | 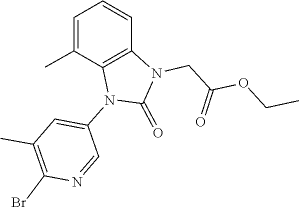 [SMILES: CCOC(=O)Cn1c(=O)n(-c2cnc(Br)c(C)c2)c2c(C)cccc21] | Method 3: 3.86 min, m/z 404/406 [M + H]⁺ | δ 8.38 (s, 1H), 7.99 (s, 1H), 7.15-7.04 (m, 2H) 6.90 (m, 1H), 4.78 (s, 2H), 4.20 (q, 2H), 2.42 (s, 3H), 1.87 (s, 3H), 1.21 (t, 3H) ppm. |
| | ethyl 2-[3-(6-bromo-5-methyl-3-pyridyl)-4- | | |
| | methyl-2-oxo-benzimidazol-1-yl]acetate | | |
| | Intermediate 1-65 | | |
| 2-fluoro-1- methyl-3- nitrobenzene and 5-bromo-6- methylpyrazin-2- amine | 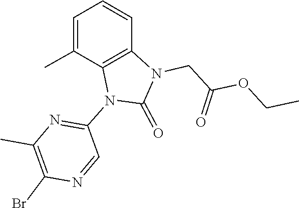 [SMILES: CCOC(=O)Cn1c(=O)n(-c2cnc(Br)c(C)n2)c2c(C)cccc21] | Method 4: 2.42 min, m/z 405/407 [M + H]⁺ | δ 8.65 (s, 1H), 7.15- 7.09 (m, 2H) 6.95 (m, 1H), 4.80 (s, 2H), 4.19 (q, 2H), 2.67 (s, 3H), 1.92 (s, 3H), 1.22 (t, 3H) ppm. |
| | ethyl 2-[3-(5-bromo-6-methyl-pyrazin- | | |
| | 2-yl)-4-methyl-2-oxo-benzimidazol-1- | | |
| | yl]acetate | | |
| | Intermediate 1-66 | | |

Intermediates synthesised following the same procedure as Intermediate iv-1 (Route 1) replacing 3-(5-bromo-2-pyridyl)-4-methyl-1H-benzimidazol-2-one in Step D for the described building block and iv-4 (Route 4) replacing ethyl 2-(2-oxo-3H-benzimidazol-1-yl)acetate in Step D for the described building blocks are described in Table 6.

| | | |
|---|---|---|
| Step D | 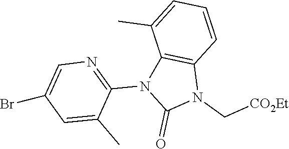 [SMILES: CCOC(=O)Cn1c(=O)n(-c2ncc(Br)cc2C)c2c(C)cccc21] | Method 3: 4.15 min, m/z, 403.1 [M]+ |
| | Intermediate 1-83 | |

### Synthesis of Intermediate I-31, 2-[3-[4-(1-methylpyrazolo[3,4-c]pyridin-4-yl)phenyl]-2-oxobenzimidazol-1-yl]acetic acid

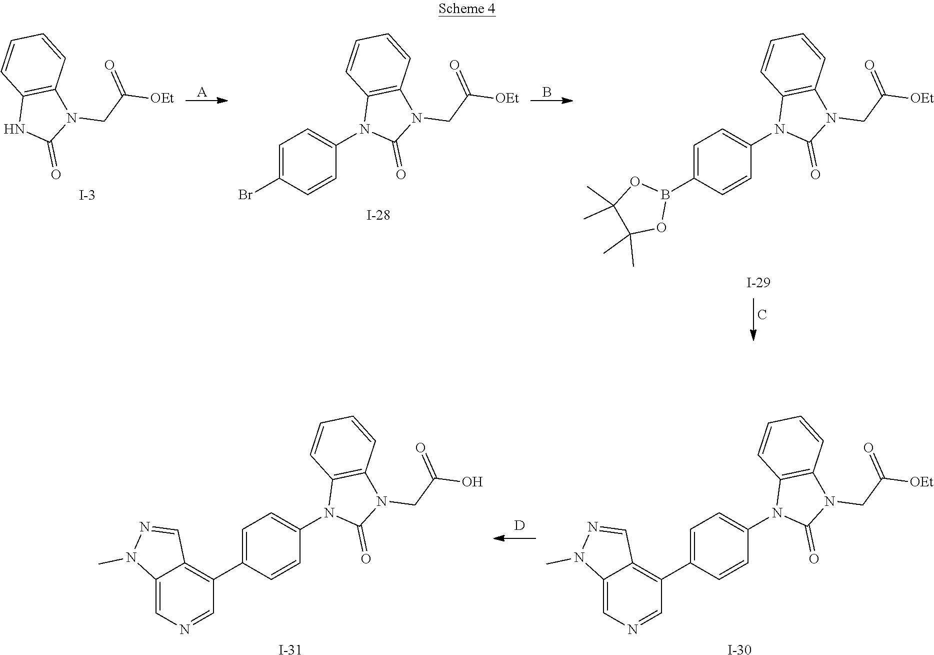

STEP A. Synthesis of Intermediate I-28, Ethyl 2-[3-(4-bromophenyl)-2-oxobenzimidazol-1-yl]acetate: In a round-bottom flask with a mounted condenser open to air 4-bromo-benzeneboronic acid (4.1 g, 20 mmol), ethyl 2-(2-oxo-3H-benzimidazol-1-yl)acetate (Intermediate I-3, 2.2 g, 10 mmol) and copper (II) acetate (3.7 g, 20 mmol) were suspended in MeCN (90 mL). Et₃N (4.2 mL, 30 mmol) was added and the suspension was stirred at room temperature for 16 h. The solvent was removed in vacuo. The residue was dissolved in DCM (50 mL) and washed with H₂O (50 mL) and sat. aq. NH₄Cl (50 mL). The aqueous layer was extracted with DCM (3×50 mL). The combined organic layers were dried with Na₂SO₄ and concentrated in vacuo. The crude product was dry loaded onto silica and purified by flash chromatography (25 g SiO₂, 5 to 90% EtOAc in pet. ether) to give ethyl 2-[3-(4-bromophenyl)-2-oxobenzimidazol-1-yl]acetate (Intermediate I-28, 3.4 g, 9.2 mmol, 90% yield) as a white solid. UPLC-MS (ES⁺, Method 2): 1.88 min, m/z 375.0/376.9 [M+H]⁺. ¹H NMR (400 MHz, DMSO-d₆): δ 7.79 (td, J=2.5, 9.6, 2H), 7.54 (td, J=2.5, 9.6, 2H), 7.30 (d, J=7.6 Hz, 1H), 7.07-7.20 (m, 3H), 4.82 (s, 2H), 4.19 (q, J=7.1 Hz, 2H), 1.24 (t, J=7.1 Hz, 3H) ppm.

STEP B. Synthesis of Intermediate I-29, ethyl 2-[2-oxo-3-[4-(4,4,5,5-tetramethyl-1,3,2-dioxaborolan-2-yl)phenyl]benzimidazol-1-yl]acetate: A mixture of ethyl 2-[3-(4-bromophenyl)-2-oxobenzimidazol-1-yl]acetate (Intermediate I-28, 500 mg, 1.33 mmol), bis(pinacolato)diboron (508 mg, 2.0 mmol), Pd₂(dba)₃ (122 mg, 0.13 mmol), Xphos (127 mg, 0.27 mmol) and KOAc (392 mg, 4.0 mmol) in THE (10 mL) was stirred at 70° C. under N₂ for 16 h. The mixture was diluted with EtOAc (100 mL) and washed with water (100 mL) and brine (100 mL). The combined aqueous layers were extracted with EtOAc (2×100 mL). The organic layers were combined, dried with MgSO₄ and concentrated in vacuo to give ethyl 2-[2-oxo-3-[4-(4,4,5,5-tetramethyl-1,3,2-dioxaborolan-2-yl)phenyl]benzimidazol-1-yl]acetate (Intermediate I-29, 1.2 g, 1.14 mmol, 40% yield) as a brown solid which was used in the next step without further purification. LC-MS (ES⁺, Method 4): 4.55 min, m/z 423.3 [M+H]⁺

STEP C. Synthesis of Intermediate I-30, ethyl 2-[3-[4-(1-methylpyrazolo[3,4-c]pyridin-4-yl)phenyl]-2-oxobenzimidazol-1-yl]acetate: A degassed mixture of ethyl 2-[2-oxo-3-[4-(4,4,5,5-tetramethyl-1,3,2-dioxaborolan-2-yl)phenyl]benzimidazol-1-yl]acetate (Intermediate I-29, 1.00 g, 2.37 mmol), 4-bromo-1-methyl-1H-pyrazolo[3,4-c]pyridine (502 mg, 2.37 mmol), Pd(dppf)Cl₂-DCM (193 mg, 0.24 mmol), and KOAc (930 mg, 9.47 mmol) in 1,4-dioxane (30 mL) and water (3 mL) was stirred at 90° C. under N₂ for 16 h. The mixture was concentrated in vacuo and purified by normal phase chromatography (1 to 2.5% MeOH in DCM) to give ethyl 2-[3-[4-(1-methylpyrazolo[3,4-c]pyridin-4-yl)phenyl]-2-oxobenzimidazol-1-yl]acetate (Intermediate I-30, 400 mg, 0.94 mmol, 40% yield) as an off-white solid. LC-MS (ES⁺, Method 4): 1.533 min, m/z, 428.15 [M+H]⁺. ¹H NMR (400 MHz, DMSO-d₆): δ 9.24 (s, 1H), 8.52 (s, 1H), 8.44 (s, 1H), 8.05 (d, J=8.4 Hz, 2H), 7.78 (d, J=8.4 Hz, 2H), 7.34 (dd, J=1.0, 7.5, 1H), 7.25 (dd, J=1.0, 7.5 Hz, 1H,), 7.20 (dt, J=1.0, 7.5 Hz, 1H), 7.15 (dt, J=1.0, 7.5 Hz, 1H), 4.87 (s, 2H), 4.27 (s, 3H), 4.22 (q, J=7.1 Hz, 2H), 1.26 (t, J=7.1 Hz, 3) ppm.

STEP D. Synthesis of Intermediate I-31, 2-[3-[4-(1-methylpyrazolo[3,4-c]pyridin-4-yl)phenyl]-2-oxobenzimidazol-1-yl]acetic acid: A mixture of ethyl 2-[3-[4-(1-methylpyrazolo[3,4-c]pyridin-4-yl)phenyl]-2-oxobenzimidazol-1-yl]acetate (Intermediate I-30, 470 mg, 1.10 mmol) and LiOH (58 mg, 2.42 mmol) in THE (15 mL), MeOH (10 mL) and water (5 mL) was stirred at room temperature for 16 h. The mixture was concentrated in vacuo, diluted with water and extracted with ethyl acetate (×3). The combined organics were washed with brine, dried with Na₂SO₄ and concentrated in vacuo to give 2-[3-[4-(1-methylpyrazolo[3,4-c]pyridin-4-yl)phenyl]-2-oxobenzimidazol-1-yl]acetic acid (Intermediate I-31, 446 mg, 1.10 mmol, 100% yield) as white solid. LC-MS (ES⁺, Method 4): 1.967 min, m/z, 400.05 [M+H]⁺. ¹H NMR (400 MHz, DMSO-d₆): δ 9.39 (s, 1H), 8.59 (s, 1H), 8.55 (s, 1H), 8.08 (d, J=8.6 Hz, 2H), 7.81 (d, J=8.6 Hz, 2H), 7.34 (d, J=7.7 Hz, 1H), 7.25 (d, J=7.7 Hz, 1H), 7.20 (t, J=7.6 Hz, 1H), 7.15 (t, J=7.6 Hz, 1H), 4.76 (s, 2H), 4.31 (s, 3H) ppm.

### Synthesis of Intermediate I-34, 2-[4-methyl-3-[4-(1-methylpyrazolo[3,4-c]pyridin-4-yl)phenyl]-2-oxobenzimidazol-1-yl]acetic acid

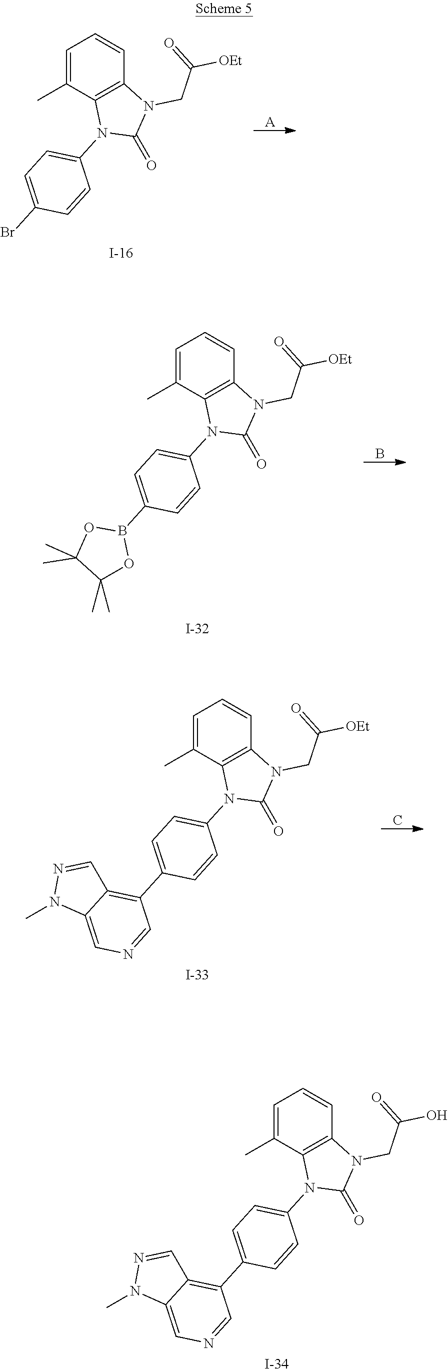

STEP A. Synthesis of Intermediate I-32, Ethyl 2-[4-methyl-2-oxo-3-[4-(4,4,5,5-tetramethyl-1,3,2-dioxaborolan-2-yl)phenyl]benzimidazol-1-yl]acetate: A mixture of ethyl ethyl 2-(3-(4-bromophenyl)-4-methyl-2-oxo-2,3-dihydro-1H-benzo[d]imidazol-1-yl)acetate (Intermediate I-16, 2.00 g, 5.15 mmol), B₂pin₂ (2.617 g, 10.31 mmol), Pd₂(dba)₃ (472 mg, 0.5154 mmol), XPhos (491.5 mg, 1.031 mmol) and KOAc (1.517 g, 15.46 mmol, 3.0 eq) in degassed THE (30 mL) was stirred at 70° C. under N₂-atmosphere overnight. The mixture was cooled to room temperature, filtered, concentrated in vacuo and purified by flash chromatography (SiO₂, EtOAc in pet. ether 0-50%) to give ethyl 2-[4-methyl-2-oxo-3-[4-(4,4,5,5-tetramethyl-1,3,2-dioxaborolan-2-yl)phenyl]benzimidazol-1-yl]acetate (Intermediate I-32, 2.20 g, 5.04 mmol, 98%) as off-white solid. LC-MS (Method 3): 4.579 min, m/z 437.2 [M+H]⁺. ¹H NMR (400 MHz, DMSO-d₆): δ 7.84 (d, J=8.2 Hz, 2H), 7.45 (d, J=8.2 Hz, 2H), 7.12 (d, J=7.8 Hz, 1H), 7.04 (t, J=7.8 Hz, 1H), 6.86 (d, J=7.8 Hz, 1H), 4.78 (s, 2H), 4.19 (q, J=7.1 Hz, 2H), 1.81 (s, 3H), 1.35 (s, 12H), 1.24 (t, J=7.1 Hz, 3H) ppm.

STEP B. Synthesis of Intermediate I-33, ethyl 2-[4-methyl-3-[4-(1-methylpyrazolo[3,4-c]pyridin-4-yl)phenyl]-2-oxobenzimidazol-1-yl]acetate: A mixture of 4-bromo-1-methyl-1H-pyrazolo[3,4-c]pyridine (709 mg, 3.36 mmol), ethyl 2-(4-methyl-2-oxo-3-(4-(4,4,5,5-tetramethyl-1,3,2-dioxaborolan-2-yl)phenyl)-2,3-dihydro-1H-benzo[d]imidazol-1-yl)acetate (Intermediate I-32, 2.2 g, 5.04 mmol), Pd(dppf)Cl₂ (245 mg, 0.336 mmol), and K₂CO₃ (1.39 g, 10.08 mmol) in degassed DMF (15 mL) was stirred at 80° C. under N₂-atmosphere overnight. The reaction was diluted with EtOAc (200 mL) and was washed with water (50 mL×3). The combined organic layers were washed with brine (100 mL) and dried with Na₂SO₄. The mixture was purified by column chromatography (SiO₂, MeOH in DCM 0-5%) to give ethyl 2-[4-methyl-3-[4-(1-methylpyrazolo[3,4-c]pyridin-4-yl)phenyl]-2-oxo-benzimidazol-1-yl]acetate (Intermediate I-33, 710 mg, 1.61 mmol, 48%) as a white solid. LC-MS (Method 4): 1.54 min, m/z 442.15 [M+H]⁺. ¹H NMR (400 MHz, DMSO-d₆): δ 9.25 (s, 1H), 8.54 (s, 1H), 8.43 (s, 1H), 8.02 (d, J=8.3 Hz, 2H), 7.64 (d, J=8.3 Hz, 2H), 7.16 (d, J=7.8 Hz, 1H), 7.07 (t, J=7.8 Hz, 1H), 6.90 (d, J=7.8 Hz, 1H), 4.82 (s, 2H), 4.21 (s, 3H), 4.21 (q, J=7.1 Hz, 2H), 1.93 (s, 3H), 1.26 (t, J=7.1 Hz, 3H) ppm.

STEP C. Synthesis of Intermediate I-34, 2-[4-methyl-3-[4-(1-methylpyrazolo[3,4-c]pyridin-4-yl)phenyl]-2-oxobenzimidazol-1-yl]acetic acid: A mixture of ethyl 2-[4-methyl-3-[4-(1-methylpyrazolo[3,4-c]pyridin-4-yl)phenyl]-2-oxobenzimidazol-1-yl]acetate (Intermediate I-33, 710 mg, 1.61 mmol) and LiOH·H₂O (202.6 mg, 4.828 mmol) in MeOH (4 mL), THE (4 mL) and H₂O (4 mL) was stirred at room temperature for 2 h. The reaction was treated with 1 M HCl (5 mL) and the precipitate was filtered to give 2-[4-methyl-3-[4-(1-methylpyrazolo[3,4-c]pyridin-4-yl)phenyl]-2-oxo-benzimidazol-1-yl]acetic acid (Intermediate I-34, 620 mg, 1.50 mmol, 93%) as off-white solid. LC-MS (Method 3): 3.050 m: m/z 414.0 [M+H]⁺

¹H NMR (400 MHz, DMSO-d₆): δ 9.26 (s, 1H), 8.55 (s, 1H), 8.44 (s, 1H), 8.02 (d, J=8.3 Hz, 2H), 7.65 (d, J=8.3 Hz, 2H), 7.16 (d, J=7.8 Hz, 1H), 7.08 (t, J=7.8 Hz, 1H), 6.90 (d, J=7.8 Hz, 1H), 4.72 (s, 2H), 4.29 (s, 3H), 1.94 (s, 3H) ppm.

Intermediates synthesised following the same procedure as Intermediate I-34 (scheme 5) replacing 2-fluoro-1-methyl-3-nitrobenzene and 4-bromoaniline in Step A of Scheme 3 with the appropriate building blocks for Step A are described in Table 7.

TABLE 7

| Building blocks | Intermediate structure and name | LC-MS (ES⁺) |
|---|---|---|
| 1-fluoro-2- nitrobenzene and 4-bromo-2- methylaniline | 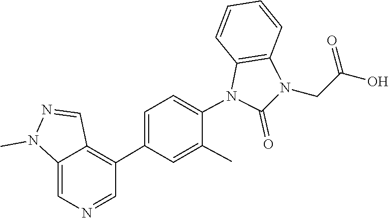 [SMILES: Cc1cc(-c2cncc3c2cnn3C)ccc1-n1c(=O)n(CC(=O)O)c2ccccc21] | Method 3: 3.78 min, m/z 414.0 [M + H]⁺ |
| | Intermediate 1-35 | |
| | 2-[3-(4-imidazo[1,5-a]pyridin-8-ylphenyl)-4-methyl-2-oxo- | |
| | benzimidazol-1-yl]acetic acid | |
| 2-fluoro-1-methyl-3- nitrobenzene and 3- bromo-6- aminopyridine | 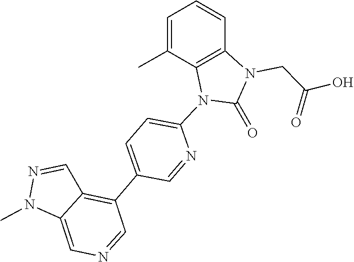 [SMILES: Cc1cccc2c1n(-c1ccc(-c3cncc4c3cnn4C)cn1)c(=O)n2CC(=O)O] | Method 3: 3.05 min, m/z 415.20 [M + H]⁺ |
| | Intermediate I-40 | |
| | 2-[4-methyl-3-[5-(1-methylpyrazolo[3,4-c]pyridin-4-yl)-2- | |
| | pyridyl]-2-oxo-benzimidazol-1-yl]acetic acid | |
| 1-fluoro-2- nitrobenzene and 2- amino-5- bromopyridine | 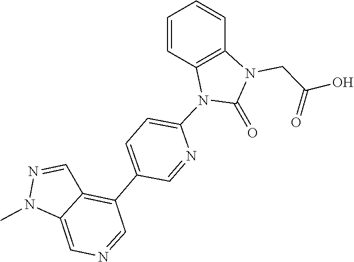 [SMILES: Cn1ncc2c(-c3ccc(-n4c(=O)n(CC(=O)O)c5ccccc54)nc3)cncc21] | Method 3: 3.11 min, m/z 401.15 [M + H]⁺ |
| | Intermediate I-42 | |
| | 2-[3-[5-(1-methylpyrazolo[3,4-c]pyridin-4-yl)-2-pyridyl]-2- | |
| | oxo-benzimidazol-1-yllacetic acid | |
| 2-fluoro-3-nitro- benzonitrile and 4- bromoaniline | 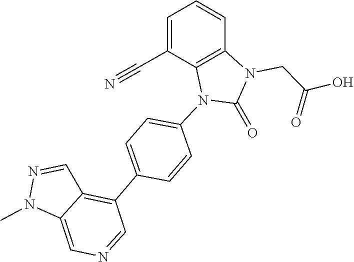 [SMILES: Cn1ncc2c(-c3ccc(-n4c(=O)n(CC(=O)O)c5cccc(C#N)c54)cc3)cncc21] | Method 3: 2.86 min, m/z 425.10 [M + H]⁺ |
| | Intermediate 1-48 | |
| | 2-[4-cyano-3-[4-(1-methylpyrazolo[3,4-c]pyridin-4- | |
| | yl)phenyl]-2-oxo-benzimidazol-1-yl]acetic acid | |
| 2-fluoro-3-nitro- pyridine and 4- bromoaniline | 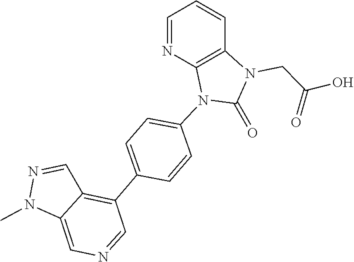 [SMILES: Cn1ncc2c(-c3ccc(-n4c(=O)n(CC(=O)O)c5cccnc54)cc3)cncc21] | Method 4: 0.96 min, m/z 401.10 [M + H]⁺ |
| | Intermediate I-49 | |
| | 2-[3-[4-(1-methylpyrazolo[3,4-c]pyridin-4-yl)phenyl]-2- | |
| | oxo-imidazo[4,5-b]pyridin-1-yl]acetic acid | |
| 3-fluoro-4-nitro- pyridine and 4- bromoaniline | 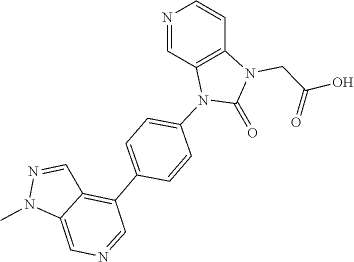 [SMILES: Cn1ncc2c(-c3ccc(-n4c(=O)n(CC(=O)O)c5ccncc54)cc3)cncc21] | Method 4: 0.78 min, m/z 401.20 [M + H]⁺ |
| | Intermediate 1-50 | |
| | 2-[3-[4-(1-methylpyrazolo[3,4-c]pyridin-4-yl)phenyl]-2- | |
| | oxo-imidazo[4,5-c]pyridin-1-yl]acetic acid | |
| 4-fluoro-5-nitro- pyridine and 4- bromoaniline | 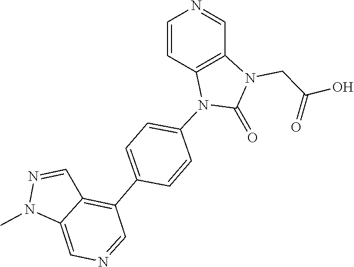 [SMILES: Cn1ncc2c(-c3ccc(-n4c(=O)n(CC(=O)O)c5cnccc54)cc3)cncc21] | Method 4: 0.33 min, m/z 401.10 [M + H]⁺ |
| | Intermediate 1-51 | |
| | 2-[1-[4-(1-methylpyrazolo[3,4-c]pyridin-4-yl)phenyl]-2- | |
| | oxo-imidazo[4,5-c]pyridin-3-yl]acetic acid | |
| 2-fluoro-N,N- dimethyl-3- nitroaniline and 4- bromoaniline | 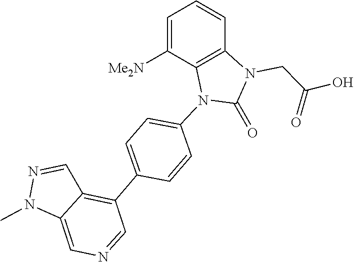 [SMILES: CN(C)c1cccc2c1n(-c1ccc(-c3cncc4c3cnn4C)cc1)c(=O)n2CC(=O)O] | Method 4: 0.99 min, m/z 443.00 |
| | Intermediate 1-52 | |
| | 2-[4-(dimethylamino)-3-[4-(1-methylpyrazolo[3,4- | |
| | c]pyridin-4-yl)phenyl]-2-oxo-benzimidazol-1-yl]acetic acid | |
| 2-fluoro-1-methoxy- 3-nitro-benzene and 4-Bromo-2- methylaniline | 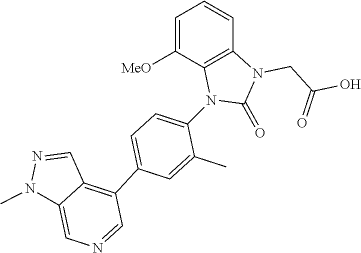 [SMILES: COc1cccc2c1n(-c1ccc(-c3cncc4c3cnn4C)cc1C)c(=O)n2CC(=O)O] | Method 4: 1.71 min, m/z 444.30 |
| | Intermediate 1-53 | |
| | 2-[4-methoxy-3-[2-methyl-4-(1-methylpyrazolo[3,4- | |
| | c]pyridin-4-yl)phenyl]-2-oxo-benzimidazol-1-yl]acetic acid | |
| 2-fluoro-1-methyl-3- nitrobenzene and 4- bromo-2- methylaniline | 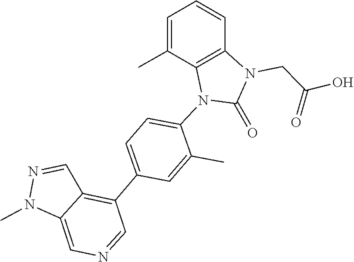 [SMILES: Cc1cc(-c2cncc3c2cnn3C)ccc1-n1c(=O)n(CC(=O)O)c2cccc(C)c21] | Method 4: 1.33 min, m/z 428.00 |
| | Intermediate 1-54 | |
| | 2-[4-methyl-3-[2-methyl-4-(1-methylpyrazolo[3,4- | |
| | c]pyridin-4-yl)phenyl]-2-oxo-benzimidazol-1-yl]acetic acid | |
| 1-chloro-2-fluoro-3- nitrobenzene and 4- bromo-2- methylaniline | 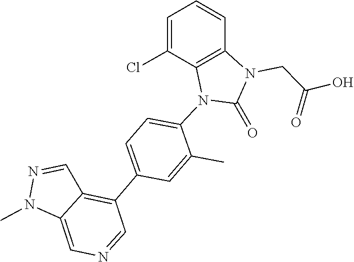 [SMILES: Cc1cc(-c2cncc3c2cnn3C)ccc1-n1c(=O)n(CC(=O)O)c2cccc(Cl)c21] | Method 3: 3.25 min, m/z 448.10 |
| | Intermediate 1-55 | |
| | 2-[4-chloro-3-[2-methyl-4-(1-methylpyrazolo[3,4- | |
| | c]pyridin-4-yl)phenyl]-2-oxo-benzimidazol-1-yl]acetic acid | |
| 2-fluoro-1-methoxy- 3-nitro-benzene and 4-bromoaniline | 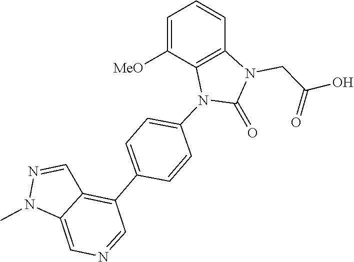 [SMILES: COc1cccc2c1n(-c1ccc(-c3cncc4c3cnn4C)cc1)c(=O)n2CC(=O)O] | Method 3: 3.05 min, m/z 430.10 |
| | Intermediate 1-56 | |
| | 2-[4-methoxy-3-[4-(1-methylpyrazolo[3,4-c]pyridin-4- | |
| | yl)phenyl]-2-oxo-benzimidazol-1-yl]acetic acid | |
| 2,3-difluoronitro- benzene and 4- bromoaniline | 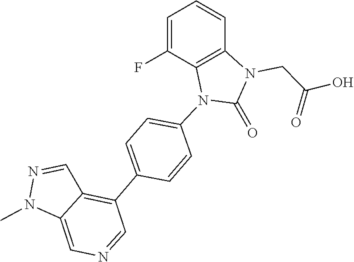 [SMILES: Cn1ncc2c(-c3ccc(-n4c(=O)n(CC(=O)O)c5cccc(F)c54)cc3)cncc21] | Method 3: 3.03 min, m/z 418.15 |
| | Intermediate 1-57 | |
| | 2-[4-fluoro-3-[4-(1-methylpyrazolo[3,4-c]pyridin-4- | |
| | yl)phenyl]-2-oxo-benzimidazol-1-yl]acetic acid | |
| 2- fluoronitrobenzene and 4-bromo-2- methoxyaniline | 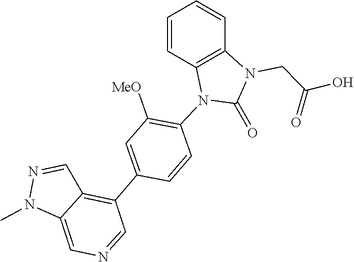 [SMILES: COc1cc(-c2cncc3c2cnn3C)ccc1-n1c(=O)n(CC(=O)O)c2ccccc21] | Method 4: 1.28 min, m/z 430.00 |
| | Intermediate 1-59 | |
| | 2-[3-[2-methoxy-4-(1-methylpyrazolo[3,4-c]pyridin-4- | |
| | yl)phenyl]-2-oxo-benzimidazol-1-yl]acetic acid | |

| Conditions | Structure and ID | LCMS (ES⁺) |
|---|---|---|
| Scheme 10, Step A using Intermediate I-43 and tributyl- (1-methylpyrazolo[3,4- c]pyridin-4-yl)stannane | 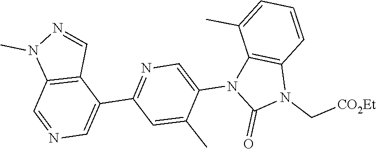 [SMILES: CCOC(=O)Cn1c(=O)n(-c2cnc(-c3cncc4c3cnn4C)cc2C)c2c(C)cccc21] | Method 3: 3.28 min, m/z, 457.30 [m + h] |
| | Intermediate 1-84 | |
| Scheme 10, Step A using Intermediate I-43 and tributyl- (1,3-dimethylpyrazolo[3,4- c]pyridin-4-yl)stannane | 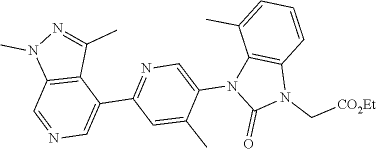 | Method 3: 2.95 min, m/z, 457.35 [M + H] |
| | Intermediate 1-85 | |
| Scheme 10, Step A using Intermediate I-38 and tributyl- (1,5-dimethylpyrazolo[3,4- c]pyridin-4-yl)stannane | 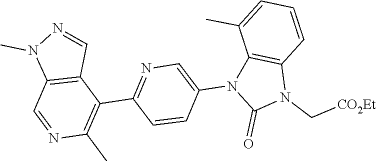 [SMILES: CCOC(=O)Cn1c(=O)n(-c2ccc(-c3c(C)ncc4c3cnn4C)nc2)c2c(C)cccc21] | Method 4: 1.23 min, m/z, 457.2 [M + H]+ (%) |
| | Intermediate 1-86 | |
| Scheme 10, Step A using Intermediate I-83 and tributyl- (1-methylpyrazolo[3,4- c]pyridin-4-yl)stannane | 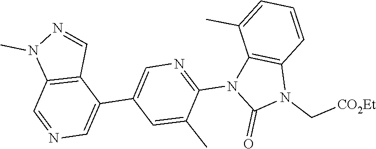 [SMILES: CCOC(=O)Cn1c(=O)n(-c2ncc(-c3cncc4c3cnn4C)cc2C)c2c(C)cccc21] | Method 3: 3.35 min, m/z, 457 [M + H] |
| | Intermediate 1-87 | |
| Scheme 10, Step A using Intermediate I-63 and tributyl- (1-methylpyrazolo[3,4- c]pyridin-4-yl)stannane | 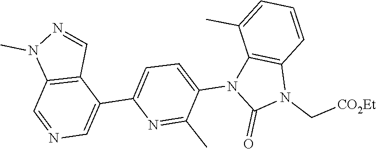 [SMILES: CCOC(=O)Cn1c(=O)n(-c2ccc(-c3cncc4c3cnn4C)nc2C)c2c(C)cccc21] | Method 4: 1.62 min, m/z, 457.2 [M + H]+ (%) |
| | Intermediate 1-88 | |
| Scheme 10, Step A using Intermediate 1-64 and tributyl- (1-methylpyrazolo[3,4- c]pyridin-4-yl)stannane | 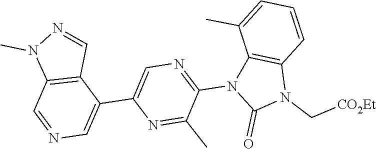 [SMILES: CCOC(=O)Cn1c(=O)n(-c2ncc(-c3cncc4c3cnn4C)nc2C)c2c(C)cccc21] | Method 3: 3.35 min, m/z, 458.25 [M + H] |
| | Intermediate 1-89 | |

Intermediates 1-90 to 1-95 were synthesised following the same procedure as Intermediate I-95 (Scheme 13, Step 6) and are described in the table below.

| ID | Structure | LCMS (ES⁺) |
|---|---|---|
| Intermediate 1-90 | 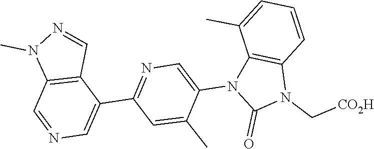 [SMILES: Cc1cc(-c2cncc3c2cnn3C)ncc1-n1c(=O)n(CC(=O)O)c2cccc(C)c21] | Method 3: 2.75 min, m/z, 429.30 [M + H] |
| Intermediate I-91 | 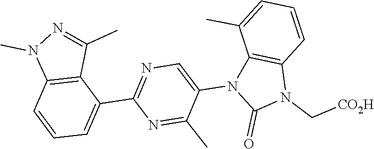 [SMILES: Cc1nc(-c2cccc3c2c(C)nn3C)ncc1-n1c(=O)n(CC(=O)O)c2cccc(C)c21] | Method 3: 1.10 min, m/z, 443.10 [M + H]+ |
| Intermediate I-92 | 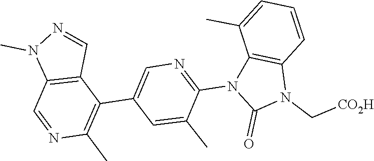 | Method 4: 0.80 min, m/z, 429.1 [M + H]+ (%) |
| Intermediate I-93 | 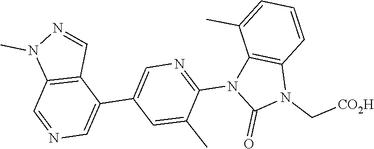 [SMILES: Cc1cc(-c2cncc3c2cnn3C)cnc1-n1c(=O)n(CC(=O)O)c2cccc(C)c21] | Method 3: 2.55 min, m/z, 429 [M + H]+. |
| Intermediate I-94 | 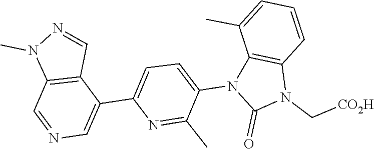 [SMILES: Cc1nc(-c2cncc3c2cnn3C)ccc1-n1c(=O)n(CC(=O)O)c2cccc(C)c21] | Method 4: 1.13 min, m/z, 429.2 [M + H]+ |
| Intermediate I-95 | 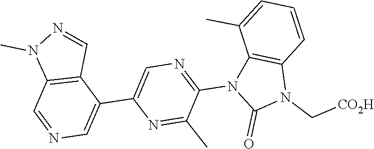 [SMILES: Cc1nc(-c2cncc3c2cnn3C)cnc1-n1c(=O)n(CC(=O)O)c2cccc(C)c21] | Method 3: 2.88 min, m/z, 430.30 [M + H]+ |

### Synthesis of tributyl-(1-methylpyrazolo[3,4-c]pyridin-4-yl)stannane (I-96)

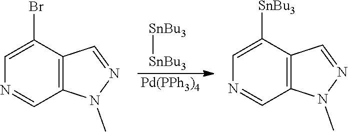

A mixture of 4-bromo-1-methyl-1H-pyrazolo[3,4-c]pyridine (5.0 g, 23.58 mmol), bis(tributyltin) (14.3 mL, 28.3 mmol) and Pd(PPh₃)₄ (2.72 g, 2.36 mmol) in m-Xylene (50 mL) was stirred at 130° C. under N2 for 48 h. The crude material was purified by flash column chromatography eluting with EtOAc in Pet. Ether to give ethyl to give tributyl-(1-methylpyrazolo[3,4-c]pyridin-4-yl)stannane (6.7 g, 15.9 mmol, 67% yield).

LC-MS (ES-API, Aglient-LCMS-01-P2): 2.82 min m/z, 422.1 [M]⁺

| ID | Structure | LCMS (ES⁺) |
|---|---|---|
| Intermediate I-97 | 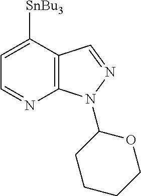 | LC-MS (ES-API, SHIMADZU-LCMS- 2020-P2): 4.450 min, m/z, 493.35 [M + H] |
| Intermediate I-98 | 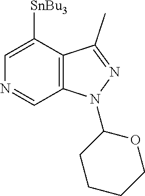 | LC-MS (ES-API, SHIMADZU-LCMS- 2020-P2): 4.183 min, m/z, 508.30 [M + H]+ |
| Intermediate I-99 | 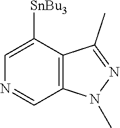 | LC-MS (ES-API, SHIMADZU-LCMS- 2020-P2): 4.550 min, m/z, 437.35 [M + H]+ |
| Intermediate I-100 | 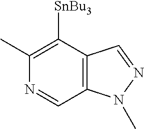 | Method 3: 2.39 min, m/z, 436.2 [M]+ |

### Synthesis of potassium trifluoro(1-methyl-1H-pyrazolo[3,4-c]pyridin-4-yl)borate (I-101)

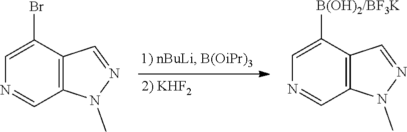

Synthesis of potassium trifluoro(1-methyl-1H-pyrazolo[3,4-c]pyridin-4-yl)borate (I-101): A mixture of 4-bromo-1-methyl-1H-pyrazolo[3,4-c]pyridine (10 g, 47.2 mmol) and B(OiPr)₃ (1.5 eq.) in dry THE (100 mL) was cooled to −78° C. and nBuLi (1.5 eq., 2.5 M in hexane) was added dropwise while maintaining the temperature below −70° C. The reaction mixture was then stirred at −78° C. for 30 min. The reaction was warm up to 0° C. Then KHF₂ (6.0 eq.) was added followed by water. The resulting mixture was stirred at RT for 12 h. The precipitate was filtered and was dried under vaccum at 55° C. for 48 h to give potassium trifluoro(1-methyl-1H-pyrazolo[3,4-c]pyridin-4-yl)borate. LC-MS (ES-API): m/z, 200.0 [M−39]

### EXAMPLES

### Example 1: 2-[3-[4-(1-methylpyrazolo[3,4-c]pyridin-4-yl)phenyl]-2-oxobenzimidazol-1-yl]-N-(2,2,2-trifluoroethyl)acetamide

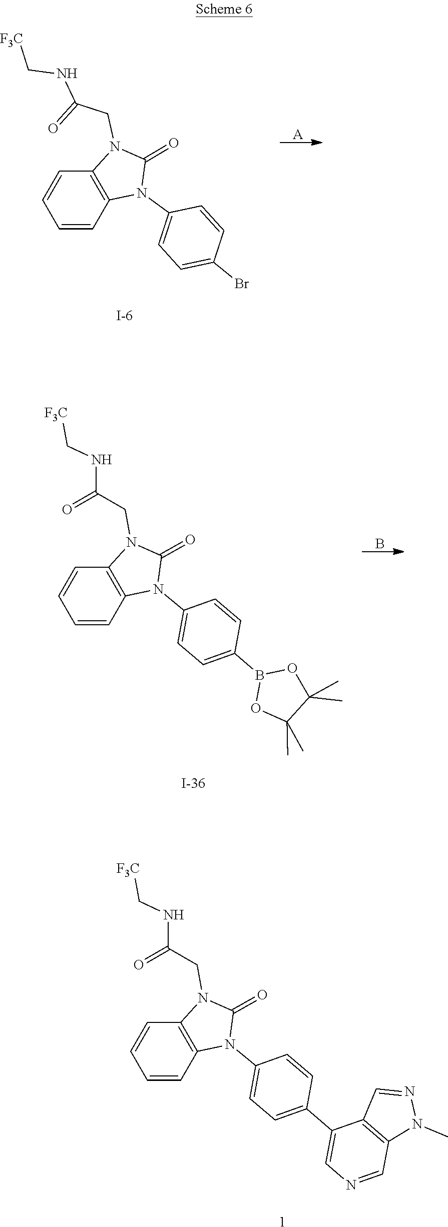

STEP A. Synthesis of Intermediate I-36, 2-[2-oxo-3-[4-(4,4,5,5-tetramethyl-1,3,2-dioxaborolan-2-yl)-phenyl]benzimidazol-1-yl]-N-(2,2,2-trifluoroethyl)acetamide: A suspension of 2-[3-(4-bromophenyl)-2-oxo-benzimidazol-1-yl]-N-(2,2,2-trifluoroethyl)acetamide (Intermediate I-6, 1.99 g, 4.65 mmol), potassium acetate (1.60 g, 16.28 mmol), bis(pinacolato)diboron (1.77 g, 6.98 mmol) in 1,4-Dioxane (80 mL) and DMF (8 mL) was purged (3× vacuum, 3× N₂). After addition of Pd(dppf)Cl₂-DCM (570 mg, 0.7 mmol) the suspension was purged again (3× vacuum, 3× N₂) and heated to 95° C. under N₂-atmosphere. After 17 h, the mixture was filtered through a paper filter. The filter was rinsed well with EtOAc and the filtrate was reduced in vacuo and purified by flash chromatography (SiO₂, 25 g, EtOAc in pet. ether, 5-95%) to give 2-[2-oxo-3-[4-(4,4,5,5-tetramethyl-1,3,2-dioxaborolan-2-yl)phenyl]benzimidazol-1-yl]-N-(2,2,2-trifluoroethyl)acetamide (Intermediate I-36, 2.10 g, 4.41 mmol, 98%) as a brown solid. UPLC-MS (ES⁺, Method 2): 1.92 min, m/z 476.0 [M+H]⁺. ¹H NMR (400 MHz, CDCl₃): δ 8.00 (dbr, J=8.3 Hz, 2H), 7.56 (dbr, J=8.3 Hz, 2H), 7.11-7.24 (m, 4H), 6.83 (t, J=6.5 Hz, 1H), 4.64 (s, 2H), 3.90 (dq, J=6.5, 8.9 Hz, 2H), 1.37 (s, 12H) ppm.

STEP B. Synthesis of Example 1, 2-[3-[4-(1-methylpyrazolo[3,4-c]pyridin-4-yl)phenyl]-2-oxobenz-imidazol-1-yl]-N-(2,2,2-trifluoroethyl)acetamide: A suspension of 2-[2-oxo-3-[4-(4,4,5,5-tetramethyl-1,3,2-dioxaborolan-2-yl)phenyl]benzimidazol-1-yl]-N-(2,2,2-trifluoroethyl)acetamide (Intermediate I-36, 80 mg, 0.17 mmol), potassium acetate (50 mg, 0.50 mmol), Pd(dppf)Cl₂-DCM (21 mg, 0.03 mmol), 4-bromo-1-methyl-1H-pyrazolo[3,4-c]pyridine (71 mg, 0.34 mmol) in 1,4-Dioxane (2.5 mL) and water (0.5 mL) was purged (3× vacuum, 3× N₂) and heated to 100° C. under N₂-atmosphere. After 16 h the mixture was cooled to room temperature and purified by flash chromatography (SiO₂, MeOH in EtOAc 0-20%, then MeOH in DCM 0-20%) to give 2-[3-[4-(1-methylpyrazolo[3,4-c]pyridin-4-yl)phenyl]-2-oxobenzimidazol-1-yl]-N-(2,2,2-trifluoroethyl)acetamide (Example 1, 23 mg, 0.05 mmol, 28%) as white solid. UPLC-MS (ES⁺, Method 1): 3.07 min, m/z 481.6 [M+H]⁺. ¹H-NMR (400 MHz, DMSO-d₆): δ 9.23 (s, 1H), 9.00 (t, J=6.3 Hz, 1H), 8.51 (s, 1H), 8.43 (d, J=0.8 Hz, 1H), 8.05 (td, J=2.3, 9.0 Hz, 2H), 7.77 (td, J=2.3, 9.0 Hz, 2H), 7.23 (dbr, J=7.3 Hz, 1H), 7.10-7.21 (m, 4H), 4.70 (s, 2H), 4.26 (s, 3H), 3.99 (dq, J=6.0, 9.9 Hz, 2H) ppm.

Examples synthesised following the same procedure as Example 1 (scheme 6) replacing Intermediate 1-6 with the appropriate building blocks for Step A and replacing 4-bromo-1-methyl-1H-pyrazolo[3,4-c]pyridine with the appropriate building blocks in Step B are described in Table 8.

TABLE 8

| | | LC-MS | ¹H-NMR (400 MHz, |
|---|---|---|---|
| Building blocks | Example structure and name | (ES⁺) | DMSO-d₆): |
| Step A: Intermediate I-7 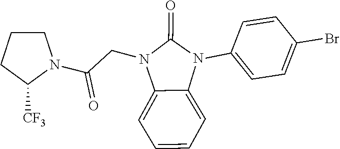 [SMILES: O=C(Cn1c(=O)n(-c2ccc(Br)cc2)c2ccccc21)N1CCC[C@H]1C(F)(F)F] Step B: 4-bromo-1-methyl- 1H-pyrazolo[3,4- c]pyridine 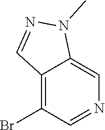 | 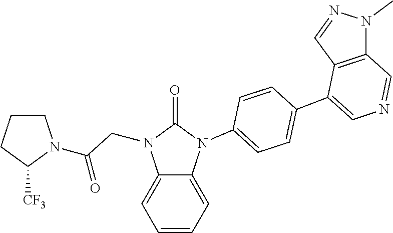 [SMILES: Cn1ncc2c(-c3ccc(-n4c(=O)n(CC(=O)N5CCC[C@H]5C(F)(F)F)c5ccccc54)cc3)cncc21] | Method 3: 3.553 min, m/z 521.4 [M + H]⁺ | δ 9.22 (s, 1H), 8.51 (s, 1H), 8.42 (s, 1H), 8.04 (d, J = 8.5 Hz, 2H), 7.76 (d, J = 8.5 Hz, 2H), 7.22 (dd, J = 7.6, 6.1 Hz, 2H), 7.16 (td, J = 7.6, 1.4 Hz, 1H), 7.11 (td, J = 7.6, 1.5 Hz, 1H), 5.16-4.84 (m, 2H), 4.81-4.70 (m, 1H), 4.25 (s, 3H), 3.96-3.63 (m, 2H), 2.36-1.91 (m, 4H) ppm. |
| | 1-[4-(1-methylpyrazolo[3,4-c]- | | |
| | pyridin-4-yl)phenyl]-3-[2-oxo-2- | | |
| | [(2S)-2-(trifluoromethyl)pyrroli- | | |
| | din-1-yl]ethyl]benzimidazol-2- | | |
| | one. Example 2 | | |
| Step A: Intermediate I-8 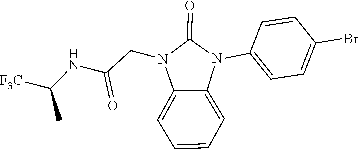 Step B: 4-bromo-1-methyl- 1H-pyrazolo[3,4- c]pyridine 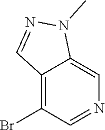 | 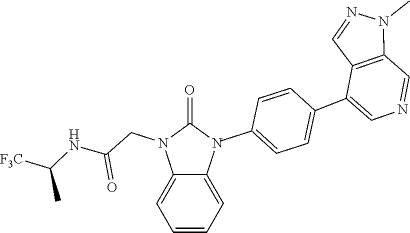 | Method 4: 1.47 min, m/z 495.10 [M + H]⁺ | δ 9.21 (s, 1H), 8.96 (d, J = 8.9 Hz, 1H), 8.50 (s, 1H), 8.42 (s, 1H), 8.03 (d, J = 8.5 Hz, 2H), 7.76 (d, J = 8.5 Hz, 2H), 7.22 (d, J = 7.6 Hz, 1H), 7.18- 7.09 (m, 3H), 4.72-4.57 (m, 3H), 4.25 (s, 3H), 1.30 (d, J = 7.0 Hz, 3H) ppm. |
| | 2-[3-[4-(1-methylpyrazolo[3,4- | | |
| | c]pyridin-4-yl)phenyl]-2- | | |
| | oxobenzimidazol-1-yl]-N-[(1S)- | | |
| | 2,2,2-trifluoro-1- | | |
| | methylethyl]acetamide | | |
| | Example 3 | | |
| Step A: Intermediate I-11 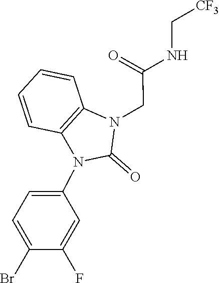 [SMILES: O=C(Cn1c(=O)n(-c2ccc(Br)c(F)c2)c2ccccc21)NCC(F)(F)F] Step B: 4-bromo-1-methyl- 1H-pyrazolo[3,4- c]pyridine 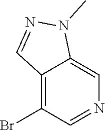 | 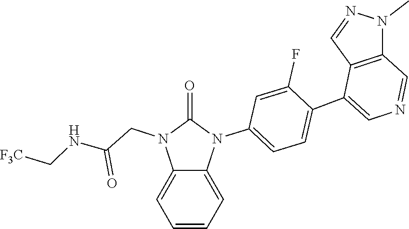 [SMILES: Cn1ncc2c(-c3ccc(-n4c(=O)n(CC(=O)NCC(F)(F)F)c5ccccc54)cc3F)cncc21] | Method 4: 1.500 min, m/z 499.10 [M + H]⁺ | δ 9.28 (s, 1H), 9.01 (t, J = 6.7 Hz, 1H), 8.43 (s, 1H), 8.21 (s, 1H), 7.93 (t, J = 8.2 Hz, 1H), 7.72 (d, J = 11.0 Hz, 1H), 7.66 (d, J = 7.2 Hz, 1H), 7.34 (d, J = 7.2 Hz, 1H), 7.23-7.10 (m, 3H), 4.71 (s, 2H), 4.27 (s, 3H), 4.00 (m_{c}, 2H) ppm. |
| | 2-[3-[3-fluoro-4-(1- | | |
| | methylpyrazolo[3,4-c]pyridin-4- | | |
| | yl)phenyl]-2-oxobenzimidazol- | | |
| | 1-yl]-N-(2,2,2- | | |
| | trifluoroethyl)acetamide | | |
| | Example 4 | | |
| Step A: Intermediate I-12 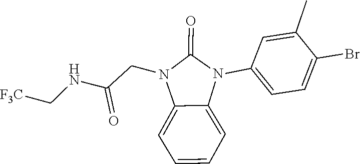 Step B: 4-bromo-1-methyl- 1H-pyrazolo[3,4- c]pyridine 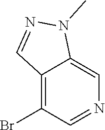 | 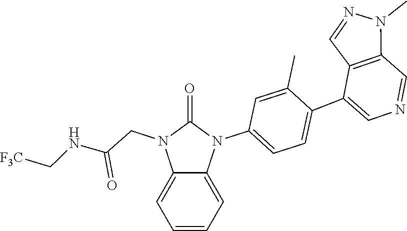 [SMILES: Cc1cc(-n2c(=O)n(CC(=O)NCC(F)(F)F)c3ccccc32)ccc1-c1cncc2c1cnn2C] | Method 4: 1.546 min, m/z 495 10[M + H]⁺ | δ 9.25 (s, 1H), 8.99 (t, J = 6.3 Hz, 1H), 8.26 (s, 1H), 8.01 (s, 1H), 7.61 (d, J = 1.8 Hz, 1H), 7.57-7.50 (m, 2H), 7.22 (d, J = 7.4 Hz, 1H), 7.19-7.07 (m, 3H), 4.69 (s, 2H), 4.24 (s, 3H), 3.99 (qd, J = 9.7, 6.2 Hz, 2H), 2.26 (s, 3H) ppm. |
| | 2-[3-[3-methyl-4-(1- | | |
| | methylpyrazolo[3,4-c]pyridin-4- | | |
| | yl)phenyl]-2-oxobenzimidazol- | | |
| | 1-yl]-N-(2,2,2- | | |
| | trifluoroethyl)acetamide | | |
| | Example 5 | | |
| Step A: Intermediate I-18 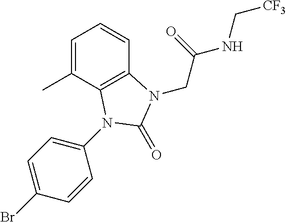 Step B: 4-bromo-1-methyl- 1H-pyrazolo[3,4- c]pyridine 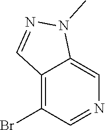 | 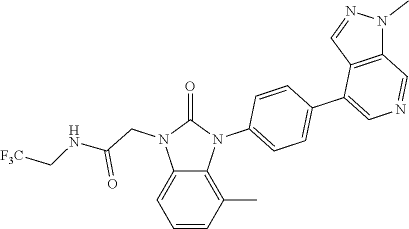 [SMILES: Cc1cccc2c1n(-c1ccc(-c3cncc4c3cnn4C)cc1)c(=O)n2CC(=O)NCC(F)(F)F] | Method 3: 3.36 min, m/z 495.25 [M + H]⁺ | δ 9.23 (s, 1H), 8.96 (t, J = 6.4 Hz, 1H), 8.52 (s, 1H), 8.42 (s, 1H), 8.0 (d, J = 6.8 Hz, 2H), 7.63 (d, J = 6.8 Hz, 2H), 7.06-6.98 (m, 2H), 6.87 (d, J = 7.2 Hz, 1H), 4.65 (s, 2H), 4.26 (s, 3H), 4.0-3.94 (m, 2H), 1.91 (s, 3H) ppm. |
| | 2-[4-methyl-3-[4-(1- | | |
| | methylpyrazolo[3,4-c]pyridin-4- | | |
| | yl)phenyl]-2-oxobenzimidazol- | | |
| | 1-yl]-N-(2,2,2- | | |
| | trifluoroethyl)acetamide | | |
| | Example 6 | | |
| Step A: Intermediate I-19 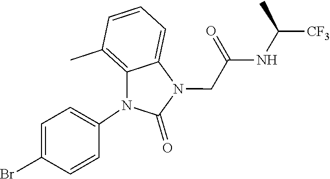 Step B: 4-bromo-1-methyl- 1H-pyrazolo[3,4- c]pyridine 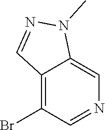 | 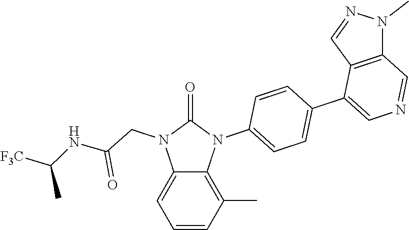 [SMILES: Cc1cccc2c1n(-c1ccc(-c3cncc4c3cnn4C)cc1)c(=O)n2CC(=O)N[C@@H](C)C(F)(F)F] | Method 4: 1.762 min, m/z 509.3 [M + H]⁺ | δ 9.22 (s, 1H), 8.90 (d, J = 12.0 Hz, 1H), 8.52 (s, 1H), 8.41 (s, 1H), 7.99 (d, J = 8.0 Hz, 2H), 7.62 (d, J = 8.0 Hz, 2H), 7.04 (t, J = 8.0 Hz, 1H), 6.98 (d, J = 8.0 Hz, 1H), 6.86 (d, J = 8.0 Hz, 1H), 4.67-4.57 (m, 3H), 4.25 (s, 3H), 1.90 (s, 3H), 1.30 (d, J = 4.0 Hz, 3H) ppm. |
| | 2-[4-methyl-3-[4-(1- | | |
| | methylpyrazolo[3,4-c]pyridin-4- | | |
| | yl)phenyl]-2-oxobenzimidazol- | | |
| | 1-yl]-N-[(1S)-2,2,2-trifluoro-1- | | |
| | methylethyl]acetamide | | |
| | Example 7 | | |
| Step A: Intermediate I-20 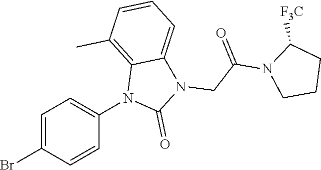 Step B: 4-bromo-1-methyl- 1H-pyrazolo[3,4- c]pyridine 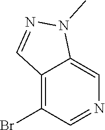 | 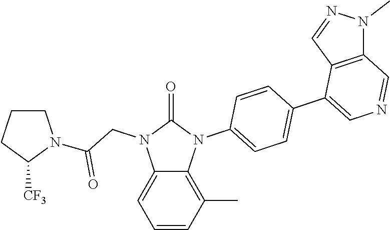 [SMILES: Cc1cccc2c1n(-c1ccc(-c3cncc4c3cnn4C)cc1)c(=O)n2CC(=O)N1CCC[C@H]1C(F)(F)F] | Method 3: 3.650 min, m/z 535.30 [M + H]⁺ | δ 9.23 (s, 1H), 8.52 (s, 1H), 8.42 (s, 1H), 8.00 (d, J = 8.3 Hz, 2H), 7.62 (d, J = 8.1 Hz, 2H), 7.03 (d, J = 4.7 Hz, 2H), 6.89-6.84 (m, 1H), 4.87 (d, J = 2.6 Hz, 2H), 4.81-4.68 (m, 1H), 4.26 (s, 3H), 3.91-3.68 (m, 2H), 2.18-1.95 (m, 4H), 1.91 (s, 3H) ppm. |
| | 4-methyl-3-[4-(1-methylpyrazo- | | |
| | lo[3,4-c]pyridin-4-yl)phenyl]-1- | | |
| | [2-oxo-2-[(2S)-2- | | |
| | (trifluoromethyl)pyrrolidin-1- | | |
| | yl]ethyl]benzimidazol-2-one. | | |
| | Example 8 | | |
| Step A: Intermediate I-21 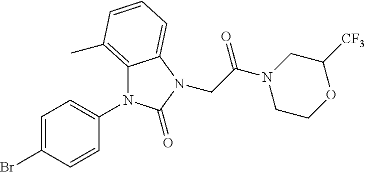 Step B: 4-bromo-1-methyl- 1H-pyrazolo[3,4- c]pyridine 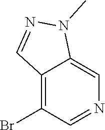 | 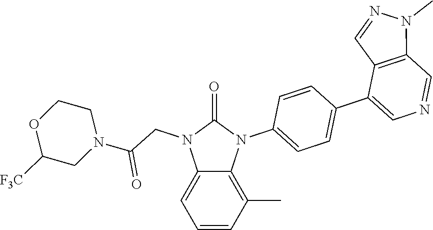 [SMILES: Cc1cccc2c1n(-c1ccc(-c3cncc4c3cnn4C)cc1)c(=O)n2CC(=O)N1CCOC(C(F)(F)F)C1] | Method 3: 3.550 min, m/z 551.25 [M + H]⁺ | δ 9.38 (s, 1H), 8.60 (s, 1H), 8.53 (s, 1H), 8.04 (d, J = 8.0 Hz, 2H), 7.65 (d, J = 8.0 Hz, 2H), 7.13-7.01 (m, 2H), 6.88 (d, J = 7.3 Hz, 1H), 5.07 (m_{c}, 1H), 4.87 (dd, J = 17.1, 10.7 Hz, 1H), 4.31 (s, 3H), 4.16 (d, J = 13.3 Hz, 1H), 4.04 (dd, J = 15.0, 14.0 Hz, 2H), 3.78 (t, J = 10.6 Hz, 1H), 3.68-3.55 (m, 1H), 3.42 (m, J = 14.1, 13.1 Hz, 1H), 2.94 (m, J = 14.7, 13.2 Hz, 1H), 1.93 (s, 3H) ppm. |
| | 4-methyl-3-[4-(1-methylpyrazo- | | |
| | lo[3,4-c]pyridin-4-yl)phenyl]-1- | | |
| | [2-oxo-2-[2-(trifluoromethyl)- | | |
| | morpholin-4- | | |
| | yl]ethyl]benzimidazol-2-one | | |
| | Example 9 | | |
| Step A: Intermediate I-22 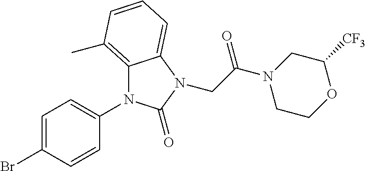 Step B: 4-bromo-1-methyl- 1H-pyrazolo[3,4- c]pyridine 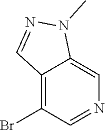 | 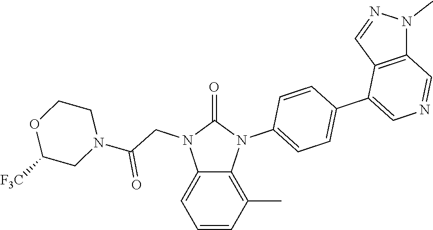 [SMILES: Cc1cccc2c1n(-c1ccc(-c3cncc4c3cnn4C)cc1)c(=O)n2CC(=O)N1CCO[C@@H](C(F)(F)F)C1] | Method 4: 1.913 min, m/z 551.2 [M + H]⁺ | δ 9.22 (s, 1H), 8.52 (s, 1H), 8.41 (s, 1H), 8.00 (d, J = 8.0 Hz, 2H), 7.61 (d, J = 8.0 Hz, 2H), 7.08-7.00 (m, 2H), 6.88-6.82 (m, 1H), 5.10-4.81 (m, 2H), 4.42 3.96 (m, 7H), 3.79-3.54 (m, 1H), 3.45-3.38 (m, 1H), 2.96-2.85 (m, 1H), 1.91 (s, 3H) ppm. |
| | 4-methyl-3-[4-(1- | | |
| | methypyrazolo[3,4-c]pyridin-4- | | |
| | yl)phenyl]-1-[2-oxo-2-[(2R)-2- | | |
| | (trifluoromethyl)morpholin-4- | | |
| | yl]ethyl]benzimidazol-2-one | | |
| | Example 10 | | |
| Step A: Intermediate I-23 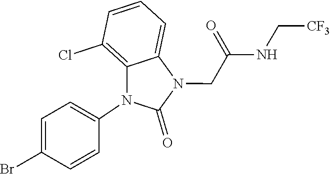 [SMILES: O=C(Cn1c(=O)n(-c2ccc(Br)cc2)c2c(Cl)cccc21)NCC(F)(F)F] Step B: 4-bromo-1-methyl- 1H-pyrazolo[3,4- c]pyridine 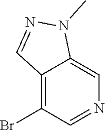 | 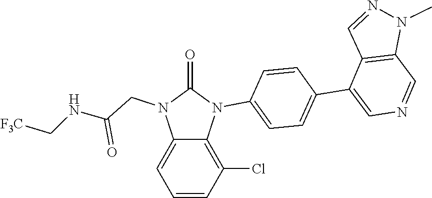 [SMILES: Cn1ncc2c(-c3ccc(-n4c(=O)n(CC(=O)NCC(F)(F)F)c5cccc(Cl)c54)cc3)cncc21] | Method 3: 3.383 min, m/z 515.10 [M + H]⁺ | δ 9.22 (s, 1H), 8.99 (S_{br}, 1H), 8.52 (s, 1H), 8.40 (s, 1H), 7.98 (d, J = 7.9 Hz, 2H), 7.62 (d, J = 7.9 Hz, 2H), 7.30-7.10 (m, 3H), 4.70 (s, 2H), 4.25 (s, 3H), 3.98 (m_{c}, 2H) ppm. |
| | 2-[4-chloro-3-[4-(1- | | |
| | methylpyrazolo[3,4-c]pyridin-4- | | |
| | yl)phenyl]-2-oxobenzimidazol- | | |
| | 1-yl]-N-(2,2,2- | | |
| | trifluoroethyl)acetamide | | |
| | Example 11 | | |
| Step A: Intermediate I-24 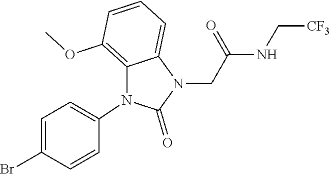 Step B: 4-bromo-1-methyl- 1H-pyrazolo[3,4- c]pyridine 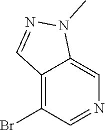 | 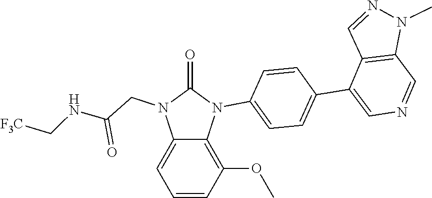 [SMILES: COc1cccc2c1n(-c1ccc(-c3cncc4c3cnn4C)cc1)c(=O)n2CC(=O)NCC(F)(F)F] | Method 3: 3.026 min, m/z 511.05 [M + H]⁺ | δ 9.42 (s, 1H), 8.97 (t, J = 6.3 Hz, 1H), 8.52 (s, 1H), 8.43 (s, 1H), 7.96 (d, J = 8.3 Hz, 2H), 7.60 (d, J = 8.3 Hz, 2H), 7.12 (t, J = 8.1 Hz, 1H), 6.86 (d_{br}, J = 8.1 Hz, 1H), 6.84 (d, 8.1 Hz, 1H), 4.65 (s, 2H), 4.31 (s, 3H), 3.97 (dq, J = 6.2, 9.7 Hz, 2H), 3.70 (s, 3H) ppm. |
| | 2-[4-methoxy-3-[4-(1- | | |
| | methylpyrazolo[3,4-c]pyridin-4- | | |
| | yl)phenyl]-2-oxobenzimidazol- | | |
| | 1-yl]-N-(2,2,2- | | |
| | trifluoroethyl)acetamide | | |
| | Example 12 | | |
| Step A: Intermediate I-25 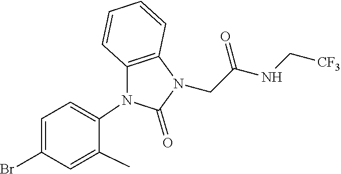 Step B: 4-bromo-1-methyl- 1H-pyrazolo[3,4- c]pyridine 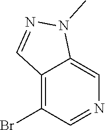 | 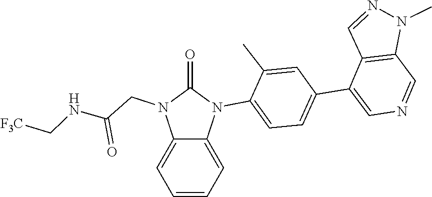 [SMILES: Cc1cc(-c2cncc3c2cnn3C)ccc1-n1c(=O)n(CC(=O)NCC(F)(F)F)c2ccccc21] | Method 4: 1.73 min, m/z 495.2 [M + H]⁺ | δ 9.21 (s, 1H), 9.00 (t, J = 6.7 Hz, 1H), 8.50 (s, 1H), 8.44 (s, 1H), 7.91 (d, J = 1.7 Hz, 1H), 7.84 (dd, J = 1.7, 8.1 Hz, 1H), 7.55 (d, J = 8.2 Hz, 1H), 7.18 (dd, J = 1.0, 8.0 Hz, 1H), 7.13 (dt, J = 1.0, 7.8 Hz, 1H), 7.05 (dt, J = 1.3, 7.5 Hz, 1H), 6.78 (d, J = 7.7 Hz, 1H), 6.78 (s, 1H), 4.70 (s, 2H), 4.25 (s, 3H), 4.00 (m_{c}, 2H), 2.21 (s, 3H) ppm. |
| | 2-[3-[2-methyl-4-(1- | | |
| | methylpyrazolo[3,4-c]pyridin-4- | | |
| | yl)phenyl]-2-oxobenzimidazol- | | |
| | 1-yl]-N-(2,2,2- | | |
| | trifluoroethyl)acetamide | | |
| | Example 13 | | |
| Step A: Intermediate I-25 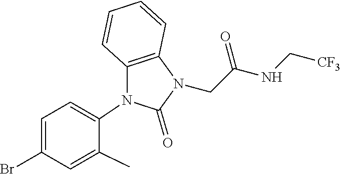 Step B: 4-bromo-1H- pyrazolo[3,4-c]pyridine 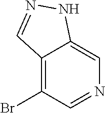 | 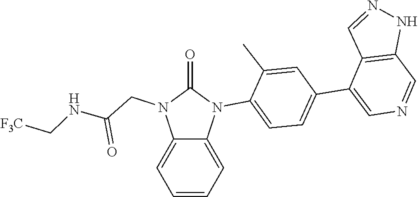 [SMILES: Cc1cc(-c2cncc3[nH]ncc23)ccc1-n1c(=O)n(CC(=O)NCC(F)(F)F)c2ccccc21] | Method 3: 3.017 min, m/z 481.10 [M + H]⁺ | δ 9.10 (s, 1H), 9.01 (t, J = 6.3 Hz, 1H), 8.48 (d, J = 4.6 Hz, 2H), 8.37-8.23 (m, 1H), 7.93 (d, J = 2.0 Hz, 1H), 7.84 (d, J = 8.1, 2.1 Hz, 1H), 7.55 (d, J = 8.1 Hz, 1H), 7.21-7.10 (m, 2H), 7.06 (t, J = 7.6 Hz, 1H), 6.79 (d, J = 7.7 Hz, 1H), 4.70 (s, 2H), 3.99 (m_{c}, 2H), 2.21 (s, 3H) ppm. |
| | 2-[3-[2-methyl-4-(1H-pyrazolo- | | |
| | [3,4-c]pyridin-4-yl)phenyl]-2- | | |
| | oxobenzimidazol-1-yl]-N- | | |
| | (2,2,2-trifluoroethyl)acetamide | | |
| | Example 14 | | |
| Step A: Intermediate I-26 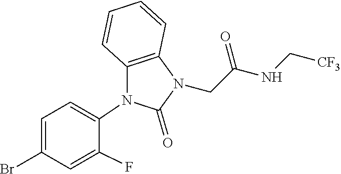 [SMILES: O=C(Cn1c(=O)n(-c2ccc(Br)cc2F)c2ccccc21)NCC(F)(F)F] Step B: 4-bromo-1-methyl- 1H-pyrazolo[3,4- c]pyridine 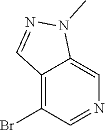 | 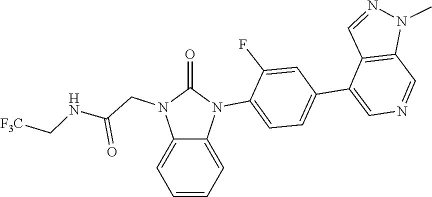 [SMILES: Cn1ncc2c(-c3ccc(-n4c(=O)n(CC(=O)NCC(F)(F)F)c5ccccc54)c(F)c3)cncc21] | Method 3: 3.41 min, m/z 499.15 [M + H]⁺ | δ 9.27 (s, 1H), 9.01 (t, J = 6.4 Hz, 1H), 8.55 (s, 1H), 8.48 (s, 1H), 7.99 (dd, J = 11.2, 1.9 Hz, 2H), 7.89 (dd, J = 8.3, 1.9 Hz, 1H), 7.80 (t, J = 8.0 Hz, 1H), 7.20- 7.15 (m, 2H), 7.11 (m, 1H), 6.97 (d, J = 7.8 Hz, 1H), 4.70 (s, 2H), 4.26 (s, 3H), 4.04-3.95 (m, 2H) ppm. |
| | 2-[3-[2-fluoro-4-(1-methyl- | | |
| | pyrazolo[3,4-c]pyridin-4-yl)- | | |
| | phenyl]-2-oxo-benzimidazol-1- | | |
| | yl]-N-(2,2,2-trifluoroethyl)- | | |
| | acetamide | | |
| | Example 15 | | |
| Step A: Intermediate I-27 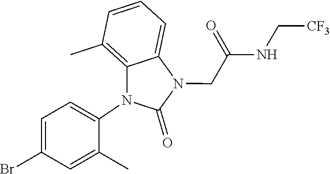 Step B: 4-bromo-1-methyl- 1H-pyrazolo[3,4- c]pyridine 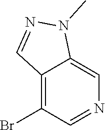 | 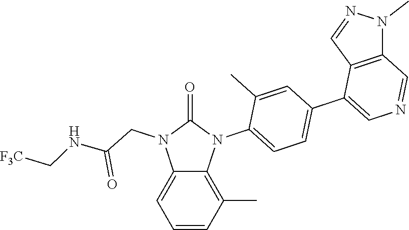 [SMILES: Cc1cc(-c2cncc3c2cnn3C)ccc1-n1c(=O)n(CC(=O)NCC(F)(F)F)c2cccc(C)c21] | Method 3: 3.403 min, m/z 509.25 [M + H]⁺ | δ 9.21 (s, 1H), 8.97 (t, J = 6.4 Hz, 1H), 8.50 (s, 1H), 8.43 (s, 1H), 7.87 (s, 1H), 7.80 (dd, J = 8.0, 2.1 Hz, 1H), 7.54 (d, J = 8.1 Hz, 1H), 7.00 (m, 2H), 6.83 (dd, J = 6.3, 2.7 Hz, 1H), 4.66 (s, 2H), 4.25 (s, 3H), 4.03-3.92 (m, 2H), 2.17 (s, 3H), 1.80 (s, 3H) ppm. |
| | 2-[4-methyl-3-[2-methyl-4-(1- | | |
| | methylpyrazolo[3,4-c]pyridin-4- | | |
| | yl)phenyl]-2-oxo-benzimidazol- | | |
| | 1-yl]-N-(2,2,2-trifluoroethyl)- | | |
| | acetamide | | |
| | Example 16 | | |
| Step A: Intermediate I-58 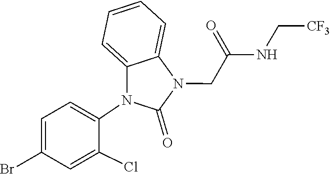 [SMILES: O=C(Cn1c(=O)n(-c2ccc(Br)cc2Cl)c2ccccc21)NCC(F)(F)F] Step B: 4-bromo-1-methyl- 1H-pyrazolo[3,4- c]pyridine 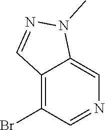 | 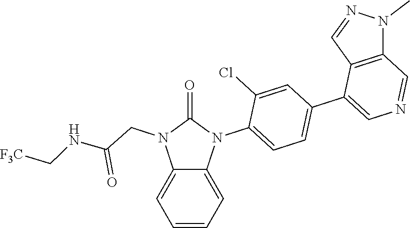 [SMILES: Cn1ncc2c(-c3ccc(-n4c(=O)n(CC(=O)NCC(F)(F)F)c5ccccc54)c(Cl)c3)cncc21] | Method 3: 3.48 min, m/z 515.1 [M + H]⁺ | δ 9.27 (s, 1H), 9.00 (t, J = 6.2 Hz, 1H), 8.55 (s, 1H), 8.45 (s, 1H), 8.17 (s, 1H), 8.02 (d, J = 8.2 Hz, 1H), 7.79 (d, J = 8.2 Hz, 1H), 7.20-7.13 (m, 2H), 7.08 (t, J = 7.6 Hz, 1H), 6.82 (d, J = 7.8 Hz, 1H), 4.75-4.66 (s, 2H), 4.26 (s, 3H), 4.02- 3.99 (m, 2H) ppm. |
| | 2-[3-[2-chloro-4-(1- | | |
| | methylpyrazolo[3,4-c]pyridin-4- | | |
| | yl)phenyl]-2-oxo-benzimidazol- | | |
| | 1-yl]-N-(2,2,2- | | |
| | trifluoroethyl)acetamide | | |
| | Example 87 | | |
| Step A: Intermediate I-60 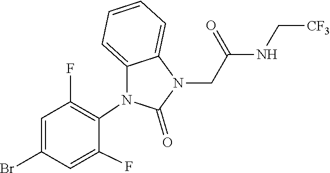 [SMILES: O=C(Cn1c(=O)n(-c2c(F)cc(Br)cc2F)c2ccccc21)NCC(F)(F)F] Step B: 4-bromo-1-methyl- 1H-pyrazolo[3,4- c]pyridine 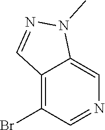 | 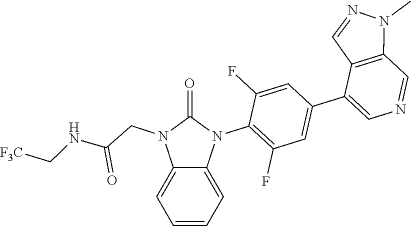 [SMILES: Cn1ncc2c(-c3cc(F)c(-n4c(=O)n(CC(=O)NCC(F)(F)F)c5ccccc54)c(F)c3)cncc21] | Method 3: 3.55 min, m/z 517.05 [M + H]⁺ | δ 9.31 (s, 1H), 9.13 (t, J = 7.2 Hz, 1H), 8.60 (s, 1H), 8.55 (s, 1H), 7.93 (d, J = 9.7 Hz, 2H), 7.24- 7.18 (m, 2H), 7.15 (t, J = 7.5 Hz, 1H), 7.00 (d, J = 7.8 Hz, 1H), 4.74 (s, 2H), 4.29 (s, 3H), 4.06- 3.98 (m, 2H) ppm. |
| | 2-[3-[2,6-difluoro-4-(1- | | |
| | methylpyrazolo[3,4-c]pyridin-4- | | |
| | yl)phenyl]-2-oxo-benzimidazol- | | |
| | 1-yl]-N-(2,2,2- | | |
| | trifluoroethyl)acetamide | | |
| | Example 89 | | |
| Step A: Intermediate I-61 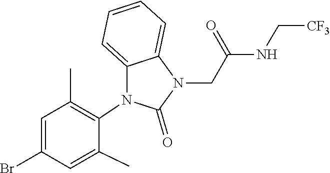 Step B: 4-bromo-1-methyl- 1H-pyrazolo[3,4- c]pyridine 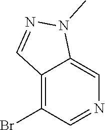 | 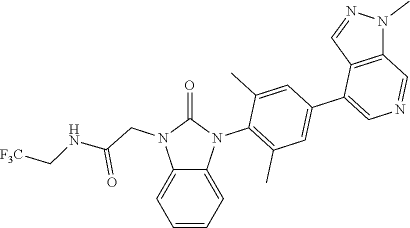 [SMILES: Cc1cc(-c2cncc3c2cnn3C)cc(C)c1-n1c(=O)n(CC(=O)NCC(F)(F)F)c2ccccc21] | Method 3: 3.42 min, m/z 509.10 [M + H]⁺ | δ 9.21 (s, 1H), 8.99 (t, J = 6.4 Hz, 1H), 8.48 (d, J = 13.0 Hz, 2H), 7.72 (s, 2H), 7.20 (d, J = 7.8 Hz, 1H), 7.13 (t, J = 7.6 Hz, 1H), 7.04 (t, J = 7.6 Hz, 1H), 6.64 (d, J = 7.8 Hz, 1H), 4.72 (s, 2H), 4.25 (s, 3H), 4.04-3.95 (m, 2H), 2.12 (s, 6H) ppm. |
| | 2-[3-[2,6-dimethyl-4-(1- | | |
| | methylpyrazolo[3,4-c]pyridin-4- | | |
| | yl)phenyl]-2-oxo-benzimidazol- | | |
| | 1-yl]-N-(2,2,2- | | |
| | trifluoroethyl)acetamide | | |
| | Example 90 | | |

### Example 17: 2-[3-[4-(1-methyl pyrazolo[3,4-c]pyridin-4-yl)phenyl]-2-oxobenzimidazol-1-yl]-N-(2,2,2-trifluoro-1-methylethyl)acetamide

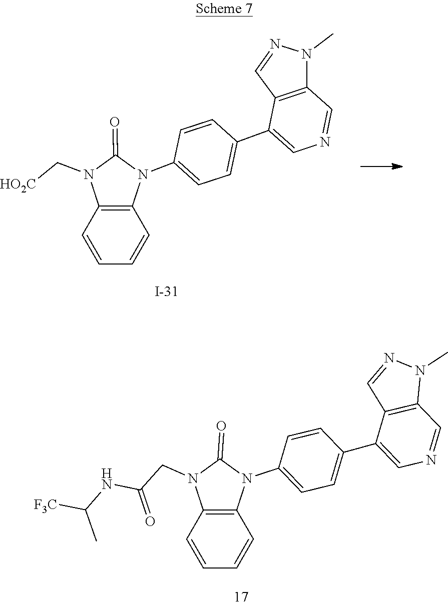

A solution of 2-[3-[4-(1-methyl pyrazolo[3,4-c]pyridin-4-yl)phenyl]-2-oxo-benzimidazol-1-yl]acetic acid (Intermediate I-31, 40 mg, 0.10 mmol), 1,1,1-trifluoro-2-propylamine (12.5 mg, 0.110 mmol), EDCI (23 mg, 0.120 mmol), HOBT (16.2 mg, 0.120 mmol) and DIPEA (39 mg, 0.300 mmol) in DMF (2 mL) was stirred at room temperature overnight. The mixture was poured into water (30 mL), extracted with EtOAc (×3), washed with brine (×3), dried with Na₂SO₄ and concentrated in vacuo and purified by prep-HPLC to give 2-[3-[4-(1-methylpyrazolo[3,4-c]pyridin-4-yl)phenyl]-2-oxobenzimidazol-1-yl]-N-(2,2,2-trifluoro-1-methyl-ethyl)acetamide (Example 17, 13 mg, 0.026 mmol, 26%) as a white solid. LC-MS (ES⁺, Method 4:): 1.533 mi, m/z 495.10 [M+H]⁺. NH-NMR (400 MHz, DMSO-d₆): δ 9.23 (s, 1H), 8.95 (d, J=8.1 Hz, 1H), 8.52 (s, 1H), 8.44 (s, 1H), 8.05 (d, J=7.9 Hz, 2H), 7.78 (d, J=7.9 Hz, 2H), 7.28-7.08 (i, 4H), 4.72-4.59 (i, 3H), 4.30 (s, 3H), 1.32 (d, J=6.6 Hz, 3H) ppm.

Examples synthesised following the same procedure as Example 17 (scheme 7) replacing Intermediate I-31, amine coupling partner and coupling reagents EDCI and HOBT with the appropriate building blocks and reagents are described in Table 9.

TABLE 9

| Building blocks and | | LC-MS | ¹H-NMR (400 MHz, |
|---|---|---|---|
| reagents | Example structure and name | (ES⁺) | DMSO-d6) |
| Intermediate I-31 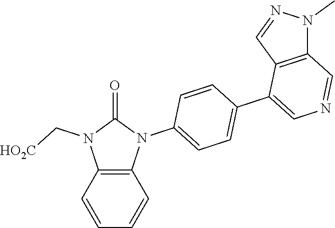 2-(trifluoromethyl)- pyrrolidine (1.1 eq) 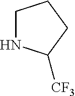 EDCl (1.2 eq) and HOBT (1.2 eq) | 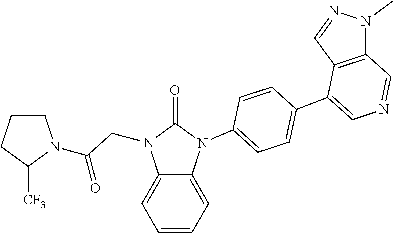 [SMILES: Cn1ncc2c(-c3ccc(-n4c(=O)n(CC(=O)N5CCCC5C(F)(F)F)c5ccccc54)cc3)cncc21] | Method 4: 1.733 min, m/z 521.20 [M + H]⁺ | δ 9.23 (s, 1H), 8.52 (s, 1H), 8.44 (s, 1H), 8.06 (d, J = 8.4 Hz, 2H), 7.78 (d, J = 8.4 Hz, 2H), 7.24 (t, J = 6.6 Hz, 1H), 7.18 (t, J = 7.3 Hz, 1H), 7.13 (t, J = 7.3 Hz, 1H), 4.72-5.31 (m, 3H), 4.27 (s, 3H), 3.92-3.62 (m, 2H), 2.40-1.88 (m, 4H) ppm. |
| | 1-[4-(1-methylpyrazolo[3,4- | | |
| | c]pyridin-4-yl)phenyl]-3-[2-oxo-2- | | |
| | [2-(trifluoromethyl)pyrolidin-1- | | |
| | yl]ethyl]benzimidazol-2-one | | |
| | Example 18 | | |
| Intermediate I-31 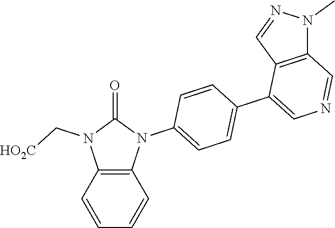 1-cyclopropylethanamine hydrochloride (2.0 eq) 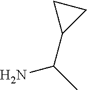 EDCl (1.5 eq) and HOBT (1.5 eq) | 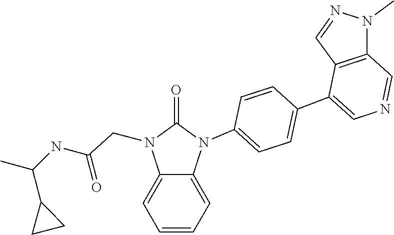 [SMILES: CC(NC(=O)Cn1c(=O)n(-c2ccc(-c3cncc4c3cnn4C)cc2)c2ccccc21)C1CC1] | Method 4: 1.667 min, m/z 467.20 [M + H]⁺ | δ 9.33 (s_{br}, 1H), 8.62 (s_{br}, 1H), 8.49 (s, 1H), 8.33 (d, J = 6.7 Hz, 1H), 8.11 (d, J = 7.4 Hz, 2H), 7.83 (d, J = 7.4 Hz, 2H), 7.35-7.12 (m, 4H), 4.62 (s, 2H), 4.33 (s, 3H), 1.38-1.28 (m, 1H), 1.22 (d, J = 6.0 Hz, 3H), 0.96 (m_{c}, 1H), 0.47 (m_{c}, 2H), 0.33 (mc, 1H), 0.26 (m_{c}, 1H) ppm. |
| | N-(1-cyclopropylethyl)-2-[3-[4-(1- | | |
| | methylpyrazolo[3,4-c]pyridin-4- | | |
| | yl)phenyl]-2-oxobenzimidazol-1- | | |
| | yl]acetamide | | |
| | Example 19 | | |
| Intermediate I-31 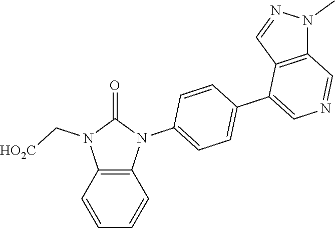 2-(trifluoromethyl)piperidine (2.0 eq) 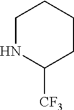 HATU (1.5 eq) | 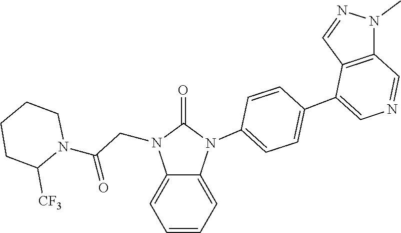 [SMILES: Cn1ncc2c(-c3ccc(-n4c(=O)n(CC(=O)N5CCCCC5C(F)(F)F)c5ccccc54)cc3)cncc21] | Method 4: 1.900 min, m/z 535.20 [M + H]⁺ | δ 9.31 (s, 1H), 8.55 (s, 1H), 8.49 (s, 1H), 8.06 (d, J = 7.0, 2H), 7.78 (d, J = 7.0 Hz, 2H), 7.30- 7.00 (m, 4H), 5.40-3.90 (m, 3H), 4.28 (s, 3H), 2.10-1.00 (m, 8H) ppm. |
| | 1-[4-(1-methylpyrazolo[3,4- | | |
| | c]pyridin-4-yl)phenyl]-3-[2-oxo-2- | | |
| | [2-(trifluoromethyl)-1- | | |
| | piperidyl]ethyl]benzimidazol-2- | | |
| | one | | |
| | Example 20 | | |
| Intermediate I-31 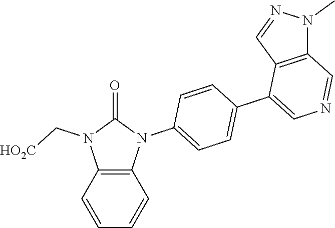 1-methylpiperazine (2.0 eq) 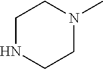 HATU (1.5 eq) | 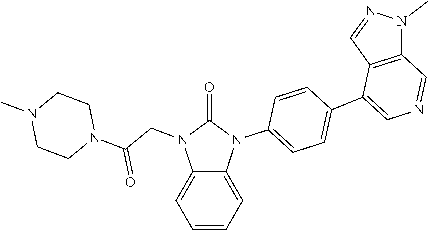 | Method 4: 1.567 min, m/z 482.20 [M + H]⁺ | δ 9.22 (s, 1H), 8.51 (s, 1H), 8.43 (s, 1H), 8.04 (d, J = 8.4 Hz, 2H), 7.76 (d, J = 8.4 Hz, 2H), 7.24-7.18 (m, 2H), 7.14 (dd, J = 1.1, 11.2 Hz, 1H), 7.09 (dd, J = 1.5, 11.2 Hz, 1H), 4.90 (s, 2H), 4.26 (s, 3H), 3.62 (m_{c}, 2H), 3.48 (m_{c}, 2H), 2.48 (m_{c}, 2H), 2.35 (m_{c}, 2H), 2.26 (s, 3H) ppm. |
| | 1-[2-(4-methylpiperazin-1-yl)-2- | | |
| | oxoethyl]-3-[4-(1-methyl- | | |
| | pyrazolo[3,4-c]pyridin-4- | | |
| | yl)phenyl]benzimidazol-2-one | | |
| | Example 21 | | |
| Intermediate I-31 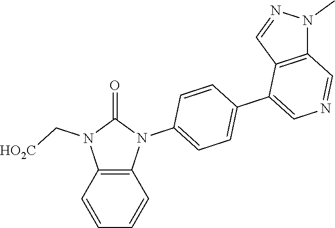 morpholine (2.0 eq) 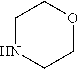 HATU (1.5 eq) | 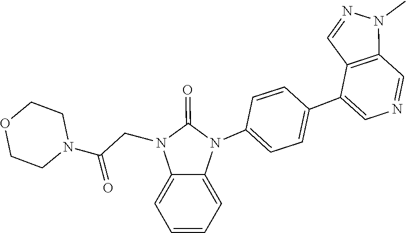 [SMILES: Cn1ncc2c(-c3ccc(-n4c(=O)n(CC(=O)N5CCOCC5)c5ccccc54)cc3)cncc21] | Method 4: 1.400 min, m/z 469.20 [M + H]⁺ | δ 9.30 (s_{br}, 1H), 8.54 (s_{br}, 1H), 8.46 (s, 1H), 8.03 (d, J = 8.1 Hz, 2H), 7.75 (d, J = 8.1 Hz, 2H), 7.24-7.04 (m, 4H), 4.90 (s, 2H), 4.25 (s, 3H), 3.70-3.40 (m, 8H) ppm. |
| | 1-[4-(1-methylpyrazolo[3,4- | | |
| | c]pyridin-4-yl)phenyl]-3-(2- | | |
| | morpholino-2-oxoethyl)- | | |
| | benzimidazol-2-one | | |
| | Example 22 | | |
| Intermediate I-31 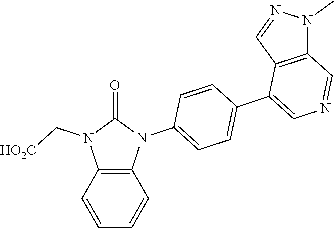 3-(trifluoromethyl)piperidine (2.0 eq) 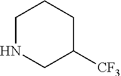 HATU (1.5 eq) | 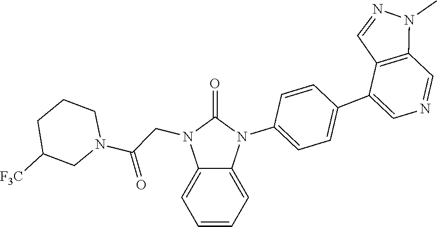 [SMILES: Cn1ncc2c(-c3ccc(-n4c(=O)n(CC(=O)N5CCCC(C(F)(F)F)C5)c5ccccc54)cc3)cncc21] | Method 4: 1.833 min, m/z 535.20 [M + H]⁺ | δ 9.23 (s, 1H), 8.52 (s, 1H), 8.44 (s, 1H), 8.05 (d, J = 8.3 Hz, 2H), 7.77 (d, J = 8.2 Hz, 2H), 7.28-7.07 (m, 4H), 5.05-4.87 (m, 3H), 4.27 (s, 3H), 2.92-2.66 (m, 1H), 2.05-1.01 (m, 5H) ppm. |
| | 1-[4-(1-methylpyrazolo[3,4- | | |
| | c]pyridin-4-yl)phenyl]-3-[2-oxo-2- | | |
| | [3-(trifluoromethyl)-1- | | |
| | piperidyl]ethyl]benzimidazol-2- | | |
| | one | | |
| | Example 23 | | |
| Intermediate I-31 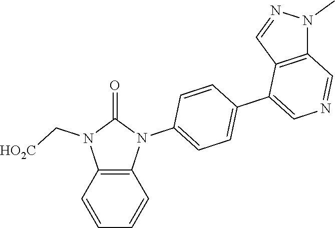 2-(trifluormethyl)morpholine hydrochloride (2.0 eq) 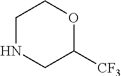 HATU (1.5 eq) | 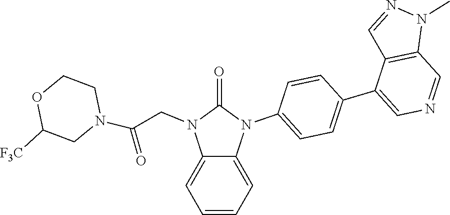 [SMILES: Cn1ncc2c(-c3ccc(-n4c(=O)n(CC(=O)N5CCOC(C(F)(F)F)C5)c5ccccc54)cc3)cncc21] | Method 3: 3.450 min, m/z 537.30 [M + H]⁺ | δ 9.12 (s_{br}, 1H), 8.46 (s_{br}, 1H), 8.38 (s, 1H), 8.02 (d, J = 8.3 Hz, 2H), 7.82 (d, J = 8.3 Hz. 2H), 7.27-7.14 (m, 4H), 5.18-4.90 (m, 1H), 4.58 (s, 2H), 4.30 (s, 3H), 4.36- 3.98 (m, 3H), 3.90-3.44 (m, 2H), 3.03 (m_{c}, 1H) ppm. |
| | 1-[4-(1-methylpyrazolo[3,4- | | |
| | c]pyridin-4-yl)phenyl]-3-[2-oxo-2- | | |
| | [2-(trifluoromethyl)morpholin-4- | | |
| | yl]ethyl]benzimidazol-2-one | | |
| | Example 24 | | |
| Intermediate I-31 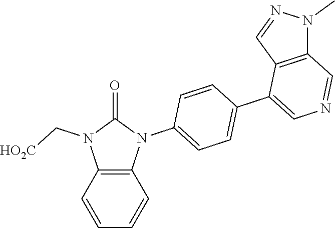 (2S)-2- (difluoromethyl)pyrrolidine (1.1 eq) 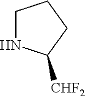 HATU (3.0 eq) | 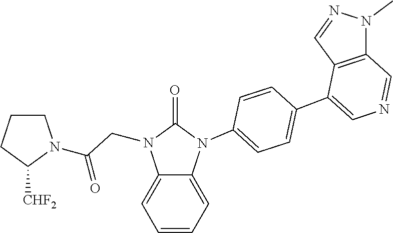 [SMILES: Cn1ncc2c(-c3ccc(-n4c(=O)n(CC(=O)N5CCC[C@H]5C(F)F)c5ccccc54)cc3)cncc21] | Method 4: 1.67 min, m/z 503.3 [M + H]⁺ | δ 9.21 (s, 1H), 8.50 (s, 1H), 8.42 (s, 1H), 8.05-8.02 (m, 2H), 7.78-7.73 (m, 2H), 7.22 (m, 2H), 7.16 (dt, J = 1.4, 7.6 Hz, 1H), 7.11 (dt, J = 1.4, 7.5 Hz, 1H), 6.35-6.03 (m, 1H), 5.07-4.63 (m, 2H), 4.25 (s, 3H), 3.74 (d, J = 5.7 Hz, 2H), 2.09-1.94 (m, 4H) ppm. |
| | 1-[2-[(2S)-2-(difluoromethyl)- | | |
| | pyrrolidin-1-yl]-2-oxo-ethyl]-3-[4- | | |
| | (1-methylpyrazolo[3,4-c]pyridin- | | |
| | 4-yl)phenyl]benzimidazol-2-one | | |
| | Example 25 | | |
| Intermediate I-31 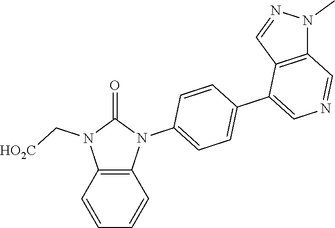 (2S)-2- (fluoromethyl)pyrrolidine (1.0 eq) 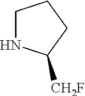 HATU (1.5 eq) | 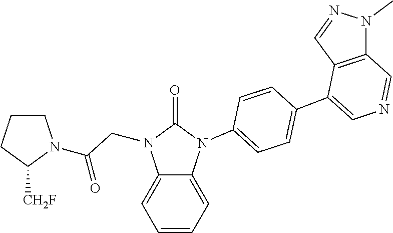 | Method 3: 3.315 min, m/z 499.45 [M + H]⁺ | δ 9.22 (s, 1H), 8.52 (s, 1H), 8.41 (s, 1H), 8.00 (d, J = 8.0 Hz, 2H), 7.61 (d, J = 7.8 Hz, 2H), 7.07-6.98 (m, 2H), 6.85 (d, J = 6.9 Hz, 1H), 4.78 (t, 2H), 4.55-4.36 (m, 2H), 4.25 (s, 3H), 4.15 (d, J = 22.7 Hz, 1H), 3.70-3.45 (m, 2H), 2.02-1.86 (m, 7H) ppm. |
| | 4-methyl-3-[4-(1- | | |
| | methylpyrazolo[3,4-c]pyridin-4- | | |
| | yl)phenyl]-1-[2-oxo-2-[(2S)-2- | | |
| | (fluoromethyl)pyrrolidin-1- | | |
| | yl]ethyl]benzimidazol-2-one | | |
| | Example 26 | | |
| Intermediate I-31 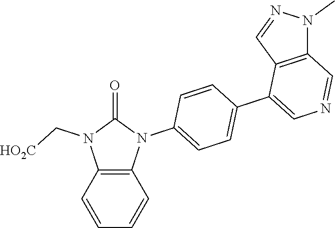 (2R)-2-methylpyrrolidine (1.5 eq) 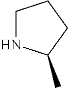 | 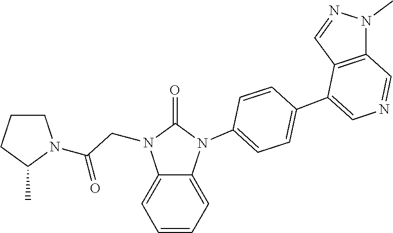 [SMILES: C[C@@H]1CCCN1C(=O)Cn1c(=O)n(-c2ccc(-c3cncc4c3cnn4C)cc2)c2ccccc21] | Method 3: 3.250 min, m/z 467.30 [M + H]⁺ | δ 9.22 (s, 1H), 8.50 (s, 1H), 8.42 (s, 1H), 8.03 (d, J = 8.1 Hz, 2H), 7.76 (d, J = 8.1 Hz, 2H), 7.25-7.18 (m, 2H), 7.15 (t, J = 7.7 Hz, 1H), 7.10 (m), 4.95-4.69 (m, 2H), 4.25 (s, 3H), 4.04 (s_{br}, 1H), 3.69 (m_{c}, 1H), 3.62 (m_{c}, 1H), 2.11-1.87 (m, 3H), 1.59- 1.50 (m, 1H), 1.29 (d, J = 6.1 Hz, 1H), 1.29 (d, J = 6.1 Hz, 2H) ppm. |
| | 1-[4-(1-methylpyrazolo[3,4- | | |
| | c]pyridin-4-yl)phenyl]-3-[2-[(2R)- | | |
| | 2-methylpyrrolidin-1-yl]-2- | | |
| | oxoethyl]benzimidazol-2-one | | |
| | Example 27 | | |
| Intermediate I-31 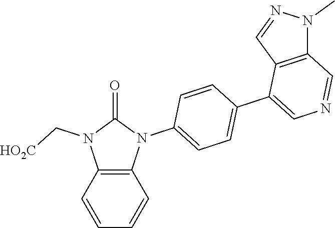 4,4,4-trifluorobutan-2-amine (1.2 eq) 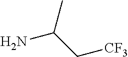 T3P (1.5 eq) | 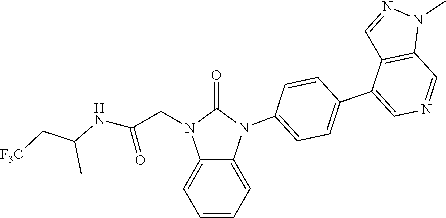 [SMILES: CC(CC(F)(F)F)NC(=O)Cn1c(=O)n(-c2ccc(-c3cncc4c3cnn4C)cc2)c2ccccc21] | Method 3: 3.317 min, m/z 509.15 [M + H]⁺ | δ 9.22 (s, 1H), 8.50 (s, 1H), 8.43-8.38 (m, 2H), 8.04 (d, J = 8.3 Hz, 2H), 7.76 (d, J = 8.3 Hz, 2H), 7.21 (d, J = 7.8 Hz, 1H), 7.19- 7.07 (m, 3H), 4.54 (q, J = 14.3 Hz, 1H), 4.25 (s, 3H), 4.16 (mc, 1H), 3.42-3.40 (m, 2H), 1.19 (d, J = 6.6 Hz, 3H) ppm. |
| | 2-[3-[4-(1-methylpyrazolo[3,4- | | |
| | c]pyridin-4-yl)phenyl]-2-oxo- | | |
| | benzimidazol-1-yl]-N-(3,3,3- | | |
| | trifluoro-1-methyl- | | |
| | propyl)acetamide | | |
| | Example 28 | | |
| Intermediate I-31 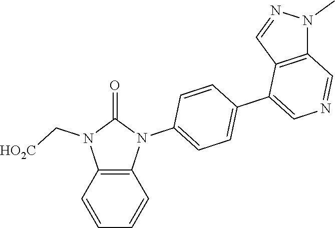 (3R)-3- (trifluoromethyl)piperidine hydrochloride (1.1 eq) 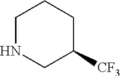 HATU (3.0 eq) | 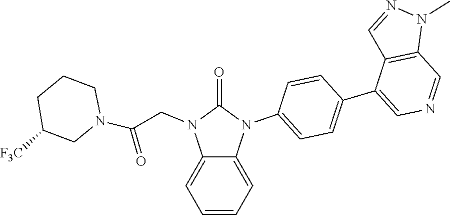 [SMILES: Cn1ncc2c(-c3ccc(-n4c(=O)n(CC(=O)N5CCC[C@@H](C(F)(F)F)C5)c5ccccc54)cc3)cncc21] | Method 4: 1.86 min, m/z 535.3 [M + H]⁺ | δ 9.22 (s, 1H), 8.50 (s, 1H), 8.42 (s, 1H), 8.03 (d, J = 8.5 Hz, 2H), 7.75 (d, J = 8.5 Hz, 2H), 7.25-7.08 (m, 4H), 5.03-4.85 (m, 2H), 4.43-3.93 (m, 2H), 4.25 (s, 3H), 3.30-2.65 (m, 3H), 2.00 (d, J = 11.0 Hz, 1H), 1.90-1.70 (m, 1H) 1.69-1.35 (m, 2H) ppm. |
| | 1-[4-(1-methylpyrazolo[3,4-c]- | | |
| | pyridin-4-yl)phenyl]-3-[2-oxo-2- | | |
| | [(3R)-3-(trifluoromethyl)-1- | | |
| | piperidyl]ethyl]benzimidazol-2- | | |
| | one | | |
| | Example 29 | | |
| Intermediate I-31 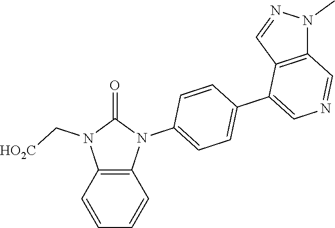 (2R)-2- (trifluoromethyl)piperidine (1.1 eq) 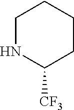 HATU (3.0 eq) | 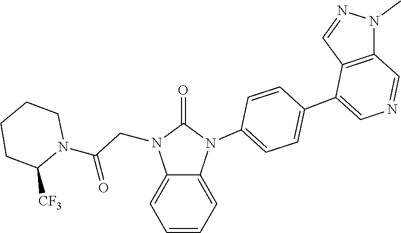 [SMILES: Cn1ncc2c(-c3ccc(-n4c(=O)n(CC(=O)N5CCCC[C@@H]5C(F)(F)F)c5ccccc54)cc3)cncc21] | Method 4: 1.89 min, m/z 535.3 [M + H]⁺ | δ 9.22 (s, 1H), 8.50 (s, 1H), 8.42 (s, 1H), 8.04 (d, J = 8.4 Hz, 2H), 7.76 (d, J = 8.4 Hz, 2H), 7.24-7.08 (m, 4H), 5.34-4.95 (m, 3H), 4.25 (s, 5H), 2.05-1.95 (m, 2H), 1.85-1.40 (m, 4H) ppm. |
| | 1-[4-(1-methylpyrazolo[3,4- | | |
| | c]pyridin-4-yl)phenyl]-3-[2-oxo-2- | | |
| | [(2R)-2-(trifluoromethyl)-1- | | |
| | piperidyl]ethyl]benzimidazol-2- | | |
| | one | | |
| | Example 30 | | |
| Intermediate I-31 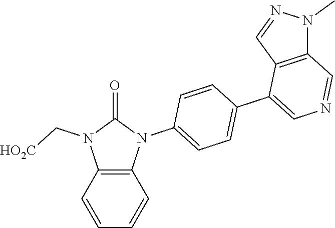 (3S)-3- (trifluoromethyl)piperidine (1.1 eq) 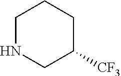 HATU (3.0 eq) | 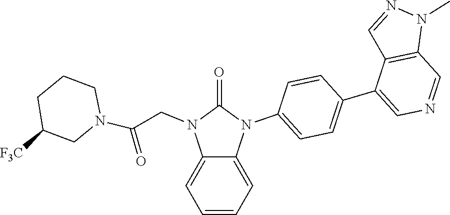 [SMILES: Cn1ncc2c(-c3ccc(-n4c(=O)n(CC(=O)N5CCC[C@H](C(F)(F)F)C5)c5ccccc54)cc3)cncc21] | Method 4: 1.80 min, m/z 535.3 [M + H]⁺ | δ 9.22 (s, 1H), 8.50 (s, 1H), 8.42 (s, 1H), 8.03 (d, J = 8.2 Hz, 2H), 7.75 (d, J = 8.2 Hz, 2H), 7.26-7.07 (m, 4H), 4.94 (dd, J = 14.6, 6.2 Hz, 2H), 4.44-3.95 (m, 5H), 3.27-3.19 (m, 1H), 2.90-2.66 (m, 2H), 2.00 (dd, J = 9.5, 4.4 Hz, 1H), 1.90-1.70 (m, 1H), 1.68-1.34 (m, 2H) ppm. |
| | 1-[4-(1-methylpyrazolo[3,4- | | |
| | c]pyridin-4-yl)phenyl]-3-[2-oxo-2- | | |
| | [(3S)-3-(trifluoromethyl)-1- | | |
| | piperidyl]ethyl]benzimidazol-2- | | |
| | one | | |
| | Example 31 | | |
| Intermediate I-31 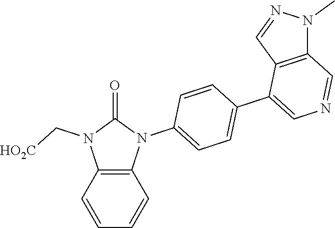 (S)-2- (trifluoromethyl)piperidine (1.1 eq) 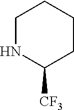 HATU (3.0 eq) | 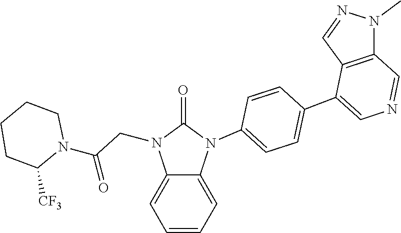 [SMILES: Cn1ncc2c(-c3ccc(-n4c(=O)n(CC(=O)N5CCCC[C@H]5C(F)(F)F)c5ccccc54)cc3)cncc21] | Method 4: 1.80 min, m/z 535.1 [M + H]⁺ | δ 9.24 (s, 1H), 8.51 (s, 1H), 8.44 (s, 1H), 8.04 (d, J = 8.5 Hz, 2H), 7.76 (d, J = 8.5 Hz, 2H), 7.26-7.09 (m, 4H), 5.13 (m_{c}, 2H), 5.04-4.94 (m, 1H), 4.26 (s, 3H), 4.80-4.00 (m, 1H), 2.07-1.55 (m, 7H) ppm. |
| | 1-[4-(1-methylpyrazolo[3,4- | | |
| | c]pyridin-4-yl)phenyl]-3-[2-oxo-2- | | |
| | [(2S)-2-(trifluoromethyl)-1- | | |
| | piperidyl]ethyl]benzimidazol-2- | | |
| | one | | |
| | Example 32 | | |
| Intermediate I-31 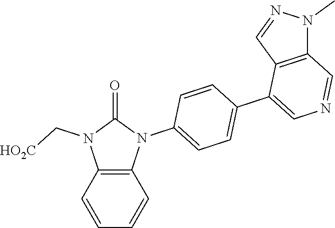 3,3,3-trifluoro-2- methylpropan-1-amine (1.2 eq) 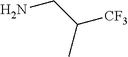 T3P (1.5 eq) | 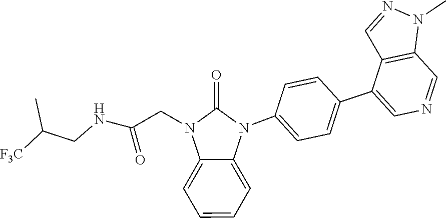 [SMILES: CC(CNC(=O)Cn1c(=O)n(-c2ccc(-c3cncc4c3cnn4C)cc2)c2ccccc21)C(F)(F)F] | Method 3: 3.417 min, m/z 509.10 [M + H]⁺ | δ 9.22 (s, 1H), 8.54-8.46 (m, 2H), 8.42 (s, 1H), 8.04 (d, J = 8.1 Hz, 2H), 7.76 (d, J = 8.1 Hz, 2H), 7.23-7.12 (m, 4H), 4.61 (s, 2H), 4.25 (s, 3H), 3.42-3.40 (m, 2H), 3.20-3.14 (m, 1H), 1.07 (d, J = 7.1 Hz, 3H) ppm. |
| | 2-[3-[4-(1-methylpyrazolo[3,4- | | |
| | c]pyridin-4-yl)phenyl]-2- | | |
| | oxobenzimidazol-1-yl]-N-(3,3,3- | | |
| | trifluoro-2-methyl- | | |
| | propyl)acetamide | | |
| | Example 33 | | |
| Intermediate I-31 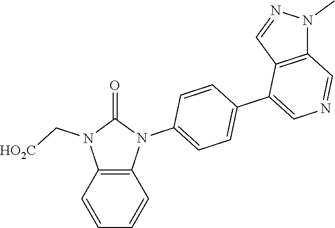 3-(trifluoromethyl)azetidine (1.5 eq) 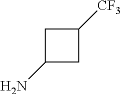 HATU (1.2 eq) | 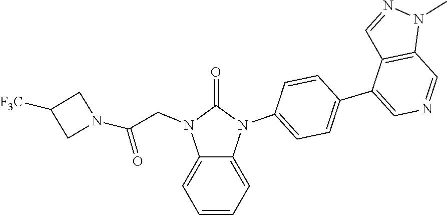 [SMILES: Cn1ncc2c(-c3ccc(-n4c(=O)n(CC(=O)N5CC(C(F)(F)F)C5)c5ccccc54)cc3)cncc21] | Method 3: 3.317 min, m/z 507.10 [M + H]⁺ | δ 9.24 (s, 1H), 8.53 (s, 1H), 8.45 (s, 1H), 8.06 (d, J = 8.2 Hz, 2H), 7.78 (d, J = 8.2 Hz, 2H), 7.27-7.23 (m, 2H), 7.19 (t, J = 7.5 Hz, 1H), 7.13 (t, J = 7.5 Hz, 1H), 4.74 (d, J = 4.4 Hz, 2H), 4.63 (t, J = 8.9 Hz, 1H), 4.44 (dd, J = 5.8, 9.4 Hz, 1H), 4.28 (s, 3H), 4.21 (t, J = 9.4 Hz, 1H), 3.96 (dd, J = 5.7, 9.4 Hz, 1H), 3.76 (m_{c}, 1H) ppm. |
| | 1-[4-(1-methylpyrazolo[3,4- | | |
| | c]pyridin-4-yl)phenyl]-3-[2-oxo-2- | | |
| | [3-(trifluoromethyl)azetidin-1-yl]- | | |
| | ethyl]benzimidazol-2-one | | |
| | Example 34 | | |
| Intermediate I-31 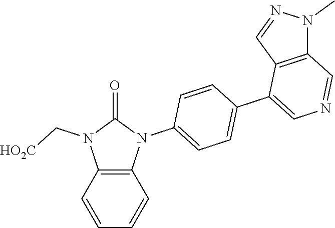 3,3-difluoroazetidine (1.5 eq) 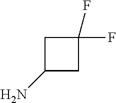 HATU (1.2 eq) | 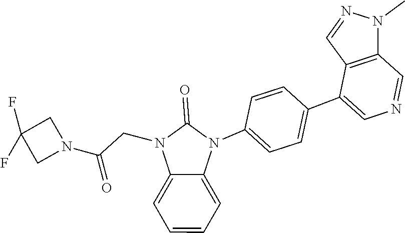 [SMILES: Cn1ncc2c(-c3ccc(-n4c(=O)n(CC(=O)N5CC(F)(F)C5)c5ccccc54)cc3)cncc21] | Method 3: 3.183 min, m/z 475.15 [M + H]⁺ | δ 9.22 (s, 1H), 8.50 (s, 1H), 8.42 (s, 1H), 8.03 (d, J = 8.5 Hz, 2H), 7.76 (d, J = 8.3 Hz, 2H), 7.27 (d, J = 7.7 Hz, 1H), 7.22 (d, J = 7.7 Hz, 1H), 7.19-7.10 (m, 2H), 4.87 (t, J = 12.4 Hz, 2H), 4.78 (s, 2H), 4.41 (t, J = 12.6 Hz, 2H), 4.25 (s, 3H) ppm. |
| | 1-[2-(3,3-difluoroazetidin-1-yl)-2- | | |
| | oxoethyl]-3-[4-(1-methyl- | | |
| | pyrazolo[3,4-c]pyridin-4-yl)- | | |
| | phenyl]benzimidazol-2-one | | |
| | Example 35 | | |
| Intermediate I-31 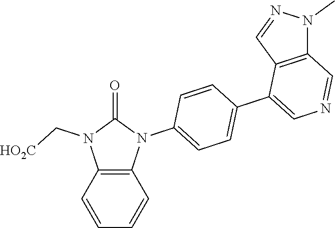 2,2- difluorocyclopropanamine hydrochloride (1.5 eq) 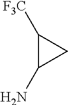 T3P (1.5 eq) | 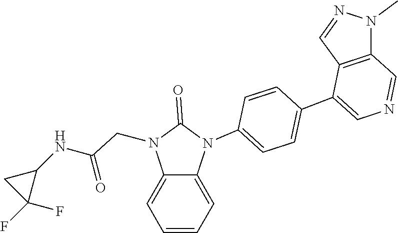 [SMILES: Cn1ncc2c(-c3ccc(-n4c(=O)n(CC(=O)NC5CC5(F)F)c5ccccc54)cc3)cncc21] | Method 3: 3.183 min, m/z 474.18 [M + H]⁺ | δ 9.22 (s, 1H), 8.79 (s_{br}, 1H), 8.51 (s, 1H), 8.42 (s, 1H), 8.05 (d, J = 8.2 Hz, 2H), 7.76 (d, J = 8.2 Hz, 2H), 7.25 (d, J = 7.8 Hz, 1H), 7.23-7.10 (m, 3H), 4.62 (m_{c}, 2H), 4.26 (s, 3H), 3.48-3.43 (m, 1H), 2.01- 1.88 (m, 1H), 1.59-1.49 (m, 1H) ppm. |
| | N-(2,2-difluorocyclopropyl)-2-[3- | | |
| | [4-(1-methylpyrazolo[3,4- | | |
| | c]pyridin-4-yl)phenyl]-2- | | |
| | oxobenzimidazol-1-yl]acetamide | | |
| | Example 36 | | |
| Intermediate I-31 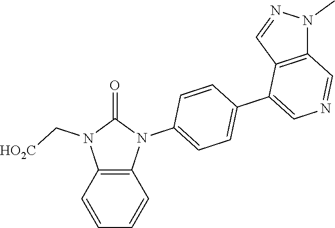 (2R)-2- (trifluoromethyl)morpholine hydrochloride (1.5 eq) 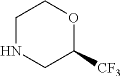 HATU (1.5 eq) | 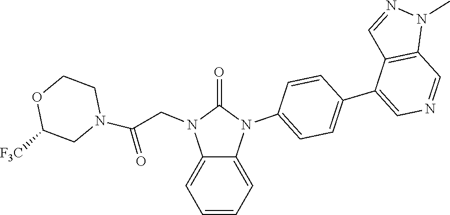 [SMILES: Cn1ncc2c(-c3ccc(-n4c(=O)n(CC(=O)N5CCO[C@@H](C(F)(F)F)C5)c5ccccc54)cc3)cncc21] | Method 3: 3.462 min, m/z 537.10 [M + H]⁺ | δ 9.41 (s, 1H), 8.62 (s, 1H), 8.60 (s, 1H), 8.07 (d, J = 8.2 Hz, 2H), 7.79 (d, J = 8.1 Hz, 2H), 7.26-7.22 (m, 2H), 7.19 (t, J = 7.5 Hz, 1H), 7.13 (t, J = 7.4 Hz, 1H), 5.16- 5.05 (m, 1H), 4.93-4.86 (m, 1H), 4.44- 4.30 (m, 0.5 H), 4.30 (s, 3H), 4.16-3.98 (m, 3H), 3.81-3.74 (m, 1H), 3.44-3.36 (m, 1.5H), 2.97-2.87 (m, 1H) ppm. |
| | 1-[4-(1-methylpyrazolo[3,4- | | |
| | c]pyridin-4-yl)phenyl]-3-[2-oxo-2- | | |
| | [(2R)-2-(trifluoromethyl)- | | |
| | morpholin-4-yl]ethyl]benz- | | |
| | imidazol-2-one | | |
| | Example 37 | | |
| Intermediate I-31 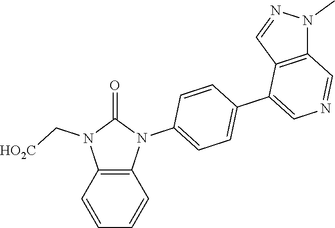 (2S)-2- (trifluoromethyl)morpholine hydrochloride (1.5 eq) 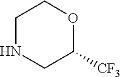 HATU (1.5 eq) | 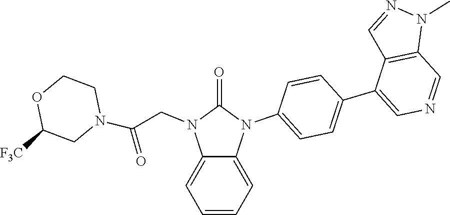 [SMILES: Cn1ncc2c(-c3ccc(-n4c(=O)n(CC(=O)N5CCO[C@H](C(F)(F)F)C5)c5ccccc54)cc3)cncc21] | Method 3: 3.464 min, m/z 537.10 [M + H]⁺ | δ 9.43 (s, 1H), 8.62 (s, 1H), 8.60 (s, 1H), 8.07 (d, J = 8.2 Hz, 2H), 7.79 (d, J = 8.2 Hz, 2H), 7.28-7.24 (m, 2H), 7.19-7.10 (m, 2H), 5.16- 5.05 (m, 1H), 4.94-4.87 (m, 1H), 4.43-4.17 (m, 0.5 H), 4.31 (s, 3H), 4.17-4.01 (m,4 H), 3.62-3.56 (m, 0.5 H), 3.49-3.37 (m, 1H), 3.97- 2.87 (m, 1H) ppm. |
| | 1-[4-(1-methylpyrazolo[3,4- | | |
| | c]pyridin-4-yl)phenyl]-3-[2-oxo-2- | | |
| | [(2S)-2-(trifluoromethyl)- | | |
| | morpholin-4-yl]ethyl]benz- | | |
| | imidazol-2-one | | |
| | Example 38 | | |
| Intermediate I-34 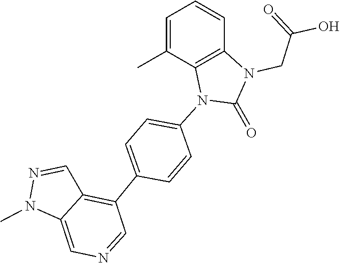 3- (trifluoromethyl)morpholine (1.0 eq) 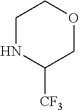 EDCl (2.0 eq) HOBT (1.1 eq) | 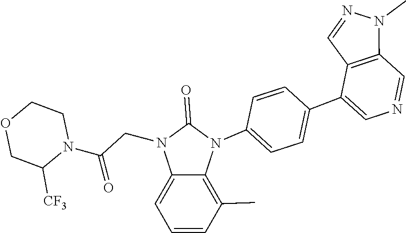 [SMILES: Cc1cccc2c1n(-c1ccc(-c3cncc4c3cnn4C)cc1)c(=O)n2CC(=O)N1CCOCC1C(F)(F)F] | Method 3: 3.483 min, m/z 551.15 [M + H]⁺ | δ 9.22 (s, 1H), 8.52 (s, 1H), 8.41 (s, 1H), 8.00 (d, J = 8.1 Hz, 2H), 7.61 (d, J = 8.0 Hz, 2H), 7.07-6.84 (m, 3H), 5.30-5.15 (m 1H), 4.99-4.69 (m, 2H), 4.26 (s, 3H), 4.20-4.10 (m, 1H), 3.99-3.85 (m, 2H), 3.77-3.43 (m, 3H), 1.91 (s, 3H) ppm. |
| | 4-methyl-3-[4-(1-methylpyrazo- | | |
| | lo[3,4-c]pyridin-4-yl)phenyl]-1-[2- | | |
| | oxo-2-[3-(trifluoromethyl)- | | |
| | morpholin-4- | | |
| | yl]ethyl]benzimidazol-2-one | | |
| | Example 39 | | |
| Intermediate I-35 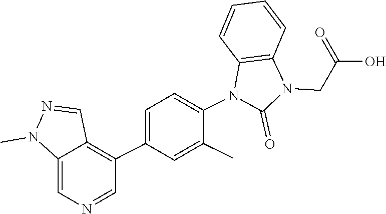 2- (trifluoromethyl)morpholine (1.5 eq) 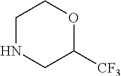 HATU (1.5 eq) | 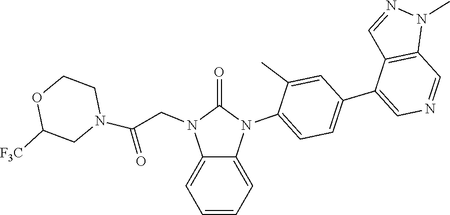 [SMILES: Cc1cc(-c2cncc3c2cnn3C)ccc1-n1c(=O)n(CC(=O)N2CCOC(C(F)(F)F)C2)c2ccccc21] | Method 3: 3.383 min, m/z 551.10 [M + H]⁺ | δ 9.22 (s, 1H), 8.51 (s, 1H) 8.41 (s, 1H), 7.92 (s, 1H), 7.84 (d, J = 8.1 Hz, 1H), 7.54 (d, J = 8.1 Hz, 1H), 7.22 (d, J = 7.6 Hz, 1H), 7.12 (t, J = 7.8 Hz, 1H), 7.05 (t, J = 7.7 Hz, 1H), 6.78 (d, J = 7.7 Hz, 1H), 5.19- 5.00 (m, 1H), 4.96-4.81 (m, 1H), , 4.45- 4.27 (m, 2H), 4.25 (s, 3H), 4.20-3.93 (m, 3H), 3.82-3.40 (m, 2H), 2.21 (s, 3H) ppm. |
| | 1-[2-methyl-4-(1- | | |
| | methylpyrazolo[3,4-c]pyridin-4- | | |
| | yl)phenyl]-3-[2-oxo-2-[2- | | |
| | (trifluoromethyl)morpholin-4- | | |
| | yl]ethyl]benzimidazol-2-one | | |
| | Example 40 | | |
| Intermediate I-34 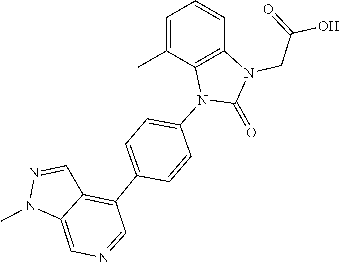 (2S)- (trifluoromethyl)morpholine (1.5 eq) 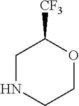 HATU (1.5 eq) | 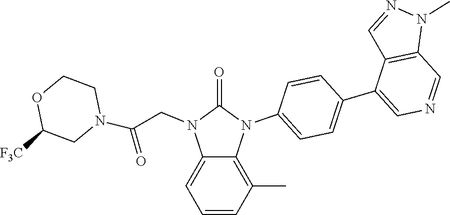 | Method 4: 1.83 min, m/z 537.2 [M + H]⁺ | δ 8.81 (d, J = 7.7 Hz, 2H), 8.67 (d, J = 2.5 Hz, 1H), 8.62-8.56 (m, 1H), 8.08 (d, J = 8 .5 Hz, 1H), 7.91 (dd, J = 8.5, 2.6 Hz, 1H), 7.51-7.45 (m, 1H), 7.12-7.00 (m, 3H), 6.85 (d, J = 7.2 Hz, 1H), 5.14-4.79 (m, 2H), 4.46-3.94 (m, 4H), 3.81-3.53 (m, 1H), 3.49-3.35 (m, 1H), 2.98-2.84 (m, 1H), 1.93 (s, 3H) ppm. |
| | 4-methyl-3-[4-(1- | | |
| | methylpyrazolo[3,4-c]pyridin-4- | | |
| | yl)phenyl]-1-[2-oxo-2-[(2S)-2- | | |
| | (trifluoromethyl)morpholin-4- | | |
| | yl]ethyl]benzimidazol-2-one | | |
| | Example 42 | | |
| Intermediate I-34 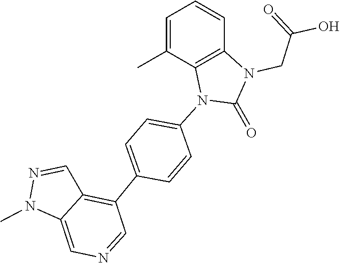 (2S)- (difluoromethyl)pyrrolidine (1.5 eq) 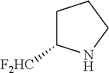 HATU (1.5 eq) | 1-[2-[(2S)-2- (difluoromethyl)pyrrolidin-1-yl]-2- oxo-ethyl]-4-methyl-3-[4-(1- methylpyrazolo[3,4-c]pyridin-4- yl)phenyl]benzimidazol-2-one 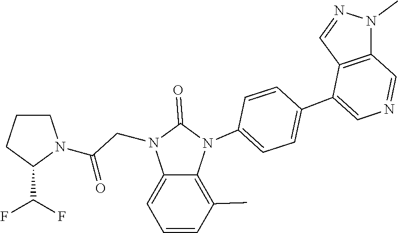 [SMILES: Cc1cccc2c1n(-c1ccc(-c3cncc4c3cnn4C)cc1)c(=O)n2CC(=O)N1CCC[C@H]1C(F)F] | Method 3: 3.52 min, m/z 517.35 [M + H]⁺ | δ 9.36 (s, 1H), 8.59 (s, 1H), 8.52 (s, 1H), 8.02 (d, J = 8.2 Hz, 2H), 7.63 (d, J = 8.1 Hz, 2H), 7.10-7.01 (m, 2H), 6.86 (d, J = 7.2 Hz, 1H), 6.42-6.05 (m, 1H), 5.02- 4.73 (m, 2H), 4.29-4.24 (m, 4 H), 3.72 (d, J = 6.3 Hz, 2H), 2.04-1.95 (m, 4H), 1.91 (s, 3H) ppm. |
| | Example 43 | | |
| Intermediate I-34 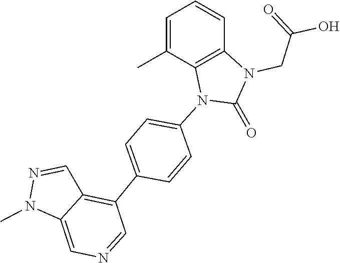 (2S)- (fluoromethyl)pyrrolidine (1.0 eq) 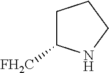 HATU (1.5 eq) | 1-[2-[(2S)-2- (fluoromethyl)pyrrolidin-1-yl]-2- oxo-ethyl]-4-methyl-3-[4-(1- methylpyrazolo[3,4-c]pyridin-4- yl)phenyl]benzimidazol-2-one 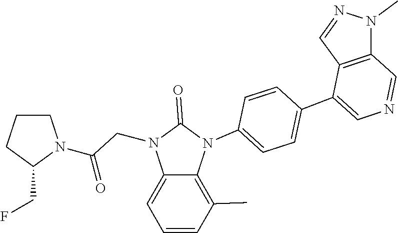 [SMILES: Cc1cccc2c1n(-c1ccc(-c3cncc4c3cnn4C)cc1)c(=O)n2CC(=O)N1CCC[C@H]1CF] | Method 3: 3.31 min, m/z 499.45 [M + H]⁺ | δ 9.22 (s, 1H), 8.52 (s, 1H), 8.41 (s, 1H), 8.00 (d, J = 8.0 Hz, 2H), 7.61 (d, J = 7.8 Hz, 2H), 7.07-6.98 (m, 2H), 6.85 (d, J = 6.9 Hz, 1H), 4.78 (t, 2H), 4.55-4.36 (m, 2H), 4.25 (s, 3H), 4.15 (d, J = 22.7 Hz, 1H), 3.70-3.45 (m, 2H), 2.02-1.86 (m, 7H) ppm. |
| | Example 45 | | |
| Intermediate I-31 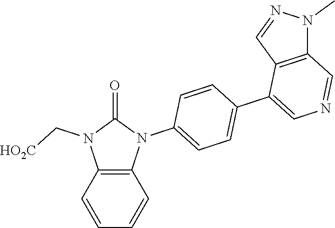 3,3,3-trifluoro-1- propanamine (1 eq) 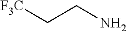 T-3-P (1.5 eq) | 2-[3-[4-(1-methylpyrazolo[3,4- c]pyridin-4-yl)phenyl]-2-oxo- benzimidazol-1-yl]-N-(3,3,3- trifluoropropyl)acetamide 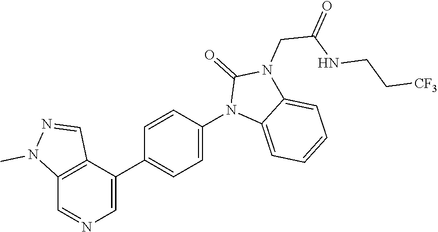 [SMILES: Cn1ncc2c(-c3ccc(-n4c(=O)n(CC(=O)NCCC(F)(F)F)c5ccccc54)cc3)cncc21] | Method 3: 3.03 min, m/z 495.10 [M + H]⁺ | δ 9.22 (s, 1H), 8.50 (d, J = 3.9 Hz, 2H), 8.42 (s, 1H), 8.04 (d, J = 8.3 Hz, 2H), 7.76 (d, J = 8.2 Hz, 2H), 7.22 (d, J = 7.5 Hz, 1H), 7.16 (d, J = 4.3 Hz, 2H), 7.14- 7.09 (m, 1H), 4.58 (s, 2H), 4.25 (s, 3H), 3.39-3.35 (m, 2H), 2.47-2.41 (m, 2H) ppm. |
| | Example 46 | | |
| Intermediate I-40 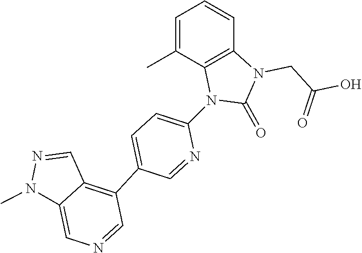 (2S)-Amino-1,1,1- trifluoropropane 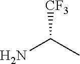 HATU (1.5 eq) | 2-[4-methyl-3-[5-(1- methylpyrazolo[3,4-c]pyridin-4- yl)-2-pyridyl]-2-oxo- benzimidazol-1-yl]-N-[(1S)-2,2,2- trifluoro-1-methyl- ethyl]acetamide 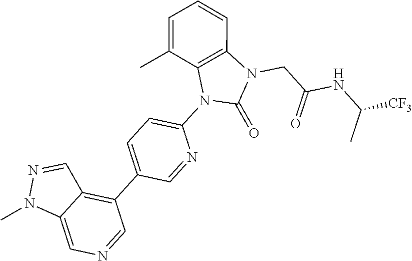 | Method 3: 3.45 min, m/z 510.30 [M + H]⁺ | δ 9.28 (s, 1H), 9.09 (d, J = 2.4 Hz, 1H), 8.92 (d, J = 8.9 Hz, 1H), 8.59 (s, 1H), 8.51 (dd, J = 8.2, 2.5 Hz, 1H), 8.47 (s, 1H), 7.82 (d, J = 8.2 Hz, 1H), 7.07 (t, J = 7.7 Hz, 1H), 7.00 (d, J = 7.8 Hz, 1H), 6.90 (d, J = 7.6 Hz, 1H), 4.81-4.58 (m, 3H), 4.27 (s, 3H), 1.94 (s, 3H), 1.30 (d, J = 7.0 Hz, 3H) ppm. |
| | Example 57 | | |
| Intermediate I-40 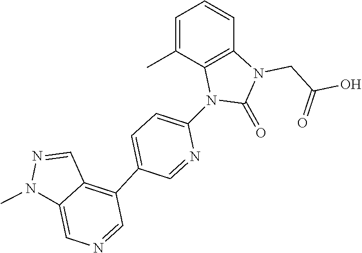 (2S)- Trifluoromethylpyrrolidine 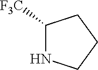 HATU (1.5 eq) | 4-methyl-3-[5-(1- methylpyrazolo[3,4-c]pyridin-4- yl)-2-pyridyl]-1-[2-oxo-2-[(2S)-2- (trifluoromethyl)pyrrolidin-1- yl]ethyl]benzimidazol-2-one 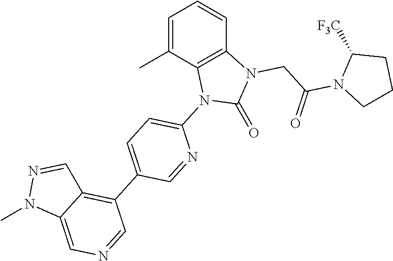 [SMILES: Cc1cccc2c1n(-c1ccc(-c3cncc4c3cnn4C)cn1)c(=O)n2CC(=O)N1CCC[C@H]1C(F)(F)F] | Method 3: 3.55 min, m/z 536.30 [M + H]⁺ | δ 9.28 (s, 1H), 9.09 (d, J = 2.4 Hz, 1H), 8.60 (s, 1H), 8.53-8.48 (m, 2H), 7.82 (d, J = 8.2 Hz, 1H), 7.09- .03 (m, 2H), 6.94-6.89 (m, 1H), 5.20-4.83 (m, 2H), 4.78-4.70 (m, 1H), 4.27 (s, 3H), 3.84-3.67 (m, 2H), 2.13-2.02 (m, 4H), 1.94 (s, 3H) ppm. |
| | Example 59 | | |
| Intermediate I-40 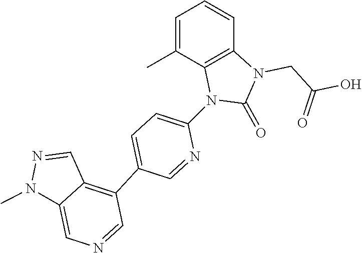 (2R)- (trifluoromethyl)morpholine 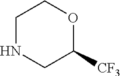 HATU (1.5 eq) | 4-methyl-3-[5-(1- methylpyrazolo[3,4-c]pyridin-4- yl)-2-pyridyl]-1-[2-oxo-2-[(2R)-2- (trifluoromethyl)morpholin-4- yl]ethyl]benzimidazol-2-one 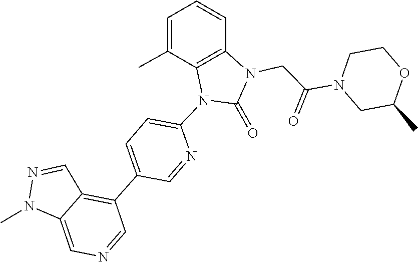 | Method 3: 3.48 min, m/z 552.25 [M + H]⁺ | δ 9.28 (s, 1H), 9.09 (d, J = 2.4 Hz, 1H), 8.59 (s, 1H), 8.52 (dd, J = 8.3, 2.5 Hz, 1H), 8.48 (s, 1H), 7.80 (d, J = 8.2 Hz, 1H), 7.10- 7.04 (m, 2H), 6.93-6.86 (m, 1H), 5.14- 4.80 (m, 2H), 4.47-3.96 (m, 7H), 3.67 (dt, J = 74.3, 11.4 Hz, 1H), 3.47-3.35 (m, 1H), 2.98-2.84 (m, 1H), 1.94 (s, 3H) ppm. |
| | Example 60 | | |
| Intermediate I-40 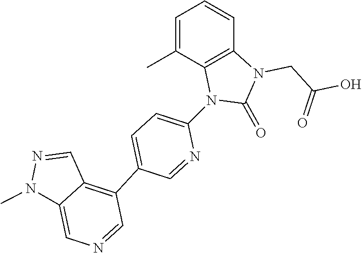 (2S)- (trifluoromethyl)morpholine 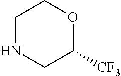 HATU (1.5 eq) | 4-methyl-3-[5-(1- methylpyrazolo[3,4-c]pyridin-4- yl)-2-pyridyl]-1-[2-oxo-2-[(2S)-2- (trifluoromethyl)morpholin-4- yl]ethyl]benzimidazol-2-one 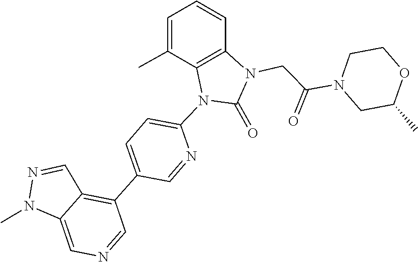 | Method 3: 3.47 min, m/z 552.30 [M + H]⁺ | δ 9.28 (s, 1H), 9.09 (d, J = 2.4 Hz, 1H), 8.59 (s, 1H), 8.52 (dd, J = 8.3, 2.5 Hz, 1H), 8.48 (s, 1H), 7.80 (d, J = 8.2 Hz, 1H), 7.10- 7.04 (m, 2H), 6.93-6.86 (m, 1H), 5.14- 4.80 (m, 2H), 4.47-3.96 (m, 7H), 3.67 (dt, J = 74.3, 11.4 Hz, 1H), 3.47-3.35 (m, 1H), 2.98-2.84 (m, 1H), 1.94 (s, 3H) ppm. |
| | Example 61 | | |
| Intermediate I-31 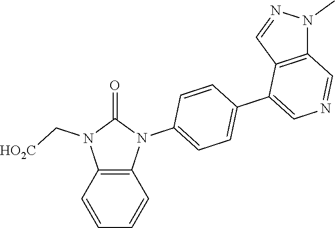 (2S)-4,4,4-trifluorobutan-2- amine 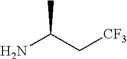 HATU (1.2 eq) | 2-[3-[4-(1-methylpyrazolo[3,4- c]pyridin-4-yl)phenyl]-2-oxo- benzimidazol-1-yl]-N-[(1S)-3,3,3- trifluoro-1-methyl- propyl]acetamide 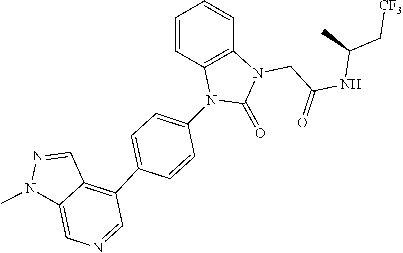 [SMILES: C[C@@H](CC(F)(F)F)NC(=O)Cn1c(=O)n(-c2ccc(-c3cncc4c3cnn4C)cc2)c2ccccc21] | Method 3: 3.45 min, m/z 509.15 [M + H]⁺ | δ 9.22 (s, 1H), 8.50 (s, 1H), 8.41 (d, J = 7.5 Hz, 2H), 8.03 (d, J = 8.1 Hz, 2H), 7.76 (d, J = 8.1 Hz, 2H), 7.22 (d, J = 7.7 Hz, 1H), 7.16-7.09 (m, 3H), 4.60-4.49 (m, 2H), 4.25 (s, 3H), 4.19-4.12 (m, 1H), 2.47-4.43 (m, 2H), 1.20 (d, J = 6.8 Hz, 3H) ppm. |
| | Example 62 | | |
| Intermediate I-42 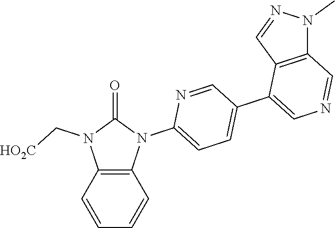 Trifluoroethylamine 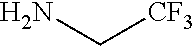 HATU (1.2 eq) | 2-[3-[5-(1-methylpyrazolo[3,4- c]pyridin-4-yl)-2-pyridyl]-2-oxo- benzimidazol-1-yl]-N-(2,2,2- trifluoroethyl) acetamide 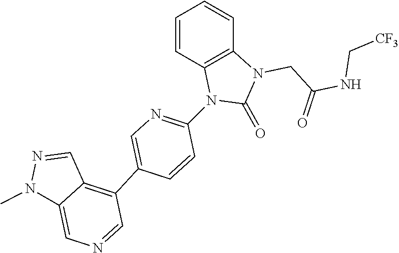 [SMILES: Cn1ncc2c(-c3ccc(-n4c(=O)n(CC(=O)NCC(F)(F)F)c5ccccc54)nc3)cncc21] | Method 3: 3.42 min, m/z 482.30 [M + H]⁺ | δ 9.25 (s, 1H), 9.10-8.97 (m, 2H), 8.52 (d, J = 28.6 Hz, 3H), 8.30 (d, J = 8.6 Hz, 1H), 8.18 (d, J = 7.5 Hz, 1H), 7.18 (d, J = 8.2 Hz, 3H), 4.72 (s, 2H), 4.26 (s, 3H), 4.05- 3.92 (m, 2H) ppm. |
| | Example 63 | | |
| Intermediate I-48 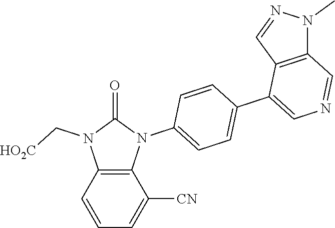 Trifluoroethylamine 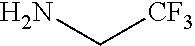 HATU (1.5 eq) | 2-[4-cyano-3-[4-(1- methylpyrazolo[3,4-c]pyridin-4- yl)phenyl]-2-oxo-benzimidazol-1- yl]-N-(2,2,2- trifluoroethyl)acetamide 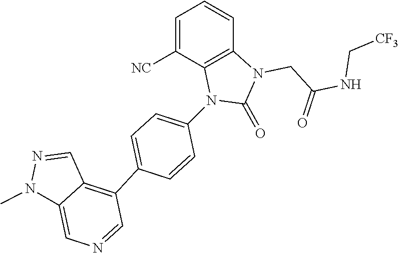 [SMILES: Cn1ncc2c(-c3ccc(-n4c(=O)n(CC(=O)NCC(F)(F)F)c5cccc(C#N)c54)cc3)cncc21] | Method 3: 3.08 min, m/z 505.45 [M + H]⁺ | δ 9.23 (s, 1H), 8.99 (t, J = 6.3 Hz, 1H), 8.51 (s, 1H), 8.33 (d, J = 0.9 Hz, 1H), 8.05- 8.00 (m, 2H), 7.75-7.70 (m, 2H), 7.53 (dd, J = 7.9, 1.1 Hz, 1H), 7.47 (dd, J = 8.0, 1.0 Hz, 1H), 7.29 (t, J = 7.9 Hz, 1H), 4.75 (s, 2H), 4.26 (s, 3H), 4.05-3.94 (m, 2H) ppm. |
| | Example 74 | | |
| Intermediate I-49 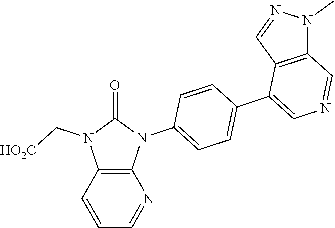 Trifluoroethylamine 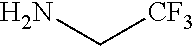 HATU (1.5 eq) | 2-[3-[4-(1-methylpyrazolo[3,4- c]pyridin-4-yl)phenyl]-2-oxo- imidazo[4,5-b]pyridin-1-yl]-N- (2,2,2-trifluoroethyl)acetamide 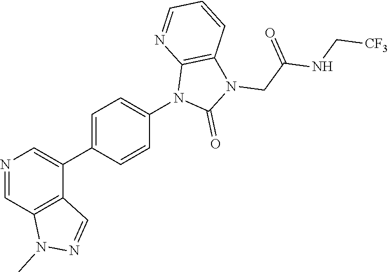 [SMILES: Cn1ncc2c(-c3ccc(-n4c(=O)n(CC(=O)NCC(F)(F)F)c5cccnc54)cc3)cncc21] | Method 3: 2.95 min, m/z 482.25 [M + H]⁺ | δ 9.21 (s, 1H), 8.99 (t, J = 6.3 Hz, 1H), 8.50 (s, 1H), 8.42 (s, 1H), 8.05 (d, J = 5.1 Hz, 1H), 8.02 (d, J = 8.3 Hz, 2H), 7.93 (d, J = 8.2 Hz, 2H), 7.57 (d, J = 7.8 Hz, 1H), 7.21 (dd, J = 7.7, 5.2 Hz, 1H), 4.74 (s, 2H), 4.25 (s, 3H), 4.03-3.94 (m, 2H) ppm. |
| | Example 76 | | |
| Intermediate I-50 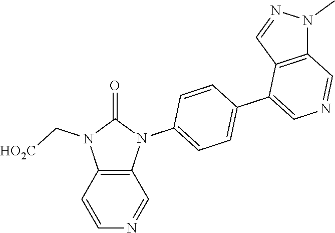 Trifluoroethylamine 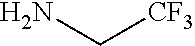 HATU (1.5 eq) | 2-[3-[4-(1-methylpyrazolo[3,4- c]pyridin-4-yl)phenyl]-2-oxo- imidazo[4,5-c]pyridin-1-yl]-N- (2,2,2-trifluoroethyl)acetamide 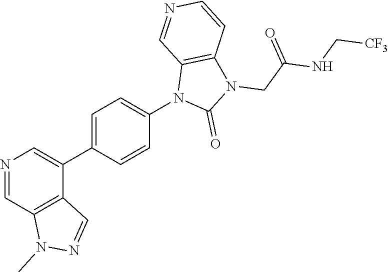 [SMILES: Cn1ncc2c(-c3ccc(-n4c(=O)n(CC(=O)NCC(F)(F)F)c5ccncc54)cc3)cncc21] | Method 4: 1.65 min, m/z 482.30 [M + H]⁺ | δ 9.21 (s, 1H), 9.03 (t, J = 6.4 Hz, 1H), 8.50 (s, 1H), 8.44 (d, J = 9.7 Hz, 2H), 8.34 (d, J = 5.3 Hz, 1H), 8.04 (d, J = 8.3 Hz, 2H), 7.82 (d, J = 8.3 Hz, 2H), 7.31 (d, J = 5.2 Hz, 1H), 4.74 (s, 2H), 4.25 (s, 3H), 4.03- 3.94 (m, 2H) ppm. |
| | Example 77 | | |
| Intermediate I-51 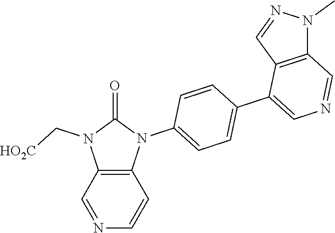 Trifluoroethylamine 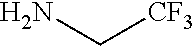 HATU (1.5 eq) | 2-[1-[4-(1-methylpyrazolo[3,4- c]pyridin-4-yl)phenyl]-2-oxo- imidazo[4,5-c]pyridin-3-yl]-N- (2,2,2-trifluoroethyl)acetamide 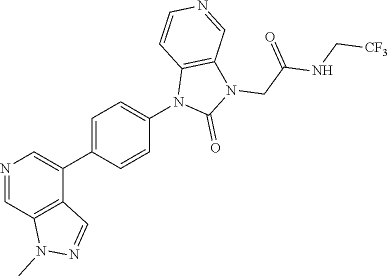 [SMILES: Cn1ncc2c(-c3ccc(-n4c(=O)n(CC(=O)NCC(F)(F)F)c5cnccc54)cc3)cncc21] | Method 4: 1.65 min, m/z 482.15 [M + H]⁺ | δ 9.23 (s, 1H), 9.03 (t, J = 6.3 Hz, 1H), 8.51 (s, 1H), 8.43 (d, J = 9.8 Hz, 2H), 8.30 (d, J = 5.3 Hz, 1H), 8.07-8.03 (m, 2H), 7.79- 7.75 (m, 2H), 7.26 (d, J = 5.3 Hz, 1H), 4.77 (s, 2H), 4.26 (s, 3H), 4.05-3.96 (m, 2H) ppm. |
| | Example 78 | | |
| Intermediate I-49 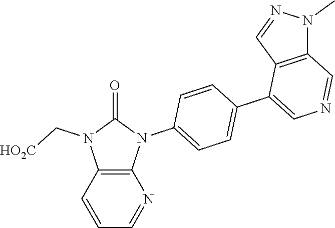 (2S)-2- (trifluoromethyl)pyrrolidine 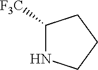 HATU (1.5 eq) | 3-[4-(1-methylpyrazolo[3,4- c]pyridin-4-yl)phenyl]-1-[2-oxo-2- [(2S)-2- (trifluoromethyl)pyrrolidin-1- yl]ethyl]imidazo[4,5-b]pyridin-2- one 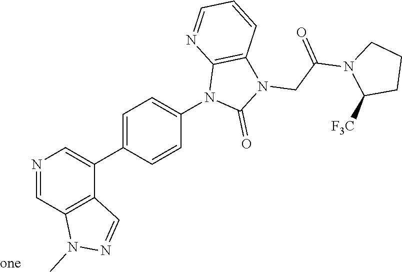 [SMILES: Cn1ncc2c(-c3ccc(-n4c(=O)n(CC(=O)N5CCC[C@H]5C(F)(F)F)c5cccnc54)cc3)cncc21] | Method 3: 2.95 min, m/z 522.25 [M + H]⁺ | δ 9.23 (s, 1H), 8.51 (s, 1H), 8.41 (s, 1H), 8.05 (dd, J = 5.2, 1.4 Hz, 1H), 8.01 (d, J = 8.5 Hz, 2H), 7.92 (d, J = 8.5 Hz, 2H), 7.60 (dd, J = 7.9, 1.4 Hz, 1H), 7.21 (dd, J = 7.8, 5.1 Hz, 1H), 5.22-4.74 (m, 3H), 4.25 (s, 3H), 3.86-3.67 (m, 2H), 2.14- 2.02 (m, 4H) ppm. |
| | Example 79 | | |
| Intermediate I-52 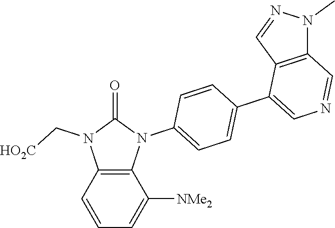 Trifluoroethylamine 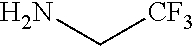 T-3-P (2.0 eq) | 2-[4-(dimethylamino)-3-[4-(1- methylpyrazolo[3,4-c]pyridin-4- yl)phenyl]-2-oxo-benzimidazol-1- yl]-N-(2,2,2- trifluoroethyl)acetamide 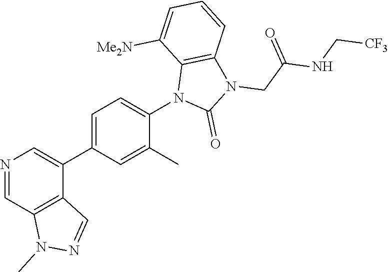 | Method 4: 1.25 min, m/z 523.8 [M + H]⁺ | δ 9.20 (s, 1H), 9.05 (t, J = 5.9 Hz, 1H), 8.49 (s, 1H), 8.37 (s, 1H), 7.55 (d, J = 8.5 Hz, 2H), 7.10 (t, J = 8.0 Hz, 1H), 6.82 (d, J = 7.8 Hz, 2H), 4.65 (s, 2H), 4.25 (s, 3H), 3.99-3.94 (m, 2H), 2.33 (s, 6H) ppm. |
| | Example 80 | | |
| Intermediate I-53 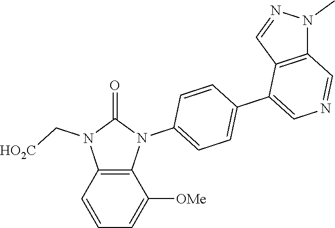 Trifluoroethylamine 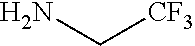 HATU (1.5 eq) | 2-[4-methoxy-3-[2-methyl-4-(1- methylpyrazolo[3,4-c]pyridin-4- yl)phenyl]-2-oxo-benzimidazol-1- yl]-N-(2,2,2- trifluoroethyl)acetamide 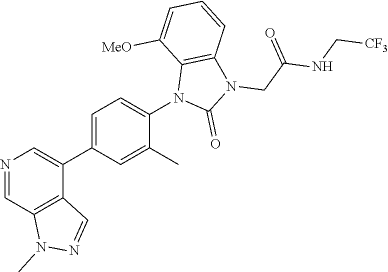 [SMILES: COc1cccc2c1n(-c1ccc(-c3cncc4c3cnn4C)cc1C)c(=O)n2CC(=O)NCC(F)(F)F] | Method 3: 3.14 min, m/z 525.20 [M + H]⁺ | δ 9.22 (s, 1H), 8.87 (m, 1H), 8.50 (s, 1H), 8.44 (s, 1H), 7.91 (s, 1H), 7.84 (m, 1H), 7.53 (d, J = 8.5 Hz, 1H), 7.02 (m, 1H), 6.83 (d, J = 7.8 Hz, 1H), 6.42 (d, J = 7.8 Hz, 1H), 4.73 (s, 2H), 4.25 (s, 3H), 4.02 (m, 2H), 3.81 (s, 3H), 2.21 (s, 3H) ppm. |
| | Example 81 | | |
| Intermediate I-54 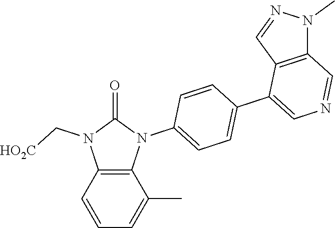 (2S)-2-Amino-1,1,1- trifluoropropane 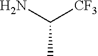 HATU (1.5 eq) | 2-[4-methyl-3-[2-methyl-4-(1- methylpyrazolo[3,4-c]pyridin-4- yl)phenyl]-2-oxo-benzimidazol-1- yl]-N-[(1S)-2,2,2-trifluoro-1- methyl-ethyl]acetamide 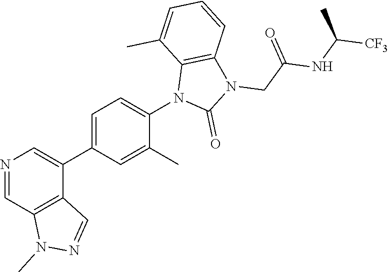 [SMILES: Cc1cc(-c2cncc3c2cnn3C)ccc1-n1c(=O)n(CC(=O)N[C@@H](C)C(F)(F)F)c2cccc(C)c21] | Method 3: 3.52 min, m/z 523.15 [M + H]⁺ | δ 9.22 (s, 1H), 8.90 (t, J = 9.0 Hz, 1H), 8.51 (s, 1H), 8.43 (s, 1H), 7.88 (s, 1H), 7.80 (dd, J = 8.1, 2.1 Hz, 1H), 7.54 (dd, J = 8.1, 5.3 Hz, 1H), 7.04-6.98 (m, 2H), 6.83 (d, J = 8.0 Hz, 1H), 4.68-4.57 (m, 3H), 4.25 (s, 3H), 2.17 (s, 3H), 1.81 (s, 3H), 1.30 (d, J = 7.0 Hz, 3H) ppm. |
| | Example 82 | | |
| Intermediate I-55 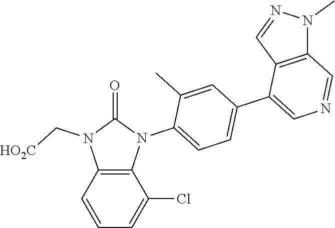 Trifluoroethylamine 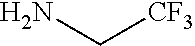 HATU (1.5 eq) | 2-[4-chloro-3-[2-methyl-4-(1- methylpyrazolo[3,4-c]pyridin-4- yl)phenyl]-2-oxo-benzimidazol-1- yl]-N-(2,2,2- trifluoroethyl)acetamide 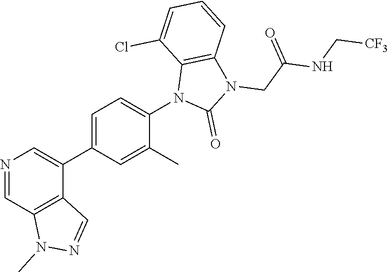 [SMILES: Cc1cc(-c2cncc3c2cnn3C)ccc1-n1c(=O)n(CC(=O)NCC(F)(F)F)c2cccc(Cl)c21] | Method 3: 3.48 min, m/z 529.10 [M + H]⁺ | δ 9.20 (s, 1H), 8.89 (t, J = 6.4 Hz, 1H), 8.51 (s, 1H), 8.38 (s, 1H), 7.85 (d, J = 2.1 Hz, 1H), 7.78 (dd, J = 8.1, 2.1 Hz, 1H), 7.53 (d, J = 8.1 Hz, 1H), 7.19-7.17 (m, 1H), 7.13 (t, J = 7.8 Hz, 1H), 7.08 (dd, J = 7.9, 1.5 Hz, 1H), 4.71 (s, 2H), 4.25 (s, 3H), 4.03-3.93 (m, 2H), 2.19 (s, 3H) ppm. |
| | Example 83 | | |
| Intermediate I-55 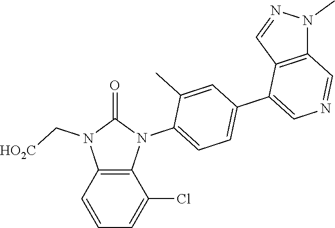 (2S)-2-Amino-1,1,1- trifluoropropane 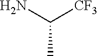 HATU (1.5 eq) | 2-[4-chloro-3-[2-methyl-4-(1- methylpyrazolo[3,4-c]pyridin-4- yl)phenyl]-2-oxo-benzimidazol-1- yl]-N-[(1S)-2,2,2-trifluoro-1- methyl-ethyl]acetamide 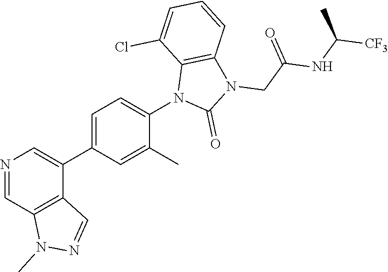 [SMILES: Cc1cc(-c2cncc3c2cnn3C)ccc1-n1c(=O)n(CC(=O)N[C@@H](C)C(F)(F)F)c2cccc(Cl)c21] | Method 3: 3.58 min, m/z 543.15 [M + H]⁺ | δ 9.22 (s, 1H), 8.91 (t, J = 9.1 Hz, 1H), 8.51 (s, 1H), 8.42 (s, 1H), 7.86 (d, J = 2.0 Hz, 1H), 7.79 (dd, J = 8.1, 2.1 Hz, 1H), 7.53 (t, J = 7.5 Hz, 1H), 7.18 (d, J = 7.7 Hz, 1H), 7.14 (t, J = 7.8 Hz, 1H), 7.08 (dd, J = 7.9, 1.5 Hz, 1H), 4.69 (d, J = 2.1 Hz, 2H), 4.64-4.62 (m, 3H), 4.25 (s, 3H), 2.17 (s, 3H), 1.30 (d, J = 7.0 Hz, 3H) ppm. |
| | Example 84 | | |
| Intermediate I-56 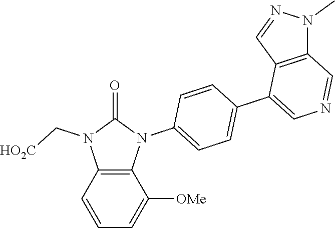 (2S)-2-Amino-1,1,1- trifluoropropane 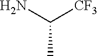 HATU (1.5 eq) | 2-[4-methoxy-3-[4-(1- methylpyrazolo[3,4-c]pyridin-4- yl)phenyl]-2-oxo-benzimidazol-1- yl]-N-[(1S)-2,2,2-trifluoro-1- methyl-ethyl]acetamide 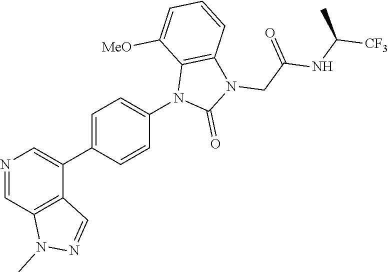 [SMILES: COc1cccc2c1n(-c1ccc(-c3cncc4c3cnn4C)cc1)c(=O)n2CC(=O)N[C@@H](C)C(F)(F)F] | Method 3: 3.41 min, m/z 525.20 [M + H]⁺ | δ 9.22 (s, 1H), 8.76 (d, J = 8.9 Hz, 1H), 8.50 (s, 1H), 8.42 (s, 1H), 8.05-8.01 (m, 2H), 7.75-7.71 (m, 2H), 7.06 (d, J = 8.2 Hz, 1H), 6.86-6.83 (m, 2H), 4.71 (d, J = 3.5 Hz, 2H), 4.62 (q, J = 7.8 Hz, 1H), 4.25 (s, 3H), 3.81 (s, 3H), 1.30 (d, J = 7.0 Hz, 3H) ppm. |
| | Example 85 | | |
| Intermediate I-57 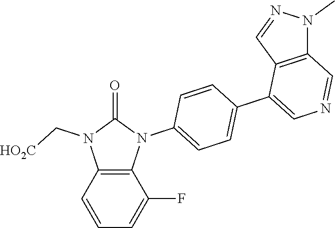 Trifluoroethylamine 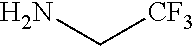 T-3-P (1.5 eq) | 2-[4-fluoro-3-[4-(1- methylpyrazolo[3,4-c]pyridin-4- yl)phenyl]-2-oxo-benzimidazol-1- yl]-N-(2,2,2- trifluoroethyl)acetamide 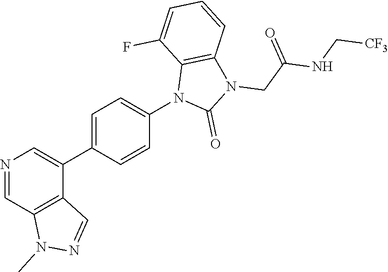 [SMILES: Cn1ncc2c(-c3ccc(-n4c(=O)n(CC(=O)NCC(F)(F)F)c5cccc(F)c54)cc3)cncc21] | Method 3: 3.30 min, m/z 499.15 [M + H]⁺ | δ 9.22 (s, 1H), 8.50 (s, 1H), 8.42 (d, J = 5.5 Hz, 2H), 8.04 (d, J = 8.2 Hz, 2H), 7.76 (d, J = 8.2 Hz, 2H), 7.22 (d, J = 7.5 Hz, 1H), 7.19-7.07 (m, 3H), 4.63-4.46 (m, 2H), 4.25 (s, 3H), 4.21-4.10 (m, 1H), 3.58-3.51 (m, 0H), 1.20 (d, J = 6.7 Hz, 3H) ppm. |
| | Example 86 | | |
| Intermediate I-59 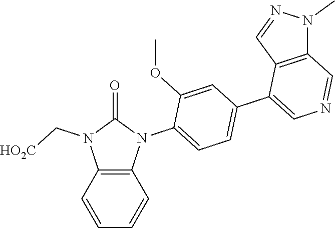 Trifluoroethylamine 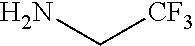 HATU (1.5 eq) | 2-[3-[2-methoxy-4-(1- methylpyrazolo[3,4-c]pyridin-4- yl)phenyl]-2-oxo-benzimidazol-1- yl]-N-(2,2,2- trifluoroethyl)acetamide 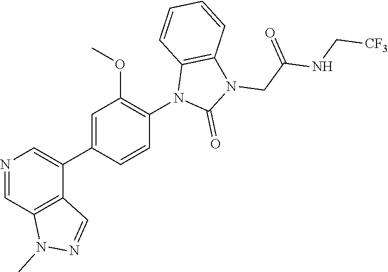 [SMILES: COc1cc(-c2cncc3c2cnn3C)ccc1-n1c(=O)n(CC(=O)NCC(F)(F)F)c2ccccc21] | Method 3: 3.10 min, m/z 511.20 [M + H]⁺ | δ 9.24 (s, 1H), 8.99 (t, J = 6.3 Hz, 1H), 8.56 (s, 1H), 8.48 (s, 1H), 7.63 (s, 1H), 7.59-7.54 (m, 2H), 7.13-7.02 (m, 3H), 6.77 (d, J = 7.6 Hz, 1H), 4.68 (s, 2H), 4.26 (s, 3H), 4.04- 3.95 (m, 2H), 3.90 (s, 3H) ppm. |
| | Example 88 | | |

### Example 41: 2-[2-oxo-3-[4-(1H-pyrazolo[3,4-c]pyridin-4-yl)phenyl]benzimidazol-1-yl]-N-(2,2,2-trifluoroethyl)acetamide

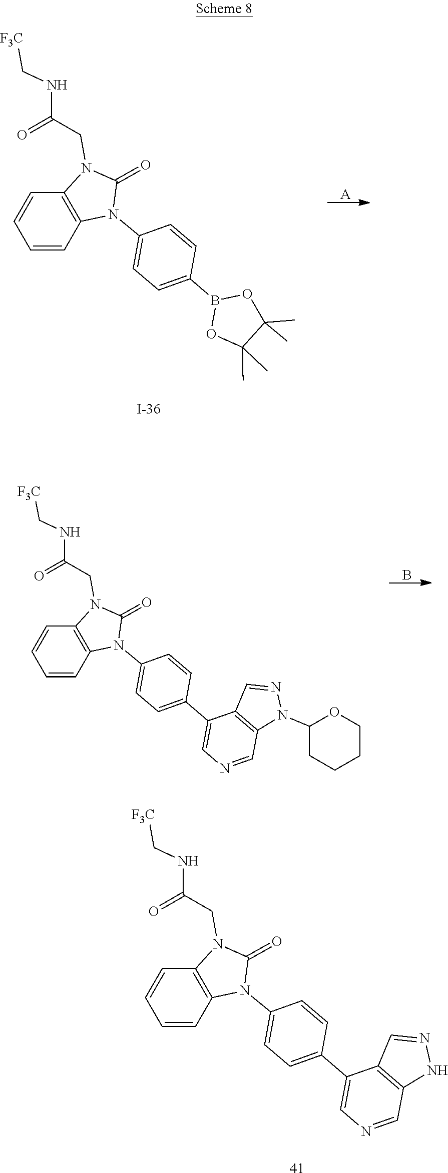

STEP A: Synthesis of 2-[2-oxo-3-[4-(1-tetrahydropyran-2-ylpyrazolo[3,4-c]pyridin-4-yl)phenyl]benzimidazol-1-yl]-N-(2,2,2-trifluoroethyl)acetamide. A mixture of 4-bromo-1-tetrahydropyran-2-yl-pyrazolo[3,4-c]pyridine (100 mg, 0.35 mmol), 2-[2-oxo-3-[4-(4,4,5,5-tetramethyl-1,3,2-dioxaborolan-2-yl)phenyl]benzimidazol-1-yl]-N-(2,2,2-trifluoroethyl)acetamide (202 mg, 0.42 mmol), Pd(dppf)Cl₂ (26 mg, 0.03 mmol), K₂CO₃ (147 mg, 1.06 mmol) in degassed 1,4-dioxane (5 mL) and degassed H₂O (0.5 mL) was stirred at 90° C. for 16 h under N₂-atmosphere. The mixture was diluted with water and extracted with EtOAc. The organic layer was dried with Na₂SO₂, concentrated in vacuo and purified by preparative TLC (EtOAc in petroleum ether 50%) to give 2-[2-oxo-3-[4-(1-tetrahydropyran-2-ylpyrazolo[3,4-c]pyridin-4-yl)phenyl]benzimidazol-1-yl]-N-(2,2,2-trifluoroethyl)acetamide (97 mg) as off-white solid. LC-MS (ES⁺, Method 4): 1.06 min, m/z 551 [M+H]⁺.

STEP B: Synthesis of Example 41, 2-[2-oxo-3-[4-(1H-pyrazolo[3,4-c]pyridin-4-yl)phenyl]benzimidazol-1-yl]-N-(2,2,2-trifluoroethyl)acetamide. A mixture of 2-[2-oxo-3-[4-(1-tetrahydropyran-2-ylpyrazolo[3,4-c]pyridin-4-yl)phenyl]benzimidazol-1-yl]-N-(2,2,2-trifluoroethyl)acetamide (97 mg, 0.18 mmol), 4N HCl (4N in HCl, 1.0 mL, 4.0 mmol) in MeOH (1.5 mL) was stirred at room temperature for 1 h. The mixture was adjusted pH>7 with NaHCO₃ (aq, sat.), diluted with water and extracted with EtOAc. The organic layer was dried with Na₂SO₄, concentrated in vacuo and purified by preparative HPLC to give 2-[2-oxo-3-[4-(1H-pyrazolo[3,4-c]pyridin-4-yl)phenyl]benzimidazol-1-yl]-N-(2,2,2-trifluoroethyl)acetamide (Example 41, 7.6 mg, 0.016 mmol, 5%) as a white solid. LC-MS (ES⁺, Method 3): 3.68 min, m/z 467.25 [M+H]⁺. ¹H NMR (400 MHz, DMSO-d₆): δ 13.88 (s, 1H), 9.06 (s, 1H), 8.96 (t, J=6.3 Hz, 1H), 8.44 (s, 1H), 8.43 (s, 1H), 8.01 (d, J=8.1 Hz, 2H), 7.73 (d, J=8.1 Hz, 2H), 7.19 (d, J=7.5 Hz, 1H), 7.16-7.03 (m, 3H), 4.66 (s, 2H), 3.95 (dq, J=6.1, 9.6 Hz, 2H) ppm.

### Example 44: 2-[4-methyl-2-oxo-3-[4-(1-tetrahydropyran-2-ylpyrazolo[3,4-c]pyridin-4-yl)phenyl]benzimidazol-1-yl]-N-(2,2,2-trifluoroethyl)acetamide

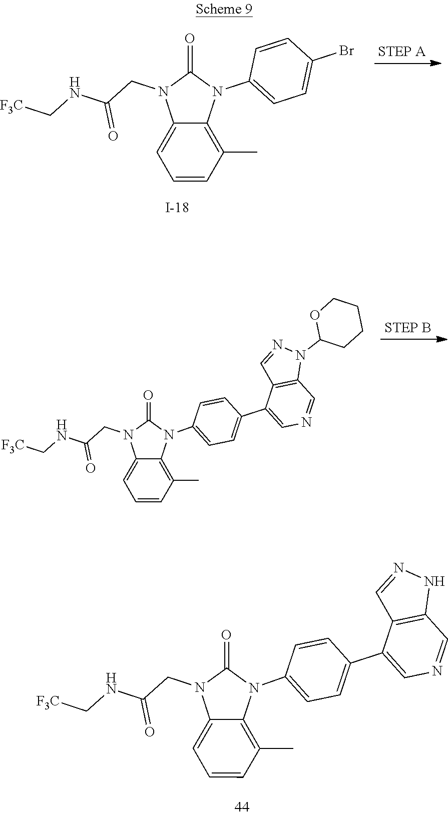

STEP A: 2-[4-methyl-2-oxo-3-[4-(1-tetrahydropyran-2-ylpyrazolo[3,4-c]pyridin-4-yl)phenyl]benzimidazol-1-yl]-N-(2,2,2-trifluoroethyl)acetamide. A mixture of 2-(3-(4-bromophenyl)-4-methyl-2-oxo-2,3-dihydro-1H-benzo[d]imidazol-1-yl)-N-(2,2,2-trifluoroethyl)acetamide (I-18) (313 mg, 0.71 mmol), 1-(tetrahydro-2H-pyran-2-yl)-4-(tributylstannyl)-1H-pyrazolo[3,4-c]pyridine (420 mg, 0.85 mmol), Pd(PPh₃)₄ (82 mg, 0.071 mmol), CuI (75 mg, 0.355 mmol) and LiCl (90 mg, 2.13 mmol) in toluene (5 ml) was stirred at 120° C. under an N₂ atmosphere overnight. The reaction was filtered and was purified by preparative TLC (DCM/MeOH, 20:1) to give 2-[4-methyl-2-oxo-3-[4-(1-tetrahydropyran-2-ylpyrazolo[3,4-c]pyridin-4-yl)phenyl]benzimidazol-1-yl]-N-(2,2,2-trifluoroethyl)acetamide as a colourless solid (30 mg, 7.5%). LC-MS (ES⁺, Method 4): 1.44 min, m/z 565.0 [M+H]⁺.

STEP B: 2-[4-methyl-2-oxo-3-[4-(1H-pyrazolo[3,4-c]pyridin-4-yl)phenyl]benzimidazol-1-yl]-N-(2,2,2-trifluoroethyl)acetamide. A solution of 2-(4-methyl-2-oxo-3-(4-(1-(tetrahydro-2H-pyran-2-yl)-1H-pyrazolo[3,4-c]pyridin-4-yl)phenyl)-2,3-dihydro-1H-benzo[d]imidazol-1-yl)-N-(2,2,2-trifluoroethyl)acetamide (25 mg, 0.044 mmol) in MeOH (0.5 mL) and 4 M HCl (in 1,4-dioxane, 1.5 ml) was sttired at room temperature for 1 h. The reaction was concentrated and was purified by preparative TLC (DCM:MeOH, 20:1) to give 2-[4-methyl-2-oxo-3-[4-(1H-pyrazolo[3,4-c]pyridin-4-yl)phenyl]benzimidazol-1-yl]-N-(2,2,2-trifluoroethyl)acetamide (example 44) (18.1 mg, 85%) as an off-white solid. LC-MS (ES⁺, Method 4): 1.25 min, m/z, 481.15 [M+H]⁺. ¹H NMR (400 MHz, DMSO-d₆) δ 9.08 (s, 1H), 8.98 (t, J=8.2 Hz, 1H), 8.45 (d, J=6.3 Hz, 2H), 8.00 (d, J=8.2 Hz, 2H), 7.62 (d, J=8.2 Hz, 2H), 7.05-6.98 (m, 2H), 6.86 (d, J=7.4 Hz, 1H), 4.65 (s, 2H), 3.99-3.95 (m, 2H), 1.90 (s, 3H) ppm.

### Example 47: 2-[4-methyl-3-[5-(1-methylpyrazolo[3,4-c]pyridin-4-yl)pyrazin-2-yl]-2-oxo-benzimidazol-1-yl]-N-(2,2,2-trifluoroethyl)acetamide

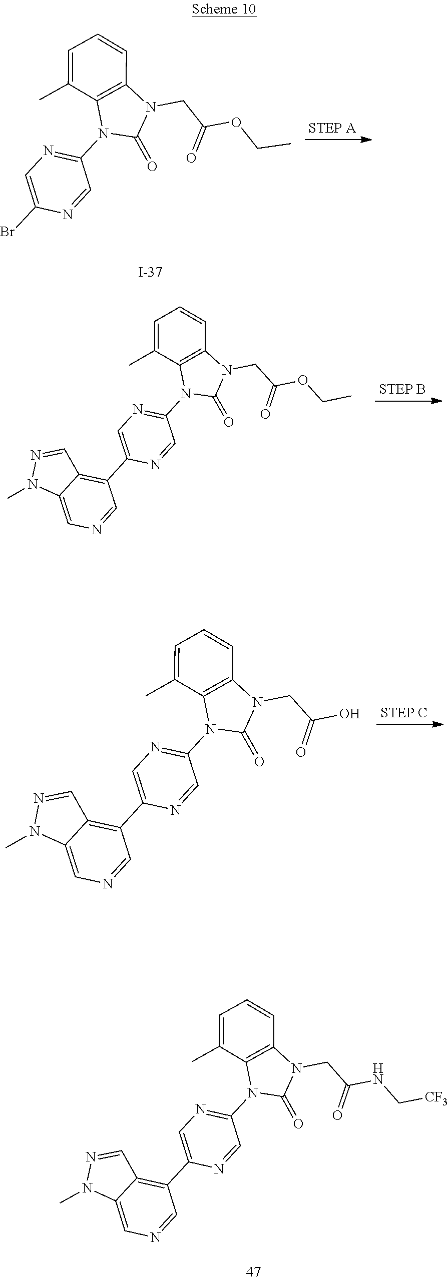

STEP A: ethyl 2-[4-methyl-3-[6-methyl-5-(1-methylpyrazolo[3,4-c]pyridin-4-yl)pyrazin-2-yl]-2-oxo-benzimidazol-1-yl]acetate. A solution of ethyl 2-[3-(5-bromo-6-methyl-pyrazin-2-yl)-4-methyl-2-oxo-benzimidazol-1-yl]acetate (Intermediate I-37) (300 mg, 0.74 mmol), tributyl-(1-methylpyrazolo[3,4-c]pyridin-4-yl)stannane (375 mg, 0.89 mmol), Pd(PPh₃)₄ (86 mg, 0.07 mmol), LiCl (94 mg, 2.22 mmol) and CuI (70 mg, 0.37 mmol) in Toluene (10 mL) was stirred at 100° C. under an N₂ atmosphere overnight. The mixture was cooled to room temperature, diluted with water (100 mL) and extracted with ethyl acetate (100 mL×3). The combined organic layers were dried over Na₂SO₄ and concentrated under vacuum and purified by silica gel column chromatography (DCM/MeOH, 100:1 to DCM/MeOH, 50:1) to provide ethyl 2-[4-methyl-3-[6-methyl-5-(1-methylpyrazolo[3,4-c]pyridin-4-yl)pyrazin-2-yl]-2-oxo-benzimidazol-1-yl]acetate (220 mg, 65%) as a yellow solid. LC-MS (ES⁺, Method 4): 1.60 min, m/z, 458.15 [M+H]⁺

STEP B: 2-[4-methyl-3-[5-(1-methylpyrazolo[3,4-c]pyridin-4-yl)pyrazin-2-yl]-2-oxo-benzimidazol-1-yl]acetic acid. A mixture of ethyl 2-[7-[(1-methylpyrazolo[3,4-b]pyridin-5-yl)amino]-1-oxo-isoindolin-2-yl]acetate (50 mg, 0.11 mmol), LiOH (9.5 mg, 0.23 mmol), THE (3 mL), MeOH (3 mL) and H₂O (0.5 mL) was stirred at 25° C. for 2 hours. The mixture was diluted with H₂O and 10% HCl was added until the pH reached 1-2, the resulting precipitate isolated by filtration and analysed as 2-[4-methyl-3-[5-(1-methylpyrazolo[3,4-c]pyridin-4-yl)pyrazin-2-yl]-2-oxo-benzimidazol-1-yl]acetic acid (40 mg, 85%) as a yellow solid. LC-MS (ES⁺, Method 3): 3.183 min, m/z, 416.10 [M+H]⁺

STEP C: 2-[4-methyl-3-[5-(1-methylpyrazolo[3,4-c]pyridin-4-yl)pyrazin-2-yl]-2-oxo-benzimidazol-1-yl]-N-(2,2,2-trifluoroethyl)acetamide. A mixture of 2-[4-methyl-3-[5-(1-methylpyrazolo[3,4-c]pyridin-4-yl)pyrazin-2-yl]-2-oxo-benzimidazol-1-yl]acetic acid (40 mg, 0.096 mmol), trifluoroethylamine (14 mg, 0.14 mmol), HATU (44 mg, 0.12 mmol) and DIEA (62.11 mg, 0.48 mmol) in DMF (5 mL) was stirred at 25° C. overnight. The mixture was purified by flash column chromatography on silica gel (dichloromethane/methanol, 50/1 to dichloromethane/methanol, 20:1) affording 2-[4-methyl-3-[5-(1-methylpyrazolo[3,4-c]pyridin-4-yl)pyrazin-2-yl]-2-oxo-benzimidazol-1-yl]-N-(2,2,2-trifluoroethyl)acetamide (example 47) (9.2 mg, 19.2%) as a white solid. LC-MS (ES⁺, Method 3): 3.46 min. m/z, 497.10 [M+H]⁺. ¹H NMR (400 MHz, DMSO-d₆) δ 9.57 (d, J=1.4 Hz, 1H), 9.35 (s, 1H), 9.15 (s, 1H), 9.11 (s, 1H), 9.01 (t, J=6.3 Hz, 1H), 8.76 (s, 1H), 7.12 (t, J=7.7 Hz, 1H), 7.05 (d, J=7.8 Hz, 1H), 6.95 (d, J=7.6 Hz, 1H), 4.69 (s, 2H), 4.28 (s, 3H), 4.03-3.94F (in, 2H), 2.00 (s, 3H) ppm.

The following examples (table 10) were prepared by analogy with example 47 (Scheme 10), replacing 1-37 in step A or trifluoroethylamine in Step C with the appropriate building blocks as described in the table.

TABLE 10

| Building blocks and | | LC-MS | ¹H-NMR (400 MHz, |
|---|---|---|---|
| reagents | Example structure and name | (ES⁺) | DMSO-d6) |
| STEP A: Intermediate I-37 STEP C: (R)-1,1,1-trifluoro-2- propylamine 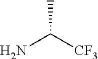 HATU (1.5 eq) | 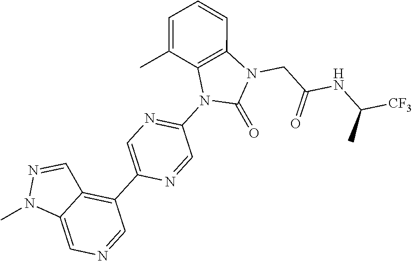 [SMILES: Cc1cccc2c1n(-c1cnc(-c3cncc4c3cnn4C)cn1)c(=O)n2CC(=O)N[C@H](C)C(F)(F)F] | Method 3: 3.58 min, m/z 511.30 [M + H]⁺ | δ 9.57 (s, 1H), 9.35 (s, 1H), 9.15 (s, 1H), 9.11 (s, 1H), 8.94 (d, J = 8.8 Hz, 1H), 8.76 (s, 1H), 7.12 (m, 1H), 7.04 (d, J = 7.8 Hz, 1H), 6.95 (d, J = 7.6 Hz, 1H), 4.66-4.60 (m, 3H), 4.28 (s, 3H), 1.99 (s, 3H), 1.30 (d, J = 7.0 Hz, 3H) ppm. |
| | 2-[4-methyl-3-[5-(1-methylpyrazolo[3,4-c]pyridin-4-yl)pyrazin- | | |
| | 2-yl]-2-oxo-benzimidazol-1-yl]-N-[(1R)-2,2,2- | | |
| | trifluoro-1-methyl-ethyl]acetamide | | |
| | Example 48 | | |
| STEP A: Intermediate I-37 STEP C: (S)-1,1,1-trifluoro-2- propylamine 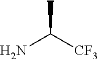 HATU (1.5 eq) | 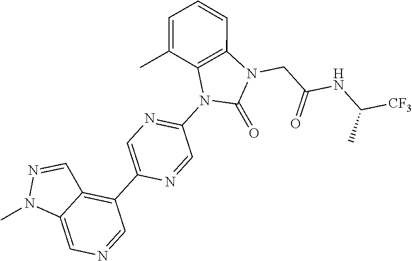 [SMILES: Cc1cccc2c1n(-c1cnc(-c3cncc4c3cnn4C)cn1)c(=O)n2CC(=O)N[C@@H](C)C(F)(F)F] | Method 3: 3.58 min, m/z 511.30 [M + H]⁺ | δ 9.57 (d, J = 1.4 Hz, 1H), 9.35 (s, 1H), 9.15 (d, J = 1.4 Hz, 1H), 9.11 (s, 1H), 8.94 (d, J = 8.8 Hz, 1H), 8.76 (s, 1H), 7.12 (m, 1H), 7.03 (d, J = 7.8 Hz, 1H), 6.95 (d, J = 7.6 Hz, 1H), 4.66-4.58 (m, 3H), 4.28 (s, 3H), 1.99 (s, 3H), 1.29 (d, J = 7.0 Hz, 3H) ppm. |
| | 2-[4-methyl-3-[5-(1-methylpyrazolo[3,4-c]pyridin-4- | | |
| | yl)pyrazin-2-yl]-2-oxo-benzimidazol-1-yl]-N-[(1S)-2,2,2- | | |
| | trifluoro-1-methyl-ethyl]acetamide | | |
| | Example 49 | | |
| STEP A: Intermediate I-38 STEP C: trifluoroethylamine 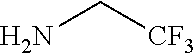 HATU (1.2 eq) | 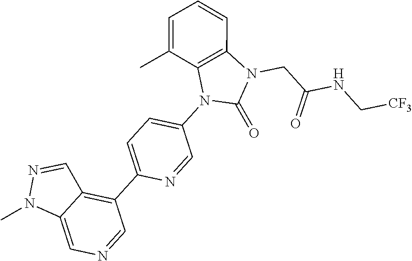 [SMILES: Cc1cccc2c1n(-c1ccc(-c3cncc4c3cnn4C)nc1)c(=O)n2CC(=O)NCC(F)(F)F] | Method 3: 3.28 min, m/z 496.10 [M + H]⁺ | δ 9.29 (s, 1H), 8.96 (m, 2H), 8.90 (d, J = 2.4 Hz, 1H), 8.74 (s, 1H), 8.40 (d, J = 8.4 Hz, 1H), 8.10 (dd, J = 8.5, 2.5 Hz, 1H), 7.08-7.02 (m, 2H), 6.89 (d, J = 7.2 Hz, 1H), 4.67 (s, 2H), 4.26 (s, 3H), 4.03- 3.94 (d, J = 34.3 Hz, 2H), 1.94 (s, 3H) ppm. |
| | 2-[4-methyl-3-[6-(1-methylpyrazolo[3,4-c]pyridin-4-yl)-3-pyridyl]- | | |
| | 2-oxo-benzimidazol-1-yl]-N-(2,2,2-trifluoroethyl)acetamide | | |
| | Example 50 | | |
| STEP A: Intermediate I-38 STEP C: (2R)-Amino-1,1,1- trifluoropropane 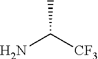 HATU (1.5 eq) | 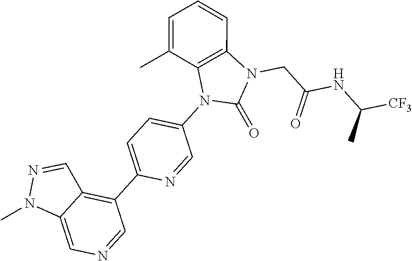 [SMILES: Cc1cccc2c1n(-c1ccc(-c3cncc4c3cnn4C)nc1)c(=O)n2CC(=O)N[C@H](C)C(F)(F)F] | Method 4: 1.43 min, m/z 510.15 [M + H]⁺ | δ 9.29 (s, 1H), 8.98 (s, 1H), 8.93-8.86 (m, 2H), 8.74 (s, 1H), 8.40 (d, J = 8.4 Hz, 1H), 8.10 (dd, J = 8.5, 2.5 Hz, 1H), 7.09-7.00 (m, 2H), 6.89 (d, J = 7.4 Hz, 1H), 4.64 (s, 3H), 4.26 (s, 3H), 1.94 (s, 3H), 1.30 (d, J = 7.0 Hz, 3H) ppm. |
| | 2-[4-methyl-3-[6-(1-methylpyrazolo[3,4-c]pyridin-4-yl)-3-pyridyl]- | | |
| | 2-oxo-benzimidazol-1-yl]-N-[(1R)-2,2,2-trifluoro-1-methyl- | | |
| | ethyl]acetamide | | |
| | Example 51 | | |
| STEP A: Intermediate I-38 STEP C: (2S)-Amino-1,1,1- trifluoropropane 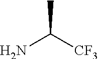 HATU (1.5 eq) | 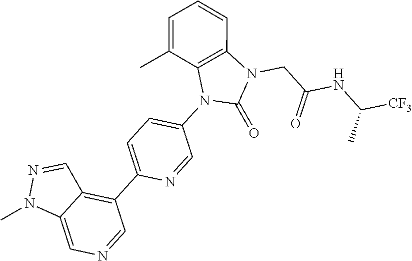 [SMILES: Cc1cccc2c1n(-c1ccc(-c3cncc4c3cnn4C)nc1)c(=O)n2CC(=O)N[C@@H](C)C(F)(F)F] | Method 4: 1.47 min, m/z 510.20 [M + H]⁺ | δ 9.29 (s, 1H), 9.03- 8.87 (m, 3H), 8.74 (s, 1H), 8.39 (d, J = 8.5 Hz, 1H), 8.10 (dd, J = 8.4, 2.5 Hz, 1H), 7.08-7.00 (m, 2H), 6.89 (d, J = 7.4 Hz, 1H), 4.69-4.58 (m, 3H), 4.26 (s, 3H), 1.94 (s, 3H), 1.30 (d, J = 7.0 Hz, 3H) ppm. |
| | 2-[4-methyl-3-[6-(1-methylpyrazolo[3,4-c]pyridin-4-yl)-3-pyridyl]- | | |
| | 2-oxo-benzimidazol-1-yl]-N-[(1S)-2,2,2-trifluoro-1-methyl- | | |
| | ethyl]acetamide | | |
| | Example 52 | | |
| STEP A: Intermediate I-38 STEP C: (3S)- (trifluoromethyl)morpholine 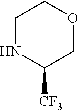 CMPI (1.5 eq) | 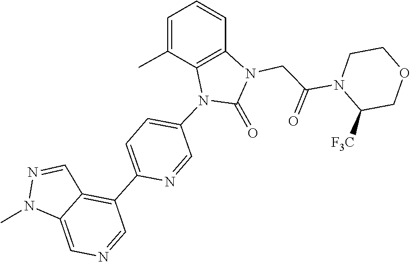 [SMILES: Cc1cccc2c1n(-c1ccc(-c3cncc4c3cnn4C)nc1)c(=O)n2CC(=O)N1CCOC[C@H]1C(F)(F)F] | Method 4: 1.46 min, m/z 552.20 [M + H]⁺ | δ 9.29 (s, 1H), 8.98 (s, 1H), 8.90 (s, 1H), 8.74 (s, 1H), 8.40 (d, J = 8.5 Hz, 1H), 8.11-8.08 (m, 1H), 7.09-7.03 (m, 2H), 6.91-6.88 (m, 1H), 5.21-4.72 (m, 3H), 4.26 (s, 3H), 4.20-4.11 (m, 1H), 3.97-3.87 (m, 2H), 3.71-3.48 (m, 3H), 1.94 (s, 3H) ppm. |
| | 4-methyl-3-[6-(1-methylpyrazolo[3,4-c]pyridin-4-yl)-3-pyridyl]- | | |
| | 1-[2-oxo-2-[(3S)-3-(trifluoromethyl)morpholin-4-yl]ethyl] | | |
| | benzimidazol-2-one | | |
| | Example 53 | | |
| STEP A: Intermediate I-38 STEP C: (2S)- (trifluoromethyl)morpholine 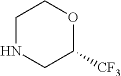 HATU (1.2 eq) | 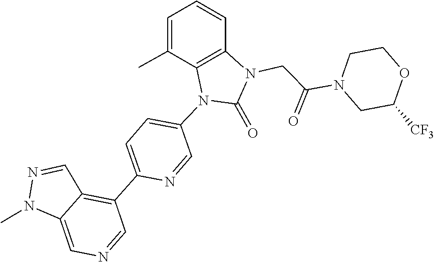 [SMILES: Cc1cccc2c1n(-c1ccc(-c3cncc4c3cnn4C)nc1)c(=O)n2CC(=O)N1CCO[C@H](C(F)(F)F)C1] | Method 4: 1.53 min, m/z 552.30 [M + H]⁺ | δ 9.29 (s, 1H), 8.98 (s, 1H), 8.89 (d, J = 2.4 Hz, 1H), 8.74 (s, 1H), 8.40 (d, J = 8.4 Hz, 1H), 8.09 (dd, J = 8.5, 2.5 Hz, 1H), 7.11-7.03 (m, 2H), 6.88 (d, J = 7.1 Hz, 1H), 5.13-5.01 (m, 1H), 4.90-4.83 (m, 1H), 4.42-4.20 (m, 5H), 4.16-3.69 (m, 2H), 3.78-3.55 (m, 1H), 3.42 (s, 1H), 2.96-2.86 (m, 1H), 1.94 (s, 3H) ppm. |
| | 4-methyl-3-[6-(1-methylpyrazolo[3,4-c]pyridin-4-yl)-3-pyridyl]- | | |
| | 1-[2-oxo-2-[(2S)-2-(trifluoromethyl)morpholin-4-yl]ethyl] | | |
| | benzimidazol-2-one | | |
| | Example 54 | | |
| STEP A: Intermediate I-38 STEP C: (2R)- (trifluoromethyl)morpholine 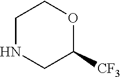 HATU (1.2 eq) | 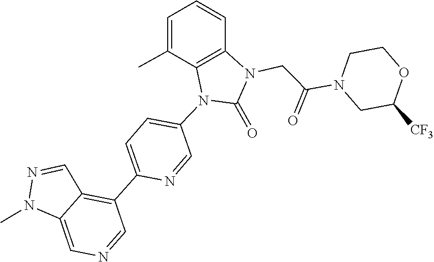 [SMILES: Cc1cccc2c1n(-c1ccc(-c3cncc4c3cnn4C)nc1)c(=O)n2CC(=O)N1CCO[C@@H](C(F)(F)F)C1] | Method 3: 3.48 min, m/z 552.30 [M + H]⁺ | δ 9.29 (s, 1H), 8.97 (s, 1H), 8.89 (d, J = 2.4 Hz, 1H), 8.74 (s, 1H), 8.40 (d, J = 8.5 Hz, 1H), 8.08 (dd, J = 8.4, 2.5 Hz, 1H), 7.10-7.03 (m, 2H), 6.88 (d, J = 7.3 Hz, 1H), 5.13-4.83 (m, 2H), 4.42-3.54 (m, 9H), 2.96-2.90 (m, 1H), 1.94 (s, 3H) ppm. |
| | 4-methyl-3-[6-(1-methylpyrazolo[3,4-c]pyridin-4-yl)-3-pyridyl]-1- | | |
| | [2-oxo-2-[(2S)-2-trifluoromethyl)morpholin-4-yl] | | |
| | ethyl]benzimidazol-2-one | | |
| | Example 55 | | |
| STEP A: Intermediate I-38 STEP C: (2S)- (trifluoromethyl)pyrrolidine 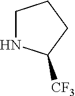 HATU (1.5 eq) | 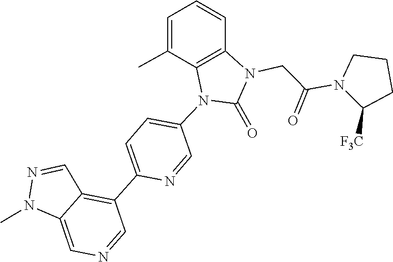 [SMILES: Cc1cccc2c1n(-c1ccc(-c3cncc4c3cnn4C)nc1)c(=O)n2CC(=O)N1CCC[C@H]1C(F)(F)F] | Method 4: 1.60 min, m/z 536.20 [M + H]⁺ | δ 9.29 (s, 1H), 8.98 (s, 1H), 8.90 (s, 1H), 8.74 (s, 1H), 8.40 (d, J = 8.2 Hz, 1H), 8.09 (m, 1H), 7.10-7.03 (m, 2H), 6.88 (m, 1H), 5.13- 4.83 (m, 3H), 4.26 (s, 3H), 3.86-3.65 (m, 2H), 2.17-1.94 (m, 7H) ppm. |
| | 4-methyl-3-[6-(1-methylpyrazolo[3,4-c]pyridin-4-yl)-3- | | |
| | pyridyl]-1-[2-oxo-2-[(2S)-2-(trifluoromethyl)pyrrolidin-1- | | |
| | yl]ethyl]benzimidazol-2-one | | |
| | Example 56 | | |
| STEP A: Intermediate I-39 STEP C: Trifluoroethylamine 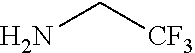 HATU (1.5 eq) | 2-[4-methyl-3-[5-(1- methylpyrazolo[3,4-c]pyridin-4-yl)- 2-pyridyl]-2-oxo-benzimidazol-1-yl]- N-[(1S)-2,2,2-trifluoro-1-methyl- ethyl]acetamide 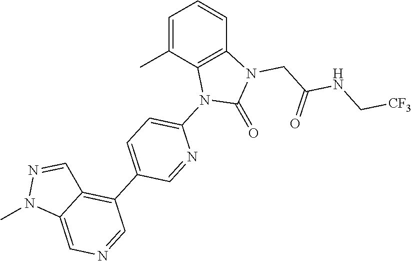 [SMILES: Cc1cccc2c1n(-c1ccc(-c3cncc4c3cnn4C)cn1)c(=O)n2CC(=O)NCC(F)(F)F] | Method 3: 3.28 min, m/z 496.10 [M + H]⁺ | δ 9.31 (s, 1H), 9.09 (d, J = 2.5 Hz, 1H), 8.99 (t, J = 6.3 Hz, 1H), 8.63 (s, 1H), 8.52 (dd, J = 8.2, 2.5 Hz, 1H), 8.48 (s, 1H), 7.82 (d, J = 8.3 Hz, 1H), 7.07 (t, J = 7.7 Hz, 1H), 7.01 (d, J = 7.7 Hz, 1H), 6.90 (d, J = 7.5 Hz, 1H), 4.66 (s, 2H), 4.26 (s, 3H), 4.02-3.93 (m, 2H), 1.94 (s, 3H) ppm. |
| | Example 58 | | |
| STEP A: Intermediate I-43 STEP C: Trifluoroethylamine 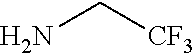 HATU (1.5 eq) | 2-[4-methyl-3-[5-(1- methylpyrazolo[3,4-c]pyridin-4-yl)- 2-pyridyl]-2-oxo-benzimidazol-1-yl]- N-[(1S)-2,2,2-trifluoro-1-methyl- ethyl]acetamide 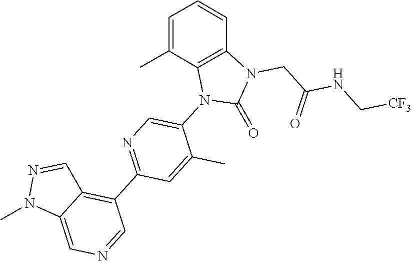 [SMILES: Cc1cc(-c2cncc3c2cnn3C)ncc1-n1c(=O)n(CC(=O)NCC(F)(F)F)c2cccc(C)c21] | Method 4: 1.40 min, m/z 510.15 [M + H]⁺ | δ 9.28 (s, 1H), 8.98 (m, 2H), 8.77 (m, 2H), 8.31 (s, 1H), 7.05 (m, 1H), 6.85 (m, 1H), 4.73 (s, 2H), 4.26 (s, 3H), 4.02-3.93 (m, 2H), 2.94 (s, 2H), 2.22 (s, 3H), 1.94 (s, 3H) ppm. |
| | Example 64 | | |
| STEP A: Intermediate I-44 STEP C: (2S)- (trifluoromethyl)pyrrolidine 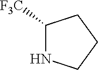 HATU (1.5 eq) | 1-[6-(1-methylpyrazolo[3,4- c]pyridin-4-yl)-3-pyridyl]-3-[2-oxo-2- [(2S)-2-(trifluoromethyl)pyrrolidin-1- yl]ethyl]benzimidazol-2-one 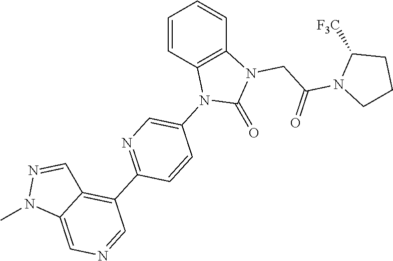 | Method 4: 1.60 min, m/z 536.20 [M + H]⁺ | δ 9.51 (s, 1H), 9.20- 8.80 (m, 3H), 8.47 (d, J = 8.5 Hz, 1H), 8.26 (dd, J = 8.6, 2.5 Hz, 1H), 7.32- 7.12 (m, 4H), 5.23- 4.88 (m, 2H), 4.80- 4.73 (m, 1H), 4.30 (s, 3H), 3.86-3.65 (m, 2H), 2.17-1.94 (m, 4H) ppm. |
| | Example 65 | | |
| STEP A: Intermediate I-46 STEP C: Trifluoroethylamine 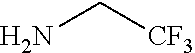 HATU (1.5 eq) | 2-[4-methyl-3-[6-(1- methylpyrazolo[3,4-c]pyridin-4- yl)pyridazin-3-yl]-2-oxo- benzimidazol-1-yl]-N-(2,2,2- trifluoroethyl)acetamide 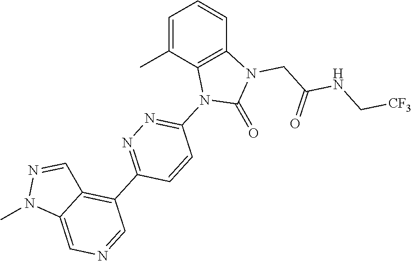 [SMILES: Cc1cccc2c1n(-c1ccc(-c3cncc4c3cnn4C)nn1)c(=O)n2CC(=O)NCC(F)(F)F] | Method : 3.32 min, m/z 497.10 [M + H]⁺ | δ 9.43 (s, 1H), 9.04 (s, 1H), 9.01 (m, 1H), 8.77 (d, J = 9.0 Hz, 1H), 8.71 (s, 1H), 8.19 (d, J = 9.0 Hz, 1H), 7.13 (m,, 1H), 7.07 (d, J = 7.8 Hz, 1H), 6.96 (d, J = 7.5 Hz, 1H), 4.70 (s, 2H), 4.29 (s, 3H), 3.99 (qd, J = 9.7, 6.2 Hz, 2H), 1.95 (s, 3H) ppm. |
| | Example 67 | | |
| STEP A: Intermediate I-63 STEP C: trifluoroethylamine 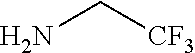 HATU (1.5 eq) | 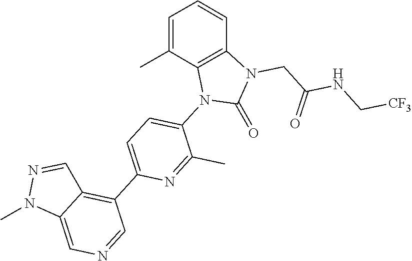 [SMILES: Cc1nc(-c2cncc3c2cnn3C)ccc1-n1c(=O)n(CC(=O)NCC(F)(F)F)c2cccc(C)c21] | Method 4: 1.44 min, m/z 510.20 [M + H]⁺ | δ 9.28 (s, 1 H), 8.98 (s, 2 H), 8.81 (s, 1 H), 8.25 (d, J = 8.0 Hz, 1 H), 8.01 (d, J = 8.0 Hz, 1 H), 7.04 (d, J = 4.0 Hz, 2 H), 6.86 (m, 1H), 4.68 (s, 2 H), 4.26 (s, 3 H), 4.04-3.94 (m, 2 H), 2.49 (s, 3 H), 1.83 (s, 3 H) ppm. |
| | 2-[4-methyl-3-[2-methyl-6-(1-methylpyrazolo[3,4-c]pyridin- | | |
| | 4-yl)-3-pyridyl]-2-oxo-benzimidazol-1-yl]-N-(2,2,2- | | |
| | trifluoroethyl)acetamide | | |
| | Example 95 | | |
| STEP A: Intermediate I-64 STEP C: trifluoroethylamine 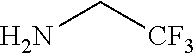 HATU (1.5 eq) | 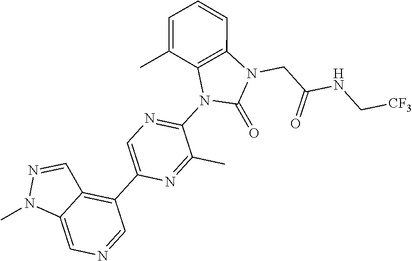 [SMILES: Cc1nc(-c2cncc3c2cnn3C)cnc1-n1c(=O)n(CC(=O)NCC(F)(F)F)c2cccc(C)c21] | Method 3: 2.99 min, m/z 511.30 [M + H]⁺ | δ 9.43 (s, 2H), 9.16 (s, 1H), 9.01 (m, 1H), 8.82 (s, 1H), 7.08 (d, J = 8.3 Hz, 2H), 6.90 (d, J = 7.1 Hz, 1H), 4.69 (d, J = 4.6 Hz, 2H), 4.28 (s, 3H), 4.03-3.96 (m, 2H), 2.64 (s, 3H), 1.81 (s, 3H) ppm. |
| | 2-[4-methyl-3-[3-methyl-5-(1-methylpyrazolo[3,4-c]pyridin-4- | | |
| | yl)pyrazin-2-yl]-2-oxo-benzimidazol-1-yl]-N-(2,2,2- | | |
| | trifluoroethyl)acetamide | | |
| | Example 96 | | |
| STEP A: Intermediate I-65 STEP C: trifluoroethylamine 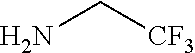 HATU (1.5 eq) | 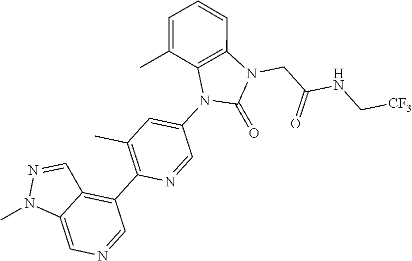 [SMILES: Cc1cc(-n2c(=O)n(CC(=O)NCC(F)(F)F)c3cccc(C)c32)cnc1-c1cncc2c1cnn2C] | Method 3: 2.95 min, m/z 510.25 [M + H]⁺ | δ 9.27 (s, 1H), 8.69 (d, J = 2.3 Hz, 1H), 8.45 (s, 1H), 8.08 (s, 1H), 8.01 (d, J = 2.3 Hz, 1H), 7.08- 7.02 (m, 2H), 6.92- 6.88 (m, 1H), 4.67 (s, 2H), 4.25 (s, 3H), 3.98 (q, J = 9.9 Hz, 2H), 2.41 (s, 3H), 1.95 (s, 3H) ppm. |
| | 2-[4-methyl-3-[5-methyl-6-(1-methylpyrazolo[3,4-c]pyridin- | | |
| | 4-yl)-3-pyridyl]-2-oxo-benzimidazol-1-yl]-N-(2,2,2-trifluoroethyl) | | |
| | acetamide | | |
| | Example 97 | | |
| STEP A: Intermediate I-66 STEP C: (2S)-Amino-1,1,1- trifluoropropane 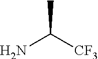 HATU (1.5 eq) | 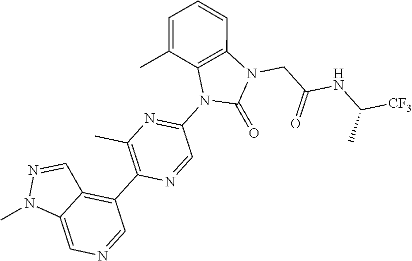 [SMILES: Cc1nc(-n2c(=O)n(CC(=O)N[C@@H](C)C(F)(F)F)c3cccc(C)c32)cnc1-c1cncc2c1cnn2C] | Method 4: 1.67 min, m/z 525.10 [M + H]⁺ | δ 9.32 (s, 1H), 8.96 (s, 1H), 8.93 (d, J = 8.5 Hz, 1H), 8.56 (s, 1H), 8.20 (s, 1H), 7.12 (m, 1H), 7.03 (d, J = 7.9 Hz, 1H), 6.95 (d, J = 7.7 Hz, 1H), 4.66 (s, 2H), 4.27 (s, 3H), 2.62 (s, 3H), 2.01 (s, 3H), 1.30 (d, J = 7.1 Hz, 3H) ppm. |
| | 2-[4-methyl-3-[6-methyl-5-(1-methylpyrazolo[3,4-c]pyridin-4- | | |
| | yl)pyrazin-2-yl]-2-oxo-benzimidazol-1-yl]-N-[(1S)-2,2,2- | | |
| | trifluoro-1-methyl-ethyl]acetamide | | |
| | Example 101 | | |

### Example 66: 2-[4-methyl-3-[5-(1-methylpyrazolo[3,4-c]pyridin-4-yl)pyrimidin-2-yl]-2-oxo-benzimidazol-1-yl]-N-(2,2,2-trifluoroethyl)acetamide

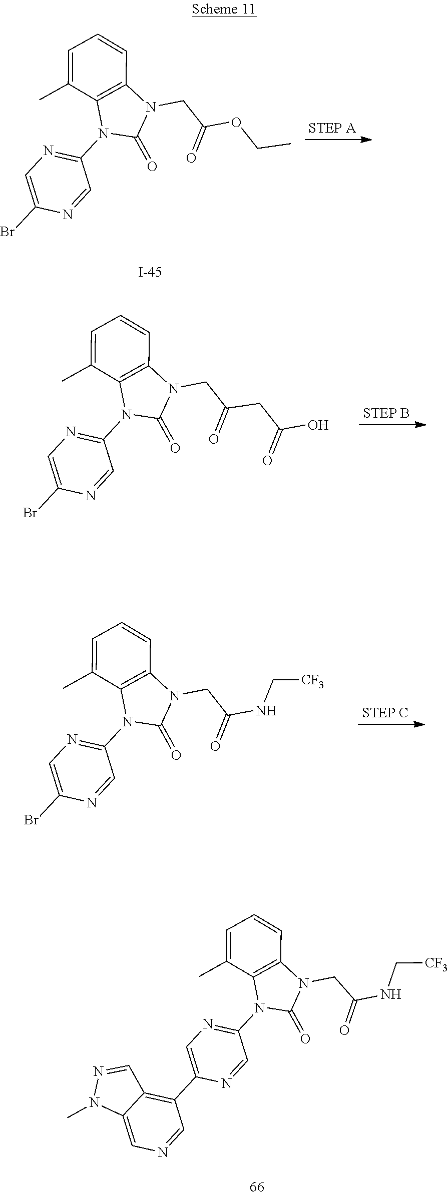

STEP A: 2-[3-(5-bromopyrimidin-2-yl)-4-methyl-2-oxo-benzimidazol-1-yl]acetic acid. A mixture of ethyl 2-(3-(5-bromopyrimidin-2-yl)-4-methyl-2-oxo-2,3-dihydro-1H-benzo[d]imidazol-1-yl)acetate (600 mg, 1.54 mmol) and LiOH·H₂O (194 mg, 4.62 mmol) in H₂O/THF (8 mL) was stirred at room temperature for 2 h. The mixture was concentrated under vacuum and the pH adjusted to 3 by slow addition of 1 M HCl. The mixture was diluted with water (50 mL) and was washed with EtOAc (100 ml×3). The combined organic layers were washed with brine (50 mL), dried over Na₂SO₄ and was concentrated to give 2-[3-(5-bromopyrimidin-2-yl)-4-methyl-2-oxo-benzimidazol-1-yl]acetic acid (440 mg, 79%) as a white solid.

LC-MS (ES⁺, Method 4): 1.13 min, m/z, 444/446 [M+H]⁺.

STEP B: 2-[3-(5-bromopyrimidin-2-yl)-4-methyl-2-oxo-benzimidazol-1-yl]-N-(2,2,2-trifluoroethyl)acetamide. A mixture of 2-(3-(5-bromopyrimidin-2-yl)-4-methyl-2-oxo-2,3-dihydro-1H-benzo[d]imidazol-1-yl)acetic acid (500 mg, 1.38 mmol), 2,2,2-trifluoroethan-1-amine (205 mg, 2.07 mmol), HATU (787 mg, 2.07 mmol) and DIPEA (535 mg, 4.14 mmol) in DMF (5 mL) was stirred at room temperature for 2 h. The mixture was treated with water (50 ml) and was filtered to give 2-[3-(5-bromopyrimidin-2-yl)-4-methyl-2-oxo-benzimidazol-1-yl]-N-(2,2,2-trifluoroethyl)acetamide (510 mg, 83%) as a white solid. LC-MS (ES⁺, Method 4): 1.31 min, m/z, 363/365 [M+H]⁺.

STEP C: 2-[4-methyl-3-[5-(1-methylpyrazolo[3,4-c]pyridin-4-yl)pyrimidin-2-yl]-2-oxo-benzimidazol-1-yl]-N-(2,2,2-trifluoroethyl)acetamide. A mixture of 2-(3-(5-bromopyrimidin-2-yl)-4-methyl-2-oxo-2,3-dihydro-1H-benzo[d]imidazol-1-yl)-N-(2,2,2-trifluoroethyl)acetamide (100 mg, 0.23 mmol), 1-methyl-4-(tributylstannyl)-1H-pyrazolo[3,4-c]pyridine (115 mg, 0.27 mmol), Pd(PPh₃)₄ (26 mg, 0.023 mmol), CuI (24 mg, 0.11 mmol), LiCl (29 mg, 0.68 mmol) in toluene (4 ml) was stirred at 120° C. under N₂ atmosphere overnight. The reaction was filtered and was purified by preparative TLC (DCM/MeOH, 20:1) affording 2-[4-methyl-3-[5-(1-methylpyrazolo[3,4-c]pyridin-4-yl)pyrimidin-2-yl]-2-oxo-benzimidazol-1-yl]-N-(2,2,2-trifluoroethyl)acetamide as a white solid (example 66) (10 mg, 8.9%). LC-MS (ES⁺, Method 3): 3.31 min, m/z, 497.25 [M+H]⁺. 1H NMR (400 MHz, DMSO-d₆) δ 9.51 (s, 2H), 9.33 (s, 1H), 9.03 (t, J=6.3 Hz, 1H), 8.68 (s, 1H), 8.58 (s, 1H), 7.09 (t, J=7.8 Hz, 1H), 7.02 (d, J=7.8 Hz, 1H), 6.92 (d, J=7.6 Hz, 1H), 4.66 (s, 2H), 4.27 (s, 3H), 4.03-3.94 (m, 2H), 1.88 (s, 3H) ppm.

### Example 68: 2-[4-methyl-2-oxo-3-[6-(1H-pyrazolo[3,4-c]pyridin-4-yl)-3-pyridyl]benzimidazol-1-yl]-N-[(1S)-2,2,2-trifluoro-1-methyl-ethyl]acetamide

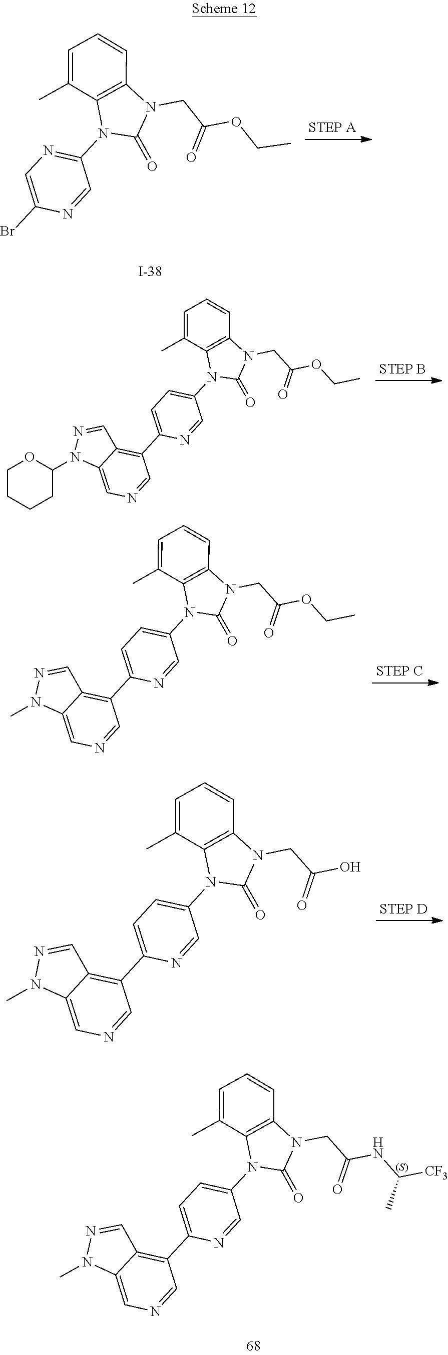

STEP A: ethyl 2-[4-methyl-2-oxo-3-[6-(1-tetrahydropyran-2-ylpyrazolo[3,4-c]pyridin-4-yl)-3-pyridyl]benzimidazol-1-yl]acetate. A solution of ethyl 2-[3-(6-bromo-3-pyridyl)-4-methyl-2-oxo-benzimidazol-1-yl]acetate (Intermediate I-38) (500 mg, 1.28 mmol), tributyl-(1-tetrahydropyran-2-ylpyrazolo[3,4-c]pyridin-4-yl)stannane (756 mg, 1.54 mmol), Tetrakis(triphenylphosphine)palladium(0) (148.57 mg, 0.13 mmol), lithium chloride (162 mg, 3.84 mmol), and CuI (122 mg, 0.64 mmol), in toluene (3 mL) was stirred at 110° C. under N₂ overnight. The mixture was diluted with EtOAc (8 mL) and washed with H₂O (10 mL×3). The organic layers were washed with EtOAc (15 mL×3), combined, dried over anhydrous Na₂SO₄ and purified by preparative TLC to give ethyl 2-[4-methyl-2-oxo-3-[6-(1-tetrahydropyran-2-ylpyrazolo[3,4-c]pyridin-4-yl)-3-pyridyl]benzimidazol-1-yl]acetate (160 mg, 24%) as a yellow solid. LC-MS (ES⁺, Method 3): 3.95 min, m/z, 513.35 [M+H]⁺

STEP B: ethyl 2-[4-methyl-2-oxo-3-[6-(1H-pyrazolo[3,4-c]pyridin-4-yl)-3-pyridyl]benzimidazol-1-yl]acetate. A solution of ethyl 2-[4-methyl-2-oxo-3-[6-(1-tetrahydropyran-2-ylpyrazolo[3,4-c]pyridin-4-yl)-3-pyridyl]benzimidazol-1-yl]acetate (160 mg, 0.31 mmol) hydrochloric acid (4N in dioxane) (3 mL, 0.01 mmol), in DCM (1.0 mL) was stirred at room temperature for 2 h. The mixture was concentrated under vacuum and purified by silica gel column chromatography (Pet.ether/EtOAc, 20/1 to Pet.ether/EtOAc, ⅓) affording ethyl 2-[4-methyl-2-oxo-3-[6-(1H-pyrazolo[3,4-c]pyridin-4-yl)-3-pyridyl]benzimidazol-1-yl]acetate (130 mg, 96%). LC-MS (ES⁺, Method 3): 3.12 min, m/z, 429.25 [M+H]⁺

STEP C: 2-[4-methyl-2-oxo-3-[6-(1H-pyrazolo[3,4-c]pyridin-4-yl)-3-pyridyl]benzimidazol-1-yl]acetic acid. A mixture of ethyl 2-[4-methyl-2-oxo-3-[6-(1H-pyrazolo[3,4-c]pyridin-4-yl)-3-pyridyl]benzimidazol-1-yl]acetate (130 mg, 0.3 mmol) and lithium hydroxide monohydrate (38.19 mg, 0.91 mmol) in THE (3 mL), Methanol (3 mL) and Water (1 mL) was stirred at room temperateure for 2 h. The mixture was dilluted with H₂O (3 mL) and 10% HCl (3 mL) to reach pH 2-3. The mixture was diluted with water (5 mL×3) and extracted with DCM (10 mL×3). After drying over Na₂SO₄, the mixture was filtered and concentrated in vacuo to obtain 2-[4-methyl-2-oxo-3-[6-(1H-pyrazolo[3,4-c]pyridin-4-yl)-3-pyridyl]benzimidazol-1-yl]acetic acid (100 mg, 82%) as a yellow solid. LC-MS (ES⁺, Method 3): 2.72 min, m/z, 401.25 [M+H]⁺

STEP D: 2-[4-methyl-2-oxo-3-[6-(1H-pyrazolo[3,4-c]pyridin-4-yl)-3-pyridyl]benzimidazol-1-yl]-N-[(1S)-2,2,2-trifluoro-1-methyl-ethyl]acetamide. A solution of 2-[4-methyl-2-oxo-3-[6-(1H-pyrazolo[3,4-c]pyridin-4-yl)-3-pyridyl]benzimidazol-1-yl]acetic acid (100 mg, 0.25 mmol), (2S)-2-Amino-1,1,1-trifluoropropane hydrochloride (33.89 mg, 0.3 mmol), HATU (113.96 mg, 0.3 mmol) and DIEA (96.83 mg, 0.75 mmol), in DMF (10 mL) was stirred at room temperature for 2 h. The mixture was diluted with EtOAc (15 mL) and washed with H₂O (3 mL×3). The organic layers were combined, dried over anhydrous Na₂SO₄. The mixture was purified by reverse-phase column chromatography to give 2-[4-methyl-2-oxo-3-[6-(1H-pyrazolo[3,4-c]pyridin-4-yl)-3-pyridyl]benzimidazol-1-yl]-N-[(1S)-2,2,2-trifluoro-1-methyl-ethyl]acetamide (49.7 mg, 40%) as an off-white solid. LC-MS (ES⁺, Method 3): 3.13 min, m/z, 496.30 [M+H]⁺. ¹H NMR (400 MHz, DMSO-d₆) δ 9.16 (s, 1H), 8.95 (s, 1H), 8.91-8.85 (m, 2H), 8.79 (s, 1H), 8.40 (d, 1H), 8.11 (m, 1H), 7.09-7.01 (m, 2H), 6.89 (d, 1H), 4.60 (m, 3H), 1.96 (s, 3H), 1.30 (d, 3H) ppm.

### Example 93: 2-[4-methyl-3-[4-methyl-6-(1H-pyrazolo[3,4-c]pyridin-4-yl)-3-pyridyl]-2-oxo-benzimidazol-1-yl]-N-(2,2,2-trifluoroethyl)acetamide

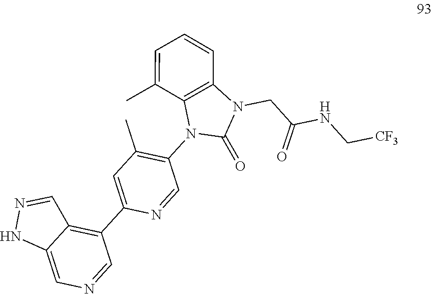 [SMILES: Cc1cc(-c2cncc3[nH]ncc23)ncc1-n1c(=O)n(CC(=O)NCC(F)(F)F)c2cccc(C)c21]

Example 93 was prepared by analogy with example 68 (Scheme 12), replacing 1-38 in step A with 1-43 and replacing (S)-2-Amino-1,1,1-trifluoropropane hydrochloride in step D with trifluoroethylamine.

LC-MS (ES⁺, Method 3): 2.85 min, m/z 496.20 [M+H]⁺. ¹H NMR (400 MHz, DMSO-d₆) δ 13.86 (s, 1H), 9.15 (s, 1H), 8.99-8.95 (m, 2H), 8.79 (d, J=10.2 Hz, 2H), 8.32 (s, 1H), 7.05 (d, J=4.5 Hz, 2H), 6.87 (m, 1H), 4.73-4.64 (m, 2H), 4.04-3.94 (m, 2H), 2.23 (s, 3H), 1.84 (s, 3H) ppm.

### Example 96: 2-[4-methyl-3-[3-methyl-5-(1-methylpyrazolo[3,4-c]pyridin-4-yl)pyrazin-2-yl]-2-oxo-benzimidazol-1-yl]-N-(2,2,2-trifluoroethyl)acetamide

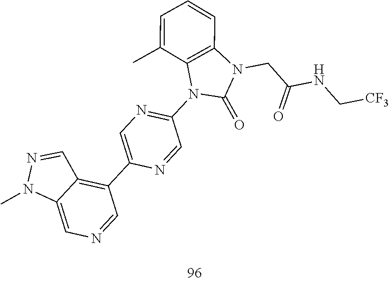 [SMILES: Cc1cccc2c1n(-c1cnc(-c3cncc4c3cnn4C)cn1)c(=O)n2CC(=O)NCC(F)(F)F]

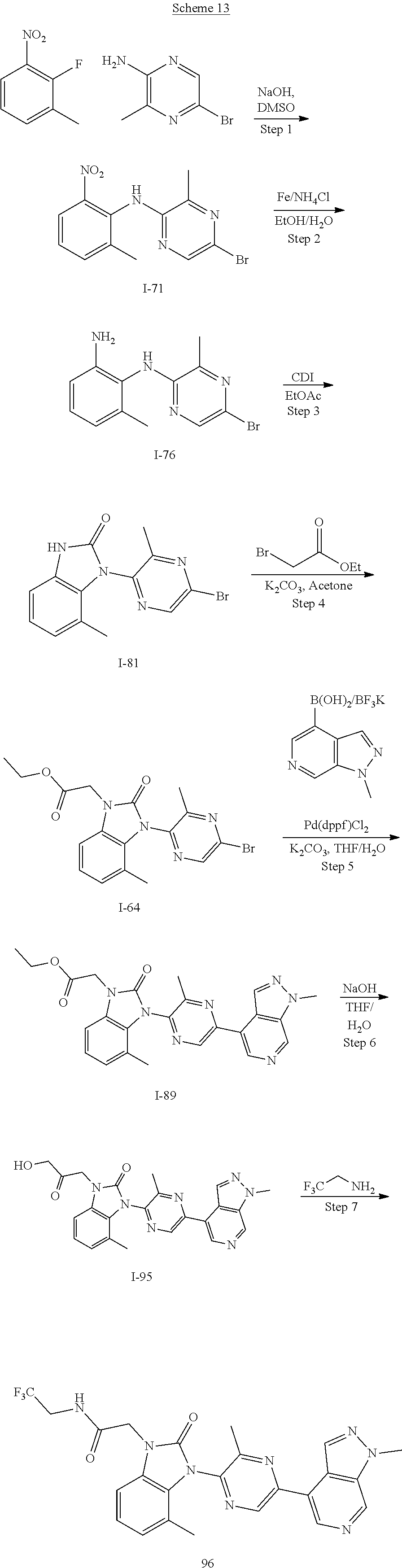

Step 1: Synthesis of 5-bromo-3-methyl-N-(2-methyl-6-nitro-phenyl)pyrazin-2-amine (I-71): NaOH (0.45 g, 3.0 eq.) was added to a solution of 5-bromo-3-methyl-pyrazin-2-amine (0.90 g, 3.72 mmol, 1.0 eq.) in DMSO (10 mL) and the resulting mixture was stirred at 25° C. under N₂ for 15 min. 2-fluoro-1-methyl-3-nitro-benzene (0.69 g, 1.2 eq.) was added and the resulting solution was kept stirring for 2 h. The mixture was diluted with water (20 mL) and extracted with EtOAc (×3). The combined organic layers were washed with brine, dried over sodium sulfate, concentrated under vacuum. The crude material was purified by flash column chromatography eluting with EtOAc in Pet. Ether to afford 1-71 (1.23 g, 78% yield). LC-MS (ES-API, Method 3): 3.88 min, m/z, 325.2 [M+2]*.

Step 2: Synthesis of N2-(5-bromo-3-methyl-pyrazin-2-yl)-3-methyl-benzene-1,2-diamine (I-76): Iron (622 mg, 6.0 eq.) was added to a mixture of 1-71 (600 mg, 1.85 mmol), NH₄Cl (6.0 eq.) in EtOH/H₂O (2:1, 6 mL) at 50° C. and the reaction was stirred for 2 h. The mixture was filtered and concentrated in vacuum. The crude material was purified by flash column chromatography eluting with EtOAc in Pet. Ether to afford 1-76 (418 mg, 77% yield). LC-MS (ES-API, Method 3): 3.05 min, m/z, 295.2 [M+2]⁺

Step 3: Synthesis of 3-(5-bromo-3-methyl-pyrazin-2-yl)-4-methyl-1H-benzimidazol-2-one (I-81): A mixture of 1-76 (500 mg, 1.7 mmol) and CDI (553 mg, 2 eq.) in EtOAc (5 mL, 10 V) was stirred at 55° C. under N2 for 2 h. The mixture was diluted with water and extracted with EtOAc. The combined organic layers were washed with water, brine, dried over Na₂SO₄ and concentrated in vacuum to give 1-81 (439 mg, 81% yield). LC-MS (ES-API, Method 3): 3.38 min, m/z, 321.2 [M+2]⁺

Step 4: Synthesis of ethyl 2-(3-(5-bromo-3-methylpyrazin-2-yl)-4-methyl-2-oxo-2,3-dihydro-1H-benzo[d]imidazol-1-yl)acetate (I-64): Ethyl bromoacetate (1.2 eq.) was added to a mixture of 1-81 (800 mg, 2.5 mmol) and K₂CO₃ (2.0 eq.) in acetone (10 mL). The reaction mixture was stirred for 12 h and then concentrated in vacuum. The crude material was purified by flash column chromatography eluting with EtOAc in Pet. Ether to give ethyl 1-64 (817 mg, 79% yield). LC-MS (ES-API, Method 3): 3.85 min, m/z, 405.10 [M]⁺

Step 5: Synthesis of ethyl 2-(4-methyl-3-(3-methyl-5-(1-methyl-1H-pyrazolo[3,4-c]pyridin-4-yl)pyrazin-2-yl)-2-oxo-2,3-dihydro-1H-benzo[d]imidazol-1-yl)acetate (I-89): A mixture of 1-81 (450 mg, 1.11 mmol, 1.0 eq.), potassium trifluoro(1-methyl-1H-pyrazolo[3,4-c]pyridin-4-yl)borate (560 mg, 48% assay, 1.0 eq.), K₂CO₃ (306 mg, 2.0 eq.) and Pd(dppf)Cl₂ (41 mg, 0.05 eq.) in THF/H₂O (10 mL, 3:1) was backfilled with N₂ three times and then warmed up to 60° C. for 12 h. The mixture was then cooled to RT and water was added. The layers were partitioned. The aqueous layer was extracted with EtOAc. The combined organic extracts were washed with water and brine and concentrated under reduced pressure. The crude material was purified by flash column chromatography eluting with EtOAc in Pet. Ether to give of ethyl 2-(4-methyl-3-(3-methyl-5-(1-methyl-1H-pyrazolo[3,4-c]pyridin-4-yl)pyrazin-2-yl)-2-oxo-2,3-dihydro-1H-benzo[d]imidazol-1-yl)acetate (425 mg, 83% yield). LC-MS (ES-API, Method 3): 2.98 min, m/z, 443.35 [M+H]⁺

Step 6: Synthesis of 2-(4-methyl-3-(3-methyl-5-(1-methyl-1H-pyrazolo[3,4-c]pyridin-4-yl)pyrazin-2-yl)-2-oxo-2,3-dihydro-1H-benzo[d]imidazol-1-yl)acetic acid (I-95): A solution of NaOH (78 mg, 2.0 eq.) in water (5 mL) was added to a solution of I-89 (450 mg, 0.98 mmol) in THF. The resulting mixture was stirred at 25° C. for 2 h. 3 M aq. HCl was added slowly to adjust pH to 3-4. The mixture was filtered and the solid was collected, dried under vacumm at 55° C. to afford 1-95 (382 mg, 80% yield). LC-MS (ES-API, Method 3): 2.88 min, m/z, 430.30 [M+H]⁺

Step 7: Synthesis of 2-(4-methyl-3-(3-methyl-5-(1-methyl-1H-pyrazolo[3,4-c]pyridin-4-yl)pyrazin-2-yl)-2-oxo-2,3-dihydro-1H-benzo[d]imidazol-1-yl)-N-(2,2,2-trifluoroethyl)acetamide (Example 96): 1-95 (100 mg, 0.23 mmol), DIEA (181 mg, 1.4 mmol), trifluoroethylamine (0.03 mL, 0.35 mmol) and HATU (106 mg, 0.28 mmol) in DMF (3 mL) was stirred at 25° C. for 2 h. The mixture was diluted with water (20 mL) and extracted with EoOAc. The combined organic phases were washed with brine, dried over sodium sulfate, concentrated under vacuum. Further purification by flash column chromatography eluting with MeOH in DCM gave example 96 (65 mg, 55% yield). LC-MS (ES-API, Method 3): 2.99 min, m/z, 511.35 [M+H]⁺. 1H NMR (400 MHz, DMSO-d₆) δ 9.43 (s, 2H), 9.16 (s, 1H), 9.01 (m, 1H), 8.82 (s, 1H), 7.08 (d, J=8.3 Hz, 2H), 6.90 (d, J=7.1 Hz, 1H), 4.69 (d, J=4.6 Hz, 2H), 4.28 (s, 3H), 4.03-3.96 (m, 2H), 2.64 (s, 3H), 1.81 (s, 3H) ppm.

### Example 99: 2-[4-methyl-3-[5-methyl-6-(1H-pyrazolo[3,4-c]pyridin-4-yl)-3-pyridyl]-2-oxo-benzimidazol-1-yl]-N-(2,2,2-trifluoroethyl)acetamide

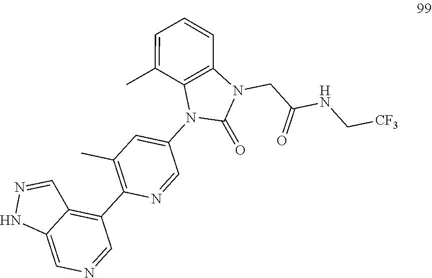 [SMILES: Cc1cc(-n2c(=O)n(CC(=O)NCC(F)(F)F)c3cccc(C)c32)cnc1-c1cncc2[nH]ncc12]

Example 99 was prepared by analogy with example 68 (Scheme 12), replacing 1-38 in step A with 1-65 and replacing (S)-2-Amino-1,1,1-trifluoropropane hydrochloride in step D with trifluoroethylamine. LC-MS (ES⁺, Method 3): 2.78 min, m/z 496.30 [M+H]⁺. ¹H NMR (400 MHz, DMSO-d₆) δ 9.62 (s, 1H), 8.99 (m, 1H), 8.75 (d, J=2.3 Hz, 1H), 8.67 (s, 1H), 8.50 (s, 1H), 8.08 (d, J=2.3 Hz, 1H), 7.12-7.01 (m, 2H), 6.94-6.86 (m, 1H), 4.68 (s, 2H), 4.04-3.94 (m, 2H), 3.57 (s, 3H), 2.46 (s, 3H), 1.97 (s, 3H) ppm.

### Example 100: 2-[4-methyl-3-[6-methyl-5-(1-methylpyrazolo[3,4-c]pyridin-4-yl)pyrazin-2-yl]-2-oxo-benzimidazol-1-yl]-N-(2,2,2-trifluoroethyl)acetamide

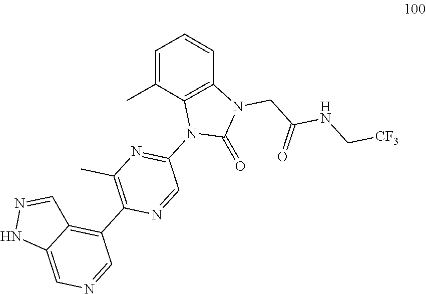 [SMILES: Cc1nc(-n2c(=O)n(CC(=O)NCC(F)(F)F)c3cccc(C)c32)cnc1-c1cncc2[nH]ncc12]

Example 100 was prepared by analogy with example 68 (Scheme 12), replacing 1-38 in step A with I-66 and replacing (S)-2-Amino-1,1,1-trifluoropropane hydrochloride in step D with trifluoroethylamine.

LC-MS (ES⁺, Method 4): 1.53 min, m/z 511.15 [M+H]⁺. ¹H NMR (400 MHz, DMSO-d₆) δ 9.35 (s, 1H), 9.01 (t, J=6.3 Hz, 1H), 8.96 (s, 1H), 8.57 (s, 1H), 8.21 (s, 1H), 7.12 (m, 1H), 7.05 (d, J=7.7 Hz, 1H), 6.96 (d, J=7.5 Hz, 1H), 4.68 (s, 2H), 4.27 (s, 3H), 4.03-3.94 (m, 2H), 2.62 (s, 3H), 2.01 (s, 3H) ppm.

### Example 69: 2-[4-methyl-2-oxo-3-[5-(1H-pyrazolo[3,4-c]pyridin-4-yl)-2-pyridyl]benzimidazol-1-yl]-N-[(1S)-2,2,2-trifluoro-1-methyl-ethyl]acetamide

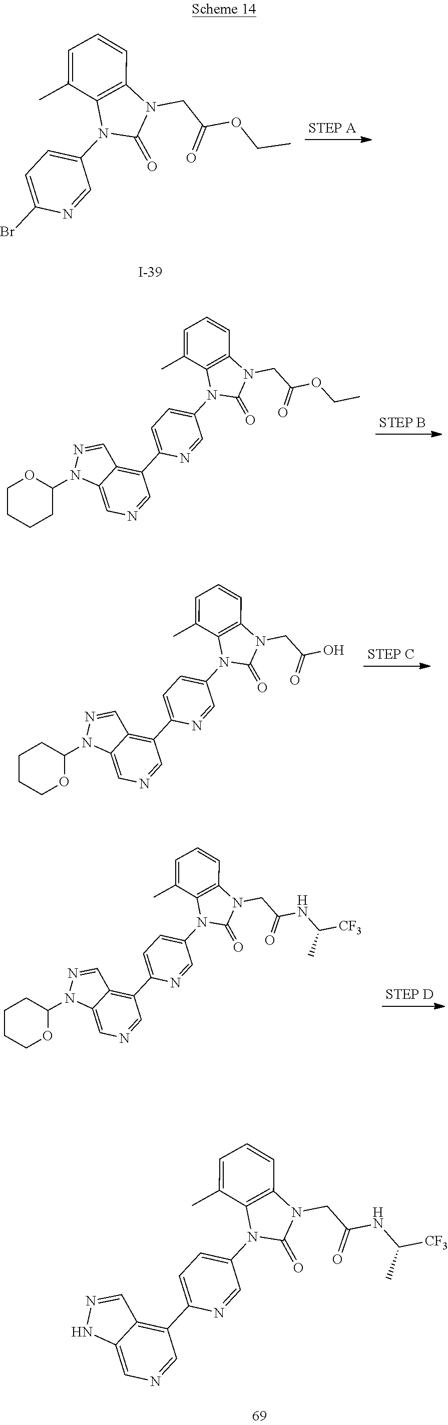

Steps A to C are analogous to steps A to C described for example 47 in Scheme 10. In step A, tributyl-(1-methylpyrazolo[3,4-c]pyridin-4-yl)stannane was replaced with tributyl-(1-tetrahydropyran-2-ylpyrazolo[3,4-c]pyridin-4-yl)stannane. In step C, trifluoroethylamine was replaced by (2S)-amino-1,1,1-trifluoropropane.

STEP D: 2-[4-methyl-2-oxo-3-[5-(1H-pyrazolo[3,4-c]pyridin-4-yl)-2-pyridyl]benzimidazol-1-yl]-N-[(1S)-2,2,2-trifluoro-1-methyl-ethyl]acetamide. A solution of 2-[4-methyl-2-oxo-3-[5-(1-tetrahydropyran-2-ylpyrazolo[3,4-c]pyridin-4-yl)-2-pyridyl]benzimidazol-1-yl]-N-[(1S)-2,2,2-trifluoro-1-methyl-ethyl]acetamide (50 mg, 0.09 mmol) in methanol (2 mL) and 4 M HCl in 1,4-dioxane (1 mL) was stirred at 25° C. for 2 h. The reaction mixture was concentrated and was purified by preparative TLC (DCM/MeOH, 30:1) to give 2-[4-methyl-2-oxo-3-[5-(1H-pyrazolo[3,4-c]pyridin-4-yl)-2-pyridyl]benzimidazol-1-yl]-N-[(1S)-2,2,2-trifluoro-1-methyl-ethyl]acetamide (19.7 mg, 47%) as a yellow solid. LC-MS (ES⁺, Method 4):1.25 min, m/z 496.15 [M+H]⁺. ¹H NMR (400 MHz, DMSO-d₆) δ 13.92 (s, 1H), 9.15 (s, 1H), 9.09 (s, 1H), 8.95 (d, J=8.8 Hz, 1H), 8.54 (d, J=18.3 Hz, 3H), 7.82 (d, J=8.2 Hz, 1H), 7.07 (m, 1H), 7.00 (d, J=7.8 Hz, 1H), 6.90 (d, J=7.7 Hz, 1H), 4.64 (s, 3H), 1.93 (s, 3H), 1.30 (d, J=7.0 Hz, 3H) ppm.

### Example 70: 4-methyl-1-[2-oxo-2-[(2S)-2-(trifluoromethyl)morpholin-4-yl]ethyl]-3-[5-(1H-pyrazolo[3,4-c]pyridin-4-yl)-2-pyridyl]benzimidazol-2-one

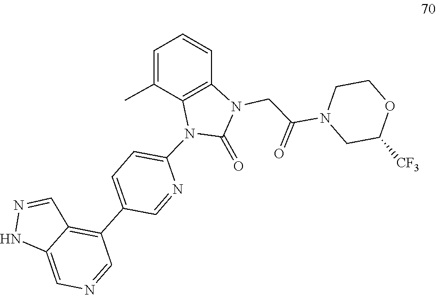 [SMILES: Cc1cccc2c1n(-c1ccc(-c3cncc4[nH]ncc34)cn1)c(=O)n2CC(=O)N1CCO[C@H](C(F)(F)F)C1]

Example 70 was prepared by analogy with example 69 (scheme 13). (2S)-amino-1,1,1-trifluoropropane was replaced by (2S)-2-(trifluoromethyl)morpholine in Step C. LC-MS (ES⁺, Method 3):3.02 min, m/z 538.30 [M+H]⁺. ¹H NMR (400 MHz, DMSO-d₆) δ δ 9.49 (s, 1H), 9.15 (d, J=2.4 Hz, 1H), 8.76 (d, J=29.7 Hz, 2H), 8.59 (dd, J=8.3, 2.4 Hz, 1H), 7.85 (d, J=8.2 Hz, 1H), 7.08 (d, J=8.3 Hz, 2H), 6.92-6.88 (m, 1H), 5.07 (q, J=27.8, 17.3 Hz, 1H), 4.91-4.82 (m, 1H), 4.43 (s, 1H), 4.28 (s, 1H), 4.14 (d, J=13.2 Hz, 1H), 4.06 (d, J=13.3 Hz, 1H), 4.00 (s, 1H), 3.39 (s, 1H), 2.92 (d, J=13.6 Hz, 1H), 1.95 (s, 3H) ppm.

### Example 71: 4-methyl-1-[2-oxo-2-[(2R)-2-(trifluoromethyl)morpholin-4-yl]ethyl]-3-[5-(1H-pyrazolo[3,4-c]pyridin-4-yl)-2-pyridyl]benzimidazol-2-one

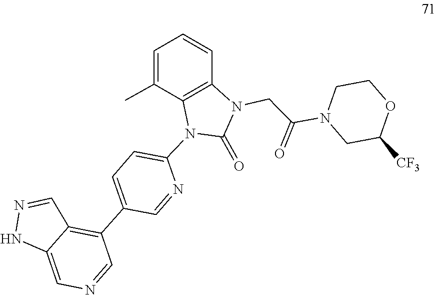 [SMILES: Cc1cccc2c1n(-c1ccc(-c3cncc4[nH]ncc34)cn1)c(=O)n2CC(=O)N1CCO[C@@H](C(F)(F)F)C1]

Example 71 was prepared by analogy with example 69 (scheme 13). (2S)-amino-1,1,1-trifluoropropane was replaced by (2R)-2-(trifluoromethyl)morpholine in Step C. LC-MS (ES⁺, Method 3):3.25 min, m/z 538.30 [M+H]⁺. ¹H NMR (400 MHz, DMSO-d₆) δ 13.96 (s, 1H), 9.15 (s, 1H), 9.10 (d, J=2.5 Hz, 1H), 8.57 (s, 1H), 8.54-8.51 (m, 2H), 7.80 (d, J=8.2 Hz, 1H), 7.08-7.06 (m, 2H), 6.90-6.88 (m, 1H), 5.12-5.01 (m, 1H), 4.90-4.83 (m, 1H), 4.43-4.16 (m, 2H), 4.13-3.97 (m, 2H), 3.79-3.55 (m, 1H), 3.45-3.35 (m, 1H), 2.96-2.86 (m, 1H), 1.93 (s, 3H) ppm.

### Example 72: 2-[4-methyl-2-oxo-3-[5-(1H-pyrazolo[3,4-c]pyridin-4-yl)-2-pyridyl]benzimidazol-1-yl]-N-(2,2,2-trifluoroethyl)acetamide

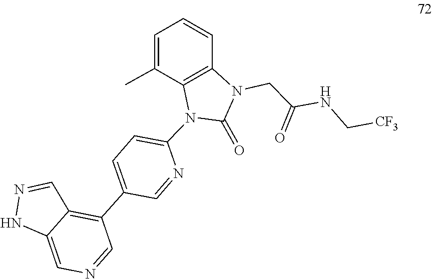 [SMILES: Cc1cccc2c1n(-c1ccc(-c3cncc4[nH]ncc34)cn1)c(=O)n2CC(=O)NCC(F)(F)F]

Example 72 was prepared by analogy with example 69 (scheme 13). (2S)-amino-1,1,1-trifluoropropane was replaced by trifluoroethylamine in Step C. LC-MS (ES⁺, Method 3):3.05 min, m/z 482.25 [M+H]⁺. ¹H NMR (400 MHz, DMSO-d₆) δ 9.15 (s, 1H), 9.09 (d, J=2.4 Hz, 1H), 9.05 (m, 1H), 8.56 (s, 1H), 8.53 (m, J=8.6 Hz, 2H), 7.82 (d, J=8.2 Hz, 1H), 7.07 (t, J=7.7 Hz, 1H), 7.01 (d, J=7.7 Hz, 1H), 6.90 (d, J=7.4 Hz, 1H), 4.67 (s, 2H), 3.98 (m, J=7.2 Hz, 2H), 1.94 (s, 3H) ppm.

### Example 73: 2-[4-methyl-2-oxo-3-[5-(1H-pyrazolo[3,4-c]pyridin-4-yl)pyrimidin-2-yl]benzimidazol-1-yl]-N-[(1S)-2,2,2-trifluoro-1-methyl-ethyl]acetamide

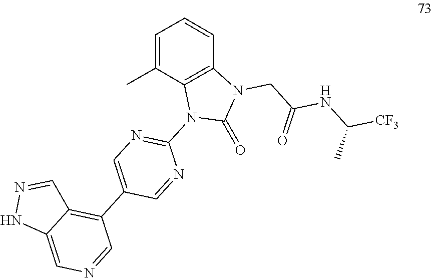 [SMILES: Cc1cccc2c1n(-c1ncc(-c3cncc4[nH]ncc34)cn1)c(=O)n2CC(=O)N[C@@H](C)C(F)(F)F]

Example 73 was prepared by analogy with example 69 (scheme 13). Intermediate I-39 in step A was replaced with ethyl 2-[3-(5-bromopyrimidin-2-yl)-4-methyl-2-oxo-benzimidazol-1-yl]acetate (intermediate 1-45). LC-MS (ES⁺, Method 3):2.95 min, m/z 497.20 [M+H]⁺. ¹H NMR (400 MHz, DMSO-d6) δ 9.52 (s, 2H), 9.20 (s, 1H), 8.62 (d, J=12.0 Hz, 2H), 7.09 (m, 1H), 7.02 (d, J=12.0 Hz, 1H), 6.91 (d, J=8.0 Hz, 1H), 4.63 (m, 3H), 1.88 (s, 3H), 1.30 (d, J=8.0 Hz, 3H) ppm.

### Example 94: 2-[4-methyl-3-[2-methyl-6-(1H-pyrazolo[3,4-c]pyridin-4-yl)-3-pyridyl]-2-oxo-benzimidazol-1-yl]-N-(2,2,2-trifluoroethyl)acetamide

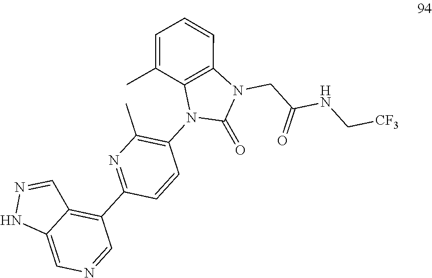 [SMILES: Cc1nc(-c2cncc3[nH]ncc23)ccc1-n1c(=O)n(CC(=O)NCC(F)(F)F)c2cccc(C)c21]

Example 94 was prepared by analogy with example 69 (scheme 13). Intermediate I-39 in step A was replaced with ethyl 2-[3-(5-bromopyrimidin-2-yl)-4-methyl-2-oxo-benzimidazol-1-yl]acetate (intermediate 1-63) and (S)-2-amino-1,1,1-trifluoropropane was replaced by trifluoroethylamine in step C. LC-MS (ES⁺, Method 4): 1.24 min, m/z 496.20 [M+H]⁺. ¹H NMR (400 MHz, DMSO-d₆) δ 9.67 (s, 1H), 9.25 (s, 1H), 9.10-9.02 (m, 2H), 8.40 (d, J=8.0 Hz, 1H), 8.13 (d, J=8.0 Hz, 1H), 7.06 (d, J=4.0 Hz, 2H), 6.87 (m, 1H), 4.69 (s, 2H), 4.03-3.93 (m, 2H), 2.48 (s, 3H), 1.82 (s, 3H) ppm.

### Example 98: 2-[4-methyl-2-oxo-3-[5-(1H-pyrazolo[3,4-c]pyridin-4-yl)pyrimidin-2-yl]benzimidazol-1-yl]-N-[(1R)-2,2,2-trifluoro-1-methyl-ethyl]acetamide

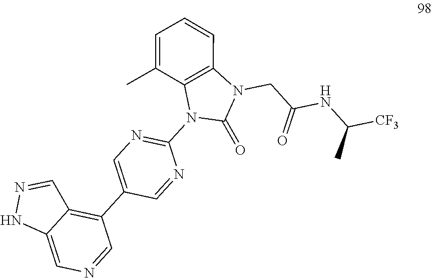 [SMILES: Cc1cccc2c1n(-c1ncc(-c3cncc4[nH]ncc34)cn1)c(=O)n2CC(=O)N[C@H](C)C(F)(F)F]

Example 98 was prepared by analogy with example 69 (scheme 13). Intermediate I-39 in step A was replaced with ethyl 2-[3-(5-bromopyrimidin-2-yl)-4-methyl-2-oxo-benzimidazol-1-yl]acetate (intermediate 1-45) and (S)-2-amino-1,1,1-trifluoropropane was replaced by (R)-2-amino-1,1,1-trifluoropropane in step C. LC-MS (ES⁺, Method 3):2.95 min, m/z 497.20 [M+H]⁺. ¹H NMR (400 MHz, DMSO-d₆) δ 9.52 (s, 2H), 9.20 (s, 1H), 8.62 (d, J=12.0 Hz, 2H), 7.09 (m, 1H), 7.02 (d, J=12.0 Hz, 1H), 6.91 (d, J=8.0 Hz, 1H), 4.63 (m, 3H), 1.88 (s, 3H), 1.30 (d, J=8.0 Hz, 3H) ppm.

### Example 75: 2-[4-methyl-3-[2-(1-methylpyrazolo[3,4-c]pyridin-4-yl)pyrimidin-5-yl]-2-oxo-benzimidazol-1-yl]-N-(2,2,2-trifluoroethyl)acetamide

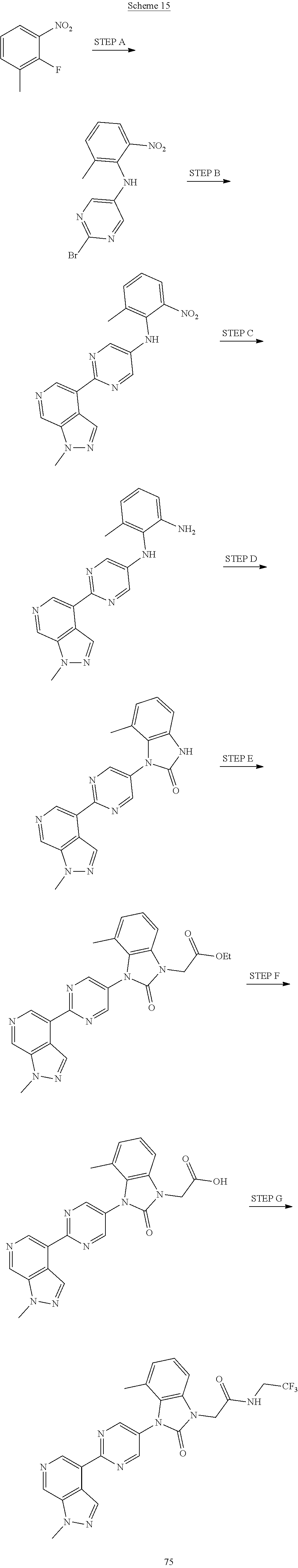

STEP A: 2-bromo-N-(2-methyl-6-nitro-phenyl)pyrimidin-5-amine. A solution of NaH (1.53 g, 38.13 mmol) in DMF (20 mL) was stirred at 0° C. under N₂ for 5 min before adding 2-bromopyrimidin-5-amine (2.95 g, 19.07 mmol) in DMF (10 mL) slowly and stirring for 0.5 h. 2-Fluoro-1-methyl-3-nitrobenzene (3.30 g, 19.07 mmol) was added slowly and the mixture stirred for 16 h. The mixture was diluted with water (50 mL) and extracted with EtOAc (200 mL×3). The combined organic layers were washed with brine (200 mL), dried over Na₂SO₄ and concentrated in vacuum to give 2-bromo-N-(2-methyl-6-nitro-phenyl)pyrimidin-5-amine (1.8 g, 25%). LC-MS (ES⁺, Method 3): 3.86 min, m/z 309/311 [M+H]⁺. ¹H NMR (400 MHz, DMSO-d₆) 8.51 (s, 1H), 7.96 (s, 2H), 7.85 (m, 1H), 7.68 (m, 1H), 7.40 (m, 1H), 2.21 (s, 3H) ppm.

STEP B: N-(2-methyl-6-nitro-phenyl)-2-(1-methylpyrazolo[3,4-c]pyridin-4-yl)pyrimidin-5-amine. A solution of 2-bromo-N-(2-methyl-6-nitro-phenyl)pyrimidin-5-amine (100 mg, 0.32 mmol), tributyl-(1-methylpyrazolo[3,4-c]pyridin-4-yl)stannane (164 mg, 0.38 mmol), Pd(PPh₃)₄ (37 mg, 0.032 mmol), LiCl (41 mg, 0.95 mmol), and CuI (34 mg, 0.16 mmol), in toluene (1.0 mL) was stirred at 120° C. under N₂ overnight. The mixture was diluted with EtOAc (30 mL) and washed with H₂O (15 mL×3). The organic layers were washed with EtOAc (5 mL×3), combined, dried over anhydrous Na₂SO₄ and concentrated to give of yellow solid purified by preparative TLC to give N-(2-methyl-6-nitro-phenyl)-2-(1-methylpyrazolo[3,4-c]pyridin-4-yl)pyrimidin-5-amine (30 mg, 26%) as a yellow solid. LC-MS (ES⁺, Method 3): 3.25 min, m/z, 362.0 [M+H]⁺. ¹H NMR (400 MHz, DMSO-d6) 9.24-9.16 (m, 2H), 8.76 (s, 1H), 8.62 (s, 1H), 8.29 (s, 2H), 7.81 (d, 1H), 7.75 (d, 1H), 7.46 (m, 1H), 4.24 (s, 3H), 2.27 (s, 3H) ppm.

STEP C: 3-methyl-N2-[2-(1-methylpyrazolo[3,4-c]pyridin-4-yl)pyrimidin-5-yl]benzene-1,2-diamine. A solution of N-(2-methyl-6-nitro-phenyl)-2-(1-methylpyrazolo[3,4-c]pyridin-4-yl)pyrimidin-5-amine (20 mg, 0.06 mmol), and palladium (2.94 mg, 0.03 mmol) in IPA (2 mL) was stirred at 50° C. for 1 hr. The mixture was diluted with water and EtOAc and phases separated, combined organic layers were washed with brine (20 mL), extracted with Ethyl acetate (30 mL) and dried over Na₂SO₄ and concentrated in vacuum to give the crude material which was purified by preparative TLC (Pet.ether/EtOAc, 1/1) affording 3-methyl-N2-[2-(1-methylpyrazolo[3,4-c]pyridin-4-yl)pyrimidin-5-yl]benzene-1,2-diamine (10 mg, 54%) as a brown oil. ¹H NMR (400 MHz, DMSO-d₆) 9.11 (s, 1H), 9.03 (s, 1H), 8.76 (s, 1H), 8.62 (s, 1H), 8.29 (s, 2H), 7.90-7.33 (m, 4H), 4.24 (s, 3H), 2.24 (s, 3H) ppm.

STEP D: 4-methyl-3-[2-(1-methylpyrazolo[3,4-c]pyridin-4-yl)pyrimidin-5-yl]-1H-benzimidazol-2-one. A solution of -methyl-N2-[2-(1-methylpyrazolo[3,4-c]pyridin-4-yl)pyrimidin-5-yl]benzene-1,2-diamine (35 mg, 0.11 mmol), CDI (85.7 mg, 0.52 mmol), in DMF (0.3 mL) was stirred at 100° C. under N₂ overnight. The mixture was diluted with EtOAc (3 mL) and washed with H₂O (5 mL×3). The organic layers were washed with EtOAc (5 mL×3), combined, dried over anhydrous Na₂SO₄ and concentrated to give a yellow solid purified by preparative TLC to afford 4-methyl-3-[2-(1-methylpyrazolo[3,4-c]pyridin-4-yl)pyrimidin-5-yl]-1H-benzimidazol-2-one (30 mg, 79%). LC-MS (ES⁺, Method 3): 3.117 min, m/z, 358.0 [M+H]⁺

STEP E: ethyl 2-[4-methyl-3-[2-(1-methylpyrazolo[3,4-c]pyridin-4-yl)pyrimidin-5-yl]-2-oxo-benzimidazol-1-yl]acetate. A solution of 4-methyl-3-[2-(1-methylpyrazolo[3,4-c]pyridin-4-yl)pyrimidin-5-yl]-1H-benzimidazol-2-one (30 mg, 0.08 mmol), ethyl bromoacetate (21.05 mg, 0.12 mmol), and Cs₂CO₃ (54.75 mg, 0.16 mmol), in MeCN (5 mL) was stirred at 50° C. for 2 h. The mixture was quenched with H₂O (3 mL) and the mixture was extracted with EtOAc (8 mL×3). The organic layers were combined, washed with H₂O (8 mL×3), then NaCl (aq.) (5 mL×3), and the organic phase was dried over anhydrous Na₂SO₄ before concentrating to afford ethyl 2-[4-methyl-3-[2-(1-methylpyrazolo[3,4-c]pyridin-4-yl)pyrimidin-5-yl]-2-oxo-benzimidazol-1-yl]acetate (20 mg, 54%) as a yellow solid. LC-MS (ES⁺, Method 3): 3.583 min, m/z, 444.25 [M+H]⁺

STEP F: 2-[4-methyl-3-[2-(1-methylpyrazolo[3,4-c]pyridin-4-yl)pyrimidin-5-yl]-2-oxo-benzimidazol-1-yl]acetic acid. A mixture ethyl 2-[4-methyl-3-[2-(1-methylpyrazolo[3,4-c]pyridin-4-yl)pyrimidin-5-yl]-2-oxo-benzimidazol-1-yl]acetate (30 mg, 0.06 mmol), and LiOH·H₂O (8.53 mg, 0.20 mmol) in THE (1.5 mL), MeOH (1.5 mL) and H₂O (1 mL) was stirred at 25° C. for 2 h. The mixture was diuted with H₂O (5 mL) and 10% HCl (3 mL) to reach pH 2-3. The mixture was diluted with water (10 mL×3) and extracted with DCM (10 mL×3). After drying over Na₂SO₄, filtered and concentrated in vacuo to obtain 2-[4-methyl-3-[2-(1-methylpyrazolo[3,4-c]pyridin-4-yl)pyrimidin-5-yl]-2-oxo-benzimidazol-1-yl]acetic acid (17 mg, 61%) as a white solid. LC-MS (ES⁺, Method 3): 2.817 min, m/z, 416.0 [M+H]⁺

STEP G: 2-[4-methyl-3-[2-(1-methylpyrazolo[3,4-c]pyridin-4-yl)pyrimidin-5-yl]-2-oxo-benzimidazol-1-yl]-N-(2,2,2-trifluoroethyl)acetamide (example 75). A solution of 2-[4-methyl-3-[2-(1-methylpyrazolo[3,4-c]pyridin-4-yl)pyrimidin-5-yl]-2-oxo-benzimidazol-1-yl]acetic acid (17 mg, 0.041 mmol), trifluoroethylamine (6.08 mg, 0.061 mmol), HATU (23.34 mg, 0.061 mmol), and DIEA (26.44 mg, 0.20 mmol), in DMF (0.5 mL) was stirred at 25° C. overnight. The mixture was diluted with EtOAc (5 mL) and washed with H₂O (2 mL×3). The organic layers were washed with EtOAc (5 mL×3), combined, dried over anhydrous Na₂SO₄ and concentrated to give a yellow solid purified by preparative TLC to afford 2-[4-methyl-3-[2-(1-methylpyrazolo[3,4-c]pyridin-4-yl)pyrimidin-5-yl]-2-oxo-benzimidazol-1-yl]-N-(2,2,2-trifluoroethyl)acetamide (example 75) (2.0 mg, 10%) as a white solid. LC-MS (ES⁺, Method 3): 3.42 min, m/z, 497.20 [M+H]⁺. ¹H NMR (400 MHz, DMSO-d₆) δ 9.39 (s, 1H), 9.33 (s, 1H), 9.21 (s, 2H), 9.06 (m, 1H), 8.86 (s, 1H), 7.11-7.05 (m, 2H), 6.93 (d, J=7.2 Hz, 1H), 4.69 (s, 2H), 4.28 (s, 3H), 4.04-3.96 (m, 2H), 2.01 (s, 3H) ppm.

### Example 91: 2-[3-[6-(1-methylpyrazolo[3,4-c]pyridin-4-yl)-3-pyridyl]-2-oxo-benzimidazol-1-yl]-N-(2,2,2-trifluoroethyl)acetamide

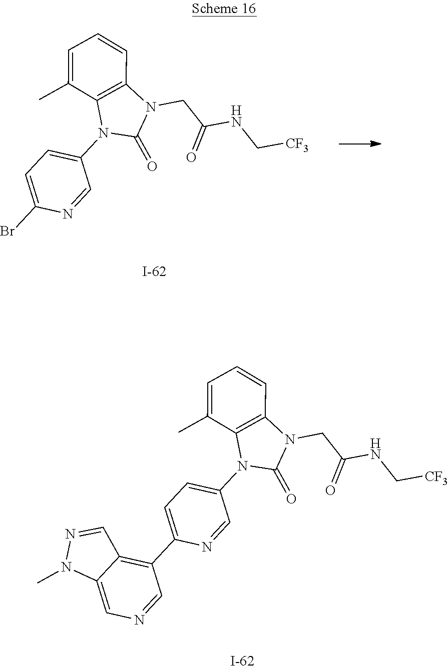

Example 91 was prepared from intermediate 1-62 using a Stille coupling with 1-methyl-4-(tributylstannyl)-1H-pyrazolo[3,4-c]pyridine under the conditions described in Step C of Scheme 11. LC-MS (ES⁺, Method 4): 1.47 min, m/z, 482.20 [M+H]⁺. ¹H NMR (400 MHz, DMSO-d₆) δ 9.28 (s, 1H), 9.05 (s, 1H), 9.03-8.96 (m, 2H), 8.75 (s, 1H), 8.42 (d, 1H), 8.22 (d, 1H), 7.30-7.12 (m, 4H), 4.71 (s, 2H), 4.26 (s, 3H), 2.24 (m, 2H) ppm.

### Example 92: 2-[3-[6-(1,3-dimethylpyrazolo[3,4-c]pyridin-4-yl)-3-pyridyl]-4-methyl-2-oxo-benzimidazol-1-yl]-N-(2,2,2-trifluoroethyl)acetamide

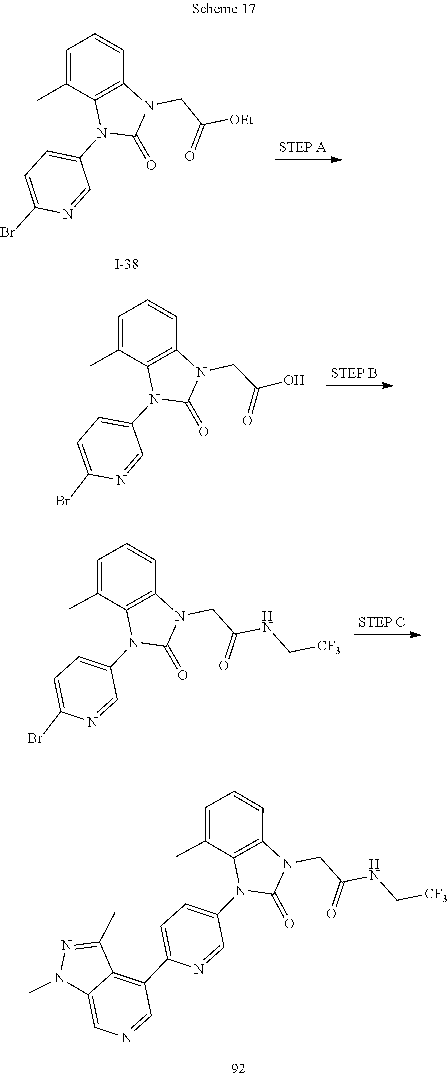

STEP A: 2-[3-(6-bromo-3-pyridyl)-4-methyl-2-oxo-benzimidazol-1-yl]acetic acid. A mixture of ethyl 2-(3-(6-bromopyridin-3-yl)-4-methyl-2-oxo-2,3-dihydro-1H-benzo[d]imidazol-1-yl)acetate (I-38) (500 mg, 1.29 mmol) and LiOH·H₂O (162 mg, 3.86 mmol) in H₂O/THF (5 mL/5 mL) was stirred at room temperature for 2 h. The mixture was concentrated under vacuum and the pH adjusted to 3 by slow addition of 1 M HCl. The mixture was diluted with water (50 mL) and was washed with EtOAc (100 mL×3). The combined organic layers were washed with brine (50 mL), dried over Na₂SO₄ and was concentrated to give 2-[3-(6-bromo-3-pyridyl)-4-methyl-2-oxo-benzimidazol-1-yl]acetic acid (380 mg, 82%) as a white solid. LC-MS (ES⁺, Method 4): 1.66 min, m/z, 361.9/363.9 [M+H]⁺. ¹H NMR (400 MHz, DMSO-d₆) δ 13.19 (bs, 1H), 8.56 (s, 1H), 7.95 (d, 1H), 7.84 (d, 1H), 7.12-7.06 (m, 2H0, 6.88 (d, 1H), 4.67 (s, 2H), 1.87 (s, 3H) ppm.

STEP B: 2-[3-(6-bromo-3-pyridyl)-4-methyl-2-oxo-benzimidazol-1-yl]-N-(2,2,2-trifluoroethyl)acetamide. A mixture of 2-(3-(6-bromopyridin-3-yl)-4-methyl-2-oxo-2,3-dihydro-1H-benzo[d]imidazol-1-yl)acetic acid (380 mg, 1.05 mmol), 2,2,2-trifluoroethan-1-amine (156 mg, 1.58 mmol), HATU (600.7 mg, 1.58 mmol) and DIPEA (408.4 mg, 3.16 mmol) in DMF (5 mL) was stirred at room temperature for 2 h. The mixture was treated with water (50 mL) and was filtered to give 2-[3-(6-bromo-3-pyridyl)-4-methyl-2-oxo-benzimidazol-1-yl]-N-(2,2,2-trifluoroethyl)acetamide (400 mg, 86%) as a white solid. LC-MS (ES⁺, Method 4): 1.38 min, m/z 442.7/444.6 [M+H]⁺. ¹H NMR (400 MHz, DMSO-d₆) δ 8.98 (m, 1H0, 8.55 (S, 1H), 7.94-7.85 (m, 2H), 7.07-7.01 (m, 2H), 6.88 (d, 1H), 4.63 (s, 2H), 2.39 (m, 2H), 1.87 (s, 3H) ppm.

STEP C: 2-[3-[6-(1,3-dimethylpyrazolo[3,4-c]pyridin-4-yl)-3-pyridyl]-4-methyl-2-oxo-benzimidazol-1-yl]-N-(2,2,2-trifluoroethyl)acetamide. A mixture of 2-(3-(6-bromopyridin-3-yl)-4-methyl-2-oxo-2,3-dihydro-1H-benzo[d]imidazol-1-yl)-N-(2,2,2-trifluoroethyl)acetamide (100 mg, 0.23 mmol), 5-bromo-3-methyl-1-tosyl-1H-pyrazolo[4,3-b]pyridine 1-methyl-4-(tributylstannyl)-1H-pyrazolo[3,4-c]pyridine (115 mg, 0.27 mmol), Pd(PPh₃)₄ (26 mg, 0.023 mmol), CuI (24 mg, 0.11 mmol), LiCl (29 mg, 0.68 mmol) in toluene (3 mL) was stirred at 110° C. under N₂ overnight. The mixture was filtered and was concentrated. The crude was purified by preparative TLC (DCM/MeOH, 20:1) to give 2-[3-[6-(1,3-dimethylpyrazolo[3,4-c]pyridin-4-yl)-3-pyridyl]-4-methyl-2-oxo-benzimidazol-1-yl]-N-(2,2,2-trifluoroethyl)acetamide (example 92) (20.4 mg, 15%) as an off-white solid. LC-MS (ES⁺, Method 3): 3.28 min, m/z 496.10 [M+H]⁺. ¹H NMR (400 MHz, DMSO-d₆) δ 9.29 (s, 1H), 8.98-8.94 (m, 2H), 8.90 (d, J=2.4 Hz, 1H), 8.74 (s, 1H), 8.40 (d, J=8.5 Hz, 1H), 8.10 (dd, J=8.4, 2.5 Hz, 1H), 7.08-7.02 (m, 2H), 6.89 (d, J=7.1 Hz, 1H), 4.67 (s, 2H), 4.26 (s, 3H), 4.03-3.94 (m, 2H), 1.94 (s, 3H) ppm.

### Example 102: 2-[4-methyl-3-[5-methyl-6-(3-methyl-1H-pyrazolo[3,4-c]pyridin-4-yl)-3-pyridyl]-2-oxo-benzimidazol-1-yl]-N-(2,2,2-trifluoroethyl)acetamide

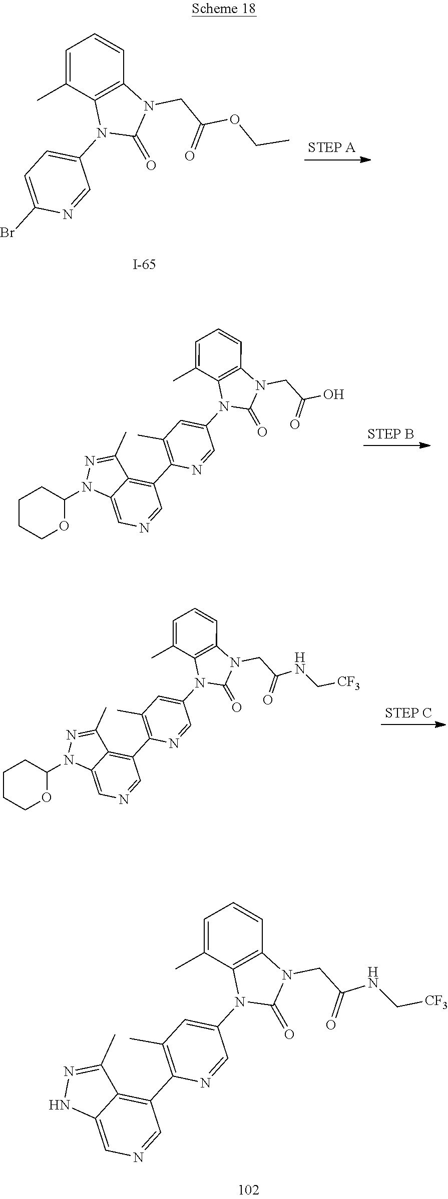

STEP A: 2-[4-methyl-3-[5-methyl-6-(3-methyl-1-tetrahydropyran-2-yl-pyrazolo[3,4-c]pyridin-4-yl)-3-pyridyl]-2-oxo-benzimidazol-1-yl]acetic acid. A solution of ethyl 2-[3-(6-bromo-5-methyl-3-pyridyl)-4-methyl-2-oxo-benzimidazol-1-yl]acetate (I-65) (150 mg, 0.37 mmol), tributyl-(3-methyl-1-tetrahydropyran-2-yl-pyrazolo[3,4-c]pyridin-4-yl)stannane (375 mg, 0.74 mmol), LiCl (47 mg, 1.1 mmol), CuI (39 mg, 0.19 mmol) and Pd(PPh₃)₄ (86 mg, 0.07 mmol) in xylene (5 mL) was stirred at 130° C. overnight. The mixture was cooled to room temperature, filtered through celite and concentrated in vacuo affording crude 2-[4-methyl-3-[5-methyl-6-(3-methyl-1-tetrahydropyran-2-yl-pyrazolo[3,4-c]pyridin-4-yl)-3-pyridyl]-2-oxo-benzimidazol-1-yl]acetic acid as a yellow oil (180 mg), used without further purification. LC-MS (ES⁺, Method 3): 3.10 min, m/z 513.3 [M+H]⁺.

STEP B: 2-[4-methyl-3-[5-methyl-6-(3-methyl-1-tetrahydropyran-2-yl-pyrazolo[3,4-c]pyridin-4-yl)-3-pyridyl]-2-oxo-benzimidazol-1-yl]-N-(2,2,2-trifluoroethyl)acetamide. A solution of 2-[4-methyl-3-[5-methyl-6-(3-methyl-1-tetrahydropyran-2-yl-pyrazolo[3,4-c]pyridin-4-yl)-3-pyridyl]-2-oxo-benzimidazol-1-yl]acetic acid (15 mg, 0.03 mmol), DIEA (11 mg, 0.09 mmol) and HATU (17 mg, 0.04 mmol) in DMF (0.5 mL) was stirred at 25° C. for 3 h. The mixture was partitioned between water and EtOAc, phases were separated and the organics dried over Na₂SO₄, filtered and concentrated under reduced pressure to afford crude product which was purified by preparative TLC (DCM/MeOH, 30:1) to give 2-[4-methyl-3-[5-methyl-6-(3-methyl-1-tetrahydropyran-2-yl-pyrazolo[3,4-c]pyridin-4-yl)-3-pyridyl]-2-oxo-benzimidazol-1-yl]-N-(2,2,2-trifluoroethyl)acetamide (10 mg, 59%) as a yellow solid. LC-MS (ES⁺, Method 3): 3.98 min m/z, 594 [M+H]⁺.

STEP C: 2-[4-methyl-3-[5-methyl-6-(3-methyl-1H-pyrazolo[3,4-c]pyridin-4-yl)-3-pyridyl]-2-oxo-benzimidazol-1-yl]-N-(2,2,2-trifluoroethyl)acetamide. To a solution of 2-[4-methyl-3-[5-methyl-6-(3-methyl-1-tetrahydropyran-2-yl-pyrazolo[3,4-c]pyridin-4-yl)-3-pyridyl]-2-oxo-benzimidazol-1-yl]-N-(2,2,2-trifluoroethyl)acetamide (10 mg, 0.02 mmol) in DCM (1 mL) was added 4 M HCl in dioxane (0.5 mL, 0.02 mmol) at 25° C., the mixture was allowed to stir for 3 h. The mixture was concentrated and purified by preparative TLC (DCM/MeOH, 30/1) affording 2-[4-methyl-3-[5-methyl-6-(3-methyl-1H-pyrazolo[3,4-c]pyridin-4-yl)-3-pyridyl]-2-oxo-benzimidazol-1-yl]-N-(2,2,2-trifluoroethyl)acetamide (2 mg, 18%) as a white solid. LC-MS (ES⁺, Method 3): 2.783 min, m/z 510.30 [M+H]⁺. ¹H NMR (400 MHz, Methanol-d4) δ 9.09 (s, 1H), 8.72-8.69 (m, 1H), 8.22 (s, 1H), 8.10-8.07 (m, 1H), 7.17-6.96 (m, 4H), 4.78 (s, 2H), 4.01 (q, J=9.3 Hz, 2H), 2.31 (s, 3H), 2.14 (s, 3H), 2.07 (s, 3H) ppm.

### Example 103: 2-[4-methyl-3-[2-methyl-6-(3-methyl-1H-pyrazolo[3,4-c]pyridin-4-yl)-3-pyridyl]-2-oxo-benzimidazol-1-yl]-N-(2,2,2-trifluoroethyl)acetamide

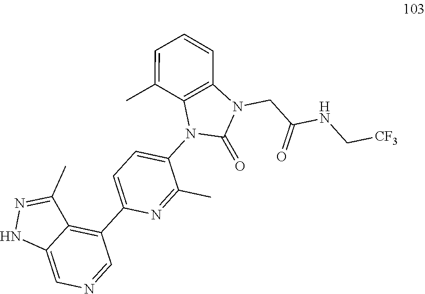 [SMILES: Cc1nc(-c2cncc3[nH]nc(C)c23)ccc1-n1c(=O)n(CC(=O)NCC(F)(F)F)c2cccc(C)c21]

Example 103 was prepared by analogy with example 102 (Scheme 17), replacing intermediate 1-65 in step A with Intermediate I-63. LC-MS (ES⁺, Method 4): 1.12 min, m/z 510.20 [M+H]⁺. ¹H NMR (400 MHz, DMSO-d₆) δ 9.55 (s, 1H), 9.04 (s, 1H), 8.58 (s, 1H), 8.13 (d, J=8.0 Hz, 1H), 7.93 (d, J=8.0 Hz, 1H), 7.06 (d, J=4.0 Hz, 2H), 6.89 (brs, 1H), 4.69 (s, 2H), 3.99 (brs, 2H), 2.44 (s, 3H), 2.39 (s, 3H), 1.83 (s, 3H) ppm.

### Example 104: 2-[4-methyl-3-[3-methyl-5-(3-methyl-1H-pyrazolo[3,4-c]pyridin-4-yl)pyrazin-2-yl]-2-oxo-benzimidazol-1-yl]-N-(2,2,2-trifluoroethyl)acetamide

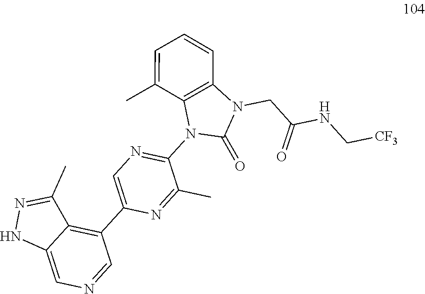 [SMILES: Cc1nc(-c2cncc3[nH]nc(C)c23)cnc1-n1c(=O)n(CC(=O)NCC(F)(F)F)c2cccc(C)c21]

Example 104 was prepared by analogy with example 102 (Scheme 17), replacing intermediate 1-65 in step A with Intermediate I-64. LC-MS (ES⁺, Method 3): 2.95 min, m/z 511.30 [M+H]⁺. ¹H NMR (400 MHz, MeOD-d₄) δ 9.11 (s, 1H), 8.96 (s, 1H), 8.49 (m, 1H), 7.16 (m, 1H), 7.05 (d, J=7.9 Hz, 1H), 6.98 (d, J=7.7 Hz, 1H), 4.78 (s, 2H), 4.01 (dd, J=9.4, 5.7 Hz, 2H), 2.69 (s, 3H), 2.50 (s, 3H), 1.91 (s, 3H) ppm.

### Example 105: 2-[4-methyl-3-[6-methyl-5-(3-methyl-1H-pyrazolo[3,4-c]pyridin-4-yl)pyrazin-2-yl]-2-oxo-benzimidazol-1-yl]-N-(2,2,2-trifluoroethyl)acetamide

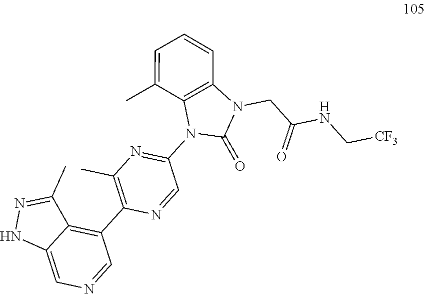 [SMILES: Cc1nc(-n2c(=O)n(CC(=O)NCC(F)(F)F)c3cccc(C)c32)cnc1-c1cncc2[nH]nc(C)c12]

Example 105 was prepared by analogy with example 102 (Scheme 17), replacing intermediate 1-65 in step A with Intermediate I-66. LC-MS (ES⁺, Method 3): 2.91 mi m/z 511.35 [M+H]⁺. ¹H NMR (400 MHz, MeOD-d₄) δ 9.59 (s, 1H), 9.07 (s, 1H), 8.57 (z, 1H), 7.21 (t, J=7.8 Hz, 1H), 7.05 (d, J=4.0 Hz, 2H), 4.80 (s, 2H), 4.05-3.98 (m, 2H), 3.38 (5, 2H), 2.59 (s, 3H), 2.29 (s, 3H), 2.14 (m, 3H) ppm. Examples 106 to 115 were prepared by analogy with example 75 (Scheme 15).

| | LC-MS | |
|---|---|---|
| Structure and name | (ES⁺) | ¹H NMR (400 MHz, DMSO-d6) |
| 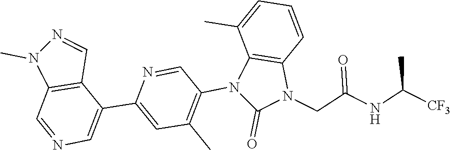 [SMILES: Cc1cc(-c2cncc3c2cnn3C)ncc1-n1c(=O)n(CC(=O)N[C@@H](C)C(F)(F)F)c2cccc(C)c21] | Method 3: 3.22 min, m/z, 524.35 [M + H]+. | 1H NMR (400 MHz, DMSO-d6) δ 9.28 (s, 1H), 8.97 (s, 1H), 8.92 (s, 1H), 8.78-8.75 (m, 2H), 8.31 (s, 1H), 7.04 (d, J = 8.0 Hz, 2H), 6.86 (d, J = 8.0 Hz, 1H), 4.66-4.61 (m, 3H), 4.26 (s, 3H), 2.22 (s, 3H), 1.83 (s, 3H), 1.30 (d, J = 4.0 Hz, 3H). |
| Example 106 | | |
| 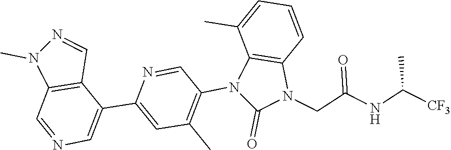 [SMILES: Cc1cc(-c2cncc3c2cnn3C)ncc1-n1c(=O)n(CC(=O)N[C@H](C)C(F)(F)F)c2cccc(C)c21] | Method 3: 3.02 min, m/z, 524.35 [m + h]. | 1H NMR (400 MHz, DMSO-d6) δ 9.31 (s, 1H), 8.98 (s, 1H), 8.91 (t, J = 8.0 Hz, 1H), 8.78 (d, J = 4.0 Hz, 2H), 8.32 (s, 1H), 7.07-7.02 (m, 2H), 6.86 (d, J = 8.0 Hz, 1H), 4.66-4.58 (m, 3H), 4.26 (s, 3H), 2.23 (s, 3H), 1.84 (s, 3H), 1.30 (d, J = 8.0 Hz, 3H). |
| Example 107 | | |
| 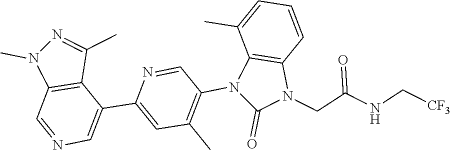 [SMILES: Cc1cc(-c2cncc3c2c(C)nn3C)ncc1-n1c(=O)n(CC(=O)NCC(F)(F)F)c2cccc(C)c21] | Method 3: 2.98 min, m/z, 524.40 [M + H] | 1H NMR (400 MHz, DMSO-d6) δ 9.22 (s, 1H), 8.97 (t, J = 6.3 Hz, 1H), 8.74 (s, 1H), 8.41 (s, 1H), 7.89 (s, 1H), 7.05 (d, J = 4.0 Hz, 2H), 6.87 (t, 1H), 4.73-4.64 (m, 2H), 4.16 (s, 3H), 4.01- 3.96 (m, 2H), 2.32 (s, 3H), 2.22 (s, 3H), 1.83 (s, 3H). |
| Example 108 | | |
| 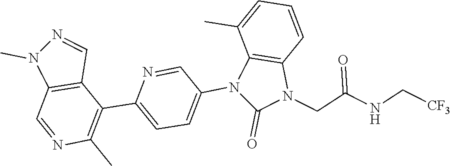 [SMILES: Cc1ncc2c(cnn2C)c1-c1ccc(-n2c(=O)n(CC(=O)NCC(F)(F)F)c3cccc(C)c32)cn1] | Method 4: 1.117 min, m/z, 510.2 [M + H]+ (%) | 1H NMR (400 MHz, DMSO-d6): 9.15 (s, 1H), 8.90-8.86 (m, 2H), 8.09 (dd, J = 4.0 Hz, J = 8.0 Hz, 1H), 7.95 (s, 1H), 7.88 (d, J = 8.0 Hz, 1H), 7.09-1.01 (m, 2H), 6.90 (d, J = 8.0 Hz, 1H), 4.67 (s, 2H), 4.21 (s, 3H), 4.02-3.93 (m, 2H), 2.64 (s, 3H), 1.95 (s, 3H). |
| Example 109 | | |
| 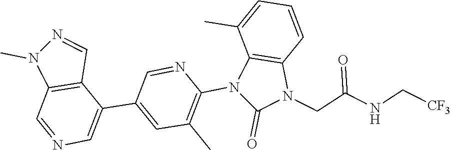 [SMILES: Cc1cc(-c2cncc3c2cnn3C)cnc1-n1c(=O)n(CC(=O)NCC(F)(F)F)c2cccc(C)c21] | Method 3: 3.98 min, m/z, 510 [M + H]+. | 1H NMR (400 MHz, DMSO-d6) δ 9.27 (s, 1H), 9.00 (t, J = 6.3 Hz, 1H), 8.91 (d, J = 2.3 Hz, 1H), 8.59 (s, 1H), 8.52 (s, 1H), 8.39 (d, J = 2.3 Hz, 1H), 7.07-7.00 (m, 2H), 6.89-6.85 (m, 1H), 4.67 (d, J = 3.4 Hz, 2H), 4.27 (s, 3H), 4.04- 3.94 (m, 2H), 2.33 (s, 3H), 1.77 (s, 3H). |
| Example 110 | | |
| 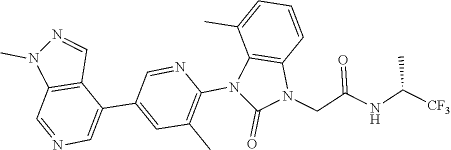 [SMILES: Cc1cc(-c2cncc3c2cnn3C)cnc1-n1c(=O)n(CC(=O)N[C@H](C)C(F)(F)F)c2cccc(C)c21] | Method 3: 3.18 min, m/z, 524.40 [M + H]. | 1H NMR (400 MHz, DMSO-d6) δ 9.28 (s, 1H), 8.92 (dd, J = 9.4, 3.1 Hz, 2H), 8.59 (s, 1H), 8.52 (s, 1H), 8.39 (d, J = 2.3 Hz, 1H), 7.08-6.98 (m, 2H), 6.86 (d, J = 7.3 Hz, 1H), 4.64 (d, J = 4.6 Hz, 3H), 4.27 (s, 3H), 2.33 (s, 3H), 1.77 (s, 3H), 1.30 (dd, J = 7.1, 3.0 Hz, 3H). |
| Example 111 | | |
| 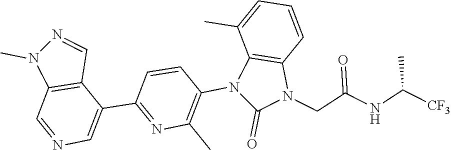 [SMILES: Cc1nc(-c2cncc3c2cnn3C)ccc1-n1c(=O)n(CC(=O)N[C@H](C)C(F)(F)F)c2cccc(C)c21] | Method 4: 1.58 min, m/z, 524.2 [M + H]+ (%) | 1H NMR (400 MHz, DMSO-d6): 9.28 (s, 1H), 8.98 (s, 1H), 8.91 (t, J = 8.0 Hz, 1H), 8.81 (s, 1H), 8.24 (d, J = 8.0 Hz, 1H), 8.03-7.99 (m, 1H), 7.07-7.01 (m, 2H), 6.86 (d, J = 8.0 Hz, 1H), 4.66 (s, 3H), 4.26 (s, 3H), 2.43 (s, 3H), 1.83 (s, 3H), 1.30 (d, J = 8.0 Hz, 3H). |
| Example 112 | | |
| 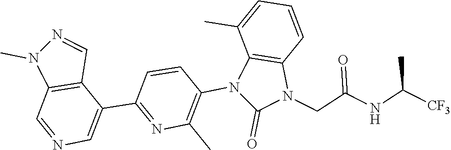 [SMILES: Cc1nc(-c2cncc3c2cnn3C)ccc1-n1c(=O)n(CC(=O)N[C@@H](C)C(F)(F)F)c2cccc(C)c21] | Method 4: 1.57 min, m/z, 524.2 [M + H]+ (%) | 1H NMR (400 MHz, DMSO-d6): 9.28 (s, 1H), 8.98 (s, 1H), 8.91 (t, J = 8.0 Hz, 1H), 8.81 (s, 1H), 8.24 (d, J = 8.0 Hz, 1H), 8.03-8.00 (m, 1H), 7.07-7.01 (m, 2H), 6.86 (d, J = 8.0 Hz, 1H), 4.66 (s, 3H), 4.26 (s, 3H), 2.43 (s, 3H), 1.83 (s, 3H), 1.30 (d, J = 8.0 Hz, 3H). |
| Example 113 | | |
| 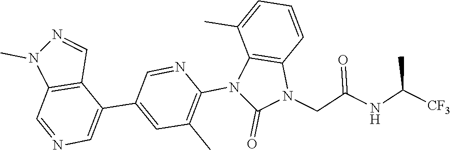 | Method 3: 3.08 min, m/z, 510.35 [M + H]. | 1H NMR (400 MHz, DMSO-d6): 9.28 (s, 1H), 8.94 (dd, J = 8.7, 3.8 Hz, 1H), 8.91 (d, J = 1.7 Hz, 1H), 8.59 (s, 1H), 8.52 (s, 1H), 8.39 (d, J = 1.5 Hz, 1H), 7.08-6.98 (m, 2H), 6.86 (d, J = 7.4 Hz, 1H), 4.71-4.56 (m, 3H), 4.27 (s, 3H), 2.33 (s, 3H), 1.77 (s, 3H), 1.30 (dd, J = 7.0, 2.8 Hz, 3H) |
| Example 114 | | |
| 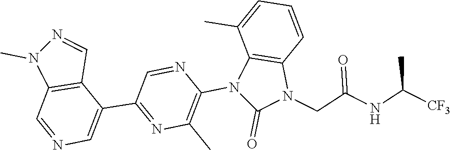 [SMILES: Cc1nc(-c2cncc3c2cnn3C)cnc1-n1c(=O)n(CC(=O)N[C@@H](C)C(F)(F)F)c2cccc(C)c21] | Method 3: 3.35 min, m/z, 525.35 [M + H]+. | 1H NMR (400 MHz, DMSO-d6) δ 9.42 (s, 1H), 9.35 (s, 1H), 9.12 (s, 1H), 8.93 (dd, J = 8.7, 3.6 Hz, 1H), 8.82 (s, 1H), 7.07 (dt, J = 15.7, 7.8 Hz, 2H), 6.90 (d, J = 7.3 Hz, 1H), 4.74-4.57 (m, 3H), 4.27 (s, 3H), 2.64 (s, 3H), 1.81 (s, 3H), 1.30 (dd, J = 7.0, 3.9 Hz, 3H). |
| Example 115 | | |

### DDR1 and DDR2 Biochemical Assay Method

The capacity of compounds to bind to DDR1 and DDR2 was quantified using a LanthaScreen Eu Kinase Binding Assay. Recombinant human DDR1 (2.5 nM; aa440-876 containing a GST tag) and DDR2 (1.75 nM; aa427-855 containing a GST tag) were diluted in assay buffer (50 mM HEPES pH7.3, 10 mM MgCl2, 1 mM EGTA and 0.01% Tween) with various concentrations of compound in a 384-well plate and a volume of 5 uL. After a 30-minute incubation at room temperature, 2.5 uL of Eu-anti GST antibody (diluted to 1 nM in assay buffer) plus Kinase Tracer 178 (diluted in assay buffer to 5 nM for DDR1 and 10 nM for DDR2) were added to the plate. Following 60-minute incubation at room temperature, time-resolved fluorescence was measured on a BMG Labtech PHERAstar plate reader. DMSO (1%) and reference compound (1 μM) were used to generate the Max and Min assay signals, respectively. Data was analysed using a four-parameter logistic model to calculate IC₅₀ values, with at least two independent replicates were performed for each compound.

### Biological Activity Values

The following table shows the pIC₅₀ values for the above examples against the DDR1 and DDR2 kinases (A: pIC₅₀>8; B: 8≥pIC₅₀>7; C: 7≥pIC₅₀>6; D: pIC₅₀≤6; ND: not determined).

TABLE 11

| Example | DDR1 pIC₅₀ | DDR2 pIC₅₀ |
|---|---|---|
| 1 | B | D |
| 2 | A | C |
| 3 | A | D |
| 4 | B | D |
| 5 | D | D |
| 6 | A | B |
| 7 | A | A |
| 8 | A | A |
| 9 | A | B |
| 10 | A | B |
| 11 | A | B |
| 12 | B | D |
| 13 | B | D |
| 14 | A | C |
| 15 | C | D |
| 16 | A | C |
| 17 | B | C |
| 18 | A | B |
| 19 | B | D |
| 20 | A | C |
| 21 | D | D |
| 22 | D | D |
| 23 | A | D |
| 24 | A | D |
| 25 | A | B |
| 26 | B | D |
| 27 | B | D |
| 28 | A | D |
| 29 | A | D |
| 30 | C | D |
| 31 | A | D |
| 32 | A | C |
| 33 | B | D |
| 34 | C | D |
| 35 | D | D |
| 36 | C | D |
| 37 | B | D |
| 38 | B | D |
| 39 | A | B |
| 40 | B | D |
| 41 | B | C |
| 42 | A | B |
| 43 | A | B |
| 44 | A | B |
| 45 | A | B |
| 46 | B | D |
| 47 | A | C |
| 48 | B | D |
| 49 | A | B |
| 50 | A | B |
| 51 | B | C |
| 52 | A | B |
| 53 | A | B |
| 54 | A | B |
| 55 | A | C |
| 56 | A | B |
| 57 | A | C |
| 58 | B | C |
| 59 | A | B |
| 60 | B | C |
| 61 | A | C |
| 62 | A | C |
| 63 | C | D |
| 64 | B | D |
| 65 | A | C |
| 66 | B | D |
| 67 | B | C |
| 68 | A | B |
| 69 | A | B |
| 70 | A | C |
| 71 | A | C |
| 72 | A | C |
| 73 | A | C |
| 74 | B | D |
| 75 | B | C |
| 76 | D | D |
| 77 | D | D |
| 78 | D | D |
| 79 | B | D |
| 80 | B | D |
| 81 | D | D |
| 82 | A | B |
| 83 | A | C |
| 84 | A | B |
| 85 | D | D |
| 86 | C | D |
| 87 | A | D |
| 88 | C | D |
| 89 | B | D |
| 90 | C | D |
| 91 | C | D |
| 92 | B | D |
| 93 | A | C |
| 94 | A | B |
| 95 | A | C |
| 96 | A | C |
| 97 | D | D |
| 98 | C | D |
| 99 | C | D |
| 100 | D | D |
| 101 | C | D |
| 102 | C | D |
| 103 | B | D |
| 104 | D | D |
| 105 | B | C |
| 106 | B | C |
| 107 | C | D |
| 108 | C | D |
| 109 | D | D |
| 110 | B | D |
| 111 | C | D |
| 112 | B | C |
| 113 | B | C |
| 114 | B | C |
| 115 | B | B |

1. A compound of formula (I) or a pharmaceutically acceptable salt thereof: 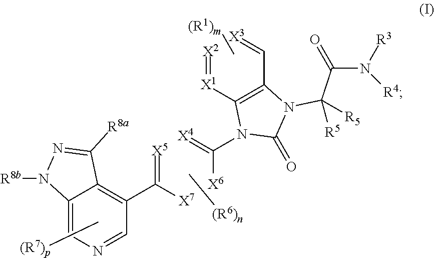
wherein
X¹, X² and X³ are each independently selected from carbon and nitrogen, wherein at least two of X¹, X² and X³ are carbon;
X⁴, X⁵, X⁶ and X⁷ are each independently selected from carbon and nitrogen, wherein at least two of X⁴, X⁵, X⁶ and X⁷ are carbon;
R¹ is independently selected at each occurrence from halo, nitro, cyano, NR⁹R¹⁰, OR¹¹, SR⁹, SO₂NR⁹R⁹, SO₂R⁹, CO₂R⁹, C(O)R⁹, CONR⁹R⁹, C₁-C₄-alkyl, C₁-C₄-alkyl substituted with NR⁹R¹⁰, C₁-C₄-alkyl substituted with OR¹¹, C₂-C₄-alkenyl, C₂-C₄-alkynyl, C₁-C₄-haloalkyl and cyclopropyl;
R³ is independently selected from H and C₁-C₄-alkyl;
R⁴ is independently selected from C₁-C₆-alkyl, C₁-C₆-haloalkyl, C₀-C₄-alkylene-R^{4a}; wherein R^{4a} is independently selected from: C₃-C₈-cycloalkyl, phenyl, 5-, 6-, 9- or 10-membered heteroaryl and 4- to 10-membered heterocycloalkyl; wherein said heterocycloalkyl or heteroaryl group may be monocyclic or bicyclic; wherein said cycloalkyl or heterocycloalkyl group is optionally substituted with a single R¹² group and/or from 1 to 4 R¹³ groups and wherein said phenyl or heteroaryl group is optionally substituted with a single R¹² group and/or from 1 to 3 R¹⁴ groups;
or R³ and R⁴, together with the nitrogen atom to which they are attached together form a 4- to 10-membered heterocycloalkyl group or a 5-, or 9-membered heteroaryl group; wherein said heterocycloalkyl or heteroaryl group may be monocyclic or bicyclic; wherein said heterocycloalkyl group is optionally substituted with a single R¹² group and/or from 1 to 4 R¹³ groups and wherein said heteroaryl group is optionally substituted with a single R¹² group and/or from 1 to 3 R¹⁴ groups;
R⁵ is independently at each occurrence selected from H, halo and C₁-C₄-alkyl, or the two R⁵ groups and the carbon atom to which they are attached may together form a C₃-C₆ cycloalkyl ring;
R⁶ is independently at each occurrence selected from halo, nitro, cyano, NR⁹R¹⁰, OR¹¹, SR⁹, SO₂NR⁹R⁹, SO₂R⁹, CO₂R⁹, C(O)R⁹, CONR⁹R⁹, C₁-C₄-alkyl, C₁-C₄-alkyl substituted with NR⁹R¹⁰, C₁-C₄-alkyl substituted with OR¹¹, C₂-C₄-alkenyl, C₂-C₄-alkynyl, C₁-C₄-haloalkyl and cyclopropyl;
R⁷ is independently at each occurrence selected from halo, nitro, cyano, NR⁹R¹⁰, OR¹¹, SR⁹, SO₂NR⁹R⁹, SO₂R⁹, CO₂R⁹, C(O)R⁹, CONR⁹R⁹, C₁-C₄-alkyl, C₁-C₄-alkyl substituted with NR⁹R¹⁰, C₁-C₄-alkyl substituted with OR¹¹, C₂-C₄-alkenyl, C₂-C₄-alkynyl, C₁-C₄-haloalkyl and cyclopropyl;
R^{8a} is independently selected from H, halo, nitro, cyano, NR⁹R¹⁰, OR¹¹, SR⁹, SO₂NR⁹R⁹, SO₂R⁹, CO₂R⁹, C(O)R⁹, CONR⁹R⁹, C₁-C₄-alkyl, C₁-C₄-alkyl substituted with NR⁹R¹⁰, C₁-C₄-alkyl substituted with OR¹¹, C₂-C₄-alkenyl, C₂-C₄-alkynyl, C₁-C₄-haloalkyl and C₀-C₄-alkylene-R^{8c};
R^{8b} is independently selected from H, C₁-C₄-alkyl, C₁-C₄-alkyl substituted with CONR⁹R⁹, C₂-C₄-alkyl substituted with NR⁹R¹⁰, C₂-C₄-alkyl substituted with OR¹¹, C₃-C₄-alkenyl, C₃-C₄-alkynyl, C₁-C₄-haloalkyl and C₀-C₄-alkylene-R^{8c};
R^{8c} is independently selected from C₃-C₆-cycloalkyl and 3- to 7-membered heterocycloalkyl; wherein said heterocycloalkyl group is attached to the C₀-C₄-alkylene via a carbon atom in the heterocycloalkyl ring; wherein said cycloalkyl or heterocycloalkyl group is optionally substituted with from 1 to 4 R¹³ groups;
R⁹ is independently at each occurrence selected from H and C₁-C₄-alkyl; or two R⁹ groups, together with the nitrogen atom to which they are attached together form a C₅-C₈-heterocycloalkyl group optionally substituted with from 0 to 4 R¹³ groups;
R¹⁰ is independently at each occurrence selected from H, C₁-C₄-alkyl, C(O)—C₁-C₄-alkyl and S(O)₂—C₁-C₄-alkyl; or R⁹ and R¹⁰, together with the nitrogen atom to which they are attached together form a C₅-C₈-heterocycloalkyl group optionally substituted with from 0 to 4 R¹³ groups;
R¹¹ is independently at each occurrence selected from H, C₁-C₄-alkyl, C(O)—C₁-C₄-alkyl and C₁-C₄-haloalkyl;
R¹² is independently selected from C₃-C₆-cycloalkyl, phenyl, 5- or 6-membered heteroaryl and 3- to 6-membered-heterocycloalkyl; wherein said cycloalkyl or heterocycloalkyl group is optionally substituted with from 1 to 4 R¹³ groups and wherein said phenyl or heteroaryl group is optionally substituted with from 1 to 3 R¹⁴ groups;
R¹³ is independently at each occurrence selected from ═O, halo, nitro, cyano, NR⁸R⁹, OR¹⁴, SR⁸, SO₂NR⁸R⁸, CO₂R⁸, C(O)R⁸, CONR⁸R⁸, C₁-C₄-alkyl, C₁-C₄-alkyl substituted with OR¹¹, C₁-C₄-alkyl substituted with NR⁹R¹⁰, C₂-C₄-alkenyl, C₂-C₄-alkynyl, C₁-C₄-haloalkyl, C₆-Clo-aryl, and C₃-C₆-cycloalkyl;
R¹⁴ is independently at each occurrence selected from halo, nitro, cyano, NR⁸R⁹, OR¹⁰, SR⁸, SO₂R⁸, SO₂NR⁸R⁸, CO₂R⁸, C(O)R⁸, CONR⁸R⁸, C₁-C₄-alkyl, C₂-C₄-alkenyl, C₂-C₄-alkynyl, C₁-C₄-haloalkyl, C₁-C₄-alkyl substituted with OR¹¹, C₁-C₄-alkyl substituted with NR⁸R⁹ and cyclopropyl;
m is an integer selected from 0, 1, 2, 3 and 4;
n is an integer selected from 0, 1, 2, 3 and 4;
p is an integer selected from 0, 1 and 2;
wherein any of the aforementioned alkyl, alkylene or cyclopropyl groups is optionally substituted, where chemically possible, by 1 to 5 substituents which are each independently at each occurrence selected from the group consisting of: halo, oxo, fluoro, nitro, cyano, NR^{a}R^{b}, OR^{a}, SR^{a}, CO₂R^{a}, C(O)R^{a}, CONR^{a}R^{a}, C₁-C₄-alkyl, C₁-C₄-haloalkyl and cyclopropyl; wherein R^{a} is independently at each occurrence selected from H, C₁-C₄-alkyl and C₁-C₄-haloalkyl; and R^{b} is independently at each occurrence selected from H, C₁-C₄-alkyl, C(O)—C₁-C₄-alkyl and S(O)₂—C₁-C₄-alkyl.

2. A compound of claim 1, wherein m is 0 or 1.

3. A compound of claim 1, wherein m is at least 1, X¹ is carbon and an R¹ group is attached to the X¹ carbon.

4. A compound of any one of claims 1 to 3, wherein n is 0 or 1.

5. A compound of any one of claims 1 to 4, wherein p is 0.

6. A compound of any one of claims 1 to 5, wherein R⁵ is at each occurrence H.

7. A compound of any one of claims 1 to 6, wherein R⁶ is at each occurrence C₁-C₄-alkyl.

8. A compound of any one of claims 1 to 7, wherein R^{8a} is H.

9. A compound of any one of claims 1 to 8, wherein R^{8b} is selected from H and C₁-C₄-alkyl.

10. A compound of any one of claims 1 to 9, wherein X¹, X² and X³ are each carbon.

11. A compound of any one of claims 1 to 10, wherein X⁴, X⁵, X⁶ and X⁷ are each carbon.

12. A compound of any one of claims 1 to 10, wherein at least one of X⁴, X⁵, X⁶, and X⁷ is nitrogen.

13. A compound of any one of claims 1 to 12, wherein NR³R⁴ has the formula 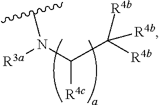
wherein
a is an integer selected from 1 and 2;
R^{4b} is at each occurrence selected from H and F; wherein at least one R^{4c} group is F;
R^{3a} is independently selected from H and C₁-C₄-alkyl;
R^{4c} is independently at each occurrence selected from H, C₁-C₄-alkyl and C₄-C₆-cycloalkyl; or
R^{3a} and a single R^{4c}, together with the carbon and nitrogen to which they are attached, form a 4- to 6-membered heterocycloalkyl group.

14. A compound of claim 13, wherein a is 1.

15. A compound of claim 13 or claim 14, wherein at least two R^{4c} groups are F.

16. A compound of any one of claims 13 to 15, wherein R^{3a} is H.

17. A compound of any one of claims 13 to 16, wherein R^{4c} is selected from H and C₁-C₄-alkyl.

18. A compound of any one of claims 13 to 15, wherein R^{3a} and R^{4c}, together with the carbon and nitrogen to which they are attached, form a 4- to 6-membered heterocycloalkyl group.

19. A compound of any one of claims 1 to 12, wherein R³ is H and R⁴ is selected from C₁-C₆-alkyl, C₁-C₆-haloalkyl and C₀-C₄-alkylene-R^{4a}.

20. A compound of claim 14, wherein R⁴ is selected from C₁-C₆-alkyl and C₁-C₆-haloalkyl.

21. A compound of claim 14, wherein R⁴ is C₀-C₄-alkylene-R^{4a}.

22. A compound of claim 16, wherein R⁴ is R^{4a}.

23. A compound of claim 16 or claim 17, wherein R^{4a} is selected from C₃-C₈-cycloalkyl and 4- to 10-membered heterocycloalkyl, wherein said cycloalkyl or heterocycloalkyl group is optionally substituted with from 1 to 4 R¹³ groups.

24. A compound of claim 16 or claim 17, wherein R^{4a} is independently selected from: phenyl and 5- or 6-membered heteroaryl; wherein said phenyl or heteroaryl group is optionally substituted with a single R¹² group and/or from 1 to 3 R¹⁴ groups.

25. A compound of any one of claims 1 to 12, wherein R³ and R⁴, together with the nitrogen atom to which they are attached together form a monocyclic 4- to 7-membered heterocycloalkyl group optionally substituted with from 1 to 4 R¹³ groups.

26. A compound of formula (I) selected from:

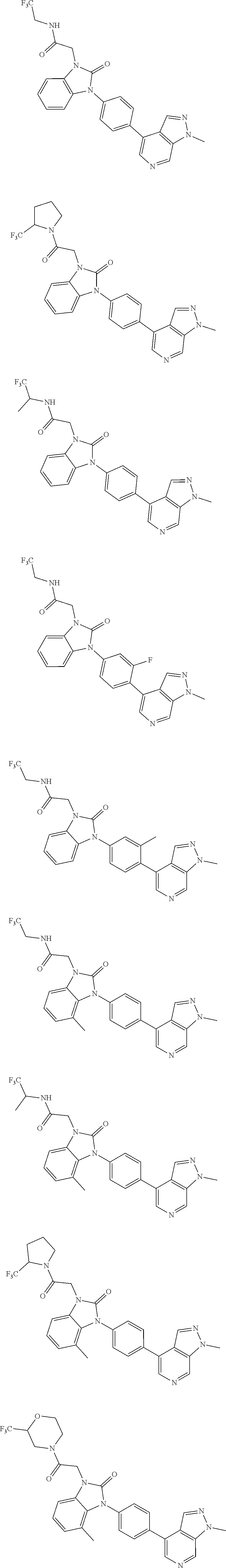

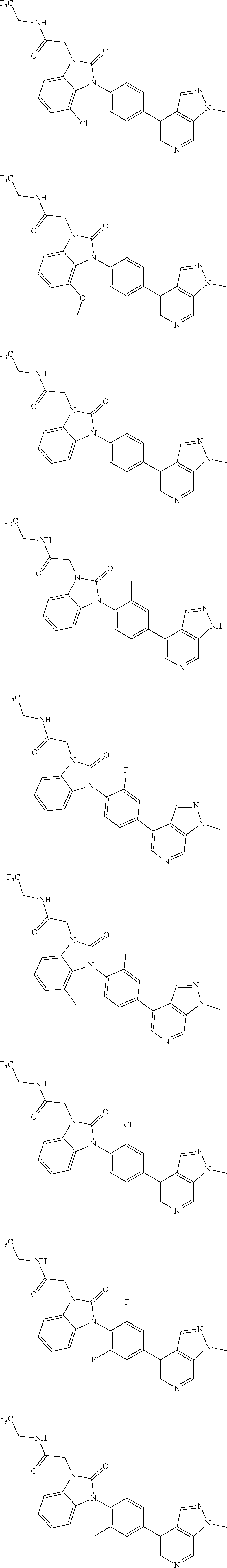

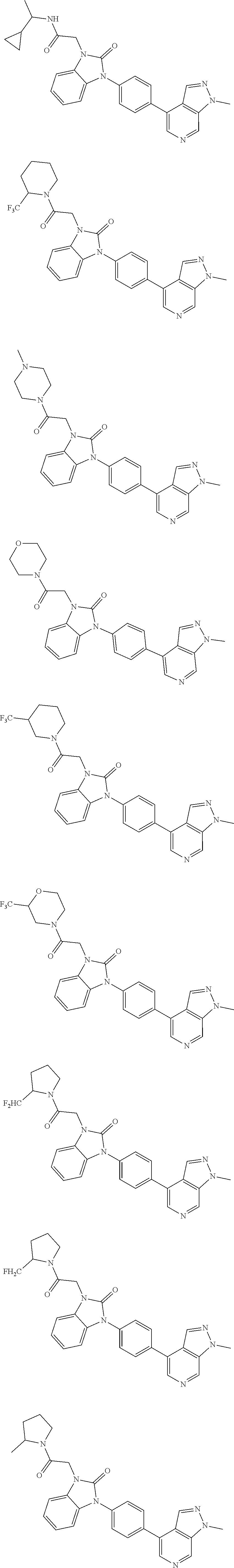

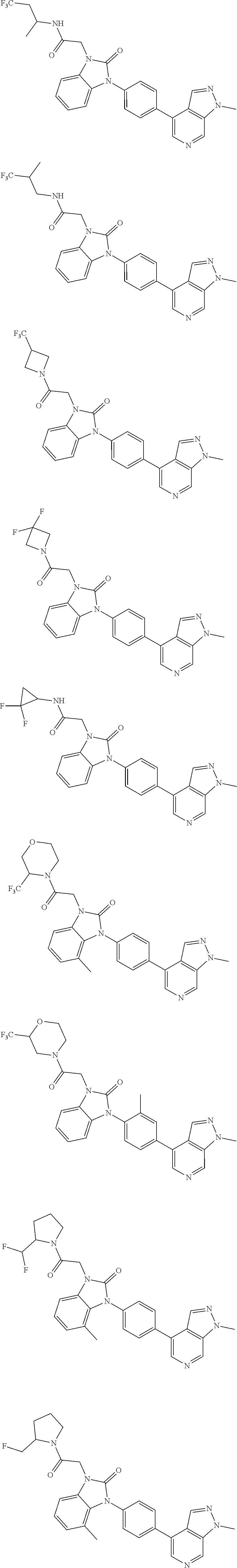

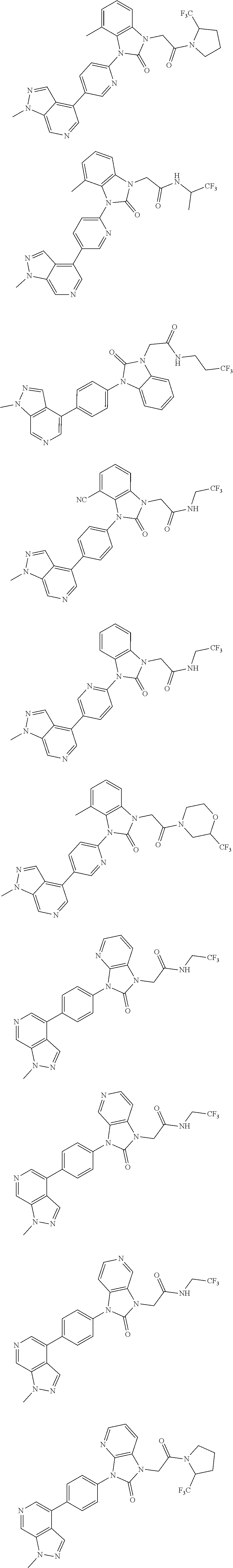

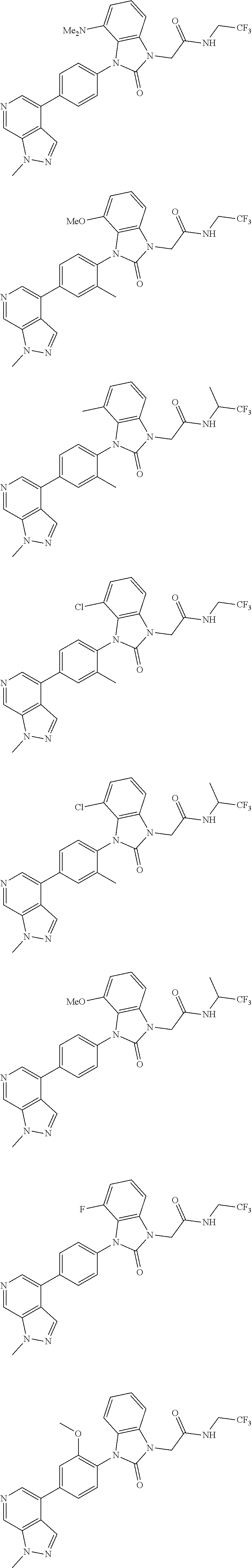

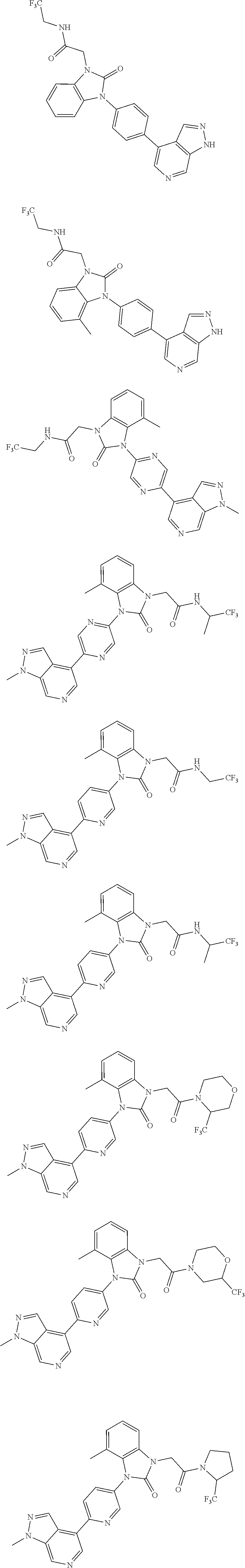

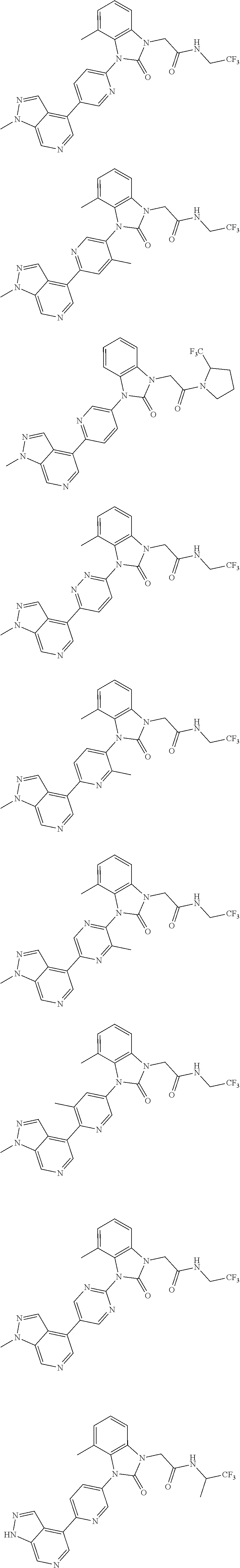

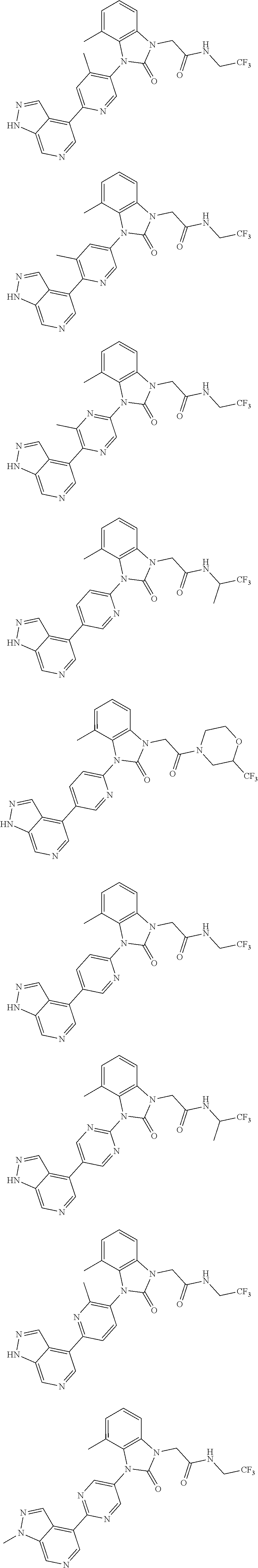

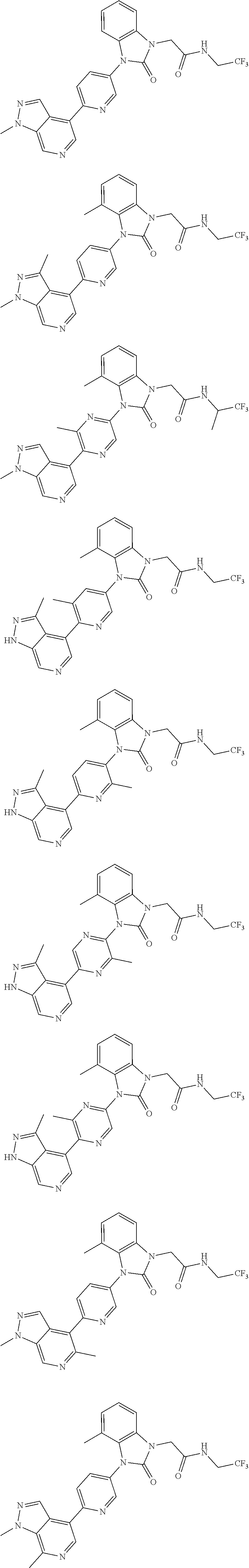

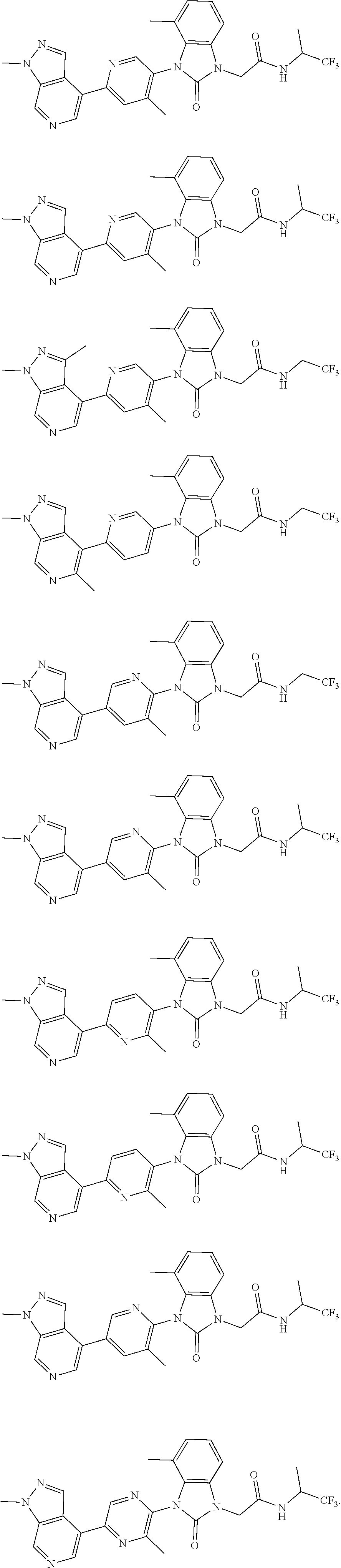

27. A pharmaceutical formulation comprising a compound of any one of claims 1 to 26 and a pharmaceutically acceptable excipient.

28. A compound of any one of claims 1 to 26 for use as a medicament.

29. A compound of any one of claims 1 to 26 for use in treating a disease or disorder selected from renal conditions, liver conditions, inflammatory conditions, cardiovascular conditions, acute and chronic organ transplant rejection, fibrotic diseases and cancer.
